(19) United States
(12) Patent Application Publication
(10) Pub. No.: US 2015/0057180 A1
(43) Pub. Date: Feb. 26, 2015

(54) Methods and Kits for Analyzing Biomarkers in a Signal Transduction Pathway

(71) Applicant: BioScale, Inc., Lexington, MA (US)

(72) Inventors: Edward Marshall Alderman, Framingham, MA (US); Matthew William Dickerson, Dorchester, MA (US); Lee Anne Beausang, Norfolk, MA (US); Brett Peter Masters, Belmont, CA (US); Martin Latterich, San Marcos, CA (US)

(21) Appl. No.: 14/478,637

(22) Filed: Sep. 5, 2014

Related U.S. Application Data
(63) Continuation of application No. PCT/US2013/029891, filed on Mar. 8, 2013.
(60) Provisional application No. 61/608,348, filed on Mar. 8, 2012.
(60) Provisional application No. 61/617,833, filed on Mar. 30, 2012.
(60) Provisional application No. 61/661,388, filed on Jun. 19, 2012.
(60) Provisional application No. 61/668,490, filed on Jul. 6, 2012.
(60) Provisional application No. 61/668,497, filed on Jul. 6, 2012.
(60) Provisional application No. 61/669,956, filed on Jul. 10, 2012.

(51) Int. Cl. G01N 33/543

(52) U.S. Cl. CPC G01N 33/54306; G01N 2333/5756; G01N 2333/96433; G01N 2333/91205; G01N 2800/52; G01N 2446/20
USPC 506/9; 435/7.1; 435/7.21; 435/7.4; 435/7.23; 435/6.13; 435/5

(57) ABSTRACT

Provided are methods and kits for analyzing biomarkers in one or more signal transduction pathways in a cell. In some embodiments, the methods and kits of the invention permit simultaneous analysis of more than one biomarker and/or more than one signal transduction pathway. In some embodiments, the invention provides methods for detecting whether a cell treated with an agent targeting a targeted biomarker is responding to the agent, or whether the cell is developing resistance to the agent. In some embodiments, the invention provides methods for determining which biomarker to target in a diseased or damaged cell, or which pathway an agent is targeting in an agent-treated cell. The invention provides kits for carrying out the described methods.

### CROSS-REFERENCE TO RELATED APPLICATIONS

This application is a continuation of PCT Application No. PCT/US2013/029891 filed Mar. 8, 2013, which itself claims priority to U.S. provisional application No. 61/608,348 filed Mar. 8, 2012, and to U.S. provisional application No. 61/617,833 filed Mar. 30, 2012, and to U.S. provisional application No. 61/661,388 filed Jun. 19, 2012, and to U.S. provisional application No. 61/668,490 filed Jul. 6, 2012, and to U.S. provisional application No. 61/668,497 filed Jul. 6, 2012, and to US provisional application 61/669,956 filed Jul. 10, 2012. The entire disclosures (including the claims) of each of these applications are hereby by reference.

### TECHNICAL FIELD

The present invention relates to biology and biochemistry, and more particularly to intracellular signal transduction pathways.

### BACKGROUND ART

Detection of small concentrations of biomarkers (e.g., biological molecules, such as proteins) is essential for quickly and accurately diagnosing disease states and potential disease states (e.g., cancer, degenerative diseases) in patients. Such detection is also critical to understanding the biological responses of drug candidates and for evaluating their efficacy and potential adverse effects in patients. Moreover, in drug development, detection of biomarkers aids in determining beneficial and adverse biological responses to drug-on-target interactions.

The development of custom assays for such unique proteins is often limited to time-consuming manual techniques using optical detection and quantitation procedures, such as ELISA or Western blotting. Western blotting analysis is extremely time- and labor-intensive. Optical methods, such as immunofluorescence staining, have been used effectively for biomarker detection, but they carry significant disadvantages. Quantification of biomarkers using the ELISA assay can be complicated by sample matrix effects. Furthermore, optical techniques can require time-consuming and labor-intensive sample preparation, as well as harsh denaturing conditions. These optical techniques also may introduce operator-dependent errors in the observation of results.

The complexity of development on alternate platforms, and the difficulty of using conventional assays to detect biomarkers in complex biological matrices contribute to difficulties in developing assays to reliably detect small concentrations, and/or small changes in concentrations of biomarkers relative to themselves, reference standards or reference biomarkers

Thus, there is a need to develop new assay technologies that can be used to quickly and accurately detect and/or quantify small concentrations, and/or small changes in concentrations of biomarkers.

### SUMMARY OF THE EMBODIMENTS

In some embodiments, the invention provides new assay technologies that can be used to detect and/or quantify small concentrations of biomarkers. Such methodologies are useful to determine, for example, if a patient with a diseased or damaged cell is responding to a therapy, or which therapy should be used to treat the patient. In some embodiments, the assays disclosed herein can be used to detect and/or quantify a secreted biomarker in blood, and in blood serum. In some embodiments, the assays disclosed herein can be used to detect and/or quantify a cell surface biomarker in cell lysates, in intact fixed cells, and in living cells. In some embodiments, the assays disclosed herein can be used to detect and/or quantify cytosolic and/or cell membrane biomarkers in a cellular signal transduction pathway of interest in cells, and also to detect and/or quantify dimers of two or more biomarkers in a cellular signal transduction pathway of interest.

Accordingly, in one aspect, the invention provides a method for determining if a cell is responding to treatment with an agent. The method comprises: (a) providing a sample of a cell treated with the agent; (b) providing a first binding agent that specifically binds to an entity selected from the group consisting of a first epitope on a first biomarker in a first signal transduction pathway and a noninterface epitope on the first biomarker, wherein said first biomarker is in the first signal transduction pathway in the first signal transduction pathway and is the same as or is different from the targeted biomarker, and wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain an agent number; and (f) repeating steps (b)-(e) with a control sample of an untreated cell or a cell treated with a control agent to obtain a control number; wherein a change in the agent number of step (e) as compared to the control number of step (f) that is a positive number or that is greater than a predetermined fold change indicates the agent-treated cell is responding to the agent and wherein a change in the agent number of step (e) as compared to the control number of step (f) that is a negative number or that is less than a predetermined fold change indicates the agent-treated cell is not responding to the agent. In various embodiments, the predetermined fold change is either at least 10%, at least 20%, at least 25%, at least 50%, at least 100%, at least 150%, at least 200%, at least 250%, at least 500%, at least 1000%, at least 2500%, at least 5000%, or at least 10,000%.

In another aspect, the invention provides a method for determining if a cell treated with an agent is developing resistance to the agent, the method comprising: (a) providing a sample of a cell treated with the agent; (b) providing a first binding agent that specifically binds to an entity selected from the group consisting of a first epitope on a first biomarker in a first signal transduction pathway and a noninterface epitope on the first biomarker, wherein said first biomarker is in the first signal transduction pathway in the first signal transduction pathway and is the same as or is different from the targeted biomarker in the first signal transduction pathway from the targeted biomarker, and wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain an agent number; and (f) repeating steps (b)-(e) with a sample of a control cell taken from the group consisting of an untreated cell and a cell treated with a control agent to obtain a control number; wherein a change in the agent number of step (e) as compared to the control number of step (f) that is a positive number or that is greater than a predetermined fold change indicates the agent-treated cell is developing resistance to the agent and wherein a change in the agent number of step (e) as compared to the control number of step (f) that is a negative number or that is less than a predetermined fold change indicates the agent-treated cell is not developing resistance to the agent. In various embodiments, the predetermined fold change is either at least 10%, at least 20%, at least 25%, at least 50%, at least 100%, at least 150%, at least 200%, at least 250%, at least 500%, at least 1000%, at least 2500%, at least 5000%, or at least 10,000%.

As used above, the term “interface” simply means the position on a biomarker and a biomarker partner where the two molecules touch one another to form a dimer.

In some embodiments, the output signal (also referred to as an acoustic output signal) is calculated by subtracting a background output signal from the sensor presented only with the first binding sample from an initial acoustic output signal from the sensor presented with the mixture of the sample, first binding agent, and second binding agent.

In another aspect, the invention provides a method for analyzing two or more signal transduction pathways in a cell treated with an agent. In some embodiments, the two or more signal transduction pathways are analyzed simultaneously. The method comprises (a) providing a sample comprising a cell treated with an agent; (b) providing a first binding agent that specifically binds to an entity selected from the group consisting of a first epitope on a first biomarker in a first signal transduction pathway and a noninterface epitope on the first biomarker, wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a first sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain a first number; (f) providing a third binding agent that specifically binds to an entity selected from the group consisting of a second epitope on a second biomarker in a second signal transduction pathway, wherein the second biomarker is different than the first biomarker, and a noninterface epitope on the second biomarker, and wherein the third binding agent is attached to a solid support; (g) providing a fourth binding agent that specifically binds to an entity selected from the group consisting of a second alternate epitope on the second biomarker in the second signal transduction pathway, wherein the second alternate epitope is different from the second epitope, and a noninterface epitope on a second biomarker partner, wherein said second biomarker partner is in the second signal transduction pathway, and wherein said fourth binding agent is capable of attaching to a sensor; (h) mixing the sample, the third binding agent, and the fourth binding agent together under conditions whereby a second sandwich assay formation will be formed if the sample contains a component selected from the group consisting of the second biomarker comprising the second epitope and the second alternate epitope, and the second biomarker complexed with the second biomarker partner; (i) presenting the result of step (h) to a sensor that emits an output signal that relates to the sensor's acoustic response to obtain a second number; (j) repeating steps (b)-(i) with a control sample of an untreated cell or a cell treated with a control agent to obtain a first control number and a second control number; and (k) comparing the first number to the first control number to obtain a first difference and the second number to the second control number to obtain a second difference, wherein said first difference and said second difference are represented as positive numbers; wherein a first difference larger than a second difference identifies the first signal transduction pathway as the pathway that will be preferentially selected to be targeted by the agent and wherein a second difference larger than a first difference identifies the second signal transduction pathway as the pathway that will be preferentially selected to be targeted by the agent.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response to obtain a third number, a fourth number, a third control number and a fourth control number.

In yet another aspect, the invention provides a method for identifying a signal transduction pathway to target with an agent in a diseased or damaged cell, comprising (a) providing a sample of a diseased or damaged cell; (b) providing a first binding agent that specifically binds to an entity selected from the group consisting of a first epitope on a first biomarker in a first signal transduction pathway and a noninterface epitope on the first biomarker, wherein said first biomarker is in a first signal transduction pathway, wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a first sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain a first number; (f) providing a third binding agent that specifically binds to an entity selected from the group consisting of a second epitope on a second biomarker and a noninterface epitope on the second biomarker, wherein the second biomarker is in a second signal transduction pathway, wherein the second biomarker is different than the first biomarker, and wherein the third binding agent is attached to a solid support; (g) providing a fourth binding agent that specifically binds to an entity selected from the group consisting of a second alternate epitope on the second biomarker in the second signal transduction pathway, wherein the second alternate epitope is different from the second epitope, and a noninterface epitope on a second biomarker partner, wherein said second biomarker partner is in the second signal transduction pathway, and wherein said fourth binding agent is capable of attaching to a sensor; (h) mixing the sample, the third binding agent, and the fourth binding agent together under conditions whereby a second sandwich assay formation will be formed if the sample contains a component selected from the group consisting of the second biomarker comprising the second epitope and the second alternate epitope, and the second biomarker complexed with the second biomarker partner; and (i) presenting the result of step (h) to a sensor that emits an output signal that relates to the sensor's acoustic response to obtain a second number; (j) repeating steps (b)-(i) with a sample of a healthy cell to obtain a first control number and a second control number; and (k) comparing the first number to the first control number to obtain a first difference and the second number to the second control number to obtain a second number, wherein said first difference and said second difference are represented as positive numbers; wherein a first difference larger than a second difference identifies the first signal transduction pathway as the pathway to target with the agent in the diseased or damaged cell and wherein a second difference larger than a first difference identifies the second signal transduction pathway as the pathway to target with the agent in the diseased or damaged cell.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response to obtain a third number, a fourth number, a third control number and a fourth control number.

In still another aspect, the invention provides a method for determining if a damaged or diseased cell treated with an agent targeting a biomarker is responding to the agent comprising: (a) providing a sample of a damaged or diseased cell treated with the agent; (b) providing a first binding agent that specifically binds to a first epitope on a first biomarker in a first signal transduction pathway, wherein said first biomarker is in the first signal transduction pathway in the first signal transduction pathway and is the same as or is different from the targeted biomarker in the first signal transduction pathway from the targeted biomarker, and wherein the first binding agent is attached to a solid support; (c) providing a second binding agent that specifically binds to a first alternate epitope on the first biomarker, wherein the first alternate epitope is different than the first epitope and wherein the first epitope and the first alternate epitope are both present only on an activated form or an inhibited form of the first biomarker, and the wherein said second binding agent is capable of attaching to a sensor; (d) mixing the sample, the first binding agent, and the second binding agent together under conditions whereby a sandwich assay formation will be formed if the sample contains a the first biomarker comprising the first epitope and the first alternate epitope; (e) presenting the result of step (d) to a sensor that emits an output signal that relates to the sensor's acoustic response to obtain an activated agent number or an inhibited agent number; and (f) repeating steps (b)-(e), with a first reference binding agent that specifically binds to a first reference epitope on a reference biomarker within the sample, or a portion of the sample, and a second reference binding agent that specifically binds to a second reference epitope on the reference biomarker, wherein the first reference epitope and the second reference epitope are not the same, to obtain an agent reference number; (g) repeating steps (b)-(f) with a control sample to obtain an activated control number or an inhibited control number and a control reference number, the control sample selected from the group consisting of an untreated cell and a cell treated with a control agent; and (h) comparing the activated agent number or the inhibited agent number to the agent reference number to obtain a first ratio and comparing the activated control number or the inhibited control number to the control reference number to obtain a control ratio; wherein a change in the first ratio to the control ratio that is a positive number or that is greater than an predetermined fold change indicates the agent-treated cell is responding to the agent and wherein a change in the first ratio to the control ratio that is a negative number or that is less than the predetermined fold change indicates the agent-treated cell is not responding to the agent. In various embodiments, the predetermined fold change is either at least 10%, at least 20%, at least 25%, at least 50%, at least 100%, at least 150%, at least 200%, at least 250%, at least 500%, at least 1000%, at least 2500%, at least 5000%, or at least 10,000%.

In still another aspect, the invention provides a method for determining if a damaged or diseased cell treated with an agent targeting a biomarker is developing resistance to the agent comprising: (a) providing a sample of a damaged or diseased cell treated with the agent; (b) providing a first binding agent that specifically binds to a first epitope on a first biomarker in a first signal transduction pathway, wherein said first biomarker is in the first signal transduction pathway in the first signal transduction pathway and is the same as or is different from the targeted biomarker in the first signal transduction pathway from the targeted biomarker, and wherein the first binding agent is attached to a solid support; (c) providing a second binding agent that specifically binds to a first alternate epitope on the first biomarker, wherein the first alternate epitope is different than the first epitope and wherein the first epitope and the first alternate epitope are both present only on an activated form or an inhibited form of the first biomarker, and the wherein said second binding agent is capable of attaching to a sensor; (d) mixing the sample, the first binding agent, and the second binding agent together under conditions whereby a sandwich assay formation will be formed if the sample contains a the first biomarker comprising the first epitope and the first alternate epitope; (e) presenting the result of step (d) to a sensor that emits an output signal that relates to the sensor's acoustic response to obtain an activated agent number or an inhibited agent number; and (f) repeating steps (b)-(e), with a first reference binding agent that specifically binds to a first reference epitope on a reference biomarker within the sample, or a portion of the sample, and a second reference binding agent that specifically binds to a second reference epitope on the reference biomarker, wherein the first reference epitope and the second reference epitope are not the same, to obtain an agent reference number; (g) repeating steps (b)-(f) with a control sample to obtain an activated control number or an inhibited control number and a control reference number, the control sample selected from the group consisting of an untreated cell and a cell treated with a control agent; and (h) comparing the activated agent number or the inhibited agent number to the agent reference number to obtain a first ratio and comparing the activated control number or the inhibited control number to the control reference number to obtain a control ratio; wherein a change in the first ratio to the control ratio that is a positive number or that is greater than an predetermined fold change indicates the agent-treated cell is developing resistance to the agent and wherein a change in the first ratio to the control ratio that is a negative number or that is less than the predetermined fold change indicates the agent-treated cell is not developing resistance to the agent. In various embodiments, the predetermined fold change is either at least 10%, at least 20%, at least 25%, at least 50%, at least 100%, at least 150%, at least 200%, at least 250%, at least 500%, at least 1000%, at least 2500%, at least 5000%, or at least 10,000%.

In another aspect, the invention provides a method for analyzing two or more signal transduction pathways in a cell treated with an agent comprising: (a) providing a sample comprising a cell treated with an agent; (b) providing a first binding agent that specifically binds to a first epitope on a first biomarker in a first signal transduction pathway, wherein the first binding agent is attached to a solid support; (c) providing a second binding agent that specifically binds to a first alternate epitope on the first biomarker, wherein the first alternate epitope is different than the first epitope, wherein the first alternate epitope and the first epitope are both present only on an activated form or an inhibited form of the first biomarker, and wherein said second binding agent is capable of attaching to a sensor; (d) mixing the sample, the first binding agent, and the second binding agent together under conditions whereby a first sandwich assay formation will be formed if the sample contains the first biomarker comprising the first epitope and the first alternate epitope; (e) presenting the result of step (d) to a sensor that emits an output signal that relates to the sensor's acoustic response to obtain a first number; (f) repeating steps (b)-(e), with a first reference binding agent that specifically binds to a first reference epitope on a first reference biomarker within the sample, or a portion of the sample, and a second reference binding agent that specifically binds to a first alternate reference epitope on the first reference biomarker, wherein the first reference epitope and the first alternate reference epitope are not the same, to obtain a first reference number; (g) providing a third binding agent that specifically binds to second epitope on a second biomarker in a second signal transduction pathway, wherein the second biomarker is different than the first biomarker, and wherein the third binding agent is attached to a solid support; (h) providing a fourth binding agent that specifically binds to a second alternate epitope on the second biomarker in the second signal transduction pathway, wherein the second alternate epitope is different from the second epitope, wherein the second alternate epitope and the second epitope are both present only on an activated form or an inhibited form of the second biomarker, and wherein said fourth binding agent is capable of attaching to a sensor; (i) mixing the sample, the third binding agent, and the fourth binding agent together under conditions whereby a second sandwich assay formation will be formed if the sample contains the second biomarker comprising the second epitope and the second alternate epitope; (j) presenting the result of step (h) to a sensor that emits an output signal that relates to the sensor's acoustic response to obtain a second number; (k) repeating steps (g)-(j), with a third reference binding agent that specifically binds to a second reference epitope on a second reference biomarker within the sample, or a portion of the sample, and a fourth reference binding agent that specifically binds to a second alternate reference epitope on the reference biomarker, wherein the second reference epitope and the second alternate reference epitope are not the same, to obtain an second reference number; (l) repeating steps (b)-(k) with a control sample to obtain a control first number, a control first reference number, a control second number, and a control second reference number, the control sample selected from the group consisting of an untreated cell and a cell treated with a control agent; and (m) comparing the first number to the first reference number to obtain a first ratio, comparing the second number to the second reference number to obtain a second ratio, comparing the control first number to the control first reference number to obtain a control first ratio, and comparing the control second number to the control second reference number to obtain a control second ratio; wherein a change in the first ratio to the control first ratio is greater than the change in the second ratio to the control second ratio identifies the first signal transduction pathway as the pathway being preferentially targeted by the agent and wherein a change in the first ratio to the control first ratio is less than the change in the second ratio to the control second ratio identifies the second signal transduction pathway as the pathway being preferentially targeted by the agent. In various embodiments, the first reference biomarker and the second reference biomarker are the same.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response, and a fifth reference binding agent that specifically binds to a third reference epitope on a third reference biomarker within the sample, or a portion of the sample, and a sixth reference binding agent that specifically binds to a third alternate reference epitope on the third reference biomarker, wherein the third reference epitope and the third alternate reference epitope are not the same, to obtain a third number, a third reference number, a control third number, a control third reference number.

In yet another aspect, the invention provides a method for identifying a signal transduction pathway to target with an agent in a diseased or damaged cell, comprising (a) providing a sample of a diseased or damaged cell; (b) providing a first binding agent that specifically binds to a first epitope on a first biomarker in a first signal transduction pathway, wherein the first binding agent is attached to a solid support; (c) providing a second binding agent that specifically binds to a first alternate epitope on the first biomarker, wherein the first alternate epitope is different than the first epitope, wherein the first alternate epitope and the first epitope are both present only on an activated form or an inhibited form of the first biomarker, and wherein said second binding agent is capable of attaching to a sensor; (d) mixing the sample, the first binding agent, and the second binding agent together under conditions whereby a first sandwich assay formation will be formed if the sample contains the first biomarker comprising the first epitope and the first alternate epitope; (e) presenting the result of step (d) to a sensor that emits an output signal that relates to the sensor's acoustic response to obtain a first number; (f) repeating steps (b)-(e), with a first reference binding agent that specifically binds to a first reference epitope on a first reference biomarker within the sample, or a portion of the sample, and a second reference binding agent that specifically binds to a first alternate reference epitope on the first reference biomarker, wherein the first reference epitope and the first alternate reference epitope are not the same, to obtain a first reference number; (g) providing a third binding agent that specifically binds to second epitope on a second biomarker in a second signal transduction pathway, wherein the second biomarker is different than the first biomarker, and wherein the third binding agent is attached to a solid support; (h) providing a fourth binding agent that specifically binds to a second alternate epitope on the second biomarker in the second signal transduction pathway, wherein the second alternate epitope is different from the second epitope, wherein the second alternate epitope and the second epitope are both present only on an activated form or an inhibited form of the second biomarker, and wherein said fourth binding agent is capable of attaching to a sensor; (i) mixing the sample, the third binding agent, and the fourth binding agent together under conditions whereby a second sandwich assay formation will be formed if the sample contains the second biomarker comprising the second epitope and the second alternate epitope; (j) presenting the result of step (h) to a sensor that emits an output signal that relates to the sensor's acoustic response to obtain a second number; (k) repeating steps (g)-(j), with a third reference binding agent that specifically binds to a second reference epitope on a second reference biomarker within the sample, or a portion of the sample, and a fourth reference binding agent that specifically binds to a second alternate reference epitope on the reference biomarker, wherein the second reference epitope and the second alternate reference epitope are not the same, to obtain a second reference number; (l) repeating steps (b)-(k) with a control sample to obtain a control first number, a control first reference number, a control second number, and a control second reference number, the control sample selected from the group consisting of an untreated cell and a cell treated with a control agent; and (m) comparing the first number to the first reference number to obtain a first ratio, comparing the second number to the second reference number to obtain a second ratio, comparing the control first number to the control first reference number to obtain a control first ratio, and comparing the control second number to the control second reference number to obtain a control second ratio; wherein a change in the first ratio to the control first ratio is greater than the change in the second ratio to the control second ratio identifies the first signal transduction pathway as the pathway as the pathway to target with the agent in the diseased or damaged cell and wherein a change in the first ratio to the control first ratio is less than the change in the second ratio to the control second ratio identifies the second signal transduction pathway as the pathway to target with the agent in the diseased or damaged cell. In various embodiments, the first reference biomarker and the second reference biomarker are the same.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response, and a fifth reference binding agent that specifically binds to a third reference epitope on a third reference biomarker within the sample, or a portion of the sample, and a sixth reference binding agent that specifically binds to a third alternate reference epitope on the third reference biomarker, wherein the third reference epitope and the third alternate reference epitope are not the same, to obtain a third number, a third reference number, a control third number, a control third reference number.

In various embodiments of the invention, the reference biomarker may be a house-keeping protein (i.e., encoded by a house-keeping gene), the total first biomarker, or the total second biomarker. The total first biomarker (or total second biomarker) includes all forms of the biomarker. For example, if the first biomarker can exist in an activated form (e.g., phosphorylated) and a non-activated form, the total first biomarker includes both activated and non-activated forms of the first biomarker. Similarly, if the first biomarker can exist in an inhibited form (e.g., a methylated form) and a non-inhibited form, the total first biomarker includes both inhibited and non-inhibited forms of the first biomarker.

In various embodiments, a reference biomarker is a house-keeping protein such as β-actin.

In various embodiments of the various aspects of the invention, the agent may be a cocktail of agents targeting a signaling pathway (e.g., the signaling pathway comprising the targeted biomarker). For example, the cocktail of agents may target both the first biomarker and the second biomarker. In another example, the cocktail of agents may comprise agents targeting the first biomarker (e.g., one agent is a binding agent that specifically bind to the C-terminus of the biomarker and the other agent is a small molecule that binds to the N-terminus of the biomarker).

In some embodiments, the acoustic output signal is calculated by subtracting a background acoustic output signal from the sensor presented only with the first binding sample from an initial acoustic output signal from the sensor presented with the mixture of the sample, first binding agent, and second binding agent.

In some embodiments, the agent is an siRNA molecule that targets the biomarker by hybridizing to a nucleic acid molecule encoding the targeted biomarker. In some embodiments, the agent is an siRNA hybridizes under stringent conditions or under high stringent conditions to a nucleic acid molecule encoding the targeted biomarker.

In some embodiments, the agent is a small molecule that targets the biomarker. In some embodiments, the agent is a binding agent that specifically binds to biomarker. In some embodiments, the agent is a biologic (e.g., a fusion protein).

In some embodiments, the damaged or diseased cell is human. In some embodiments, the damaged or diseased cell is a tumor cell, an injured cell, and infected cell, or a degenerative cell. In some embodiments, the tumor cell is selected from the group consisting of a colon cancer cell, a breast cancer cell, a prostate cancer cell, a leukemia cell, a lymphoma cell, and glioblastoma cell, a pancreatic cancer cell, a liver cancer cell, a lung cancer cell, a skin cancer cell, and a stomach cancer cell.

In some embodiments, the damaged or diseased cell is a human colorectal carcinoma cell. In some embodiments, the first biomarker and the targeted biomarker are the same. In some embodiments, the first biomarker and the targeted biomarker are different.

In some embodiments, the first biomarker is downstream of the targeted biomarker in the signal transduction pathway. In other words, the downstream biomarker participates in the signal transduction pathway after participation of the targeted biomarker. In some embodiments, the earlier biomarker is activated and its activation leads to the involvement of the downstream biomarker.

In some embodiments, the targeted biomarker is KRAS. In some embodiments, the targeted biomarker is MEK. In some embodiments, the targeted biomarker is PI3 kinase. In some embodiments, the targeted biomarker is epidermal growth factor receptor (EGFR), raf, braf, MEK1, MEK2, ERK1, ERK2, ERK3, ERK4, ERK5, ERK7, ERK8, JNK1, JNK2, JNK3, MKK3, MKK6, p38α, p38β, p38γ, p38δ, MKK4, MKK7, MTK1, DLK, TAO1, TAO2, RSK1, RSK2, RSK3, MNK1, MNK2, MSK1, MEK5, Fos, Jun, CDK2, p27, SMAD, AKT, PI3K, Bcl-x1, STAT3, STAT5, caspase 8, caspase 9, Bad, Bim, Bax, cdc42, Akkα, FADD, JAK, CDK4, Rb, or NF-κB.

In some embodiments, the damaged or diseased cell is treated with a second agent targeting an additional biomarker in an additional signal transduction pathway.

In some embodiments, the first biomarker comprises the first epitope and the second epitope when the first biomarker is in an activated form. In some embodiments, at least one of the first epitope and the second epitope comprises a phosphorylated amino acid residue. For example, the phosphorylated amino acid residue may be a phosphorylated tyrosine residue, a phosphorylated serine residue, or a phosphorylated threonine residue.

In some embodiments of the various aspects of the invention, the first binding agent binds to a first epitope on the biomarker and the second binding agent binds to a second epitope on the biomarker, wherein the first epitope and second epitope are not the same. In some embodiments, the first biomarker comprises the first epitope and the second epitope when the first biomarker is in an activated form.

In some embodiments of the various aspects of the invention, the acoustic output signal is calculated by subtracting a background acoustic output signal from the sensor presented only with the first binding sample from an initial acoustic output signal from the sensor presented with the mixture of the sample, first binding agent, and second binding agent.

In various embodiments of the various aspects of the invention, conclusions can be drawn by observing a change in the acoustic output signal from one sample as compared to an acoustic output signal from another sample (e.g., from a reference biomarker). The change can be quantified by any means including, for example, a positive number versus a negative number, an increase or decrease in percentage, a change in ratio, etc. When the change is represented by a percentage change, the percentage change may be 10% (one number is one-tenth of the value of the other number), or 25% (i.e., one number is one-fourth of the value of the other number), or 50% (i.e., one number is half the value of the other number), or 75% (i.e., one number is three-fourth of the value of the other number), or 150% (one number is 1.5 times the value of the other number), or 200% (i.e., one number is twice the value of the other number), and so forth.

Accordingly, in various embodiments of the various aspects of the invention, the numbers (e.g., the first number, the second number, the control numbers, the reference numbers, etc.) are normalized numbers. In some embodiments, the numbers are normalized against the number from a house-keeping protein (e.g., β-actin) in the sample. In some embodiments, the numbers are normalized by being taken from an approximately equivalent or equal mass of sample (e.g., 1.25 ug). In embodiments, the numbers are normalized by being taken from approximately equivalent or equal number of cells.

Of course the ordinarily skilled person will understand that the reference biomarker can be any biomarker including, without limitation, total amounts of that biomarker (e.g., including both activated form and non-activated form such as in the examples below), or a house-keeping protein (i.e., a protein that is expressed in all cells of the same organism and type (e.g., all B lymphocytes) at relatively constant levels). In addition to β-actin, some other non-limiting house-keeping proteins include heat shock protein 90 (HSP90) and glyceraldehyde-3-phosphate dehydrogenase (GAPDH), β-tubulin, and other proteins involved in the basic functioning of the cell such as those proteins involved in processing DNA and RNA.

In some embodiments of the various aspects of the invention, the first biomarker comprises the first epitope and the first alternate epitope when the first biomarker is in an activated form. In some embodiments, the first biomarker comprises the first epitope and the first alternate epitope when the first biomarker is in an inhibited form.

In some embodiments of the various aspects of the invention, the second biomarker comprises the second epitope and the second alternate epitope when the second biomarker is in an activated form. In some embodiments, the second biomarker comprises the second epitope and the first alternate epitope when the second biomarker is in an inhibited form.

In various embodiments of the various aspects of the invention, the damaged or diseased cell (and/or an agent-treated cell) is human. In some embodiments, the cell is a tumor cell, an infected cell, an injured cell, or a degenerative cell. In some embodiments, the tumor cell is selected from the group consisting of a colon cancer cell, a breast cancer cell, a prostate cancer cell, a leukemia cell, a lymphoma cell, and glioblastoma cell, a pancreatic cancer cell, a liver cancer cell, a lung cancer cell, a skin cancer cell, and a stomach cancer cell.

In some embodiments, the sample is selected form the group consisting of a tissue biopsy sample, a bodily fluid sample a xenograft cell sample, a living cell sample, a fixed cell sample, a fine needle aspirate sample, a circulating tumor cell sample, a blood sample, an exosome sample, a cell lysate sample, a conditioned culture media sample, a cultured cell sample, a cell lysate sample, a diseased cell sample, a saliva sample, a mucous sample, a tears sample, a tumor cell sample, a synovial fluid sample, a serum sample, a tissue sample, a marrow sample, a lymphatic fluid sample, an interstitial fluid sample, a buccal cell sample, a pleural effusion sample, a mucosal cell sample, a cerebrospinal fluid sample, a breast milk sample, a semen sample, a feces sample, a plasma sample, and a urine sample.

### BRIEF DESCRIPTION OF THE DRAWINGS

The patent or application file contains at least one drawing executed in color. Copies of this patent with color drawing(s) will be provided by the Patent and Trademark Office upon request and payment of necessary fee.

The foregoing features of embodiments will be more readily understood by reference to the following detailed description, taken with reference to the accompanying drawings, in which:

FIG. 1 is a schematic diagram showing the AMMP technology in an antibody sandwich assay, one non-limiting type of assay that can be run using the AMMP technology.

FIGS. 2A and 2B are line graphs showing the standard curves obtained by measuring the concentration of recombinant prolactin using AMMP technology (FIG. 2A) and proximity ligation assay (isPLA; FIG. 2B).

FIGS. 3A and 3B are line graphs showing the concentration of prolactin in human blood serum obtained using AMMP technology (FIG. 2A) and proximity ligation assay (isPLA; FIG. 2B).

FIGS. 4A and 4B are line graphs showing the standard curves obtained by measuring the concentration of recombinant cMET using AMMP technology (FIG. 4A) and proximity ligation assay (isPLA; FIG. 4B).

FIG. 5 is a line graph showing the standard curves obtained by measuring the concentration of cMET in the MPER lysis buffer (square symbols) or the TPER lysis buffer (triangle symbols) using AMMP technology.

FIG. 6 is a line graph showing the standard curves obtained by measuring the concentration of endogenous cMET in HepG2 cell lysates after cell lysis in the MPER lysis buffer (square symbols) or the TPER lysis buffer (triangle symbols) using AMMP technology.

FIGS. 7A and 7B are line graphs showing the showing the concentrations of cMET obtained by measuring the concentration of standard cMET (FIG. 7A) or endogenous cMET in HepG2 cell lysates (FIG. 7B) following post-optimization of antibody bead loading to increase assay sensitivity using AMMP technology.

FIG. 8 is a line graph showing detection of endogenous cMET in living HepG2 cells with low antibody loaded onto beads (square symbols) or high antibody loaded onto beads (triangle symbols) using AMMP technology.

FIG. 9 is a schematic diagram showing a part of the MAP kinase signal transduction pathway.

FIGS. 10A and 10B are a schematic diagrams showing a part of the MAP kinase signal transduction pathway in a cell, and the potential biological effects of such signaling.

FIGS. 11A and 11B are line graphs showing the standard curves obtained by measuring the concentration of MEK using AMMP technology (FIG. 11A) and proximity ligation assay (isPLA; FIG. 11B).

FIG. 12 is a line graph showing the detection of endogenous phosphorylated MEK (ser218/ser222) in HepG2 cell lysates with (triangle symbols) or without (square symbols) competitive free pMEK(ser218/ser222) peptide using AMMP technology.

FIGS. 13A and 13B are line graphs showing the detection of ERK/MEK dimers using AMMP technology. FIG. 13A shows the standard curve while FIG. 13B shows the concentration endogenous ERK/MEK dimer from HepG2 cell lysates in the presence (triangle symbols) or absence (square symbols) of 20 ng peptide inhibitor.

FIGS. 14A and 14B are bar graphs showing the detection and quantification of MEK-ERK heterodimers in EGF-treated (white bars) or non-treated (purple bars) MCF-7 cell lysates in two separate experiments.

FIGS. 15A and 15B are bar graphs showing the detection and quantification of MEK-ERK heterodimers in EGF-treated (white bars) or non-treated (purple bars) A431 cell lysates in two separate experiments.

FIGS. 16A and 16B are bar graphs showing the detection and quantification of MEK-ERK heterodimers in EGF-treated (white bars) or non-treated (purple bars) MCF-7 cell lysates in two separate experiments.

FIGS. 17A and 17B are bar graphs showing the detection and quantification of MEK-ERK heterodimers in EGF-treated (white bars) or non-treated (purple bars) A431 cell lysates in two separate experiments.

FIGS. 18A and 18B are bar graphs showing the detection and quantification of pMEK-ERK heterodimers in EGF-treated (white bars) or non-treated (purple bars) MCF-7 cell lysates in two separate experiments.

FIGS. 19A and 19B are bar graphs showing the detection and quantification of pMEK-ERK heterodimers in EGF-treated (white bars) or non-treated (purple bars) A431 cell lysates in two separate experiments.

FIGS. 20A and 20B are bar graphs showing the detection and quantification of pMEK-ERK heterodimers in EGF-treated (white bars) or non-treated (purple bars) MCF-7 cell lysates in two separate experiments.

FIGS. 21A and 21B are bar graphs showing the detection and quantification of pMEK-ERK heterodimers in EGF-treated (white bars) or non-treated (purple bars) A431 cell lysates in two separate experiments.

FIG. 22 is a bar graph the amount of phospho-p38 in the indicated cell lysate and tumor lysate samples, with recombinant p38 (green) as a positive control. Protein levels tested and calculated cell numbers are indicated on the X axis. Untreated A431 cells are shown in blue and EGF-treated A431 cells are shown in red. C2 tumor (purple) is a random tumor from the “C” group of mice and D1 tumor (yellow) is a random tumor from the D group of mice.

FIG. 23 is a bar graph showing the amount of phosphorylated EGFR in the indicated samples, with recombinant phosphorylated EGFR (green) as a positive control. Protein levels tested and calculated cell numbers are indicated on the X axis. Untreated A431 cells are shown in blue and EGF-treated A431 cells are shown in red. C2 tumor (purple) is a random tumor from the “C” group of mice and D1 tumor (yellow) is a random tumor from the D group of mice.

FIG. 24 is a bar graph showing the amount of total EGFR in the indicated samples. Protein levels tested and calculated cell numbers are indicated on the X axis. Untreated A431 cells are shown in blue and EGF-treated A431 cells are shown in red. C2 tumor (purple) is a random tumor from the “C” group of mice and D1 tumor (yellow) is a random tumor from the D group of mice.

FIG. 25 is a Western blotting analysis showing phospho-p38 (which runs at 38 kDa) in EGF-stimulated A431 cells (lane 2) and a random xenograft tumor (lane 3). The molecular weight marker is in lane 1. Both EGF-stimulated A431 cells and the xenograft tumor are positive for phospho-p38 expression

FIG. 26 is a Western blotting analysis showing phospho-EGFR (which runs at 135 kDa) in EGF-stimulated A431 cells (lane 2) and a random xenograft tumor (lane 3). The molecular weight marker is in lane 1. While the EGF-stimulated A431 cells are positive for expression of phospho-EGFR, the xenograft tumor is negative.

FIG. 27 is a Western blotting analysis showing total EGFR (which runs at 135 kDa) in EGF-stimulated A431 cells (lane 2) and a random xenograft tumor (lane 3). The molecular weight marker is in lane 1. Both EGF-stimulated A431 cells and the xenograft tumor are positive for expression of total EGFR

FIG. 28 is a bar graph showing the reproducibility of the phospho-p38 AMMP assay over 21 days.

FIG. 29 is a bar graph showing the reproducibility of the phospho-EGFR AMMP assay over 15 days.

FIG. 30 is a bar graph showing the reproducibility of the phospho-EGFR AMMP assay, measuring the phospho-EGFR from cell lysates of EGF-stimulated A431 cells, over 15 days.

FIG. 31 is a bar graph showing the amount of phospho-p38 measured in cell lysates of A431 tumors taken from the indicated mice. Error bars represent one standard deviation of triplicate analysis of each sample.

FIG. 32 is a bar graph showing the amount of total EGFR measured in cell lysates of A431 tumors taken from the indicated mice. Error bars represent one standard deviation of triplicate analysis of each sample.

FIG. 33 is a bar graph showing the amount of phospho-EGFR measured from cell lysates prepared from A431 tumors from the C5 mouse, a randomly selected mouse, that had been spiked (i.e., had been supplemented with) the recombinant p-EGFR at amounts indicated on the X axis (i.e., 0.0156 ng/ml, 0.0313 ng/ml, 0.0625 ng/ml, 0.125 ng/ml, 0.25 ng/ml, 0.5 ng/ml, 1 ng/ml, or 2 ng/ml). The recombinant p-EGFR was added to either MPER buffer alone (green bars), 1 ug of C5 tumor lysate mass (purple bars), 2 ug of C5 tumor lysate mass (orange bars), or 4 ug of C5 tumor lysate mass (blue bars).

FIG. 34 a bar graph showing the amount of phospho-EGFR measured from cell lysates prepared from the A431 tumor taken from the C4 mouse, a randomly selected mouse, that had been spiked (i.e., had been supplemented with) the recombinant p-EGFR at amounts indicated on the X axis (i.e., 0.156 ng/ml, 0.313 ng/ml, 0.625 ng/ml, 1.25 ng/ml, 2.5 ng/ml, 5 ng/ml, or 10 ng/ml). The recombinant p-EGFR was added to either MPER buffer alone (green bars), 10 ug of C4 tumor lysate mass (purple bars), 20 ug of C4 tumor lysate mass (orange bars), or 40 ug of C4 tumor lysate mass (blue bars).

FIG. 35 a bar graph showing the amount of phospho-p38 measured from cell lysates prepared from the A431 tumor taken from the D5 mouse, a randomly selected mouse, that had been spiked (i.e., had been supplemented with) the recombinant p-p38 at amounts indicated on the X axis (i.e., 0.156 ng/ml, 0.313 ng/ml, 0.625 ng/ml, 1.25 ng/ml, 2.5 ng/ml, 5 ng/ml, or 10 ng/ml). The recombinant phospho-p38 was added to either MPER buffer alone (green bars), 10 ug of D5 tumor lysate mass (purple bars), 20 ug of D5 tumor lysate mass (orange bars), or 40 ug of D5 tumor lysate mass (blue bars).

FIG. 36 is a bar graph showing the amount of total Akt kinase at the indicated concentrations of protein per well. Recombinant Akt1 is shown as a green bar, while Akt from the following cell lysates are as follows: light blue bars—IgF-1 stimulated WM266.4 cells plus LY; dark blue bars—IgF-1 stimulated WM266.4 cells; pink bars—IgF-1 stimulated MCF-7 cells plus LY; red bars—IgF-1 stimulated MCF-7 cells; light yellow bars—A431 cells lysed in TPER buffer; orange bars—A431 cells stimulated with EGF.

FIG. 37 is a bar graph showing the amount of EGFR in lysates made from EGF-stimulated A431 cells (red bars) or unstimulated A431 cells (blue bars). The green bars show recombinant EGFR.

FIG. 38 is a bar graph showing the amount of phosphorylated p38 (recombinant; green bars) in lysates made from A431 cells (using TPER buffer; blue bars), lysates from EGF stimulated A431 cells (red bars); lysates taken from the C2 tumors (purple bars) or lysates taken from D1 tumors (orange bars).

FIG. 39 is a bar graph showing the amount of total EGFR in A431 cells lysed with TPER buffer (blue bars); lysates of EGF-stimulated A431 cells (red bars); lysates taken from the C2 tumors (purple bars) or lysates taken from D1 tumors (orange bars).

FIG. 40 is a bar graph showing the amount of phosphorylated Akt (recombinant, green bars) in lysates made from untreated HEK cell lysates (light blue bars), lysates from HEK cells treated with anisomycin (dark blue bars), lysates from G1 tumors (red bars), lysates from G5 tumors (orange bars), and lysates from H3 tumors (purple bars).

FIG. 41 is a bar graph showing the amount of total Akt (recombinant, green bars) in lysates made from untreated HEK cell lysates (light blue bars), lysates from HEK cells treated with anisomycin (dark blue bars), lysates from G1 tumors (red bars), lysates from G5 tumors (orange bars), and lysates from H3 tumors (purple bars).

FIG. 42 is a bar graph showing the amount of phosphorylated Akt in lysates made from H1 tumors (0.39 grams; green bars); H2 tumors (0.11 grams; blue bars); H3 tumors (0.35 grams; red bars); H4 tumors (0.08 grams; orange bars); and H5 tumors (0.05 grams; purple bars).

FIG. 43 is a bar graph showing the amount of total Akt in lysates made from H1 tumors (0.39 grams; green bars); H2 tumors (0.11 grams; blue bars); H3 tumors (0.35 grams; red bars); H4 tumors (0.08 grams; orange bars); and H5 tumors (0.05 grams; purple bars).

FIG. 44 is a line graph showing the standard curve for phosphorylated Akt1. The standards are shown in open squares while the unknowns are shown as open diamonds.

FIG. 45 is a line graph showing the standard curve for total Akt1. The standards are shown in open squares while the unknowns are shown as open diamonds.

FIG. 46A is a bar graph showing the direct comparison of the amounts of phosphorylated Akt and total Akt in lysates from G1-G5 mice bearing HEK293 tumors.

FIG. 46B is a bar graph showing the direct comparison of the amounts of phosphorylated Akt and total Akt in lysates from H1-H5 mice bearing HEK293 tumors.

FIG. 47 is a bar graph showing the ratio of phosphorylated Akt1 to total Akt1 in quantified signal (light purple bars) and raw signal (dark purple bars).

FIG. 48 is a line graph showing the correlation between phosphorylated Akt1 to total Akt1 as plotted by quantified value (ng/ml in 0.625 ug lysates) versus raw signal.

FIG. 49 is a schematic diagram showing the biomarkers involved in two non-limiting representative signal transduction pathways, namely the ras/MEK/ERK pathway and the PI3K/AKT pathway.

FIG. 50A is a bar graph showing the AMMP detection and quantification of high levels of total EGFR expression from A431 and low level of EGFR expression from MCF-7 cell lysates regardless of the being stimulated (EGF) or left untreated (NT).

FIG. 50B is a depiction of a Western blot for EGFR expression in MCF-7 non-treated and EGF (lanes 1 & 2) and A431 non-treated and EGF (lanes 3 & 4) cell lysates.

FIG. 50C is a bar graph showing the AMMP detection and quantification of the phosphorylation of the EGFR on A431 cells following EGF stimulation and in the untreated population of A431 cells.

FIG. 50D is a depiction of a Western blot using the phospho-specific antibody in MCF-7 non-treated and EGF (lanes 1 & 2) and A431 non-treated and EGF (lanes 3 & 4) cell lysates.

FIGS. 51A-51D are bar graphs showing the AMMP detection and quantification of phosphorylation activity in each of the four kinase pathways: FIG. 51A for ERK1/2 in Jurkat cells; FIG. 51B for JNK in HEK293; FIG. 51C for p38 in 3T3 cells; and FIG. 51D for AKT1 in WM266.4. For all of FIGS. 51A-51D, stimulated cells are shown in bars with black and white squares, and untreated cells are shown in black and white dotted bars.

FIG. 52 is a bar graph showing the AMMP detection and quantification of the activation state of ERK1/2, p38, JNK and AKT1 from untreated MCF-7 cells (black bars), EGF stimulated MCF-7 cells (bars with black and white squares), untreated A431 cells (black bars with white X's), and EGF stimulated A431 cells (black bars with vertical white line).

FIGS. 53A-53E are bar graphs showing the AMMP detection and quantification of phosphorylation activity in the MEK/ERK pathways: FIG. 53A, total ERK1/2 expression; FIG. 53B, total MEK1 expression; FIG. 53C, phospho-MEK1 expression; FIG. 53D phosphor-ERK1/2 expression; and FIG. 53E, phospho-HER3 expression from untreated MCF-7 cells (black bars), EGF stimulated MCF-7 cells (bars with black and white squares), untreated A431 cells (black bars with white X's), and EGF stimulated A431 cells (black bars with vertical white line).

FIGS. 54A-54D are bar graphs showing side to side comparison of the AMMP detection and quantification of phosphorylation activity for EGF stimulated kinase activation in A431 (EGFR high expressing) cell line for p38, AKT1, JNK, MEK1, ERK, EGFR, and HER3: FIG. 54A, EGF stimulated (bars with black and white squares) compared to no treatment (black and white dotted bars); FIG. 54B, EGF stimulated (bars with black and white squares) compared to EGF stimulated with Gefitinib (black and white dotted bars); FIG. 54C, EGF stimulated (bars with black and white squares) compared to EGF-stimulated with Afatinib (black and white dotted bars); and FIG. 54D, EGF stimulated (bars with black and white squares) compared to EGF stimulated with Neratinib (black and white dotted bars).

FIGS. 55A-55D are bar graphs showing side to side comparison of the AMMP detection and quantification of phosphorylation activity for EGF stimulated kinase activation in MCF-7 (EGFR low expressing) cell line for p38, AKT1, JNK, MEK1, ERK, EGFR, and HER3: FIG. 55A, EGF stimulated (bars with black and white squares) compared to no treatment (black and white dotted bars); FIG. 55B, EGF stimulated (bars with black and white squares) compared to EGF-stimulated with Gefitinib (black and white dotted bars); FIG. 55C, EGF stimulated (bars with black and white squares) compared to EGF-stimulated with Afatinib (black and white dotted bars); and FIG. 55D, EGF stimulated (bars with black and white squares) compared to EGF-stimulated with Neratinib (black and white dotted bars).

FIG. 56 is a schematic diagram showing the signaling pathways involved in melanoma tumor cells.

FIG. 57 is a schematic diagram showing the signaling pathways initiated by the PDGFR-bet receptor tyrosine kinase.

FIG. 58 is a bar graph showing the amount of phosphorylated p38 in SV48 human colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated p38 is shown in green bars. The samples were tested (in duplicate) at two dilutions, with saturation at lower dilutions.

FIGS. 59 and 60 are a bar graphs showing the amount of phosphorylated JNK (FIG. 59) and total JNK (FIG. 60) in SV48 human colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated JNK is shown in green bars in FIG. 59 and recombinant total JNK is shown in green bars in FIG. 60. The samples were tested (in duplicate) at two dilutions, with saturation at the lower dilutions.

FIGS. 61A and 61B are a bar graphs showing the amount of phosphorylated epidermal growth factor (EGFR; FIG. 61A) and total EGFR (FIG. 61B) in SV48 human colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated EGFR is shown in green bars in FIG. 61A and recombinant total EGFR is shown in green bars in FIG. 61B. The samples were tested (in duplicate) at two dilutions, with saturation at the lower dilutions.

FIGS. 62A and 62B are a bar graphs showing the amount of phosphorylated AKT kinase (FIG. 62A) and total AKT (FIG. 62B) in SV48 human colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated AKT is shown in green bars in FIG. 62A and recombinant total AKT is shown in green bars in FIG. 62B. The samples were tested (in duplicate) at two dilutions, with saturation at the lower dilutions.

FIGS. 63A and 63B are a bar graphs showing the amount of phosphorylated MEK kinase (FIG. 63A) and total MEK (FIG. 63B) in SV48 colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated MEK is shown in green bars in FIG. 63A and recombinant total MEK is shown in green bars in FIG. 63B. The samples were tested (in duplicate) at two dilutions, with saturation at the lower dilutions.

FIGS. 64A and 64B are a bar graphs showing the amount of phosphorylated ERK kinase (FIG. 64A) and total ERK (FIG. 64B) in SV48 human colorectal cancer cells treated with an siRNA that selectively targets KRAS (Sample 1; blue bars); an siRNA that selectively targets MEK (Sample 2; red bars); an siRNA that selectively targets PI3 Kinase (Sample 3; gray bars); concurrent treatment with an siRNA that selectively targets MEK and an siRNA that selectively targets PI3 Kinase (Sample 4; purple bar), and an irrelevant siRNA (orange bars). Recombinant phosphorylated ERK is shown in green bars in FIG. 64A and recombinant total ERK is shown in green bars in FIG. 64B. The samples were tested (in duplicate) at two dilutions, with saturation at the lower dilutions.

FIGS. 65A and 65B are bar graphs showing the amounts of the indicated phosphorylated proteins (FIG. 65A) or the amounts of total (i.e., phosphorylated and non-phosphorylated) proteins (FIG. 65B) in SV48 human colorectal cancer cells treated with an siRNA that selectively targets KRAS. In FIG. 65A, the following phosphorylated proteins are shown as follows: p38 is shown in green bars; JNK is shown in as blue bars; EGFR is shown as red bars, AKT is shown as gray bars; MEK is shown as purple bars; and ERK is shown as orange bars. In FIG. 65B, the following total proteins are shown as follows: JNK is shown in as green bars; EGFR is shown as blue bars, AKT is shown as red bars, ERK is shown as gray bars, and MEK is shown as purple bars.

FIGS. 66A and 66B are bar graphs showing the amounts of the indicated phosphorylated proteins (FIG. 66A) or the amounts of total (i.e., phosphorylated and non-phosphorylated) proteins (FIG. 66B) in SV48 human colorectal cancer cells treated with an siRNA that selectively targets MEK kinase. In FIG. 66A, the following phosphorylated proteins are shown as follows: p38 is shown in green bars; JNK is shown in as blue bars; EGFR is shown as red bars, AKT is shown as gray bars; MEK is shown as purple bars; and ERK is shown as orange bars. In FIG. 66B, the following total proteins are shown as follows: JNK is shown in as green bars; EGFR is shown as blue bars, AKT is shown as red bars, ERK is shown as gray bars, and MEK is shown as purple bars.

FIGS. 67A and 67B are bar graphs showing the amounts of the indicated phosphorylated proteins (FIG. 67A) or the amounts of total (i.e., phosphorylated and non-phosphorylated) proteins (FIG. 67B) in SV48 human colorectal cancer cells treated with an siRNA that selectively targets PI3 kinase (PI3K). In FIG. 67A, the following phosphorylated proteins are shown as follows: p38 is shown in green bars; JNK is shown in as blue bars; EGFR is shown as red bars, AKT is shown as gray bars; MEK is shown as purple bars; and ERK is shown as orange bars. In FIG. 67B, the following total proteins are shown as follows: JNK is shown in as green bars; EGFR is shown as blue bars, AKT is shown as red bars, ERK is shown as gray bars, and MEK is shown as purple bars.

FIGS. 68A and 68B are bar graphs showing the amounts of the indicated phosphorylated proteins (FIG. 68A) or the amounts of total (i.e., phosphorylated and non-phosphorylated) proteins (FIG. 68B) in SV48 human colorectal cancer cells treated concurrently with an siRNA that selectively targets PI3 kinase (PI3K) and an siRNA that selectively targets MEK kinase. In FIG. 68A, the following phosphorylated proteins are shown as follows: p38 is shown in green bars; JNK is shown in as blue bars; EGFR is shown as red bars, AKT is shown as gray bars; MEK is shown as purple bars; and ERK is shown as orange bars. In FIG. 68B, the following total proteins are shown as follows: JNK is shown in as green bars; EGFR as blue bars, AKT as red bars, ERK as gray bars, and MEK as purple bars.

FIGS. 69A and 69B are bar graphs showing the amounts of the indicated phosphorylated proteins (FIG. 69A) or the amounts of total (i.e., phosphorylated and non-phosphorylated) proteins (FIG. 69B) in SV48 human colorectal cancer cells treated with an siRNA that selectively targets an irrelevant molecule. In FIG. 69A, the following phosphorylated proteins are shown as follows: p38 is shown in green bars; JNK is shown in as blue bars; EGFR is shown as red bars, AKT is shown as gray bars; MEK is shown as purple bars; and ERK is shown as orange bars. In FIG. 69B, the following total proteins are shown as follows: JNK is shown in as green bars; EGFR is shown as blue bars, AKT is shown as red bars, ERK is shown as gray bars, and MEK is shown as purple bars.

FIGS. 70A, 70B, and 70C are bar graphs showing the fold change in phoshorylated AKT1 (FIG. 70A), total AKT1 (FIG. 70B), and the ratio of phosphorylated AKT1/total AKT1 (FIG. 70C) in human colorectal cancer cells treated with siRNA targeting KRAS, siRNA targeting MEK, siRNA targeting PI3k (PI-3-kinase), siRNAs targeting both MEK and PI3K, and a luciferase control. Each bar represents samples loaded at 1.25 ug.

FIGS. 71A, 71B, and 71C are bar graphs showing the fold change in phoshorylated MEK1 (FIG. 71A), total MEK1 (FIG. 71B), and the ratio of phosphorylated MEK1/total MEK1 (FIG. 71C) in human colorectal cancer cells treated with siRNA targeting KRAS, siRNA targeting MEK, siRNA targeting PI3k (PI-3-kinase), siRNAs targeting both MEK and PI3K, and a luciferase control. Each bar represents samples loaded at 1.25 ug.

FIGS. 72A, 72B, and 72C are bar graphs showing the fold change in phoshorylated ERK1/2 (FIG. 72A), total ERK1/2 (FIG. 72B), and the ratio of phosphorylated ERK1/2/total ERK1/2 (FIG. 72C) in human colorectal cancer cells treated with siRNA targeting KRAS, siRNA targeting MEK, siRNA targeting PI3k (PI-3-kinase), siRNAs targeting both MEK and PI3K, and a luciferase control. Each bar represents samples loaded at 1.25 ug.

### DETAILED DESCRIPTION OF SPECIFIC EMBODIMENTS

In some embodiments, the present invention is based upon the development of methods and systems for accurately detecting levels of biological molecules in samples, and for detecting dimers of two biological molecules in samples.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The further aspects, advantages, and embodiments of the invention are described in more detail below. The definitions used in this specification and the accompanying claims shall following terms shall have the meanings indicated, unless the context clearly otherwise requires. Any conflict between an art-understood definition of a word or phrase and a definition of the word or phrase as specifically taught in this specification shall be resolved in favor of the latter. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology, all of which are incorporated herein by reference in their entirety, include Ausubel et al. Current Protocols in Molecular Biology, John Wiley & Sons, New York, N.Y. (1989 and updates through September 2011), Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed. Directed Mutagenesis: A Practical Approach, IRL Press, Oxford (1991).

Accordingly, in some embodiments, the invention provides novel methods and kits for detecting small concentrations of biomarkers in multiple sources including human serum, cell lysates, and cell surfaces. In some embodiments, using the methods described herein, the invention provides an assay for detecting the presence and level of expression of a secreted biomarker in human blood serum, and for detecting the presence and level of expression of a cell membrane biomarker in cell lysates, in intact fixed cells, and in living cells. In some embodiments, using the methods described herein, the invention provides an assay for detecting cytosolic and/or cell surface biomarkers associated with a particular cellular signal transduction pathway (e.g., the mitogen-activated protein kinase (MAPK) signaling pathway). In some embodiments, using the methods described herein, the invention provides an assay for detecting dimers of two biomarkers in a particular cellular signal transduction pathway (e.g., dimers of two biomarkers in the MAPK signaling pathway). In some embodiments, the solid support is magnetic.

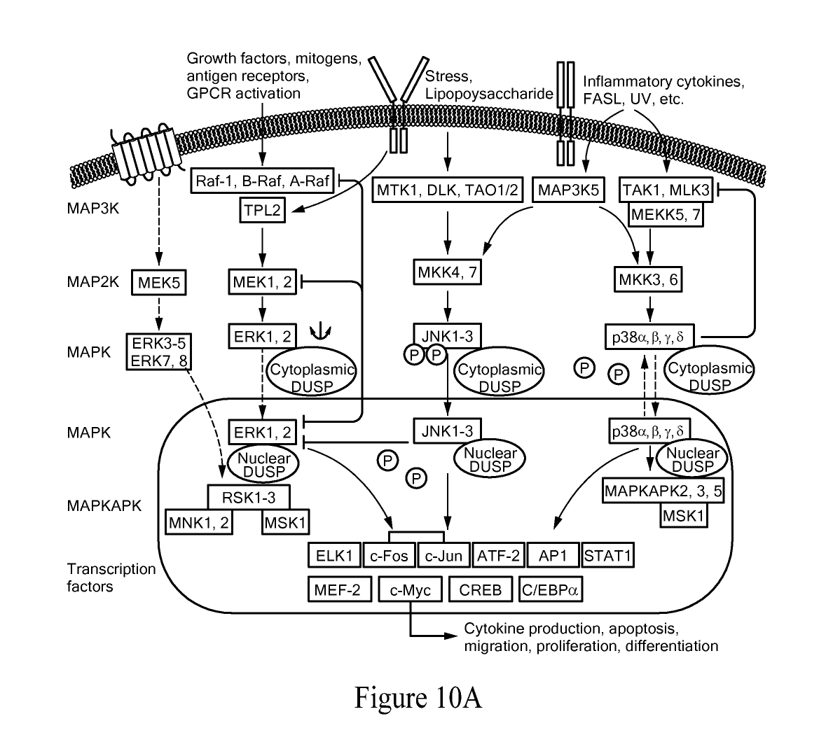

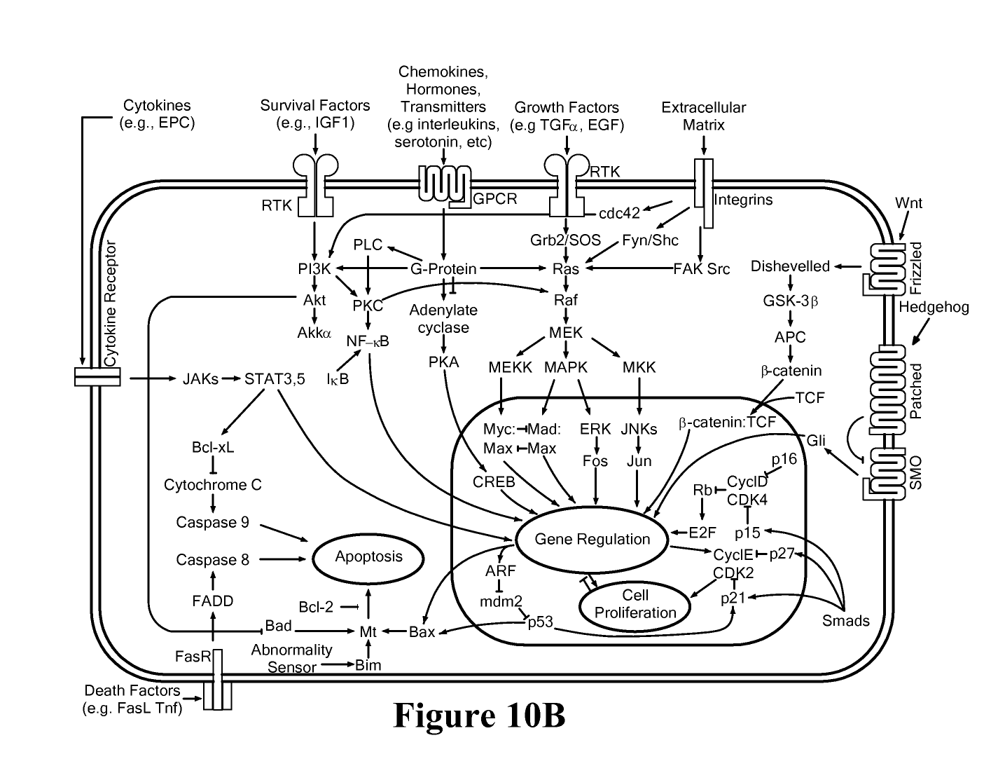

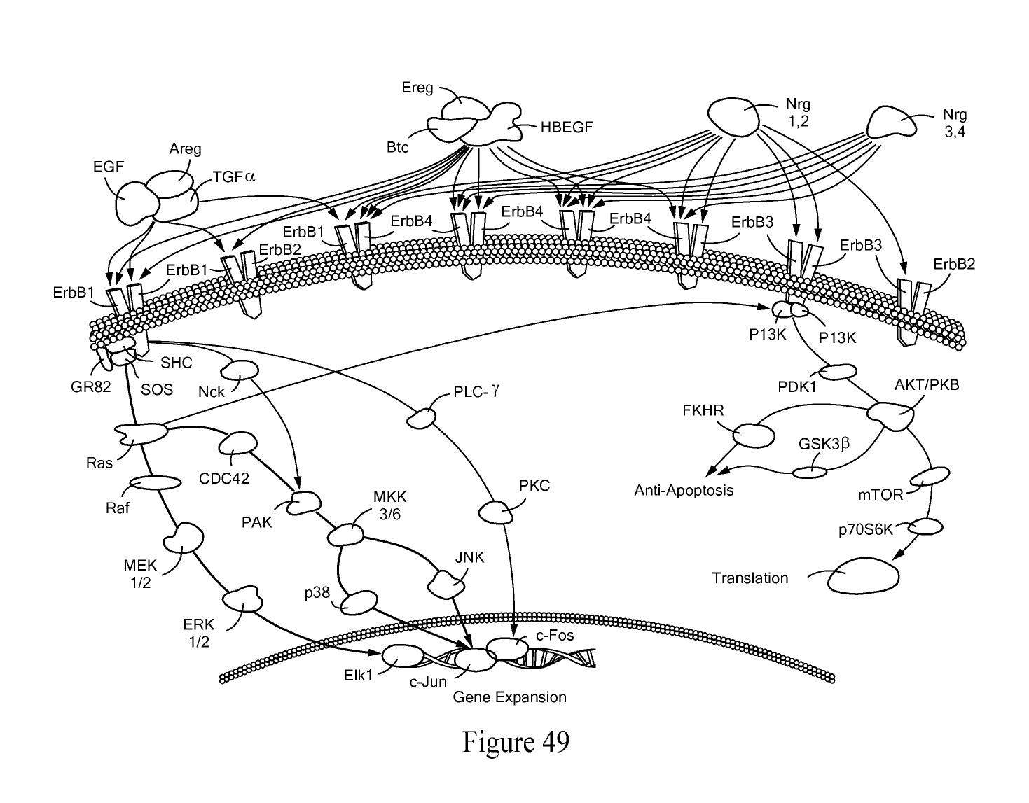

Particularly, in some embodiments, the invention provides methods and kits for detecting the presence of a biomarker, or a complex of two or more biomarkers, in a sample. The methods are kits are useful, for example, in determining which pathway to target in a diseased or damaged cell. Or, if the cell is already being treated by an agent, determining whether the cell is responding to the agent or developing resistance to the agent. By “targeting” in reference to a biomarker or a pathway, is meant that an agent binds to the biomarker in the pathway, binds to a biomarker upstream of the “targeted” biomarker in the pathway, or even binds to a biomarker in another pathway other than the targeted pathway, but that binding by the agent influences the targeted biomarker or the targeted pathway. As can be seen in FIGS. 10A-10B, and FIG. 49, signal transduction pathways are not necessarily linear. Note that if an agent is an siRNA, an siRNA targeting a biomarker (e.g., KRAS) can be referred to as an siRNA that hybridizes to a KRAS-encoding nucleic acid molecule or an siRNA that knocks down KRAS. Likewise, if the agent is an antibody, an antibody targeting a biomarker can be referred to as an antibody that specifically binds to the biomarker.

Some of the methods described herein comprise providing a sample suspected of containing the biomarker or the complex; providing a first binding agent that specifically binds to the biomarker (or first biomarker in the complex), wherein the first binding agent is attached to a solid support; providing a second binding agent that specifically binds to the biomarker (or second biomarker in the complex), wherein said second binding agent is capable of attaching to a sensor; mixing the sample, the first binding agent, and the second binding agent together under conditions whereby a sandwich assay formation will be formed if the sample contains the biomarker or the complex; presenting the mixture to a sensor that emits an acoustic output signal and detecting a change in the acoustic output signal, wherein a change in the acoustic output signal indicates the attachment of the sandwich assay formation to the sensor and thereby indicates the presence of the biomarker or the complex in the sample.

Accordingly, in one aspect, the invention provides a method for determining if a cell treated with an agent is responding to the agent or developing resistance to the agent. The method comprises: (a) providing a sample of a cell treated with the agent; (b) providing a first binding agent that specifically binds to an entity selected from the group consisting of a first epitope on a first biomarker in a first signal transduction pathway and a noninterface epitope on the first biomarker, wherein said first biomarker is in the first signal transduction pathway in the first signal transduction pathway and is the same as or is different from the targeted biomarker in the first signal transduction pathway from the targeted biomarker, and wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain an agent number; and (f) repeating steps (b)-(e) with a control sample of an untreated cell or a cell treated with a control agent to obtain a control number; wherein a change in the agent number of step (e) as compared to the control number of step (f) that is a positive number or that is greater than a predetermined fold change indicates the agent-treated cell either developing resistance to the agent or is responding to the agent and wherein a change in the agent number of step (e) as compared to the control number of step (f) that is a negative number or that is less than a predetermined fold change indicates the agent-treated cell is either not developing resistance to the agent or is not responding to the agent.

In still another aspect, the invention provides a method for determining if a damaged or diseased cell treated with an agent targeting a biomarker is responding to the agent comprising: (a) providing a sample of a damaged or diseased cell treated with the agent; (b) providing a first binding agent that specifically binds to a first epitope on a first biomarker in a first signal transduction pathway, wherein said first biomarker is in the first signal transduction pathway in the first signal transduction pathway and is the same as or is different from the targeted biomarker in the first signal transduction pathway from the targeted biomarker, and wherein the first binding agent is attached to a solid support; (c) providing a second binding agent that specifically binds to a first alternate epitope on the first biomarker, wherein the first alternate epitope is different than the first epitope and wherein the first epitope and the first alternate epitope are both present only on an activated form or an inhibited form of the first biomarker, and the wherein said second binding agent is capable of attaching to a sensor; (d) mixing the sample, the first binding agent, and the second binding agent together under conditions whereby a sandwich assay formation will be formed if the sample contains a the first biomarker comprising the first epitope and the first alternate epitope; (e) presenting the result of step (d) to a sensor that emits an output signal that relates to the sensor's acoustic response to obtain an activated agent number or an inhibited agent number; and (f) repeating steps (b)-(e), with a first reference binding agent that specifically binds to a first reference epitope on a reference biomarker within the sample, or a portion of the sample, and a second reference binding agent that specifically binds to a second reference epitope on the reference biomarker, wherein the first reference epitope and the second reference epitope are not the same, to obtain an agent reference number; (g) repeating steps (b)-(f) with a control sample to obtain an activated control number or an inhibited control number and a control reference number, the control sample selected from the group consisting of an untreated cell and a cell treated with a control agent; and (h) comparing the activated agent number or the inhibited agent number to the agent reference number to obtain a first ratio and comparing the activated control number or the inhibited control number to the control reference number to obtain a control ratio; wherein a change in the first ratio to the control ratio that is a positive number or that is greater than an predetermined fold change indicates the agent-treated cell either developing resistance to the agent or is responding to the agent and wherein a change in the first ratio to the control ratio that is a negative number or that is less than the predetermined fold change indicates the agent-treated cell is either not developing resistance to the agent or is not responding to the agent.

As used herein, by “acoustic output signal” (or simply “output signal”) is meant a signal that is based on sound. For example, the acoustic output signal may be from a sensor that emits an output signal that relates to the sensor's acoustic response. In some embodiments, an acoustic output signal is not based on light (e.g., fluorescence) or color.

The change in the acoustic output signal (or a number or a value derived therefrom) from a tested sample (e.g., of a diseased or damaged cell) as compared to a control sample (e.g., of a healthy cell) or a control biomarker will take a form that will depend upon the biomarker (or heterodimer of two biomarkers, or multimer) being tested and the control being tested. The acoustic output signal may either be increased or decreased depending on the nature of the response of the cell to disease, damage or treatment.

It should be noted that the change can be represented by any means. The ordinarily skilled person would know how to calculate such changes based, for example, on inclusion of proper controls, normalization over protein concentration, reduction of background signal, ratios based on total marker signal to a reference marker signal, and so forth.

For example, to control for the amount of an analyzed biomarker (or heterodimer of two biomarkers, or multimer), two acoustic output signals can be obtained from an analyzed sample (e.g., from a diseased or damaged cell): the first acoustic output signal from a biomarker of choice (e.g., to see if the biomarker is phosphorylated) and the second acoustic output signal from a known house-keeping protein (e.g., β-actin). The data from the analyzed sample can then be normalized for, for example, protein content by representing the acoustic output signal from the analyzed sample as a ratio of the acoustic output signal from the biomarker of choice to the acoustic output signal of the house-keeping protein. A ratio can similarly be obtained from another sample (e.g., from a healthy cell, from a diseased or damaged cell treated with a therapeutic, etc.). The change in the ratios of the acoustic output signals will provide information on the signal transduction pathways of both tested samples.

Yet another way to represent change in the acoustic output signals of two different samples is by representing the change as a percentage change.

Yet another way to represent change in the acoustic output signals of two different samples is to simply compare the hard numbers of the acoustic output signal form one sample to the acoustic output signal of another sample.

In accordance with the various embodiments of the invention, two different signal transduction pathways can be analyzed (e.g., simultaneously), and their acoustic output signals compared. For example, the first acoustic output signal may result from detecting the output signal from a biomarker of choice in a first signal transduction pathway (e.g., the acoustic output signal is given if the biomarker of choice is phosphorylated) in a diseased or damaged cell as compared to the biomarker of choice in the first signal transduction pathway in a healthy cell. The acoustic output signals of the two samples is represented either as a ratio, a percentage, or a difference (i.e., the result when the value of one of the output signals is subtracted from the other). The second acoustic output signal may result from detecting the output signal from a biomarker of choice in a second signal transduction pathway (e.g., the acoustic output signal is given if the biomarker of choice is phosphorylated) in a diseased or damaged cell as compared to the biomarker of choice in the second signal transduction pathway in a healthy cell. The acoustic output signals of the two samples is represented either as a ratio, a percentage, or a difference (i.e., the result when the value of one of the acoustic output signals is subtracted from the other). If a therapeutic regimen is desired to be designed for the patient with the disease (e.g., from whom the diseased or damaged cell was taken), the signal transduction pathway (i.e., the first or the second signal transduction pathway) that had the greater change between the diseased or damaged cell and the healthy cell is the signal transduction pathway that is selected to be blocked by the therapeutic regimen.

Accordingly, in further aspects, the invention provides a method for analyzing two or more signal transduction pathways in a diseased or a damaged cell, or in a cell treated with an agent. In some embodiments, the two or more signal transduction pathways are analyzed simultaneously. The method comprises (a) providing a sample comprising the cell; (b) providing a first binding agent that specifically binds to an entity selected from the group consisting of a first epitope on a first biomarker in a first signal transduction pathway and a noninterface epitope on the first biomarker, wherein the first binding agent is attached to a solid support; (c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is in the first signal transduction pathway, and wherein said second binding agent is capable of attaching to a sensor; (d) mixing the sample, the first binding agent, and the second binding agent together under conditions whereby a first sandwich assay formation will be formed if the sample contains a component selected from the group consisting of the first biomarker comprising the first epitope and the first alternate epitope and the first biomarker complexed with the first biomarker partner; (e) presenting the result of step (d) to a sensor that emits an output signal that relates to the sensor's acoustic response to obtain a first number; (f) providing a third binding agent that specifically binds to an entity selected from the group consisting of a second epitope on a second biomarker in a second signal transduction pathway, wherein the second biomarker is different than the first biomarker, and a noninterface epitope on the second biomarker, and wherein the third binding agent is attached to a solid support; (g) providing a fourth binding agent that specifically binds to an entity selected from the group consisting of a second alternate epitope on the second biomarker in the second signal transduction pathway, wherein the second alternate epitope is different from the second epitope, and a noninterface epitope on a second biomarker partner, wherein said second biomarker partner is in the second signal transduction pathway, and wherein said fourth binding agent is capable of attaching to a sensor; (h) mixing the sample, the third binding agent, and the fourth binding agent together under conditions whereby a second sandwich assay formation will be formed if the sample contains a component selected from the group consisting of the second biomarker comprising the second epitope and the second alternate epitope, and the second biomarker complexed with the second biomarker partner; (i) presenting the result of step (h) to a sensor that emits an output signal that relates to the sensor's acoustic response to obtain a second number; (j) repeating steps (b)-(i) with a control sample of an untreated cell or a cell treated with a control agent to obtain a first control number and a second control number; and (k) comparing the first number to the first control number to obtain a first difference and the second number to the second control number to obtain a second difference, wherein said first difference and said second difference are represented as positive numbers; wherein a first difference larger than a second difference either identifies the first signal transduction pathway as the pathway that will be preferentially selected to be targeted by the agent or identifies the first signal transduction pathway as the pathway to target with the agent in the diseased or damaged cell, and wherein a second difference larger than a first difference either identifies the second signal transduction pathway as the pathway that will be preferentially selected to be targeted by the agent or identifies the second signal transduction pathway as the pathway to target with the agent in the diseased or damaged cell.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response to obtain a third number, a fourth number, a third control number and a fourth control number.

In further aspects, the invention provides methods for identifying a signal transduction pathway to target with an agent in a diseased or damaged cell, or in a cell treated with an agent. The methods comprise (a) providing a sample of the cell; (b) providing a first binding agent that specifically binds to a first epitope on a first biomarker in a first signal transduction pathway, wherein the first binding agent is attached to a solid support; (c) providing a second binding agent that specifically binds to a first alternate epitope on the first biomarker, wherein the first alternate epitope is different than the first epitope, wherein the first alternate epitope and the first epitope are both present only on an activated form or an inhibited form of the first biomarker, and wherein said second binding agent is capable of attaching to a sensor; (d) mixing the sample, the first binding agent, and the second binding agent together under conditions whereby a first sandwich assay formation will be formed if the sample contains the first biomarker comprising the first epitope and the first alternate epitope; (e) presenting the result of step (d) to a sensor that emits an output signal that relates to the sensor's acoustic response to obtain a first number; (f) repeating steps (b)-(e), with a first reference binding agent that specifically binds to a first reference epitope on a first reference biomarker within the sample, or a portion of the sample, and a second reference binding agent that specifically binds to a first alternate reference epitope on the first reference biomarker, wherein the first reference epitope and the first alternate reference epitope are not the same, to obtain a first reference number; (g) providing a third binding agent that specifically binds to second epitope on a second biomarker in a second signal transduction pathway, wherein the second biomarker is different than the first biomarker, and wherein the third binding agent is attached to a solid support; (h) providing a fourth binding agent that specifically binds to a second alternate epitope on the second biomarker in the second signal transduction pathway, wherein the second alternate epitope is different from the second epitope, wherein the second alternate epitope and the second epitope are both present only on an activated form or an inhibited form of the second biomarker, and wherein said fourth binding agent is capable of attaching to a sensor; (i) mixing the sample, the third binding agent, and the fourth binding agent together under conditions whereby a second sandwich assay formation will be formed if the sample contains the second biomarker comprising the second epitope and the second alternate epitope; (j) presenting the result of step (h) to a sensor that emits an output signal that relates to the sensor's acoustic response to obtain a second number; (k) repeating steps (g)-(j), with a third reference binding agent that specifically binds to a second reference epitope on a second reference biomarker within the sample, or a portion of the sample, and a fourth reference binding agent that specifically binds to a second alternate reference epitope on the reference biomarker, wherein the second reference epitope and the second alternate reference epitope are not the same, to obtain a second reference number; (l) repeating steps (b)-(k) with a control sample to obtain a control first number, a control first reference number, a control second number, and a control second reference number, the control sample selected from the group consisting of an untreated cell and a cell treated with a control agent; and (m) comparing the first number to the first reference number to obtain a first ratio, comparing the second number to the second reference number to obtain a second ratio, comparing the control first number to the control first reference number to obtain a control first ratio, and comparing the control second number to the control second reference number to obtain a control second ratio; wherein a change in the first ratio to the control first ratio is greater than the change in the second ratio to the control second ratio either identifies the first signal transduction pathway as the pathway as the pathway to target with the agent in the diseased or damaged cell or identifies the first signal transduction pathway as the pathway to target with the agent in the diseased or damaged cell, and wherein a change in the first ratio to the control first ratio is less than the change in the second ratio to the control second ratio either identifies the second signal transduction pathway as the pathway that will be preferentially selected to be targeted by the agent or identifies the second signal transduction pathway as the pathway to target with the agent in the diseased or damaged cell.

In some embodiments, a third pathway is analyzed by repeating steps (b)-(j) above with a fifth binding agent that specifically binds to an entity selected from the group consisting of a third epitope on a third biomarker in a third signal transduction pathway and a noninterface epitope on the third biomarker, wherein said third biomarker is in a third signal transduction pathway, wherein the fifth binding agent is attached to a solid support; a sixth binding agent that specifically binds to an entity selected from the group consisting of a third alternate epitope on the third biomarker, wherein the third alternate epitope is different than the third epitope, and a noninterface epitope on a third biomarker partner, wherein said third biomarker partner is in the third signal transduction pathway, and wherein said sixth binding agent is capable of attaching to a sensor that emits an output signal that relates to the sensor's acoustic response, and a fifth reference binding agent that specifically binds to a third reference epitope on a third reference biomarker within the sample, or a portion of the sample, and a sixth reference binding agent that specifically binds to a third alternate reference epitope on the third reference biomarker, wherein the third reference epitope and the third alternate reference epitope are not the same, to obtain a third number, a third reference number, a control third number, a control third reference number.

As used herein, by “biomarker” is simply meant a biological molecule. Non-limiting biomarkers include proteins (including non-derivatized and derivatized proteins such as glycoproteins or lipoproteins), carbohydrates, lipids, and nucleic acid molecules (e.g., RNA or DNA). In some embodiments, a biomarker may be associated with a disease or a potential disease. By “disease” is simply meant any condition (i.e., disease state) that may affect a subject such that the subject is not healthy. An affected cell from a diseased subject is referred to as a damaged or diseased cell. Non-limiting diseases include cancer (both benign and metastatic), degenerative diseases (e.g., Alzheimer's disease, Parkinson's disease, multiple sclerosis), immune diseases (e.g., Graves' disease, celiac disease, Type I diabetes, rheumatoid arthritis), infectious diseases (e.g., tuberculosis, AIDS, leishmaniasis, tapeworm), inflammatory diseases (e.g., Multiple Sclerosis, atherosclerosis, Crohn's disease), and heart disease. Of course, some diseases fit more than one category. For example, rheumatoid arthritis is an immune disease, but is also an inflammatory disease.

In various embodiments of the methods and kits described herein, the damaged or diseased cell is human. In various embodiments, the damaged or diseased cell is a tumor cell, an injured cell, and infected cell, or a degenerative cell. In some embodiments, the tumor cell is selected from the group consisting of a colon cancer cell, a breast cancer cell, a prostate cancer cell, a leukemia cell, a lymphoma cell, and glioblastoma cell, a pancreatic cancer cell, a liver cancer cell, a lung cancer cell, a skin cancer cell, and a stomach cancer cell. As used herein, by “sample” is meant any sample that may contain a biomarker. Thus, a sample includes, without limitation, living intact cells (e.g., healthy cells or diseased or damaged cells such as cancer or tumor cells), cells from a xenograft, cells from genetically engineered mouse models (GEMs, see, e.g., Richmond and Su, Dis Model Mech. 1:78-82, 2008), fixed intact cells, cell lysates, conditioned culture media (i.e., culture media in which cells are grown in vitro), saliva, mucous, tears, blood, circulating tumor cells, synovial fluid, serum, tissues, marrow, lymph/interstitial fluids, buccal cells, pleural effusion, fine needle aspirate, mucosal cells, cerebrospinal fluid, breast milk, semen, feces, plasma, urine, a suspension of cells, a biopsy sample (e.g., taken from normal or diseased tissue), or a suspension of cells and viruses or extracts or any of the foregoing, In some embodiments, samples useful in the practice of the methods described herein may be obtained from any mammal.

In some embodiments, the sample of damaged or diseased cells is selected from the group consisting of a cultured cell sample, a circulating tumor cell, a tissue biopsy sample, a fine needle aspirate sample, and a blood sample.

In some embodiments, the sample of a tumor cell comprises cell parts or portions of a tumor cell that have been shed, for example, by a dying tumor cell, a dividing tumor cell, or a healthy tumor cell. For example, a sample of a tumor cell may be an exosome (see, e.g., Henderson and Azorsa, Frontiers in Oncology 2, Article 38 (2012)).

Any sample obtained from a subject having or suspected of having a disease is suitable for use in the methods described herein. In one embodiment, the sample comprises cells obtained from a biopsy. The biopsy may be obtained, according to standard clinical techniques, from tissues occurring in an organ of the animal having or suspected of having the disease. Where the disease is cancer, the biopsy may be taken from secondary tumors that have metastasized in other tissues. In another embodiment, the biological sample comprises cells obtained from a fine needle aspirate taken from a tumor, and techniques for obtaining such aspirates are well known in the art (see Cristallini et al., Acta Cytol. 36(3): 416-22 (1992)).

It shall be understood that any biological sample comprising cells (or extracts or parts thereof, such as exosomes) from a diseased or damaged cells (or normal control cells) is suitable for use in the methods and kids described herein. For example, the biological sample may be obtained from an effusion, such as a pleural effusion. Pleural effusions (liquid that forms outside the lung in the thoracic cavity and which contains cancerous cells) are known to form in many patients and the presence of such effusion is predictive of a poor outcome and short survival time. Standard techniques for obtaining pleural effusion samples have been described and are well known in the art (see Sahn, Clin Chest Med. 3(2): 443-52 (1982)). Serum and bone marrow samples may be particularly preferred for patients with leukemia. For cancers involving solid tumors, such as sarcomas and carcinomas, the biological sample may comprise cells obtained from a tumor biopsy, which maybe be obtained according to standard clinical techniques.

In addition, samples of circulating tumor cells may also be obtained from serum using tumor markers, cytokeratin protein markers or other methods of negative selection as described (see Ma et al., Anticancer Res. 23(1A): 49-62 (2003)). Circulating tumor cells (“CTCs”) may also be purified, for example, using the kits and reagents sold under the trademarks Vita-Assays™, Vita-Cap™, and CellSearch™. (commercially available from Vitatex, LLC (a Johnson and Johnson corporation). Other methods for isolating CTCs are described (see, for example, PCT Publication No. WO/2002/020825, Cristofanilli et al., New Engl. J. of Med. 351 (8):781-791 (2004), and Adams et al., J. Amer. Chem. Soc. 130(27): 8633-8641 (July 2008)).

As used herein, by “subject” is meant any animal including, without limitation, humans, non-human mammals (e.g., primates, rodents, ungulates), companion animals (e.g., horses, cats, dogs), livestock animals (e.g., cows, sheep, pigs, goats), birds, and reptiles. A subject may also be referred to as an individual.

In some embodiments, the sample of a damaged or diseased cell (or a normal control cell) contains a purified biomarker.

As used herein, by “purify” or “purified” (or “isolate” or “isolated”) refers to a biomarker (e.g., a protein or an nucleic acid) that is removed or separated from other components present in its natural environment. For example, a purified cell surface biomarker is one that is separated from other components of the cell membrane. A purified cytosolic biomarker is one that is separated from other components of the cell (e.g., separated from the endoplasmic reticulum, cytoplasmic proteins and/or RNA). A purified secreted biomarker is one that that is separated from other components of the extracellular space of the cell or other components of the culture media in which the cell is being grown. A purified biomarker is at least 60% free, or at least 75% free, or at least 90% free, or at least 95% free from other components present in natural environment of the indicated biomarker. For example, a recombinant biomarker (e.g., a human protein produced by prokaryotic cell) is purified.

In some embodiments, the methods of the invention may utilize the acoustic membrane and microparticle (AMMP®) technology. AMMP assays are described in U.S. Pat. Nos. 7,300,631; 7,598,094; 7,611,908; and 7,615,381, 7,629,137; as well as U.S. Patent Application Publication No. 2010/023031, all of which are hereby incorporated by reference in their entireties. In one example of using the AMMP technology, which can be performed on the ViBE® instruments sold by BioScale, Inc., Lexington, Mass., magnetic solid supports (e.g., magnetic microparticles or beads) are attached (e.g., via conjugation or specific binding) to a first binding agent that specifically binds to a target or analyte of interest. A second binding agent that also specifically binds to the target of interest (e.g., at an epitope on the target that is different from the epitope specifically bound by the first binding agent) can be tagged, with a hapten tag for example, that in turn provides a specific means for bridging captured targets on microparticles to anti-tag functionalized sensors (e.g., the sensor is coated with anti-tag antibodies). The functionalized sensors respond acoustically to loading of the magnetic microparticles and this is response is sensitively transduced by piezoelectric material. In the embodiment where the AMMP assay is used, a “sensor” (as used herein) is the specific surface connected to an acoustically sensitive chip and the “acoustic output signal” is the signal derived primarily from acoustic interaction with the sensors through the use of the piezoelectric transduction. In some embodiments, the portion of the binding agent that does not contain the binding fragment is attached to the solid support (e.g., either the sensor surface or the microparticle bead surface). For example, if the binding agent is an antibody, the portion of the antibody attached to the solid support may be, for example, the Fc portion of the antibody leaving the antigen binding domain of the antibody (i.e., the binding fragment of the antibody) free to interact with any target present in the sample.

In some embodiments, the Acoustic Membrane MicroParticle (AMMP) assay technology (BioScale Inc., Lexington, Mass.), performed on the ViBE protein analysis platform (BioScale Inc.), permits the use of a single technology for detecting oncology biomarkers in serum, cell lysates, cell surfaces and a wide variety of other sample types (see, e.g., Dickerson et al., J. Vis. Exp. 48: 2493, 2011 and Yan et al. Anal Biochem 410(1): 13-18, 2011). In some embodiments, AMMP assays offer advantages over conventional approaches to assay development—most importantly, the non-optical detection of low-abundance biomarkers in complex samples with minimal sample manipulation. Other reported advantages of the AMMP technology include improved sensitivity and reproducibility, less manual sample manipulation, the capability of walk-away operation, reduced consumption of critical samples and antibodies, and the ability to measure proteins in their natural states.

AMMP technology assesses analyte concentration by measuring changes in the oscillations of a piezoelectric sensor as antibody-labeled magnetic microparticles bind to the sensor surface. Thus, in some embodiments of the non-limiting methods of the invention, the acoustic output signal is quantifiable, such that amount of the biomarker or the complex in the sample can be quantified. The biosensor membranes are resonant devices that change their oscillation frequency in direct proportion to microparticles accumulated on their surfaces, in proportion to mass and acoustic loading, thus permitting rapid and efficient determination of analyte concentration through the specific binding of microparticles. These AMMP assays have been used to analyze pharmacodynamic biomarkers for inhibition of the proteosome and phosphoinositol 3-kinase (see Yan et al. Anal Biochem 410(1): 13-18, 2011). In that context, the AMMP assay proved to be a robust method for measuring recombinant protein standards as well as endogenously derived proteins from both tissue culture and mouse xenograft tumor lysates.

Thus, in the AMMP technology, which can be performed on the VIBE instruments sold by BioScale, Inc., Lexington, Mass., magnetic solid supports (e.g., magnetic microparticles) are attached (e.g., via conjugation or specific binding) to analyte-specific binding agents (e.g., biomarker-specific antibodies). These binding agent-coupled magnetic solid supports, are mixed in sample with an additional competing or affinity reagents (i.e., binding agents such as another antibody) specific for the analyte, and the resulting captured complexes, post incubation, are detected with a specific surface applied to an acoustically sensitive chip. The AMMP technology quantifiably measures changes in the frequency emitted by a piezoelectric material proportional to the amount of an antibody-antigen sandwich that binds to the vibrating specific surface.

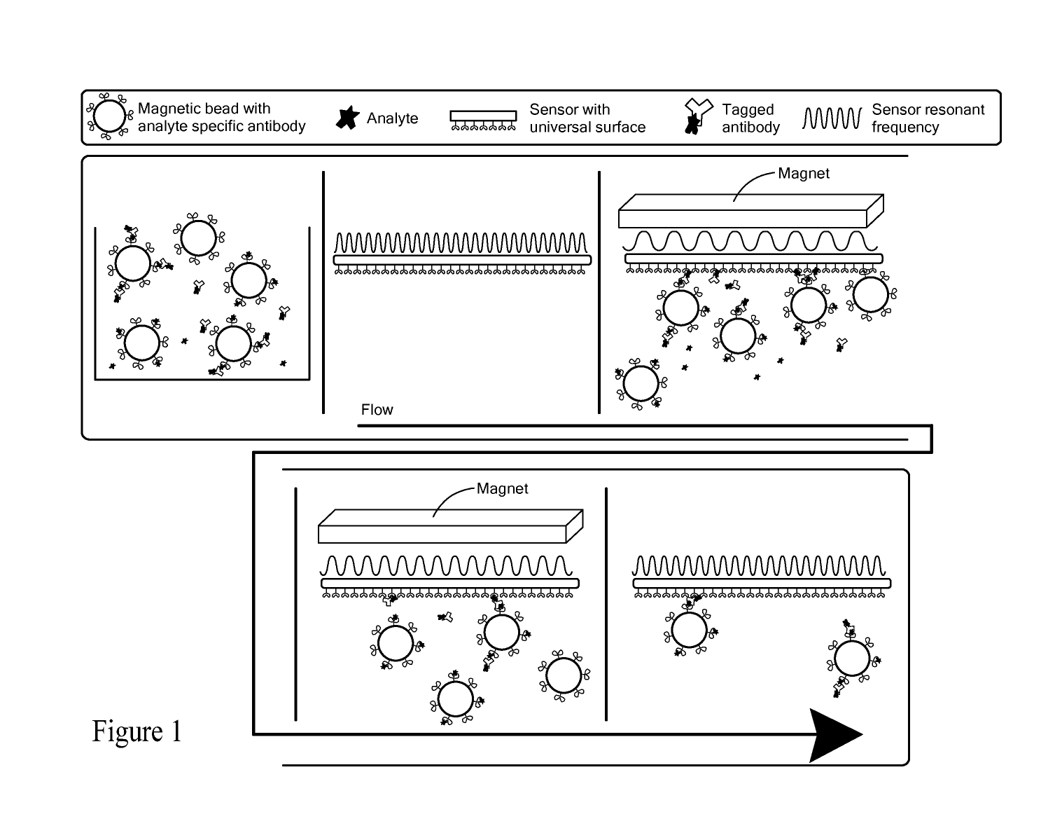

FIG. 1 shows a non-limiting embodiment of an AMMP assay. As shown in FIG. 1, in the first step for a typical sandwich assay, a sample suspected of containing the analyte (e.g., a biomarker of interest) is mixed with a solid support (e.g., magnetic beads) attached to a first binding agent (e.g., a first antibody) that specifically bind to the analyte. The solid support can be coated with binding agent by any means. For example, the solid support (e.g., magnetic beads) can be coated with streptavidin, and the first binding agent conjugated to biotin. When the streptavidin-coated solid support and the biotinylated first binding agent are allowed to interact, the strong affinity of streptavidin and biotin will result in the attachment of the first binding agent to the solid support.

Also included in the mixture of sample suspected of containing an analyte and the anti-analyte first binding agent-coated solid support is a second binding agent that specifically binds to the analyte. In some embodiments, the second binding agent (e.g., second antibody) specifically binds to the analyte on an epitope that is different than the epitope on the analyte that is specifically bound by the first binding agent (e.g., first antibody). By “epitope” is meant a portion on an analyte (e.g., a biomarker) that can be specifically bound by a binding agent. If the sample does indeed contain an analyte, the mixture of sample suspected of containing an analyte, the anti-analyte first binding agent-coated solid support, and the second binding agent that specifically binds to the analyte will form a complex comprising a solid support-attached first binding agent/analyte/second binding agent. This complex is referred to herein as a “sandwich assay formation”.

Returning to FIG. 1, this second binding agent (which, in FIG. 1, is an antibody) is tagged with FITC (fluorescein isothiocyanate). This mixture (i.e., containing the sample suspected of containing an analyte, the anti-analyte first binding agent-coated solid support, and the second binding agent that specifically binds to the analyte) is presented to one side of the sensor which, in FIG. 1, is coated with anti-FITC antibodies. A magnet is then applied to the other side of the sensor, thereby accumulating the sandwich assay formation (if the analyte is present in the mixture) or the second antibody tagged with FITC (if the analyte is not present) to the anti-FITC antibody-coated side of the sensor. The anti-FITC antibody-coated side of the sensor is then washed while the magnet is still engaged to remove non-binding, excess materials (e.g., excess magnetic beads). Next, the magnet is disengaged and incomplete bead sandwich complexes are washed away while FITC-Ab/analyte/bead-Ab complexes (i.e., the sandwich assay formation) remain specifically bound to the anti-FITC antibody-coated side of the sensor. The frequency emitted by the vibrating sensor from the bound magnetic beads is then measured and used to quantify the amount of analyte present in the sample.

One advantage of the AMMP technology is that the sensor can be regenerated and prepped for analysis of a subsequent sample (See, e.g., U.S. Pat. Nos. 7,300,631; 7,611,908; and 7,615,381).

As used herein, by “specifically bind” means that a binding agent (e.g., an antibody) binds to its intended target (e.g., the biomarker or analyte) such that the interaction between the binding agent and its specific target is dependent upon the presence of a particular structure of target; in other words, the binding agent is recognizing and binding to a specific target structure rather than to all molecules (e.g., all other biomarkers) in general. When a target (e.g., a biomarker) is bound by a binding agent such as an antibody, that target is said to be “specifically bound” by the binding agent. Binding agents that specifically bind to a particular target may be referred to as a target-specific binding agent (e.g., a cMET-specific antibody), a binding agent that is specific for that target (e.g., an antibody that is specific for cMET), or an anti-target binding agent (e.g., an anti-cMET antibody). Note that by “binding fragment” means a fragment or portion of a binding agent that specifically binds the target. For example, the Fab fragment of an antibody is a binding fragment of an antibody. In some embodiments, a binding agent (e.g., an antibody) that specifically binds its target has a dissociation constant (K_{D}) for its target of 1×10⁻⁶M or less. In some embodiments, a binding agent that specifically binds to its target (or tag) binds with a K_{D} of 1×10⁻⁷ M or less, or a K_{D} of 1×10⁻⁸ M or less, or a K_{D} of 1×10⁻⁹ M or less, or a K_{D} of 1×10⁻¹⁰M or less, of a K_{D} of 1×10⁻¹¹M or less, of a K_{D} of 1×10⁻¹²M or less. In certain embodiments, a binding agent that specifically binds to its target (or tag) binds with a K_{D} of 1 pM to 500 pM, or between 500 pM to 1 uM, or between 1 uM to 100 nM, or between 100 mM to 10 nM.

It should be noted that in the AMMP assays describe herein, any method can be used to attach the first binding agent to the solid support. For example, the first binding agent can be chemically conjugated to the solid support using standard methods (see, e.g., “Thermo Scientific Pierce Crosslinking Technical Handbook” published by Thermo Scientific (part of ThermoFisher Scientific), Article No 1601673, 04/09; copyright 2009 by Thermo Fisher Scientific, Inc.). In some embodiments, the portion of the binding agent that does not contain the binding fragment is attached to the solid support. For example, if the binding agent is an antibody, the portion of the antibody attached to the solid support may be, for example, the Fc portion of the antibody.

Similarly, any means can be used to enable the second binding to become attached to the side of the sensor to which the magnet will not be applied. Although in the above description of FIG. 1, the first binding agent is attached to the solid support via a biotin/streptavidin interaction and the second binding agent is able to attach to the side of the sensor to which the magnet will not be applied via a FITC/anti-FITC antibody interaction, any interactions may be used. For example, the two interactions described may be exchanged (e.g., the first binding agent is attached to the solid support via a FITC/anti-FITC antibody interaction and the second binding agent is attached to the sensor via a streptavidin/biotin interaction). Of course, other interactions may be used. For example, the second binding agent may be attached to a tag, and the sensor coated with a binding agent that will specifically bind to the tag. Such tag/binding agent pairs are known and include, without limitation, those listed on Table 1.

TABLE 1

| Tag | Binding Agent |
|---|---|
| Avidin or streptavidin | biotin |
| biotin | Avidin or streptavidin |
| FITC | Antibody that specifically binds to FITC |
| His tag (i.e., tag comprising 6 | Ni-NTA agarose |
| histidine residues in a row) | |
| GST tag (tag of approx. 26kDa) | glutathione |
| Myc-tag | Antibody that specifically binds myc tag |
| FLAG-tag | Antibody that specifically binds FLAG tag |

For GST-tagged binding agent specific for an analyte (e.g., a biomarker), glutatione coated solid supports are commercially available (e.g., Glutathione agarose and glutathione magnetic beads are available from Thermo Scientific (Watham, Mass.), catalog nos. 15160 and 88822, respectively). Other companies also sell glutathione coated solid supports (e.g., Qiagen (Valencia, Calif.), SigmaAldrich (St. Louis, Mo.), etc.).

For HIS-tagged binding agent specific for an analyte (e.g., a biomarker), Ni-NTA agarose beads, Ni-NTA resin and Ni-NTA magnetic agarose beads are commercially available from Qiagen (Valencia, Calif.). Other companies also sell Ni-NTA resins and sold supports (e.g., Invitrogen, Inc. (Carlsbad, Calif.), ThermoScientific, etc.).

Anti-FITC, anti-myc, and anti-FLAG antibodies are commercially available (e.g., Cell Signaling Technology, Inc. (Danvers, Mass.), ThermoScientific (Waltham, Mass.), Millipore (Billerica, Mass.), Abcam (Cambridge, Mass.), etc.).

Other tags are specifically bounds by binding agents. For example, His tag is specifically bound by Ni-NTA (e.g., Ni-NTA magnetic beads can be used to specifically bind His tagged biological molecules). Likewise, the V5 tag (i.e., derived from the Pk epitope present on P and V proteins of the paramyxovirus of simian virus 5 and having the sequence of 14 amino acids (GKPIPNPLLGLDST), or a shorter 9 amino acid sequence (IPNPLLGLD)) can be specifically bound by an anti-V5 tag antibody (i.e., an antibody that specifically binds to V5). Such a V5-specific antibody is sold as catalog no. ab9137 by Abcam, Cambridge, Mass.

In some embodiments, the biomarker (or the first or the second biomarker in the complex) is selected from the group consisting of a cell surface biomarker, a secreted biomarker, and a cytosolic biomarker.

As used herein, by “secreted” is meant that a biomarker is secreted into the extracellular space from the cell producing it. In other words, the secreted biomarker is expressed by the cell, which then transmits the secreted biomarker through the cell membrane into the extracellular space. If the cell is a blood cell, the secreted biomarker will be secreted into the serum of the blood. If the cell is grown in vitro, the secreted biomarker will be secreted into the conditioned culture media in which the cell is being grown. The secreted biomarker may then be purified. Secreted biomarkers include, without limitation, hormones, cytokines, antibodies, and growth factors.

In contrast, by “cytosolic” (or “intracellular” is meant that a biomarker is not secreted from the cell producing it. In other words, the cell must be lysed (i.e., broken open) in order to purify the cytosolic biomarker from it. Cytosolic biomarkers include, without limitation, intracellular kinase enzymes, ribosomal proteins, and DNA polymerase. Some cytosolic biomarkers may be attached to the cytoplasmic side of the cell membrane, or may be attached to an organelle membrane (e.g., a cytosolic biomarker may be attached to the endoplasmic reticulum).

By “cell surface” is meant a biomarker that is attached to the cell membrane, and part of the molecule is extracellular to the cell. In some embodiments, a portion of the cell surface molecule (i.e., the transmembrane domain) spans the cell membrane and has both an extracellular portion and an intracellular portion. For example, a biomarker such a T cell receptor is a cell surface biomarker because it is attached to the cell membrane, has an extracellular portion and a cytoplasmic tail (i.e., the intracellular portion). A cell surface molecule may be attached only to the extracellular cell surface, and not have a cytoplasmic tail.

In some embodiments of the various non-limiting methods and kits of the invention, the biomarker (or the first or the second biomarker in the complex) plays a role in a signal transduction pathway. As used herein, by “signal transduction pathway” or “signaling pathway” or “signal transduction cascade” is meant a collection of biomarkers involved in the transmission of a signal within a cell. Each of the biomarkers in a particular signal transduction pathway may be referred to as a biomarker in that pathway. The biomarker members in a signal transduction pathway may be upstream or downstream of each other. The signal transmitted by a signal transduction pathway resulting in an effector function includes, without limitation, a signal to secrete a protein in response to an external trigger (e.g., a signal from the B cell antigen receptor for the B cell to secrete antibody), a signal to grow in response to stimulus (e.g., binding of a growth factor to its receptor on the cell's surface), and a signal to undergo apoptosis or otherwise die following a lethal error within the cell such as the loss of part of a chromosome during mitosis.

A signal is passed along the signal transduction pathway by the activation of downstream biomarkers by upstream biomarkers. By “upstream” is meant that an upstream biomarker participates in the pathway before participation of a downstream biomarker. By “downstream” is meant that the downstream biomarker participates in the signal transduction pathway after participation of an upstream biomarker.

The downstream biomarker member within a signal transduction pathway effecting the effector function may be referred to as the effector biomarker. Such effector biomarkers include, without limitation, gene regulation factors (e.g., activation of NF-kB or Fos), apoptosis factors (e.g., Bax activation). The biomarker member initiating the signal transduction pathway cascade may be referred to as the initiator biomarker. Such initiator biomarkers include, without limitation, growth factor receptors (e.g., EGFR), and cytokine receptors.

Often, a signal transduction pathway is activated (i.e., the signal transduction pathway cascade is started or initiated) by a signal received extracellularly (e.g., from a growth factor) that binds to and activates a cell surface receptor biomarker. That receptor, once activates, transmits the activation signal through the cell along a signal transduction pathway to elicit a physiological response. Some cellular signal transduction pathways are well understood. For example, in a cancer cell, the MAP kinase signaling pathway is well characterized, as further described in Example 4 below. Accordingly, in some embodiments, the signal transduction pathway is the MAPK signal transduction pathway.

Other signal transduction pathways are well characterized including, without limitation, the JAK/STAT signal transduction pathway (see, e.g., Aaronson et al., Science 296(5573): 1653-1655, 2002; Shuai, K., Cell Research 16(2): 196-202, 2006), the PI3kinase/AKT/mTOR signaling pathway (see, e.g., Morgensztern et al., Anti-Cancer Drugs 16(8): 797-803, 2005; Yap et al., Current Opinion in Pharmacology 8 (4): 393-412, 2008; Di Cosimo et al., Journal of Clinical Oncology 25 (18S): 3511, 2007), and the WNT/hedgehog/notch signaling pathway (see, e.g., Liu et al., Cancer Research 66(12: 6063-6071, 2006; Bhardwaj et al., Nature Immunology 2(2): 172-180, 2001).

Much study has been done to elucidate the signal transduction pathways that are altered (i.e., have pathway members whose activation or expression is aberrant) in disease states. Indeed, the Vogelstein group identified twelve core signaling pathways that were altered in human pancreatic cancers (Jones et al., Science 321: 1801-1806, 2008). These signal transduction pathways include the following pathways: K-ras signaling, TGF-beta signaling, JNK signaling, Integrin Signaling, Wnt/Notch signaling, Hedgehog signaling, Control of G1/S phase transition, Apoptosis, DNA damage control, Small GTPase signaling, Invasion, and homophilic cell adhesion (see U.S. Patent Publication No. 20120115735, herein incorporated by reference).

As discussed herein, some of these pathways are well elucidated. For example, Kras signaling activates raf which activates MEK which activates Erk which then enters the nucleus to activate FOS to activate cells.

Similarly, TGF-beta signaling activates a number of related signal transduction pathways. For example, TGF-beta signaling activates PI3K which activates PDK1 which activates AKT which activates mTOR, which activates p70 S6Kinase. TGF-beta also activates MEKK1 which activates MKK3/6 which activates p38. TGF-beta also activates MEKK1 which activates MKK4/7 which activates JNK which activates Jun. TGF-beta also activates SOS which activates RAS which leads to activation of ERK1/2

Many signal transduction pathways in cancer and other diseased and/or damaged cells (e.g., degenerative disorders) involve the ubiquitin pathway. For example, the TGF-beta signaling pathway is regulated by ubiquitin-mediated proteolysis at different levels (see Aggrawal and Massaque, Nat. Cell Biol. 14(7):656-657, 2012).

Of course, other signal transduction pathways have been well studied. For example, the signal transduction pathway initiated by the binding of interleuekin-17 to its receptor (or the aberrant signaling by the IL-17 receptor). Interleukin-17 promotes cartilage breakdown by inducing matrix metalloproteinases (MMPs) and aggrecanases (a disintegrin and metalloproteinase with thrombospondin motif, ADAMTS) in arthritic joints (see Sylvester et al., Cell Signal. 16(4): 469-476, 2004). Interleukin-17 is also implicated in the development of T helper cells into TH1 or TH2 cells. Non-limiting biomarkers involved in the Interleukin-17 signaling pathway include phosphorylation of extracellular signal-regulated kinase (ERK), protein 38 (p38) and c-Jun N-terminal kinase (JNK).

Another signal transduction pathway that can be studied using the methods and kits described herein is the signal transduction pathway initiated by the binding of interleukin-13 to its receptor (or the aberrant signaling by the IL-13 receptor). IL-13 signaling is strongly implicated in the development and onset of asthma. Yet another signal transduction pathway that can be analyzed is the Interleukin-4 signaling pathway which is related to Interleukin-13 signaling; these two signal transduction pathways may be salvage pathways for each other. For example, if an asthma-treating agent is designed to target a biomarker involved in Interleukin-13 signaling, a resistant cell (i.e., a cell that no longer responds to the agent) may be escaping the effects of the agent by signaling through the Interleukin-4 signaling pathway. And vice versa.

Additional (but non-limiting) signal transduction pathways that can be studied using the methods and kits described herein is the signal transduction pathway initiated by the binding of interleukin-2 or interleukin-21 to their respective receptors (or the aberrant signaling by these receptors).

Using the methods and the kits described herein, one can determine whether a cell being treated with an agent targeting a biomarker or pathway (or a cocktail or agents) is actually responding to the agent. In other words, the methods and the kits described herein can be used to determine if the agent is effective at treating the cell. As used throughout, an agent is “effective” is treating a damaged or diseased cell if the cell responds as desired following treatment with the agent. For example, if the damaged or diseased cell is a tumor cell, a desirable response may be, for example, death of the tumor cell or abrogation of growth of the tumor cell. Other indicia that a cell is responding to an agent include, without limitation, shrinkage of the tumor (e.g., by reduction in the number of tumor cells in the tumor growth), resumption of the tumor cell of controlled cell growth, and loss of metastatic ability of the tumor cell. These indicia can be measured according to standard methods (e.g., haemocytometer for cell growth, calipers for tumor size, etc.). A diseased or damaged cell responding to treatment with agent is said to be “responsive.”

In some embodiments, the methods and kits described herein can be used to determine if an agent-treated cell is developing resistance to the agent. By “resistance” is meant that the cell no longer responds to the agent (e.g., the cell resumes its ability to grow in the presence of the agent). The cell can become resistant by, for example, signaling (e.g., to express proteins needed for cell division) through a signal transduction that does not include the biomarker targeted by the agent. For example, as described in Jones et al., Science 321: 1801-1806, 2008 and in U.S. Patent Publication No. 20120115735 (both herein incorporated by reference in their entireties), twelve signaling pathways, including the K-ras signaling pathway, the TGF-beta signaling pathway, the JNK signaling pathway, the Integrin Signaling pathway, the Wnt/Notch signaling pathway, the Hedgehog signaling pathway, the Apoptosis pathway, can go awry in human pancreatic cancers. If an agent is targeting a biomarker in one of these pathways, a resistant cell may start signaling through another signal transduction pathway to escape the effects of the agent.

It should be understood that in all of the methods described herein (and the kits for performing the methods), multiple signaling pathways can be analyzed, and multiple agents targeting one or more biomarkers in one or more signaling pathways can likewise be analyzed for their effectiveness in having treated cells respond to the agent. Similarly, multiple pathways can be analyzed to determine whether two or more biomarkers, possibly in more than one signal transduction pathway, are preferentially targeted in a diseased cell. For example, if a signal transduction pathway (e.g., the MEK/ERK pathway) in a diseased or damaged is identified as being the pathway to preferentially target with an agent, the diseased cell, that cell can be treated with one or more inhibitors targeting biomarkers in that pathway. For example, the cell may be treated with one agent targeting MEK (e.g., trametinib (also called GSK1120212); see Caroline Robert et al., 2012 ASCO Meeting Abstract No. LBA8509, J. Clin Oncol. 30, 2012), or two agents, one targeting MEK (e.g., trametinib) and one targeting Raf (e.g., sorafenib (also called Nexavar); see Smalley et al., Oncogene 28: 85-94, 2009; Wilhelm et al., Mol. Cancer Ther. 7(10): 3129-3140, 2008).

In some embodiments, the methods herein described demonstrate the flexibility and versatility of AMMP assay technology. Particularly, the results show the feasibility of rapidly developing novel highly sensitive drug screening methods for human cancer, hybridoma-screening protocols, and sensitive biomarker detection assays from human serum samples for use in a clinical diagnostic setting.

One of the great advantages of the methods and kits described herein lies in the multiple ways the data can be represented. For example, a fold change in a biomarker of interest can be predetermined and used as a reference against which the experimental results are compared. For example, in Example 9 below, human melanoma patients are screened for phosphorylated AKT and biomarkers in the ERK/MEK pathway. Using a standard cell line (e.g., the human SK-MEL-28 cell line commercially available from the American Type Culture Collection, Manassas, Va. (Order No. 300337), the fold change of, for example, phosphorylated AKT1, in vemurafenib-treated cells versus untreated cells or cells treated with a control agent can be predetermined. The results obtained from Example 9 can be compared to this fold change. The predetermined fold change may be at least about 10%, or at least about 20%, or at least about 25%, or at least about 50%, or at least about 100%, or at least about 150%, or at least about 200%, or at least about 250%, or at least about 500%, or at least 1000%, or at least about 2500%, or at least about 5000%, and at least about 10,000%.

The most widely used immunoassays today, including ELISA, MesoScale Discovery (Gaithersburg, Md. using, e.g., the SECTOR Imager 6000 or SECTOR Imager 2400), and AlphaLISA platforms, rely on optical detection with fluorophore-conjugated or enzyme-linked antibodies. Current diagnostic methods lack the sensitivity necessary to detect key low-abundance protein biomarkers present in bodily fluids, mainly due to interference caused by complex sample matrices. Furthermore, these methods are expensive and therefore not cost effective for large-scale biomarker screens. Many immunoassay platforms are prone to signal interference, matrix effects, cross-reactivity, antibody interference, and the hook effect, and many designs are specifically prone to particular types of interference. AMMP assay technology quantifies protein concentrations non-optically by calculating the analyte mass amplified by the bound magnetic microparticles' mass. Cross-reactivity or non-specificity occurs when a molecule in the sample competes with the analyte for antibody binding due to structural similarities. While this type of interference is highest for competitive immunoassays, two-site “sandwich” assays, such as the design of the AMMP technology, minimize cross-reactivity. Most importantly, the non-optical readout associated with the AMMP assay minimizes signal interference compared to typical optical immunoassay systems where the signal can be quenched due to sample complexity, pH, ionic strength, lipid and protein concentration, and other matrix effects. Because of the advantage AMMP has in minimizing sample effects, researchers are able to shorten the required sample preparation time and reduce operator-related error.

In yet another aspect, the invention provides a kit comprising (a) a first binding agent that specifically binds to a first biomarker, said first biomarker involved in a KRAS signaling pathway, and/or a second binding agent that specifically binds to a second biomarker, said second biomarker involved in a TGF-beta signaling pathway, and/or a third binding agent that specifically binds to a third biomarker, said third biomarker involved in a JNK signaling pathway, and/or a fourth binding agent that specifically binds to a fourth biomarker, said fourth biomarker involved in an integrin signaling pathway, and/or a fifth binding agent that specifically binds to a fifth biomarker, said fifth biomarker involved in a wnt/notch signaling pathway, and/or a sixth binding agent that specifically binds to a sixth biomarker, said sixth biomarker involved in a hedgehog signaling pathway, and/or a seventh binding agent that specifically binds to a seventh biomarker, said seventh biomarker involved in a G1/S phase transition signaling pathway, and/or an eighth binding agent that specifically binds to an eighth biomarker, said eighth biomarker involved in an apoptosis signaling pathway, and/or a ninth binding agent that specifically binds to a ninth biomarker, said ninth biomarker involved in a DNA damage control signaling pathway, and/or a tenth binding agent that specifically binds to a tenth biomarker, said tenth biomarker involved in a small GTPase signaling pathway, and/or an eleventh binding agent that specifically binds to an eleventh biomarker, said eleventh biomarker involved in an invasion signaling pathway, and/or a twelfth binding agent that specifically binds to a twelfth biomarker, said twelfth biomarker involved in a hemophilic cell adhesion signaling pathway, wherein, if present, the first binding agent, the second binding agent, the third binding agent, the fourth binding agent, the fifth binding agent, the sixth binding agent, the seventh binding agent, the eighth binding agent, the ninth binding agent, the tenth binding agent, the eleventh binding agent, and/or the twelfth binding agent is selected from the group consisting of binding agent capable of being attached to a solid support and a binding agent capable of being attached to a sensor; and instructions for utilizing the first binding agent, the second binding agent, the third binding agent, the fourth binding agent, the fifth binding agent, the sixth binding agent, the seventh binding agent, the eighth binding agent, the ninth binding agent, the tenth binding agent, the eleventh binding agent, and the twelfth binding agent to identify a signaling pathway utilized by a cell. In some embodiments, the instructions (e.g., written instructions) include a description of the AMMP technology described herein and in, for example, U.S. Pat. Nos. 7,300,631; 7,611,908; and 7,615,381. In some embodiments, the solid support is magnetic.

In various embodiments, part of, most of, and/or all of a signal transduction pathway is analyzed using the methods and kits described herein.

In various embodiments of the invention, the methods and kits are optimized for use with the acoustic membrane and microparticle (AMMP®) technology. The AMMP assay technology offers its own distinct advantages over alternative platforms. These include exploitation of antibodies without bias toward antibody orientation upon conjugation. In addition, there need not be concern about antibody species, allowing for a larger library of polyclonal and monoclonal pairings to be used. Finally, the AMMP assay offers the flexibility to detect post-translational modifications, protein/protein interactions, and even intact cells in a short amount of time. In some embodiments, the methods and kits are optimized for use with a VIBE instrument.

In further aspects, the invention provides methods for detecting the presence of a biomarker, or a complex of two or more biomarkers, in a sample. The methods comprise providing a sample suspected of containing the biomarker or the complex; providing a first binding agent that specifically binds to the biomarker (or first biomarker in the complex), wherein the first binding agent is attached to a solid support; providing a second binding agent that specifically binds to the biomarker (or second biomarker in the complex), wherein said second binding agent is capable of attaching to a sensor; mixing the sample, the first binding agent, and the second binding agent together under conditions whereby a sandwich assay formation will be formed if the sample contains the biomarker or the complex; presenting the mixture to a sensor that emits an acoustic output signal and detecting a change in the output signal as compared to a control output signal of the sensor, wherein a change in the output signal indicates the attachment of the sandwich assay formation to the sensor and thereby indicates the presence of the biomarker or the complex in the sample. In some embodiments, the sample is of a cell (e.g., of a damaged cell). In some embodiments, the control output signal is emitted from the sensor presented with the first binding agent, the second binding agent, and a control sample known to contain the biomarker. In some embodiments, the output signal is calculated by subtracting a background output signal from the sensor presented only with the first binding sample from an initial output signal from the sensor presented with the mixture of the sample, first binding agent, and second binding agent.

In some embodiments, the output signal is quantifiable, such that amount of the biomarker or the complex in the sample can be quantified. In some embodiments, the biomarker (or the first or the second biomarker in the complex) is selected from the group consisting of a cell surface biomarker, a secreted biomarker, and a cytosolic biomarker.

In some embodiments, the biomarker (or the first or the second biomarker in the complex) is a protein. In some embodiments, the biomarker (or the first or the second biomarker in the complex) plays a role in a signal transduction pathway. In some embodiments, the signal transduction pathway is the MAPK signal transduction pathway.

In some embodiments, the biomarker (or the first or the second biomarker in the complex) is associated with a disease. In some embodiments, the sample is obtained from a subject having or is suspected of having a disease. For example, the disease may be a cancer, a degenerative disease, an immune disease, an infectious disease, an inflammatory diseases, or heart disease.

In a further aspect, the invention provides a kit for detecting the presence of a complex comprising a first biomarker and a second biomarker in a sample. In some embodiments, the kit includes a first binding agent that specifically binds to the first biomarker, wherein said first binding agent is attached to a solid support; a second binding agent that specifically binds to the second biomarker, wherein said second binding agent is capable of attaching to a sensor that emits an acoustic output signal; and instructions for using the first binding agent and the second binding agent to detect the presence of the complex. In some embodiments, the first biomarker and the second biomarker are in the same signaling pathway.

In yet another aspect, the invention provides a kit for detecting the presence of a biomarker in an activated form, the activated biomarker comprising a first epitope and a second epitope in a sample. In some embodiments, the kit includes a first binding agent that specifically binds to the first epitope on the biomarker, wherein said first binding agent is attached to a solid support; a second binding agent that specifically binds to the second epitope on the biomarker, wherein said second binding agent is capable of attaching to a sensor that emits an acoustic output signal; and instructions for using the first binding agent and the second binding agent to detect the presence of the activated biomarker. In some embodiments, at least one of the first or the second epitopes is a phosphorylated amino acid residue. In some embodiments, the activated biomarker is present in a sample of a cell signaling through a signal transduction pathway involving the biomarker.

In another aspect, the invention provides a method for identifying a signal transduction pathway to target with a second agent in the treatment of a damaged or diseased cell resistant to a first agent, the method comprising (a) providing a sample of a damaged or diseased cell resistant to a first agent, wherein the first agent targets a target biomarker in a first signal transduction pathway; (b) providing a first binding agent that specifically binds to a first epitope on a second biomarker in a second signal transduction pathway, wherein the first binding agent is attached to a solid support; (c) providing a second binding agent that specifically binds to a second epitope on the second biomarker in the second signal transduction pathway, wherein said second binding agent is capable of attaching to a sensor; (d) mixing the sample, the first binding agent, and the second binding agent together under conditions whereby a sandwich assay formation will be formed if the sample contains the second biomarker comprising the first epitope and the second epitope; (d) presenting the result of step (d) to a sensor that emits an acoustic output signal; and (f) detecting the acoustic output signal, wherein a change in the acoustic output signal of step (f) as compared to an acoustic output signal of a sensor that is not presented the results of step (d) indicates the attachment of the sandwich assay formation to the sensor and indicates that the second signal transduction pathway is a target for the second agent to treat the damaged or diseased cell resistant to the first agent. In some embodiments, the first binding agent binds to a first epitope on the biomarker and the second binding agent binds to a second epitope on the biomarker, wherein the first epitope and second epitope are not the same.

In yet another aspect, the invention provides a method for treating with a damaged or diseased cell that is resistant to a first agent, comprising (a) providing a sample of a damaged or diseased cell resistant to a first agent, wherein the first agent targets a target biomarker in a first signal transduction pathway; (b) providing a first binding agent that specifically binds to a first epitope on a second biomarker in a second signal transduction pathway, wherein the first binding agent is attached to a solid support; (c) providing a second binding agent that specifically binds to a second epitope on the second biomarker in the second signal transduction pathway, wherein said second binding agent is capable of attaching to a sensor; (d) mixing the sample, the first binding agent, and the second binding agent together under conditions whereby a sandwich assay formation will be formed if the sample contains the second biomarker comprising the first epitope and the second epitope; (e) presenting the result of step (d) to a sensor that emits an acoustic output signal; (f) detecting a change in the acoustic output signal of (f) as compared to an acoustic output signal of a sensor that is not presented the results of step (d); and (g) treating the damaged or diseased cell with an agent that targets at least one biomarker in the second signal transduction pathway.

The teachings of all references cited above and below are hereby incorporated herein by reference in their entireties. The following examples are provided only to further illustrate the invention, and are not intended to limit its scope. The present invention encompasses modifications and variations of the methods taught herein which would be obvious to one of ordinary skill in the art.

### EXAMPLES

### Example 1

### Detection of a Secreted Biomarker

To determine whether the methods described herein are able to detect a secreted biomarker, an assay to detect lactation-associated peptide hormone prolactin (PRL; also known as luteotropic hormone) was developed.

Hyperprolactemia is associated with hypoestrogenism and erectile dysfunction in men, and can result from prolactinomas and excess thyrotropin-releasing hormone (TRH), usually found in primary hypothyroidism (Jacobson et al., Discov Med. 11(59):315-324, 2011; PubMed PMID: 21524385). A robust assay that quantifie PRL in serum could be used as a diagnostic tool for various conditions. To better evaluate the effectiveness of the AMMP technology in detecting PRL, we conducted parallel experiments using previously developed, in-solution proximity ligation assays (isPLA). In unpublished results, the lower limit of detection for isPLA assay was found to be 1 pM for prolactin detection.

For these studies, recombinant PRL was titrated over a 3-fold dilution series to the indicated concentrations in buffer recommended by the manufacturer to generate standard curves using AMMP assay technology and isPLA techniques. An in-house rabbit polyclonal antibody was labeled with either biotin (for conjugation to streptavidin-magnetic beads) or FITC for use with the AMMP assay as directed by the manufacturer. With the isPLA technology, the same PRL antibodies used in the AMMP assay was labeled with biotin and conjugated with streptavidin-oligo A or B according to the manufacturer's protocols. 2 uL of sample was then incubated with 2 uL of probes at a final concentration of 62.5 pM and analyzed using isPLA. The mean and standard deviation of triplicate determinations of each PRL concentration were background-corrected, then plotted.

For standard curves of PRL with AMMP assay and isPLA using recombinant PRL doped into human serum, recombinant PRL was titrated over a 3-fold dilution series to the indicated concentrations in either buffer recommended by the manufacturer or Low Cross Buffer, LCB, (Candor Biosciences GmbH) supplemented with and without 50% human serum. The same PRL antibody conjugates utilized above were used with the AMMP assay as directed by the manufacturer. Recombinant PRL was titrated in the same manner and analyzed using is PLA technology as directed by the manufacturer.

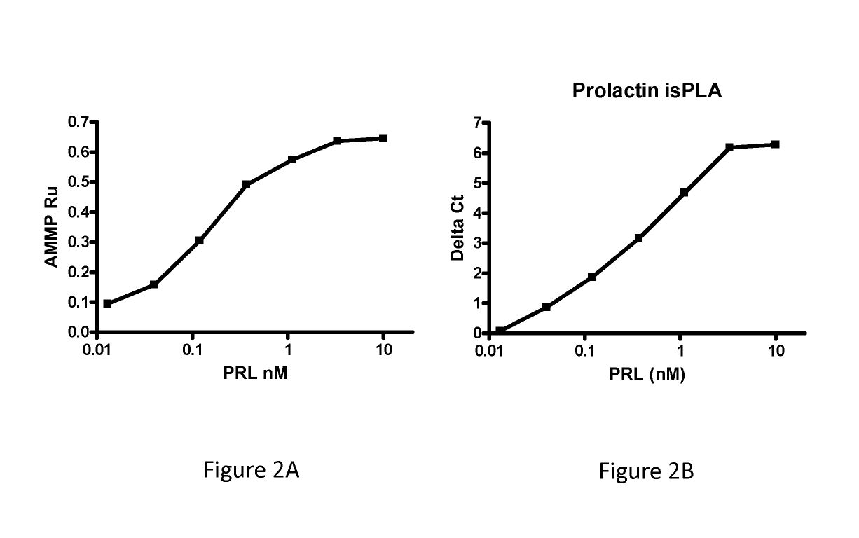

To evaluate the analysis of prolactin using AMMP assay technology (see FIG. 1), the limit of detection (LOD), dynamic range, and importantly, the effects of serum interference on assay performance was determined. The results obtained from an AMMP assay on the VIBE platform were then compared to an isPLA platform developed for PRL using the same antibody pairs. Further analysis of dilution buffer comparison to minimize sample matrix interference was also performed on both platforms and results were compared. Although Prolactin can be analyzed using other commercial technologies (e.g., ECL and ELISA), isPLA has been reported to be a sensitive detection method (see Fredriksson et al., Nat Biotechnol 20 (5): 473-477, 2002; Gullberg et al., Proc Natl Acad Sci USA 101(22): 8420-8424, 2004), so a comparison of the performance of isPLA to that of the AMMP assay technology was made. Using AMMP detection, recombinant PRL was used to generate a standard titration curve with an in-house-designed, rabbit polyclonal antibody. Both methods detected PRL over a 3-log detection range (see FIGS. 2A and 2B). The LOD of PRL detection was similar for both technologies, detecting PRL at approximately 10⁻¹² M. The results shown in FIGS. 2A and 2B represent the background-corrected mean and standard deviation of triplicate determinations for each PRL concentration.

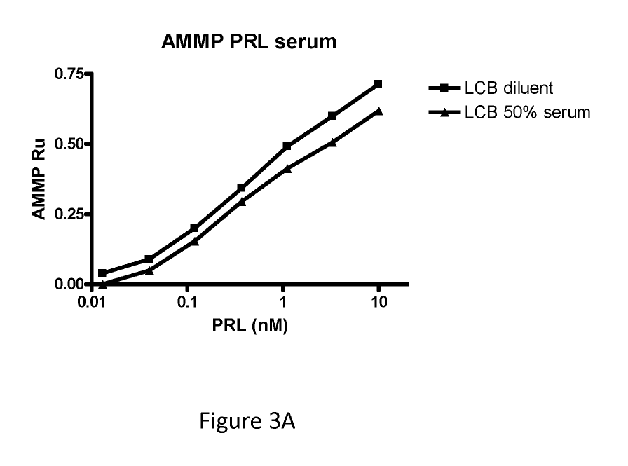

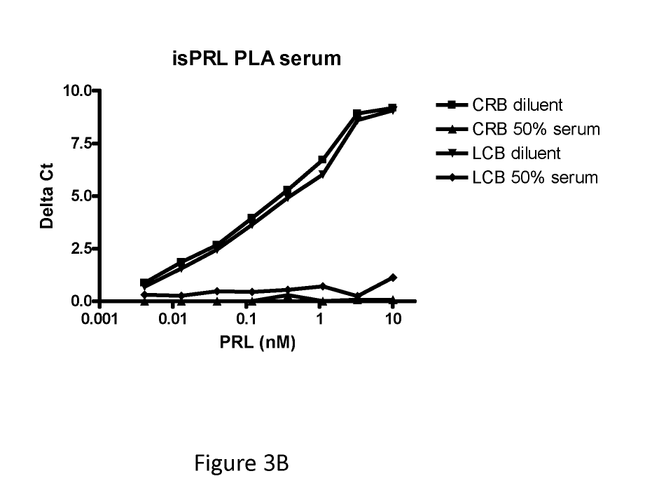

Considering that both platforms (AMMP/ViBE and isPLA) have potential to detect low-level biomarkers in a clinical setting for disease diagnosis, the abilities of both platforms to the detection of PRL doped (i.e., added) into human serum was next compared. The AMMP assay detected PRL doped into human serum samples diluted in a high salt PBS 1% BSA buffer (PBSBS) or Low Cross Buffer (LCB) with markedly less interference compared to the isPLA technology diluted in LCB or Cell Resuspension Buffer (CRB) (compare FIG. 3A (AMMP) with FIG. 3B (IsPLA)). Furthermore, serum interference was decreased in samples diluted in LCB (see LCB 50% serum) compared to those diluted in PBSBS, indicating LCB may be a valuable diluent for detection of PRL in human serum.

The results of this example demonstrated that detection of PRL by both AMMP and isPLA can be achieved at similar sensitivities over comparable dynamic ranges, but detection of PRL in serum significantly improved using the AMMP assay compared to the isPLA platform. The non-optical AMMP technology is most likely contributing to this major difference in serum interference. The isPLA platform utilizes enzymatic ligation, PCR amplification and detection with a fluorescent TaqMan Probe. The high complexity of serum samples is known to inhibit the ligation and PCR enzymes as well as quench the fluorescent readout associated with this technology.

### Example 2

### Detection of Prostate-Specific Antigen in Male Human Blood

Prostate-specific antigen (PSA) is a protein produced by normal prostate cells. Although PSA is normally found in semen, some PSA can escape the prostate and be found in blood serum. Rising levels of PSA in the serum are associated with prostate cancer in men. Using the methods described herein, if a human male has an elevated level of PSA in his serum, he may choose to have additional screenings for prostate cancer (e.g., rectal exam or a transrectal ultrasound).

For these methods, anti-PSA antibodies are purchased from Millipore, Billerica, Mass. (e.g., Catalog No. 05-761, mouse anti-human, clone 2F5). The antibodies are biotinylated according to standard methods (e.g., using the ChromaLink Biotin Labeling kit available from Solulink, Inc. (San Diego, Calif.) or the EZ-Link Amine-PEG-Biotin kit commercially available from ThermoScientific, Inc. (Waltham, Mass.)). A second anti-PSA antibody (rabbit anti-human PSA is purchased from Epitomics, Inc. (Burlingame, Calif.; Catalog No. AC-0070, clone EP109).

Magnetic beads coated with streptavidin and then mixed with the biotinylated antibodies to attach them. The sensor is coated with anti-rabbit antibodies.

A serum sample is taken from a man (e.g., by taking his blood and separating the cells from the serum), and the serum is mixed with the anti-PSA antibody coated magnetic beads and the free rabbit anti-human antibodies. If PSA is present in the man's serum, a sandwich assay formation will be formed. The mixture (i.e., possibly containing a sandwich assay formation) presented to the anti-rabbit antibody-coated sensor and, the magnet applied. Under flow conditions, the acoustic output signal from the sensor is obtained and measured. This acoustic output signal can be compared to the acoustic output signal from a sample with a known amount of PSA to determine how much PSA is in the man's serum. The acoustic output signal can also be compared to the amount of PSA in the serum of a healthy man to determine if the man whose serum is being tested has an elevated level of PSA.

### Example 3

### Detection of a Cell Surface Biomarker

To determine whether the methods described herein can be used to detect a cell surface biomarker, detection of proto-oncogene cMET was assayed.

The proto-oncogene cMet encodes the receptor tyrosine kinase (RTK) hepatocyte growth factor (Bottaro et al., Science 251(4995): 802-804. 1991). Aberrant cMet activation occurs in numerous solid tumor types, and correlates with poor prognosis (Peruzzi and Bottaro Clin Cancer Res. 12(12):3657-60, 2006; PubMed PMID: 16778093). Autocrine, paracrine, amplification, and mutational activation of cMet trigger tumor growth, angiogenesis and metastasis.

cMet and its ligand are involved in all stages of malignant progression and represent promising biomarkers for targeted cancer therapies (Wang et al., Mol Cancer Ther. 10(11): 2022-2023. 2011). Although many are being tested, a few cMet inhibitors have entered clinical trials, and cMet diagnostic tests are lacking. Additionally lacking are tests to determine whether the cMET inhibitors are initially and/or continuous decreasing the amount of aberrantly expressed cMET.

The assay described in this example is capable of measuring cMet levels in cell lysates and in intact HepG2 cells (i.e., a human liver hepatocellular carcinoma cell line commercially available from the American Type Culture Collection (ATCC), Manassas, Va.; catalog no. HP-8065). This assay can be used to diagnose patients who aberrantly express cMET, to test whether a cMET inhibitor is initially reducing cMET expression, and whether the cMET inhibitor, after repeated administration, continues to be effective in reducing cMET expression.

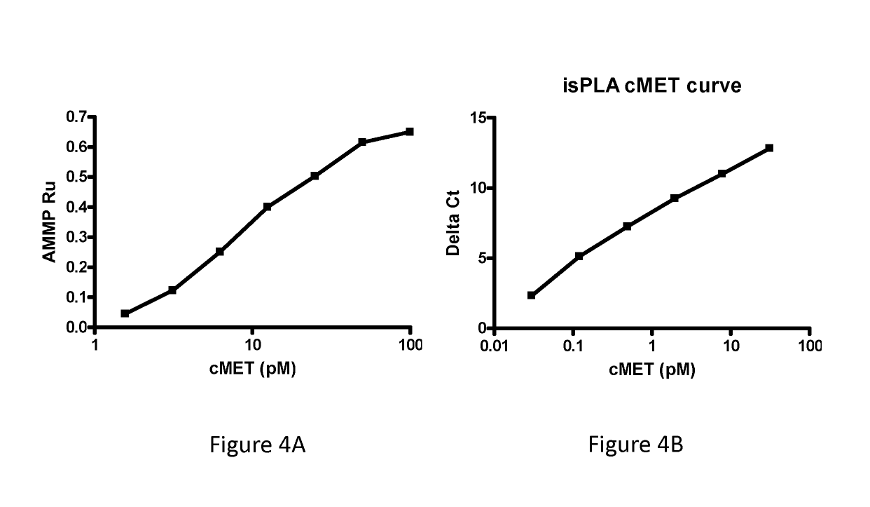

For these studies, a commercially available cMet antibody was used to further validate the AMMP assay technology. As with the PRL assay described above, a standard curve was generated using both AMMP assay technology and isPLA technology using a recombinant human HGF-R/cMet-Fc chimeric protein (see FIGS. 4A and 4B, respectively). The lower limits of detection for isPLA assay were found to be 0.1 pM for cMET detection, and fully optimized isPLA technology detected cMet at concentrations as low as 5×10⁻¹⁴M. Under similar conditions, an unoptimized AMMP assay was capable of measuring cMet at concentrations of 1×10⁻⁹ M. These experiments demonstrated that both technologies are capable of detecting cMet sensitively and that these antibodies are an appropriate pairing for use in AMMP assays. The results shown in FIGS. 4A and 4B are representative of the average of triplicate determinations of each cMET concentration with background subtracted.

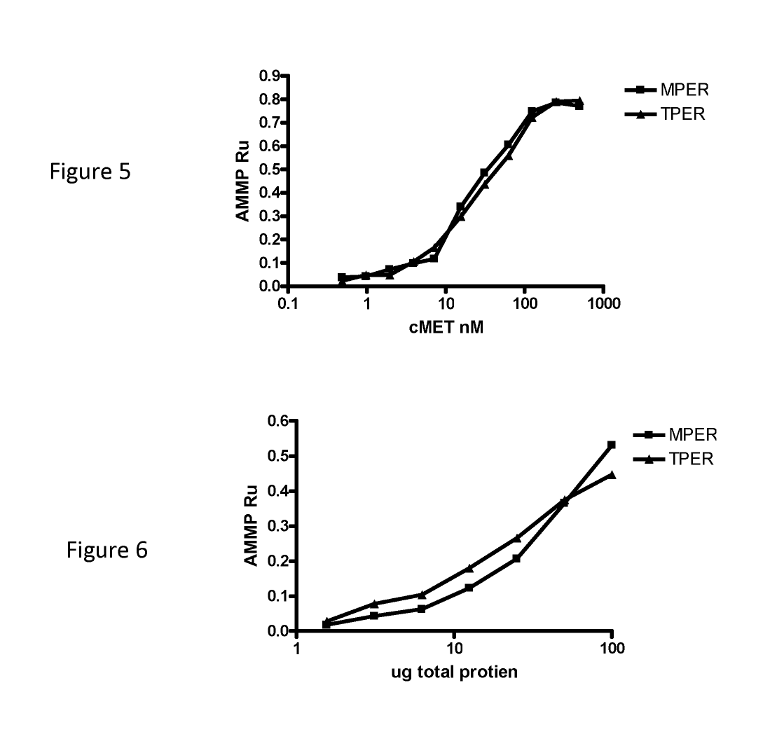

Detection of recombinant cMet was tested in two different, widely used lysis buffers: M-Per and T-Per (also called MPER and TPER, both commercially available from ThermoScientific (Pierce), Waltham, Mass.), because the ultimate goal of this assay was to detect and quantify cMet levels in cell lines of varying origin and tumor grade. Recombinant cMet was detected equally well using the AMMP technology in both M-Per and T-Per lysis buffers (see FIG. 5). Although the sensitivity was slightly diminished in these buffers compared to the sensitivity of cMet detection in the manufacturer's recommended diluent buffer, the assay is still sufficiently sensitive to detect endogenous cMet levels below 10⁻¹²M and with high confidence. Similar to the detection of recombinant cMet, the isPLA technology exhibited more sensitive detection of endogenous cMet compared to the unoptimized AMMP assay (data not shown).

Interestingly, an obvious increase in sensitivity and dynamic range for endogenous cMet was observed in samples of HepG2 cell lysates prepared with T-Per vs M-Per lysis buffer using the AMMP technology (FIG. 6). This is most likely due to the more stringent extraction capabilities of T-PER buffer towards membrane-spanning receptors. Thus, not only do these data show that the choice of buffer is critical for maximizing extraction of cMet, but they also demonstrate that even in a buffer containing more detergent and a more complex matrix, there is minimal, if any, loss in sensitivity. This is in stark contrast to buffers used in the isPLA technology, where more interference is elicited in more complex buffers with more complex and higher concentrations of detergents. The results shown in FIGS. 5 and 6 are representative of the background-corrected mean and standard deviation of triplicate determinations of each cMET concentration.

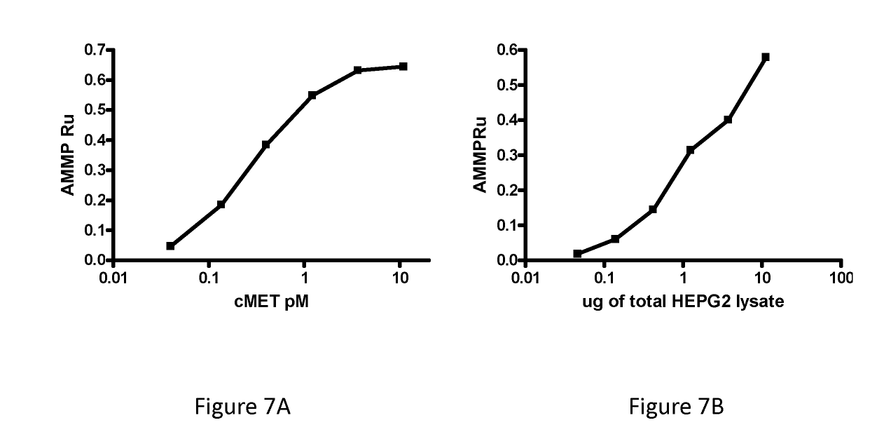

After initial range-finding experiments and lysis buffer compatibility, the AMMP assay was next optimized in order to achieve similar sensitivity to the isPLA assay described above. By increasing the amount of recombinant cMET antibody on the magnetic bead surface by an additional 150 pg per well, the AMMP assay achieved similar sensitivity to the isPLA assay (see FIG. 7A). Increasing the antibody concentration on the beads increased the sensitivity of the assay from 1 pM to below 0.1 pM. Correspondingly, the new assay was able to detect cMET in 100 ng of total HEPG2 lysates, an order of magnitude more sensitive than the isPLA assays (see FIG. 7B).

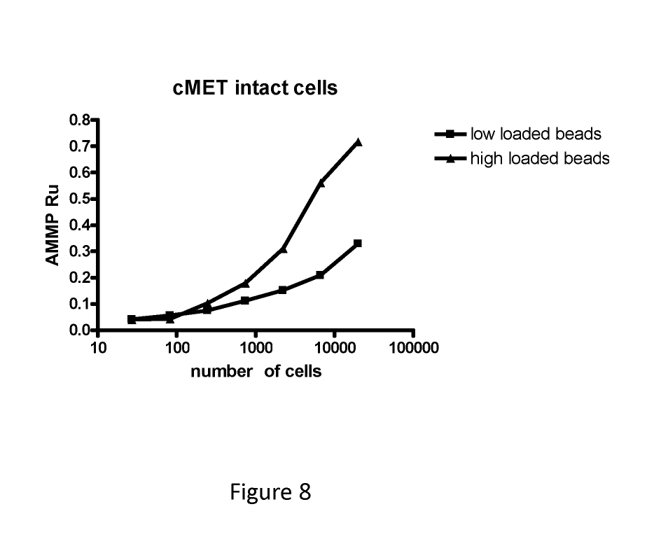

Next, an assay was performed to determine if cMET-positive HEPG2 cells could be quantified using the AMMP technology on whole, unlysed cells. In this scenario, the sandwich assay formation was comprised of anti-cMET antibody-attached magnetic beads/HepG2 cells expressing cMET at their cell surface/free anti-cMET antibody. The higher-loaded beads (see square symbols, FIG. 8) were able to detect well below 1,000 cells per sample. Beads optimally loaded with anti-cMET antibody were compared to beads with lower loads to assess sensitivity (see FIG. 8).

These experiments indicated that a single AMMP assay was able to quantify recombinant cMET, cMET in cell lysates and cMET on intact cells with greater sensitivity than the isPLA assays.

Thus, using the AMMP assay technology, endogenous cMet levels in cell lysates and in intact cells was quantifiable. This technology leads to the possibility of developing a cMet diagnostic assay for use on human samples. It also provides a very rapid method for quickly determining whether a candidate is capable of reducing cMET levels in a treated cell.

### Example 4

### Detection and Analysis of Cytosolic Biomarkers

To determine whether the methods described herein can be used to detect a cytosolic protein, a protein involved in the MAP kinase signal transduction pathway was assayed.

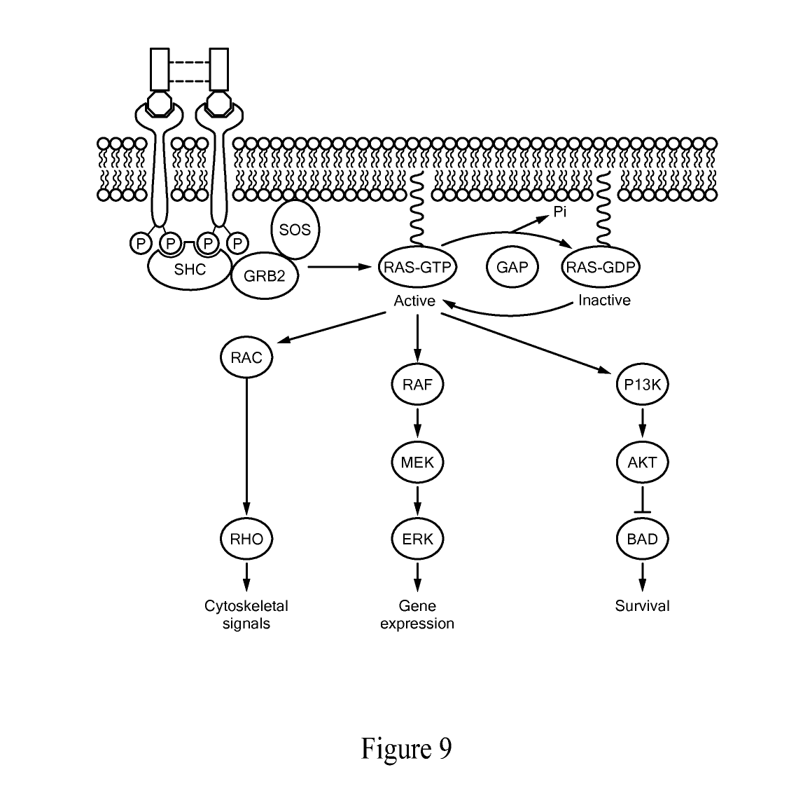

The mitogen-activated protein kinase (MAPK) cascade is a key signal transduction pathway that governs cellular communication between protein receptors on the cell surface and the cell nucleus. Mitogen-activated protein (MAP) kinases are an evolutionarily conserved family of enzymes that play an important role in modulating a variety of cellular processes, including proliferation, differentiation and apoptosis. Four major groups of MAP kinases have been identified in mammalian cells, these are known as the extracellular signal-regulated kinases (ERK 1 and 2), the c-Jun amino-terminal kinases (INK), the p38 kinases (p38) and ERK5. As shown in FIG. 9, following activation of ras, MAP kinases are part of a three-tiered cascade consisting of a MAP kinase (e.g., MAP3K or Raf), a MAP kinase kinase (MAPKK, MKK or MEK) and a MAP kinase kinase kinase (MAPKKK or MEKK).

FIG. 9 also shows the phosphoinositide 3-kinase (PI3K-AKT) cascade which, together with the MAPK cascade, is a signal transduction pathway which heavily influences the regulation of cell proliferation, differentiation, and survival, and as such, are implicated in a wide variety of cancers.

Of course FIG. 9 is just a smaller part of a larger signal transduction network contained within a cell. FIGS. 10A and 10B show a larger part of this network, but the true network is larger, and the relationships between the individual biomarkers are still not fully understood. The ability to measure the activation states and molecular interactions of the kinases comprising these pathways are crucial in the efficient and effective development of anti-oncological pharmaceuticals and therapeutics. Moreover, if different members of these pathways can be accurately measured, the activity of the entire pathway can be monitored to assess any change, transformation, or resistance during the lifetime of a cell including during carcinogenesis or during the cell's response to a treatment (e.g., treatment with a chemotherapeutic). The activity can also be monitored to determine if the cell is developing resistance to a treatment, for example, by using a salvage pathway to bypass the member of the pathway targeted by the therapeutic.

The MAPK signaling pathway is among the most frequently dysregulated in human cancers. The MAPK kinases MEK1 and MEK2 mediate the extracellular signal-regulated kinase (ERK), which has received considerable research attention in cancer treatment as a potential target for inhibitors (Ohren et al., Nature Structural and Molecular Biology 11: 1192-1197. 2004; Sebolt-Leopold et al., Nat Rev Cancer 4 (12): 937-947. 2004. PMID 15573115; Catalanotti et al., Nature Structural and Molecular Biology 16: 294-303. 2009; Roberts and Der Oncogene 26(22): 3291-3310, 2007). Although approximately 30% of all human cancers display evidence of constitutive activation of the MAPK pathway, only 1% exhibit mutations in MEK1/2, and as of yet, no mutations have been reported in ERK1/2 (Schubbert et al., Nat Rev Cancer. 7 (4): 295-308, 2007. 17384584; Fremin and Meloche, J Hematol Oncol. 3:8, 2010. PMID 20149254; Davies et al., Nature. 417 (6892): 949-954, 2002. PMID 12068308. To date, eleven MEK1/2 inhibitors have either been tested or are undergoing clinical trial evaluation (Fremin and Meloche, J Hematol Oncol. 3:8, 2010. PMID 20149254). Small molecule MEK1/2 inhibitors significantly inhibit the proliferation of numerous carcinoma and leukemia cell lines through cell cycle arrest and induction of apoptosis (Dudley et al., Proc Natl Acad Sci USA 92(17): 7686-7689. 1995 PMID 7644477; DeSilva et al., J Immunol. 160 (9): 4175-4181. 1998. PMID 9574517; Sebolt-Leopold et al., Nat Med 5(7): 810-816. 1999. PMID 10395327). It is hypothesized that down regulation of MEK1 levels may reduce signaling and disrupt the positive feedback loop to Rafkinase, which enhances MEK activity. The phosphorylation of MEK on residues 5218 and 5222 is catalyzed by Rafkinase, which leads to the activation of the MAPK pathway. MAP kinase family members are also phosphorylated on the threonine and tyrosine residues in the T-loop of a MAP kinase family member, resulting in activation.

Upon activation of MEK1 upon phosphorylation at serine218 and serine222, ERK2 is directly phosphorylated, leading to its release from MEK1 and subsequent nuclear translocation.

Sensitive detection of total MEK, pMEK (S218/S222), and ERK/MEK heterodimer levels in both human cell lines and clinical samples would be a key enabler for the development of therapeutic approaches (Hoshino et al., Oncogene. 18 (3): 813-822. 1999 PMID 9989833; Raman et al, Oncogene. 26(22): 3100-3112. 2007. PMID 17496909; Rajakulendran et al., Nature 461: 542-545. 2009). Immunoassays capable of detecting and quantifying pMEK levels, pMEK/MEK ratios, and ERK/MEK dimers could be readily utilized to screen libraries of small molecules or peptides directed toward inhibition of the MAPK pathway, including not only direct inhibitors of kinase activity, but also inhibitors of the MEK/ERK interaction in cells.

Using AMMP technology, this example describes the development of assays for pMEK and total MEK measurement. In addition, an assay to detect and quantify the MEK/ERK protein heterodimer has been developed, which could be used in conjunction with the other assays to screen drug libraries and evaluate the efficacy of drug candidates in patients.

For these studies, standard curves of MEK concentration were determined using AMMP assay technology. Baculovirus-produced MEK (using reagents commercially available from Invitrogen, Carlsbad, Calif.) was titrated over a 3-fold dilution series to the indicated concentrations in buffer recommended by the manufacturer. An anti-MEK N-terminus rabbit monoclonal antibody (Epitomics, Burlingame, Calif.) was labeled with biotin and conjugated to streptavidin-magnetic beads, and an anti-MEK C-terminus rabbit monoclonal antibody (Epitomics, Burlingame, Calif.) was labeled with FITC for use with the AMMP assay. Logarithmically growing HepG2 cells were lysed in M-PER buffer and protein concentrations were determined using the Pierce BCA Protein Assay kit (Thermo Scientific) according to manufacturer's instructions. Lysates were titrated over a 3-fold dilution series to the indicated total protein amounts in 25% M-PER buffer. The same MEK antibody pairing as in the previous experiments was employed for use with the AMMP assays.

For detection of pMEK S218/S222 in lysates with and without addition of a pMEK-specific peptide, logarithmically growing HepG2 cells were lysed in M-PER buffer and protein concentrations were determined using the Pierce BCA Protein Assay kit (ThermoScientific, Waltham, Mass.) according to manufacturer's instructions. Lysates were titrated over a 3-fold dilution series to the indicated total protein amounts in 25% M-PER buffer. A pMEK specific peptide used to immunize rabbits for production of the pMEK antibody was added to the indicated samples to a final concentration of 10 nM. An anti-MEK N-terminus rabbit monoclonal antibody (Epitomics) was labeled with FITC and an anti-pMEK S218/S222 rabbit monoclonal antibody (Epitomics) was labeled with biotin and conjugated to streptavidin-magnetic beads for use with the AMMP assay.

For the detection of ERK/MEK dimers in lysates with and without addition of a MEK/ERK interaction blocking peptide, Baculovirus-produced MEK1 (Invitrogen) and ERK2 (Protein One) were incubated together at a concentration of 10 nM, and then titrated over a 3-fold dilution series to the indicated concentrations in buffer recommended by the manufacturer. An anti-MEK C-terminus rabbit monoclonal antibody (Epitomics) was labeled with FITC and an anti-ERK rabbit polyclonal antibody (Cell Signaling Technology, Inc.) was labeled with biotin then conjugated to streptavidin-magnetic beads for use with the AMMP assay. In this assay, the sandwich assay formation, if formed, would comprise the anti-ERK antibody-coated magnetic beads/ERK-MEK dimer/anti-MEK FITC labeled antibody. For the isPLA experiments, logarithmically growing HepG2 cells were lysed in M-PER buffer and protein concentrations were determined using the Pierce BCA Protein Assay kit (Thermo Scientific) according to manufacturer's instructions. Lysates were titrated over a 3-fold dilution series to the indicated total protein amounts in 25% M-PER buffer. An ERK/MEK peptide that competes with ERK for the binding site on MEK was added to the indicated samples to a final concentration of 200 nM.

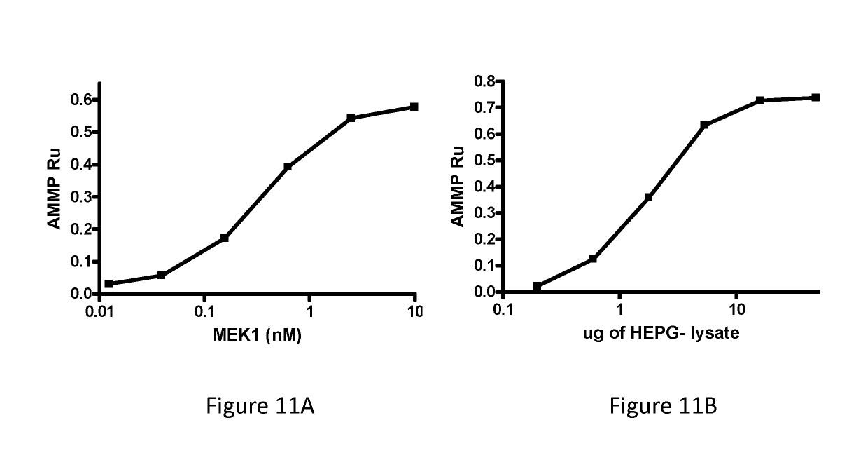

The results of in this example demonstrate the flexibility and feasibility of applying the AMMP assay technology and the VIBE platform to a pharmaceutical or drug screen setting. The MAPKinase (MAPK) pathway was chosen for these experiments due to the considerable research and drug development aimed in this direction. The MEK/ERK pathway is hyperactivated in a majority of known cancers. The AMMP assay technology was used to generate a standard curve utilizing recombinant MEK1 protein with an LOD of 0.05 nM over a 4-log dynamic range (see FIG. 11A). Endogenous MEK1 in HepG2 lysates was also detected using AMMP technology over a 3- to 4-log dynamic range with as little as 0.5 μg of lysate within the linear range of the curve (see FIG. 11B).

The AMMP assay technology was also used to develop sensitive detection assays for total MEK1, pMEK1 (S218/222), and ERK/MEK dimers. Effective assays for these analytes allow for the characterization and quantification of the activation status of the MAPK pathway. Since inhibitors of the formation of ERK/MEK heterodimers are attractive targets for oncology therapies, detection and quantification of the dimer in cells could offer a tool for assessing efficacy of drug candidates, and informed drug design and discovery.

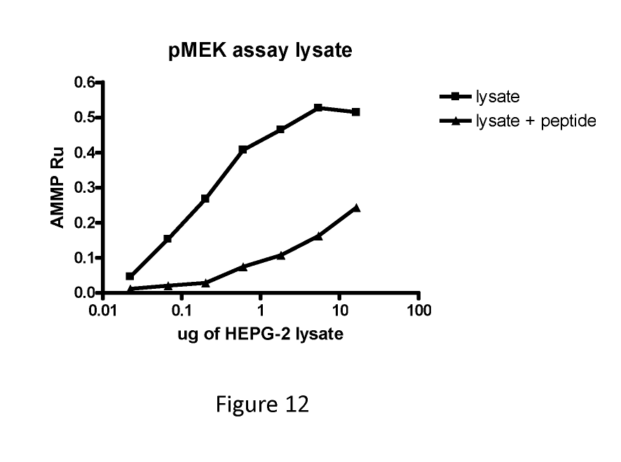

A pMEK and N-terminal MEK1 antibody pairing was utilized to detect pMEK in cell lysates. Comparison of these results with the results from the total MEK assay allows for the determination of the pMEK/MEK ratio, which is critical for evaluating the efficacy of drugs that target the MEK1 kinase. Levels of pMEK were computed with as little as 50 ng of total lysate (see FIG. 12).

To further assess the stringency of the assay, the same lysate dilution series was treated with the pMEK peptide utilized to generate the pMEK antibody used in this immunoassay at a constant concentration of 5 nM. As shown in FIG. 12, with decreasing amounts of lysate, the signal for pMEK detection was competed away with the constant concentration of peptide.

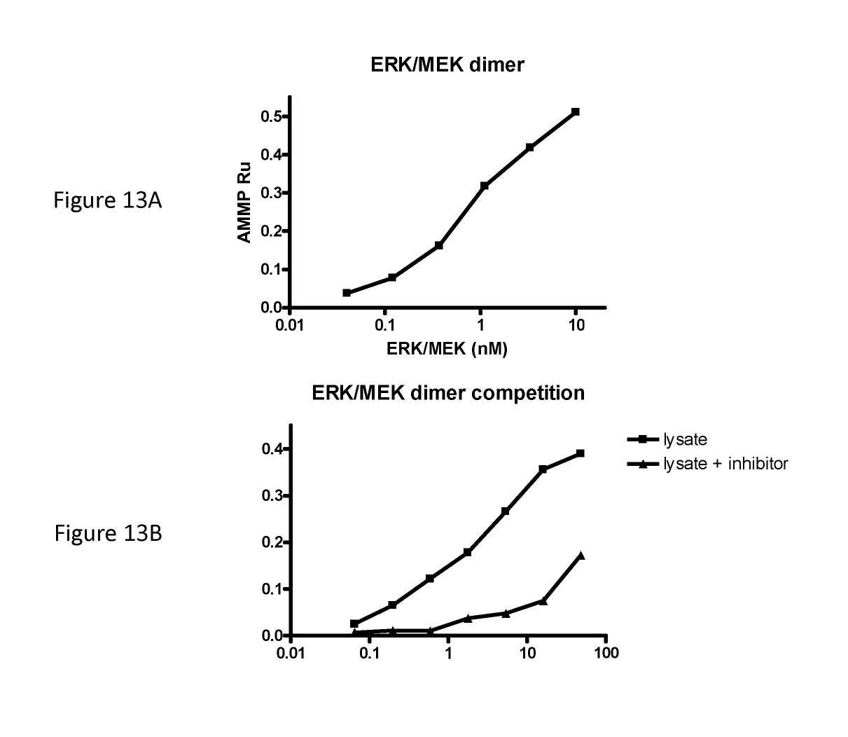

Next, an assay was performed to determine if protein-protein interactions in the MAPK signal transduction pathway could be detected using the AMMP assay technology to a level of sensitivity comparable to the total protein and phosphorylated proteins described above. Utilizing recombinant MEK1 and recombinant ERK2, a standard curve was generated for detection of ERK/MEK heterodimers, ERK/MEK dimers were detectable with a LOD of around 0.05 nM over a 3-log dynamic range (see FIG. 13A). As controls, ERK2 and MEK1 were incubated independently and were not detectable using an ERK/MEK antibody pairing (data not shown), demonstrating high antibody specificity and minimal cross reactivity. HepG2 lysates were then used to determine the concentration of ERK/MEK dimers in cells. Dimers were detectable with as little as 100 ng total lysate over a 4-log dynamic range (see FIG. 13B). Furthermore, a peptide inhibitor was utilized that mimics the ERK binding region of MEK and directly competes with endogenous ERK for MEK binding. As shown in FIG. 13B, using a constant concentration of 200 nM peptide inhibitor in each of the diluted lysate samples, the peptide reduced the endogenous ERK/MEK interaction by 60%, with 80 μg of lysate and almost completely with 5 μg of total lysate. Thus, the AMMP assay technology has proven its capability to detect low levels of biomarkers, phosphorylation events, and protein-protein interactions.

Next, the dimerization state of MEK with ERK and phosphorylated MEK with ERK was assessed using the methods described herein. Previous immunoprecipitation assays have shown that MEK and ERK exist in a heterodimer but no publication has shown that the active state of MEK in the dimer state with ERK.

For sample preparation, the materials used and the vendors from which the materials were purchased are shown in Table 2.

TABLE 2

| | Vendor from which | | |
|---|---|---|---|
| Material | material was purchased | Catalog # | Lot # |
| MCF-7 cells | ATCC, Manassas, VA | | n/a |
| A431 cells | ATCC | CCL-163 | n/a |
| Mammalian Protein | ThermoFisher/Pierce, | 78505 | 157931, |
| Extraction Reagent | Rockford, IL | | 157138 |
| Dimethyl sulfoxide | Sigma Aldrich, St. | 472301-2L | 26696MJ |
| (DMSO) Endothelial | Louis, MO | | |
| growth factor | | | |
| Phorbol 12-myristate | Sigma Aldrich | P8139 | 031M1775V |
| 13-acetate | | | |
| Cell Culture Medium | ATCC | 30-20 | 30001181 |
| RPMI 1640 | | | |
| Halt* Phosphatase | ThermoFisher/Pierce | 78428 | ME156486 |
| Inhibitor Cocktail | | | |
| Halt* Protease | ThermoFisher/Pierce | 78430 | MD156798 |
| Inhibitor Cocktails | | | |

MCF-7 human breast cancer cells and A431 human epithelial carcinoma cells (both commercially available from the American Type Culture Collection (“ATCC”), Manassas, Va.) were grown in media as specified by ATCC. Cell lines were maintained and split when appropriate. When cells were approximately 80% confluent in a T75 flask, they were prepared for analysis. Growth media was removed from each flask and replaced with 10 milliliters of basal media supplemented with or without 100 ng/ml of endothelial growth factor. Each flask was return to the 37° C. incubator for 10 minutes. Following the 10 minute incubation, 3 ml of cold T-PER lysis buffer (commercially available from ThermoScientific, supplemented with proteases and phosphatase inhibitors) was added to each flask. The cells were scrapped from the flask when necessary especially in the case of the A431 cell line. The lysates were pipetted into 50 ml conical tubes and placed on ice. Through a series of alternating vigorous vortex mixing steps and sitting on ice, the lysis was completed. Aliquots of each lysates were placed in 1.7 mL Eppendorf tubes and placed on dry ice before being transferred to −80° C. freezer.

For the AMMP assay to be performed on a VIBE platform, the materials used and the vendors from which the materials were purchased are shown in Table 3.

TABLE 3

| Material | Vendor | Catalog # | Lot # |
|---|---|---|---|
| Bead-pMEK antibody coupled | BioScale, Inc., | n/a | |
| | Lexington, MA | | |
| Fl-labeled-Antibody-total | BioScale, Inc. | n/a | |
| MEK | | | |
| Diluent | BD Biosciences, | 555213 | 84293 |
| | San Jose, CA | | |
| Mammalian Protein Extraction | ThermoFisher/ | 78505 | |
| Reagent | Pierce | | |
| Lysate (MCF-7 & A431) | BioScale, Inc. | — | — |
| PBS-Tween | BioScale, Inc. | n/a | n/a |
| 25 mM Phosphoric Acid | BioScale, Inc. | n/a | n/a |

The assay for detecting a MEK-ERK heterodimer was constructed as follows. Streptavidin-coated paramagnetic beads were coupled with 0.5 ug of biotinylated rabbit anti-MEK (Epitomics, Inc., Burlingame, Calif.; Catalog #1235) antibodies per milligram of bead. Antibodies to total ERK (Cell Signaling Technology, Inc., Danvers, Mass.; Catalog #3374) were labeled with fluorescein for ViBE assay detection reagents. These reagents were diluted for assay use in diluent (commercially available from BioScale, Inc., Lexington Mass.) to four times the final desired concentration. Beads were diluted to 8×10⁷ and fluorescein labeled antibody to 1200 ng/ml. The samples were diluted using MPER (ThermoScientific) to twice the final desired protein concentration when diluted 1:1 with Bioscale diluent. Assays were run in a sandwich format, with simultaneous incubation of the sample with the beads and fluorescein labeled antibody. To set-up the assay plate 80 ul of samples and 40 ul of reagents were added to the desired wells.

Similarly, the assay to detect pMEK-ERK heterodimer was constructed as follows. Streptavidin-coated paramagnetic beads were coupled with 2 ug of biotinylated rabbit anti-phosho MEK (Cell Signaling Technology, Inc. #9154) antibodies per milligram of bead. Antibodies to total ERK (Cell Signaling Technology, Inc. #3374) were labeled with fluorescein for ViBE assay detection reagents. The assay as constructed just as above using the same concentrations of beads and fluorescein labeled antibody.

Cell lysate samples were prepared for the assays by thawing them on ice, then clarifying by centrifugation in a microcentrifuge at 14,000 RPM for 10 minutes. Supernatants were removed from the samples and the total protein concentrations were adjusted in MPER for the assays. Serial two-fold dilutions were also prepared in MPER and subsequently assayed in triplicate. Forty microliters of lysate sample were assayed for each dilution, added to 40 ul of assay diluent (Becton Dickinson, Franklin Lakes, N.J.). The sample/reagent incubation time together was 4 hours prior to analysis on the ViBE. Elapsed time to achieve results was approximately 8 hours. Negative controls containing the bead only with the samples (no fluoresceinated antibody) were run and the ViBE response was subtracted from the sample signal readings to determine the specific assay signal.

From these experiments, results were obtained for two days in two separate cell lines for each of the MEK-ERK dimers and the pMEK-ERK dimers.

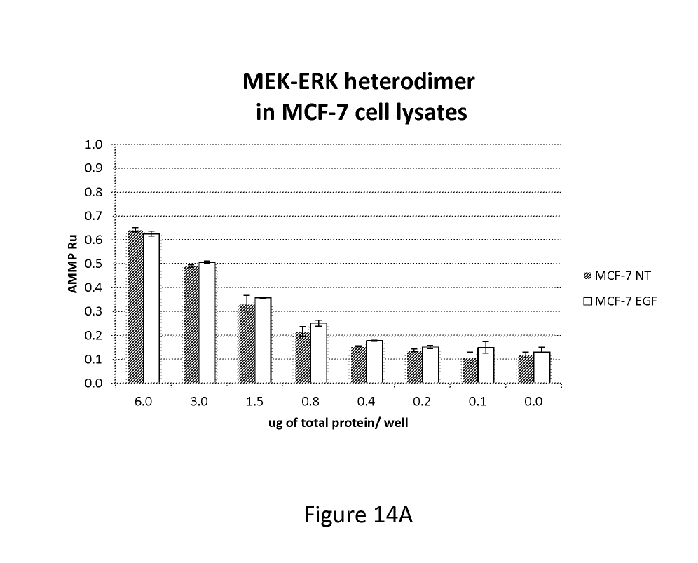

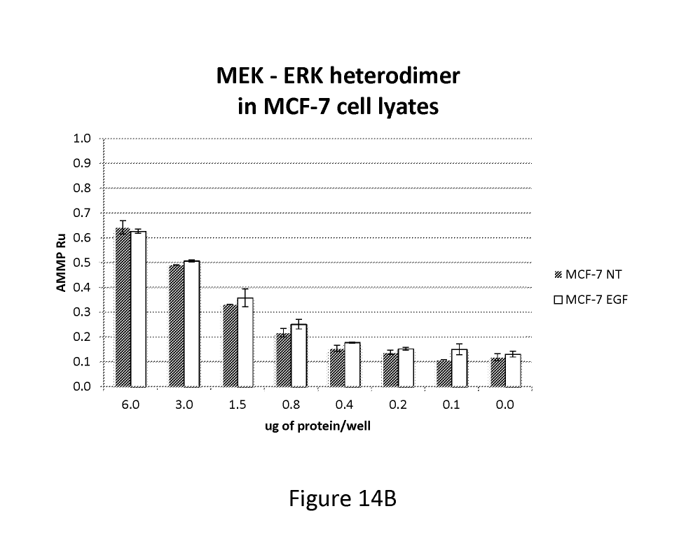

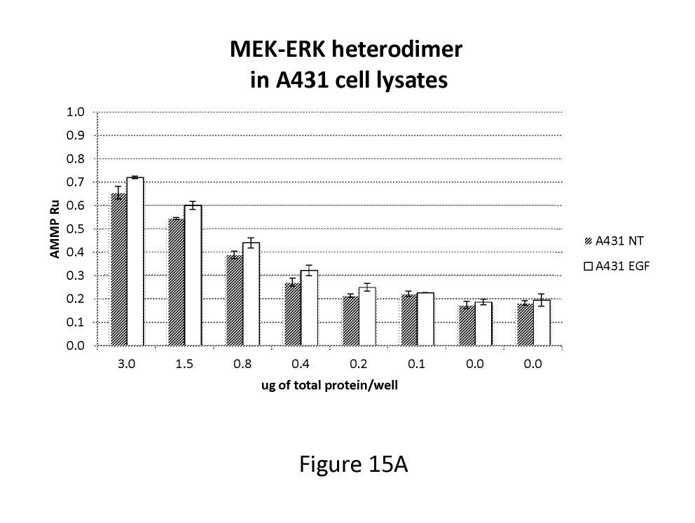

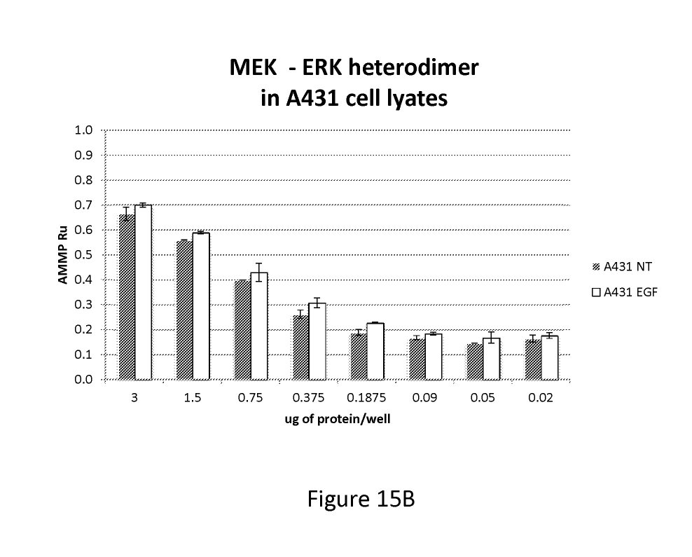

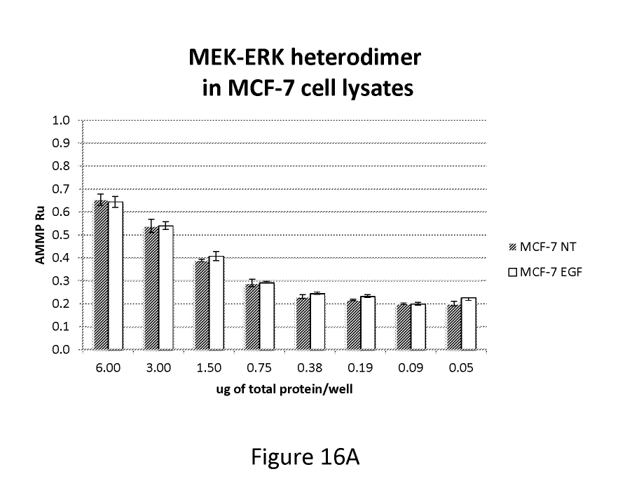

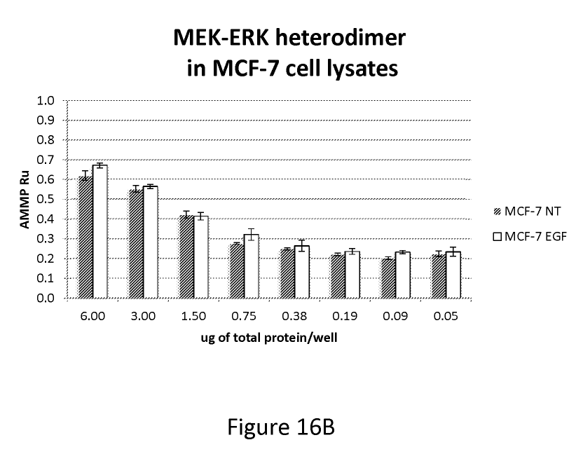

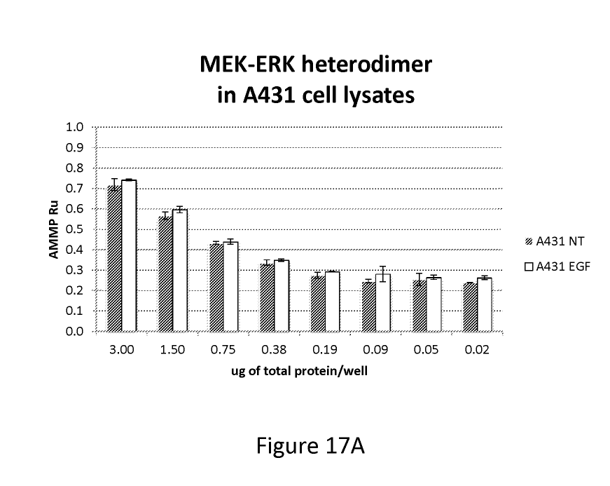

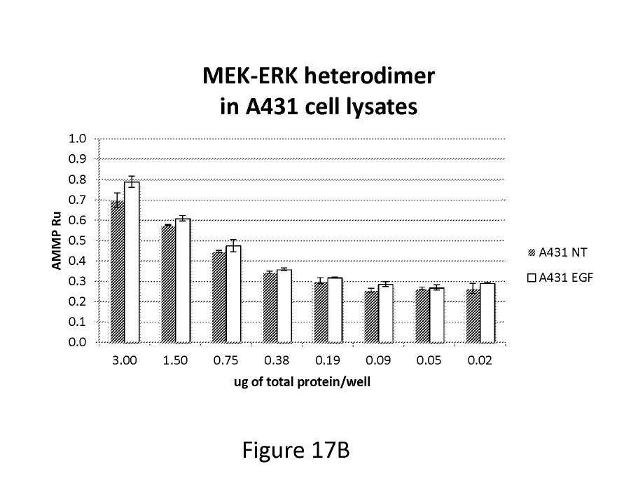

For day 1 for the MEK-ERK dimers (also called heterodimers, since the two molecules in the dimer are different), lysates from MCF-7 and A431 prepared on two separate days were analyzed for the expression of MEK-ERK heterodimers. The results from the MCF-7 cells are shown in FIGS. 14A and 14B, and the results from A431 cells are shown in FIGS. 15A and 15B. Each cell line was either treated with EGF or left untreated. For day 2 for the MEK-ERK dimers Lysates from MCF-7 and A431 prepared on two separate days were analyzed for the expression of MEK-ERK heterodimers. The results from the MCF-7 cells are shown in FIGS. 16A and 16B, and the results from A431 cells are shown in FIGS. 17A and 17B. Each cell line was either treated with EGF or left untreated. The expression results shown in FIGS. 14A-17B were analyzed at 7 dilutions and each dilution was run in duplicate, with the graphs in the figures showing mean values with standard deviation of error.

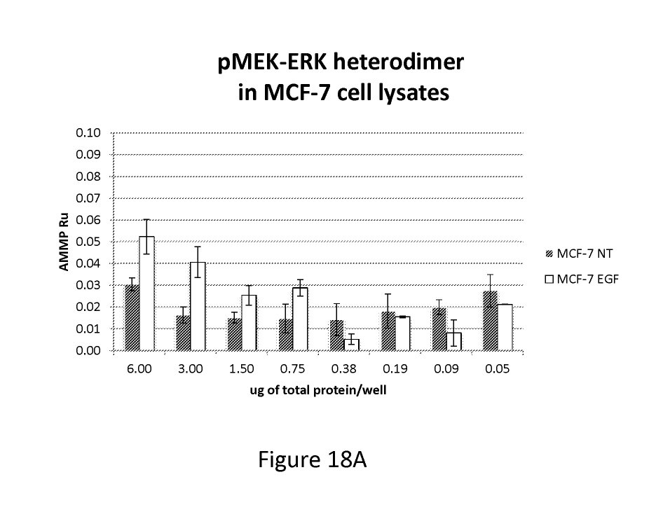

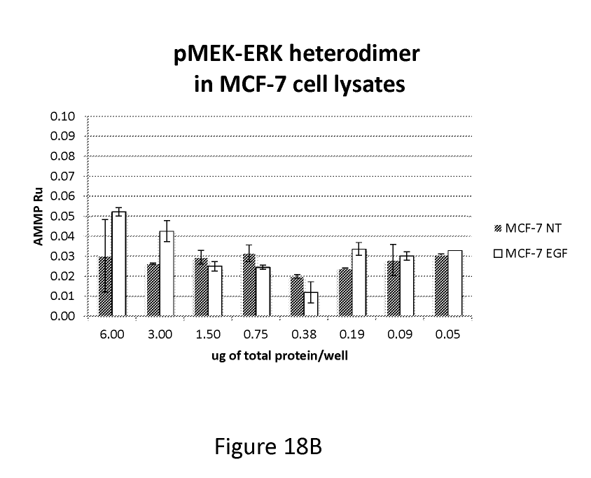

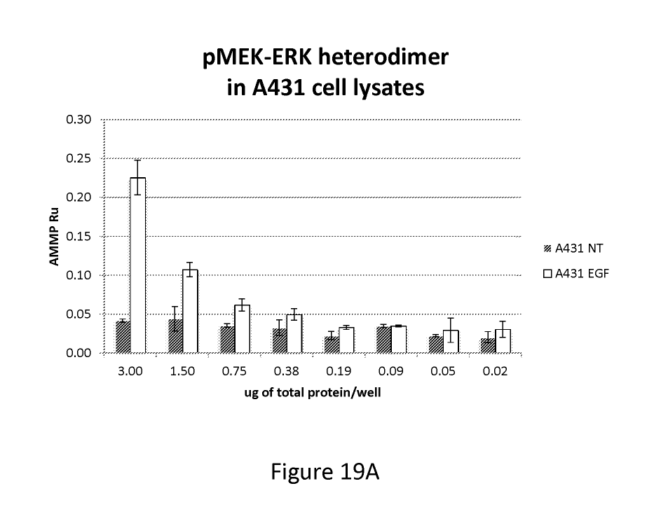

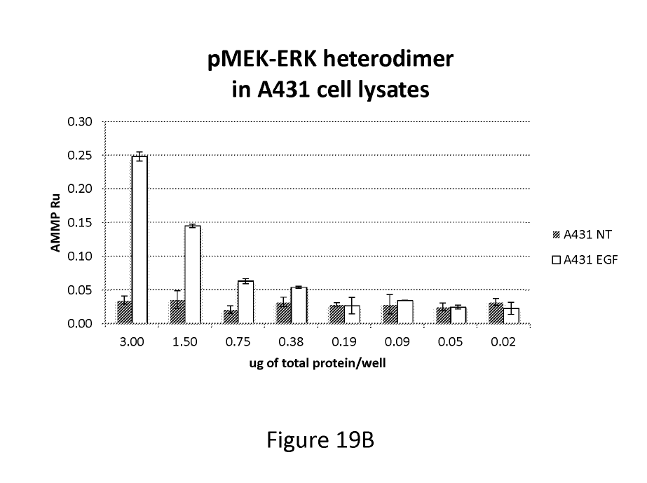

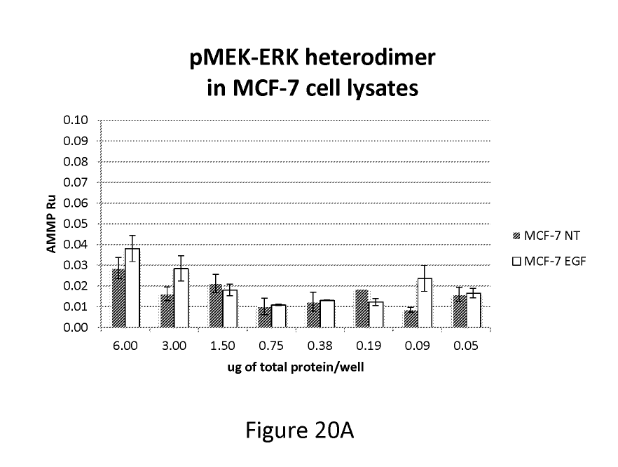

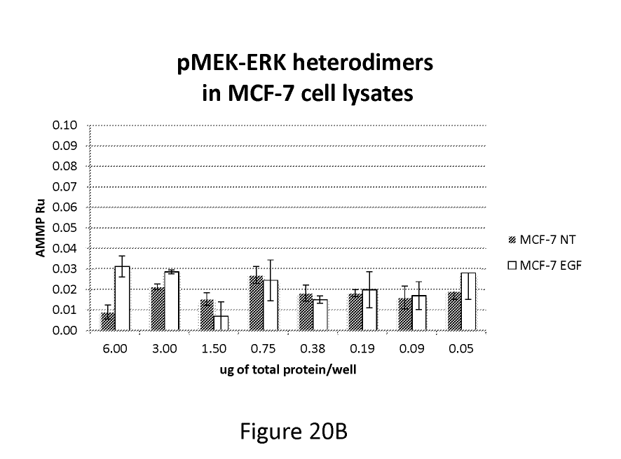

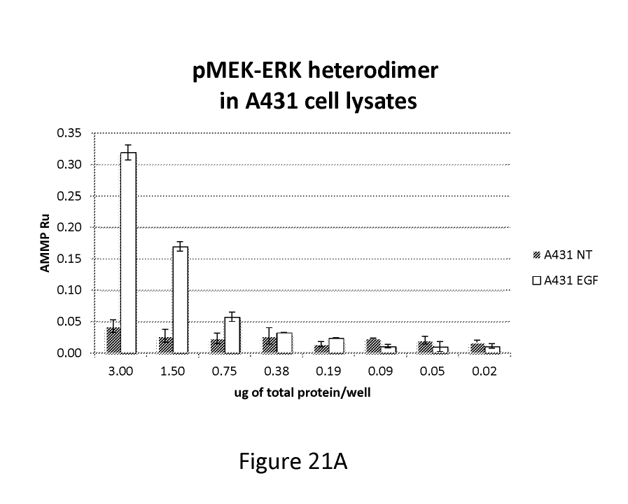

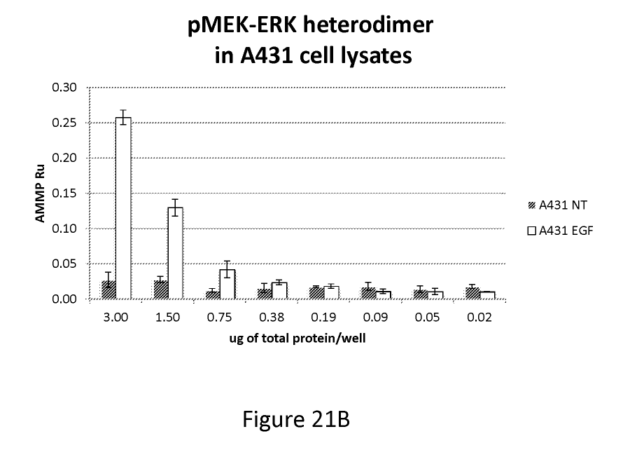

For day 1 for the pMEK-ERK dimers (also called heterodimers, since the two molecules in the dimer are different), lysates from MCF-7 and A431 prepared on two separate days were analyzed for the expression of pMEK-ERK heterodimers. The results from the MCF-7 cells are shown in FIGS. 18A and 18B, and the results from A431 cells are shown in FIGS. 19A and 19B. Each cell line was either treated with EGF or left untreated. For day 3 for the pMEK-ERK dimers, lysates from MCF-7 and A431 prepared on two separate days were analyzed for the expression of pMEK-ERK heterodimers. The results from the MCF-7 cells are shown in FIGS. 20A and 20B, and the results from A431 cells are shown in FIGS. 21A and 21B. Each cell line was either treated with EGF or left untreated. The expression results shown in FIGS. 18A-21B were analyzed at 7 dilutions and each dilution was run in duplicate, with the graphs in the figures showing mean values with standard deviation of error.

As the results from FIGS. 14A-21B show, specific assays for two dimerization states of MEK and ERK have been developed. These proteins (i.e., MEK, pMEK, and ERK) were detectable in whole cell lysates, and there was good reproducibility between the assays run together on one cartridge or independently from each other. Traditionally these types of analysis would only be done with co-immunoprecipitation which would take a couple days to perform, but using the assays described herein, this analysis was performed in a single day, although of course the assays were repeated on another day to determine whether the results were reproducible.

Notably, the analysis of these dimerization states described herein does not require the dimerized ERK-MEK or pMEK-ERK pairs to be removed and denatured as would be required by Western blot. This is important because it can be highly informative of the natural state of dimer (or heterodimer) or multimer. This data is believed to be the first to demonstrate detection of the ‘activated’ state (phospho-MEK) still dimerized to its potential target ERK.

The methods described herein provide a tool to detect activation states of kinases and other proteins in a native state. This is very important to drug development and discovery as well as toxicology. The harsh treatment of many of these tissue type samples to analyze such interactions in fact eliminates the ability to do such analysis. The interaction between proteins is just as important as the activation state and the understanding of both activation states and protein interactions in a single sample can be very impactful in the understanding of key biological events as well as drug development.

In summary, this Example shows that the quantitative detection of MAPK protein kinases can be an important tool for screening the efficacy of oncology drug candidates. As shown in this Example, AMMP assay technology represents a method to sensitively assess MEK concentrations in a complex matrix with minimal sample preparation. The non-optical basis of the technology reduces sample matrix effects experienced with fluorescence-based immunoassays. AMMP can be used on the ViBE protein analysis platform, which offers the possibility of automated walkaway operation. To date, detection of ERK/MEK heterodimers has not been directly measurable, and dimers of ERK/pMEK have never been detected. This assay measures true physiological interactions that are indicative of pathway activation states. As many drug development efforts are focused on the activation of the MAPK pathway, this technology can help researchers ascertain the level of pathway activation. It could be used in drug screening to indicate the efficiency of the upstream blockade of the MAPK pathway. In addition for a site specific protein-protein drug target, this technology offers a novel method to measure the effectiveness for these drugs. The AMMP assay described here shows promise as a tool for drug development, screening and for investigation of MAPK cascade biology.

### Example 5

### Detection and Analysis of Biomarkers in Cultured Cell Lysates and in Xenograft Tumor Cell Lysates

Mouse models of cancer are used extensively to evaluate the efficacy of new anticancer drugs. The most frequently used models are xenografts of human tumors grown in immunodeficient mice. Samples taken from such xenografts are excellent models for tumor biopsy samples taken from patients. These models are sometimes referred to as in vivo models as opposed to cells grown in tissue culture which are in vitro models.

In this example, the use of the BioScale ViBE instrumentation and AMMP assay technology to study the expression of proteins that are central to cancer research in a nude mouse tumor model are described. As shown in the results, reproducible measurement were obtained of natural and recombinant phospho-P38 in lysates of xenografts tumors of A431 epidermoid carcinoma cells in nu/nu mice. These results also demonstrate the reproducible measurement of total and phosphorylated EGFR in the same tumor lysates. ViBE measurement of both analytes in tumors compare favorably with lysates of the same cells grown in vitro. The assays are robust, with minimal interference from cell and tumor lysate matrix when comparing to analyte detection levels in detergent lysis buffer.

For these studies, p38 and EGFR (total and phosphorylated), non-limiting analytes in accordance with the methods described herein, were detected and analyzed.

P38, also known as mitogen activated protein kinase or MAPK (pT180/pY182), is responsive to cellular stress such as osmotic shock, heat shock or infection, and irradiation. P38 modulates the cell cycle, activation of DNA repair, regulation of protein translation and initiation of the immune response. The p38 pathway includes a cascade of protein kinases which ultimately leads to its phosphorylation on specific tyrosine and threonine residues. In mammals there are four isoforms of p38; alpha, beta, gamma and delta, the most abundant of which is p38 alpha. P38 is important in cell cycle regulation, apoptosis, and cancer research.

The Epidermal Growth Factor Receptor (EGFR) is a cell surface receptor which is activated by the binding of epidermal growth factor or another related ligand, transforming growth factor alpha. Upon ligand binding, the EGFR transforms from an inactive monomer state to an active dimer state in which its protein tyrosine kinase activity is stimulated. This stimulation results in phosphorylation of a series of tyrosine residues in the C terminus of the molecule. Hence, it is useful to detect and measure both total EGFR and phosphorylated EGFR (i.e., stimulated EGFR).

For these studies, sample was prepared for the detection of phospho-p38, and phospho- and total EGFR from A431 cell line lysates and from Xenograft tumor cell lysates. Table 4 lists some materials and their sources used for these methods.

TABLE 4
Materials-Lysates

| Material | Vendor | Catalog # | Lot # |
|---|---|---|---|
| A-431 Cells | ATCC | CRL-1555 | n/a |
| Mammalian Protein | ThermoFisher/ | 78505 | 157931, |
| Extraction Reagent | Pierce | | 157138 |
| Dimethyl sulfoxide (DMSO) | Sigma Aldrich | 472301-2L | 26696MJ |
| Recombinant Human EGF | R&D Systems | 236-EG | HLM2911041 |
| Cell Culture Medium RPMI | ATCC | 30-20 | 30001181 |
| 1640 | | | |
| Halt* Phosphatase Inhibitor | ThermoFisher/ | 78428 | ME156486 |
| Cocktail | Pierce | | |
| Halt* Protease Inhibitor | ThermoFisher/ | 78430 | MD156798 |
| Cocktails | Pierce | | |

For the sample preparation from A431 cells, the following methods were used. Human epidermoid carcinoma cells A431 (American Type Culture Collection (ATCC), Manassas, Va.; ATCC Catalog No. CRL-1555) were grown to approximately 80% confluence in T75 flasks according to standard cell culture techniques. Cells were stimulated for EGFR production with 100 μg recombinant epidermal growth factor (R&D Systems, Minneapolis, Minn.) in serum free medium for 10 minutes. Negative control cells were incubated with serum free medium without stimulant, also for 10 minutes.

Following treatment of the A431 cells with epidermal growth factor (or no treatment), the medium was aspirated and replaced with 3 ml TPER (Tissue Protein Extraction Reagent, Pierce) to lift the cells from the plastic flasks. Additional scraping was required to release the cells from the plastic. The resulting cell suspension was incubated for approximately 15 minutes on ice with vortexing every five minutes for effective cell lysis. Phosphatase and protease inhibitors (HALT, Thermo Fisher (Pierce)) were included in the detergent cell treatment preparation. Cell lysates were aliquoted and snap frozen on dry ice, and then transferred to a −80° C. freezer until analysis. A BCA protein assay (Thermo-Fisher/Pierce, e.g., Catalog No. 23225) was used to determine total protein concentration of the lysates.

For sample preparation from tumor cell lysates, multiple steps were involved to create the xenograft models, and then harvest xenograft tissue and prepare lysates. For creation of the xenograft models, A431 cells were used to implant nu/nu mice (from Charles River Laboratories, Wilmington, Mass.). For these methods, BD Matrigel™ Matrix HC was obtained from BD Biosciences (San Jose, Calif.; e.g., Catalog No. 354248). As this is stored frozen, the BD Matrigel™ Matrix HC was thawed at 4° C. on ice overnight. All pipette tips, pipettes, and microcentrifuge tubes used in the implantation procedure were also chilled overnight at 4° C. A431 cells were obtained from the Cell Culture Department at TGA Sciences, Inc. (Medford, Mass.). These cells, are referenced above, are also available from the ATCC. A 1:2 dilution of cells in the BD Matrigel™ Matrix was prepared on ice immediately prior to injection of the cells into the nu/nu mice. The final concentration of the cells in the 1:2 dilution was approximately 1×10⁶ cells/100 uL. Using a syringe needle, a subcutaneous pocket was formed in each nu/nu mouse by swaying the needlepoint to the right and left after needle insertion. A volume of 100 uL of the matrix/cell dilution was injected into the subcutaneous pocket formed with the needle. The syringe was rotated during withdrawal to prevent leakage of the matrix/cell dilution. Ten mice were injected and split into two groups, namely Groups C and D, with 5 mice per group. Group C mice were housed in one cage and Group D mice were housed in another cage.

Immediately after injection of all 10 mice, the length and width (cm) of the tumor site was measured using a caliper measuring device. The tumor length and width was recorded daily. The average of the length and width was calculated for each day of measurement. The tumors were harvested when the tumor average exceeded 0.5 cm in size or when the tumor site threatened to break through the skin. The harvested tumors were immediately flash frozen in liquid nitrogen and stored in 2 ml cryo-vials at −80° C. until processing.

For preparation of tumor lysates, each frozen tumor was transferred to a 15 ml polypropylene round bottom conical, and the weight of each individual tumor was recorded. The tumor samples were allowed to thaw at room temperature. During the thaw time, the amount of complete lysis buffer was calculated based on the recorded tumor weight. The Complete Lysis buffer was prepared (75 total) by adding 750 μL 100× Halt PIC and 750 μL 100×0.5M EDTA to 73.5 ml of T-PER buffer (Thermo Fisher). The appropriate volume of Complete Lysis Buffer was added to each individual thawed tumor sample. The sample tumor in Complete Lysis Buffer was next homogenized using a handheld homogenizer. The samples were allowed to settle at 2-8° C., and then re-homogenized. This step was repeated two times for each sample. The homogenizer probe was changed between each tumor sample. The samples were then centrifuged samples at 3300 RCF for 5 minutes at 4° C. Six 150 uL portions of sample were aliquoted into 0.5 ml microcentrifuge tubes per tumor sample. The remaining sample was stored in bulk. All lysate samples were stored at −80° C. until lysate processing.

Next, AMMP Assays were performed on the A431 cell lysates and the A431 xenograft lysates. Table 5 lists some materials and their sources used for these methods.

TABLE 5

| Material | Vendor | Catalog # | Lot # |
|---|---|---|---|
| Bead-Phospho-P38 | BioScale | n/a | 11-047, 13- |
| Antibody coupled | | | 025, 13-126 |
| Bead-Phospho-EGFR | BioScale | n/a | 12-083, 13- |
| Antibody coupled | | | 026 |
| Bead-Total-EGFR Antibody | BioScale | n/a | 12-161, 13- |
| coupled | | | 119 |
| Fl-labeled Antibody-P38 | BioScale | n/a | 11-013 |
| Fl-labeled Antibody-EGFR | BioScale | n/a | 12-173, 13- |
| | | | 024 |
| Diluent | BD Biosciences | 555213 | 84293 |
| Mammalian Protein | ThermoFisher/ | 78505 | 158971; |
| Extraction Reagent | Pierce | | 163779 |
| Lysate (A431) | BioScale- | — | 20111220; |
| | prepared | | 20120110; |
| | | | 20120118 |
| Recombinant Human | R&D Systems | 5477-KS | 1260044 |
| Active p38alpha | | | |
| Phospho-EGFR (Y1068) | R&D Systems | DYC3570-2 | 1236924 |
| Standard | | | |
| PBS-Tween | BioScale | n/a | n/a |
| 25 mM Phosphoric Acid | BioScale | n/a | n/a |

For the AMMP Assays, streptavidin-coated paramagnetic beads (Dynal beads, from Invitrogen, Carlsbad, Calif.) were coupled with biotinylated rabbit anti-analyte antibodies (Cell Signaling Technology, Inc., Danvers, Mass.). A mouse or rabbit anti-analyte antibody (Cell Signaling Technology) was labeled with fluorescein for use as the ViBE assay detection reagent (BioScale, Inc., Lexington, Mass.). These reagents were diluted for assay use in standard biological diluents (Diluent, Becton Dickenson) to ViBE assay-specific concentrations. Assays were run in a sandwich format, with simultaneous incubation of the sample with the beads and fluorescein labeled antibody.

Cell lysate samples were prepared for the assays by thawing them on ice then clarifying by centrifugation in a microcentrifuge at 14,000 RPM for 10 minutes. Supernatants were removed from the samples and the total protein concentrations were adjusted in MPER (Mammalian Protein Extraction Reagent (Thermo Fisher/Pierce) for the assays. Serial two-fold dilutions were also prepared in MPER and subsequently assayed in triplicate. Forty microliters of lysate sample were assayed for each dilution, added to 40 μl of assay diluent (Becton Dickinson). The samples and assay reagents were combined and then loaded onto the ViBE apparatus (BioScale, Inc.) for analysis. The ViBE apparatus was programmed to provide an incubation time of 4 hours, prior to analysis of the samples on the ViBE. Elapsed time to achieve results for a two 96 well plate assay was approximately 8 hours. Negative controls containing the bead only with the samples (no fluoresceinated antibody) were run and the ViBE response was subtracted from the sample signal readings to determine the specific assay signal.

Samples were also analysed by Western blotting analysis using phospho-p38, phopho-EGFR, and total EGFR-specific antibodies. For these studies, cell lysate samples were prepared as described above, and total protein concentrations were adjusted to a specific ug total per well, to compare to the ViBE assay total protein/test. The samples were diluted 1:1 in SDS-PAGE sample preparation buffer and run on 8-16% Tris-glycine gels under constant current for 2 hours. Chemiluminescent molecular weight markers (Invitrogen) were run on each gel. Proteins were then transferred to 0.45 μm pore size PVDF membranes according to standard techniques. The membranes were blocked with 5% BSA/or 5% milk in Tris buffered saline/0.1% tween then probed with the analyte specific antibody at the manufacturer's recommended concentration overnight with constant agitation. The following day the membranes were washed then probed with a secondary rabbit or mouse IgG specific perioxidase antibody (Jackson Immunoresearch, West Grove, Pa.) diluted to a working concentration in normal donkey serum (Equitech Bio, Inc., Kerrville, Tex.). The membranes were developed with chemiluminescent substrate (ChemiGlow reagent from Protein Simple (formerly Cell Biosciences), Santa Clara, Calif.). The membranes were developed for 40 seconds then they were photographed for image preservation. Elapsed time to achieve results was approximately 30 hours.

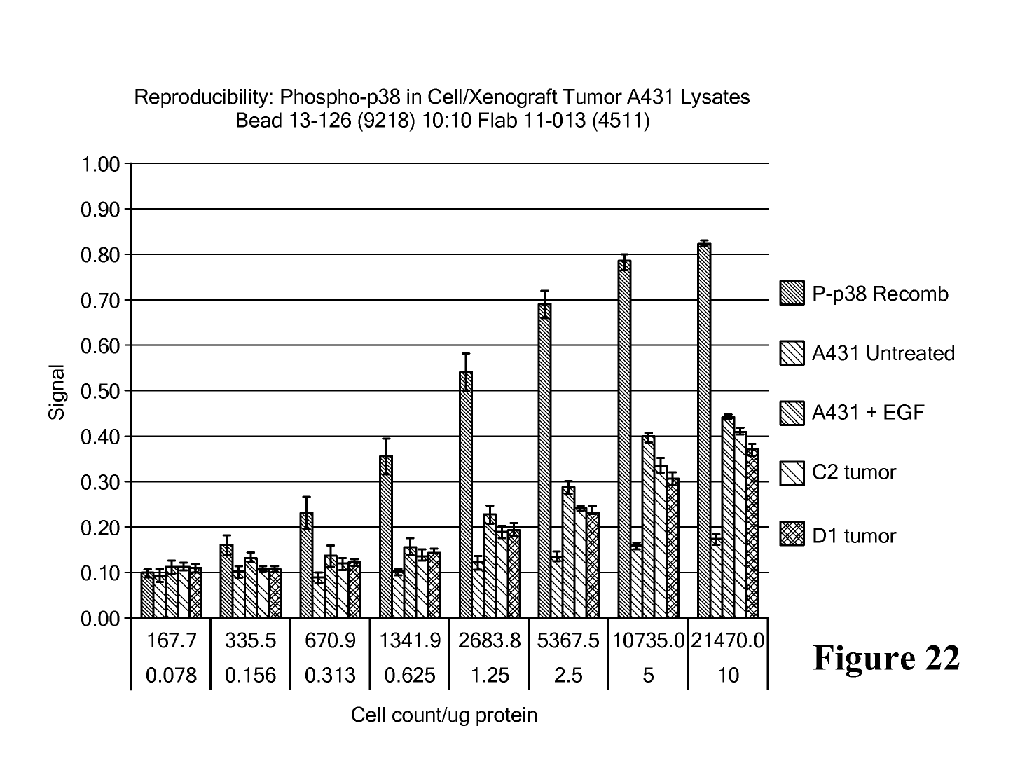

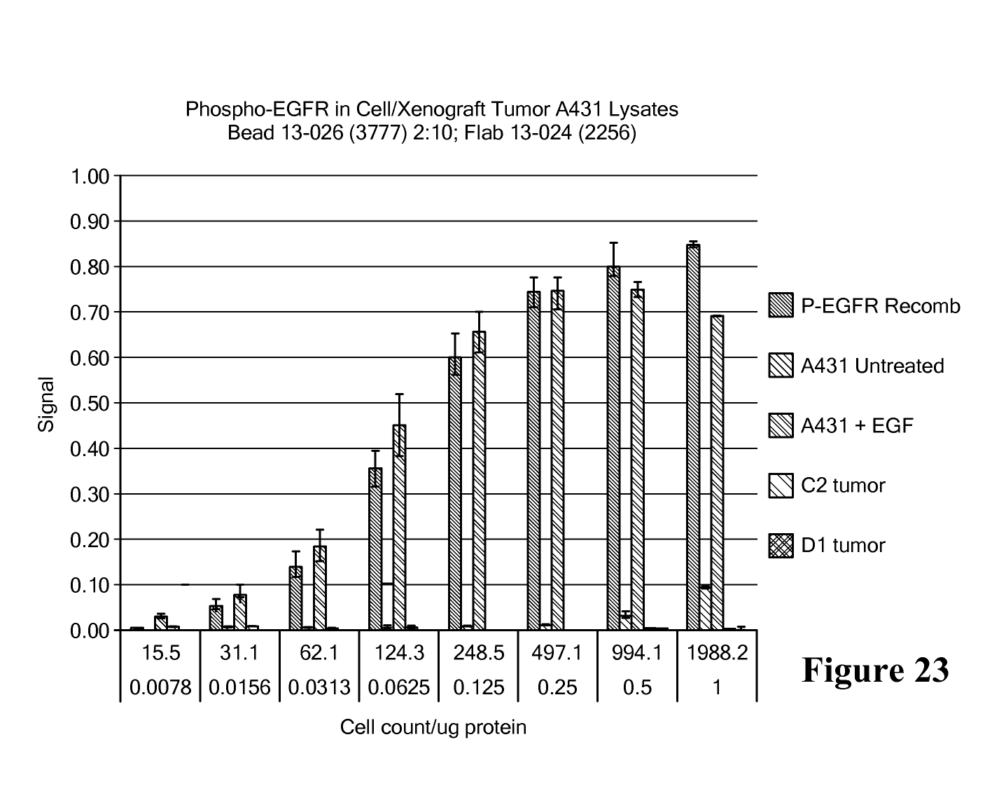

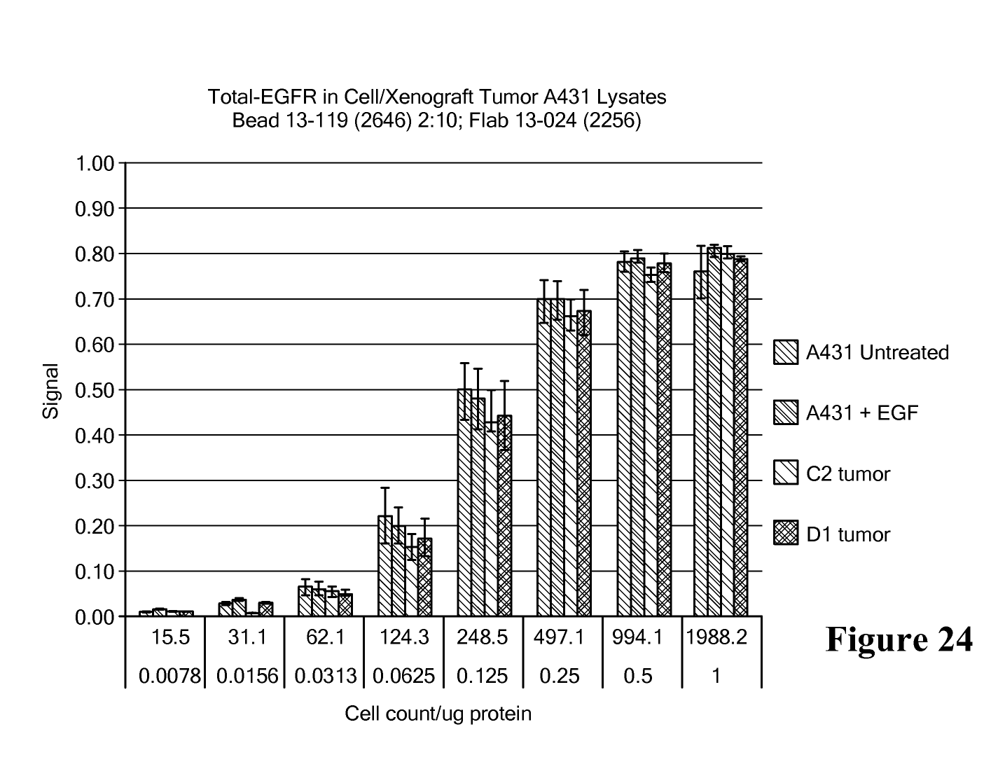

The results from the AMMP analyses on the ViBE apparatus are shown in FIGS. 22-24. FIG. 22 shows the results for detection of phospho-p38; FIG. 23 shows the results for the detection of phospho-EGFR; and FIG. 24 shows the results for the detection of total EGFR. Note that in FIG. 23, phosphorylated EGFR was not found in tumor lysates using the AMMP assay method. Also note in FIG. 24 that recombinant total EGFR was not available to serve as a positive control.

FIG. 22 demonstrates reproducible measurement of natural and recombinant phospho-P38 in lysates of xenograft tumors of A431 epidermoid carcinoma cells in nu/nu mice (Charles River Laboratories). Also demonstrated in FIG. 24 is the reproducible measurement of total EGFR in the same cell and tumor lysates. ViBE measurement of both analytes in tumors compare favorably with lysates of the same cells grown in vitro. The following conclusions were made from these experiments.

First, the lowest cell numbers detected in each tumor or cell lysate approach the 100-1000 cell range, based on correlation of protein concentration to cell number. An example of this is shown in Table 6, which shows the number of cells and total protein for the samples analyzed on different days using the BCA assay from Thermo Fisher/Pierce.

TABLE 6

| | | | | Protein | Cells/ |
|---|---|---|---|---|---|
| | | Cells/ml | Cells/ul | (ug/ml) by | 5 ug |
| Culture | Date | MPER | lysate | BCA | protein |
| A431 | 28-Oct. | 1.19E+06 | 1190 | 498.6 | 11948 |
| A431 | 4-Nov. | 1.25E+06 | 1250 | 496 | 12600 |
| A431 | 11-Nov. | 1.33E+06 | 1330 | 422.1 | 15755 |
| A431 | 21-Nov. | 5.20E+05 | 520 | 287.5 | 9043 |
| A431 | 5-Dec. | 8.80E+05 | 880 | 335.7 | 13107 |
| A431 | 9-Dec. | 2.90E+05 | 290 | 300 | 4833 |
| A431 | 20-Dec. | 5.40E+05 | 540 | 401.9 | 6718 |
| A431 | 10-Jan. | 6.40E+05 | 640 | 644.6 | 4964 |
| A431 Stim | 18-Jan. | 1.25E+06 | 1250 | 628.7 | 9941 |
| A431 Unstim | 18-Jan. | 1.25E+06 | 1250 | 582.2 | 10735 |

Secondly, the measurements from the AAMP assay were reproducible, with triplicate analysis of each sample. And finally, comparable signal levels were observed for P-P38 and Total EGFR in A431 cell and tumor lysates.

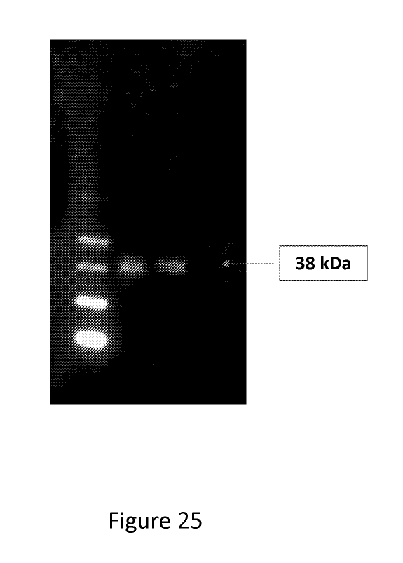

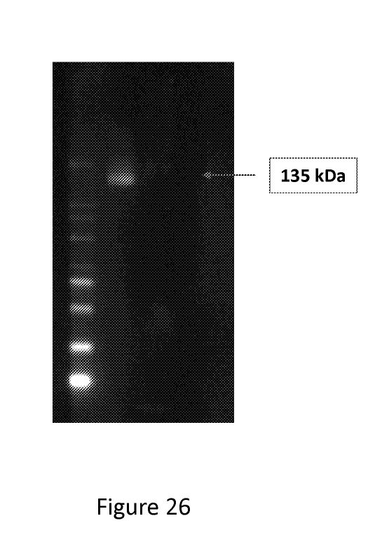

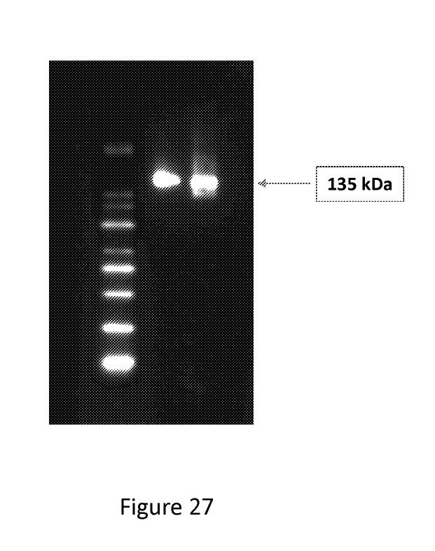

Western blotting analysis of EGF-stimulated A431 cells and tumor lysate (randomly chosen) was performed using phospho-p38 specific antibody (See FIG. 25), phospho-EGFR specific antibody (FIG. 26) and total EGFR-specific antibody (FIG. 27). The results of the p38 ViBE assay and the Western blots were compared for detection in stimulated/unstimulated A431 cell and unstimulated tumor lysates. The signal on the VIBE (FIG. 22) was detectable over background at 0.156 μg of protein while the western blot signal (FIG. 25) was visible at the 10 μg level tested. The results of the p-EGFR ViBE assay (FIG. 23) and the western blot (FIG. 26) were compared for detection in EGF stimulated A431 cell and tumor lysates. For the stimulated cell lysate, the signal on the VIBE (FIG. 23) was detectable over background at 0.0625 μg of cell lysate protein while the western blot signal (FIG. 25) was visible at the level tested (5 μg). There was no detection of phospo-EGFR in the tumor lysate by either method (see FIG. 23 and FIG. 26). The results of the Total-EGFR ViBE assay (FIG. 24) and the western blot (FIG. 27) were compared for detection in EGF stimulated A431 cells and unstimulated tumor lysates. For both the cell and tumor lysates, the signal on the VIBE (FIG. 24) was detectable over background at 0.0156 μg of lysate protein. The western blot signal (FIG. 27) showed strong positive reactivity for each of the samples tested. Recombinant total EGFR was not available for these studies.

The Western blots (FIGS. 25-27) confirmed the presence or absence of the ViBE measured analytes in the xenograft lysates (compare to FIGS. 22-24).

Next, recombinant p-P38 (R&D Systems), recombinant p-EGFR (R&D Systems) and natural total EGFR were measured in each of the kinase analyte assays described above over several days with multiple cartridge lots and multiple ViBE instruments. The assay responses in signal units were assessed. Protein concentrations were adjusted to 10 ng/ml (for phospho-P38), 10 ng/ml (for phospho-EGFR) or 0.5 μg (for natural total EGFR) in MPER buffer (Thermo Scientific/Pierce) for the top point of dose response curves, then these samples were serially diluted two-fold in MPER for the remaining points on the curves.

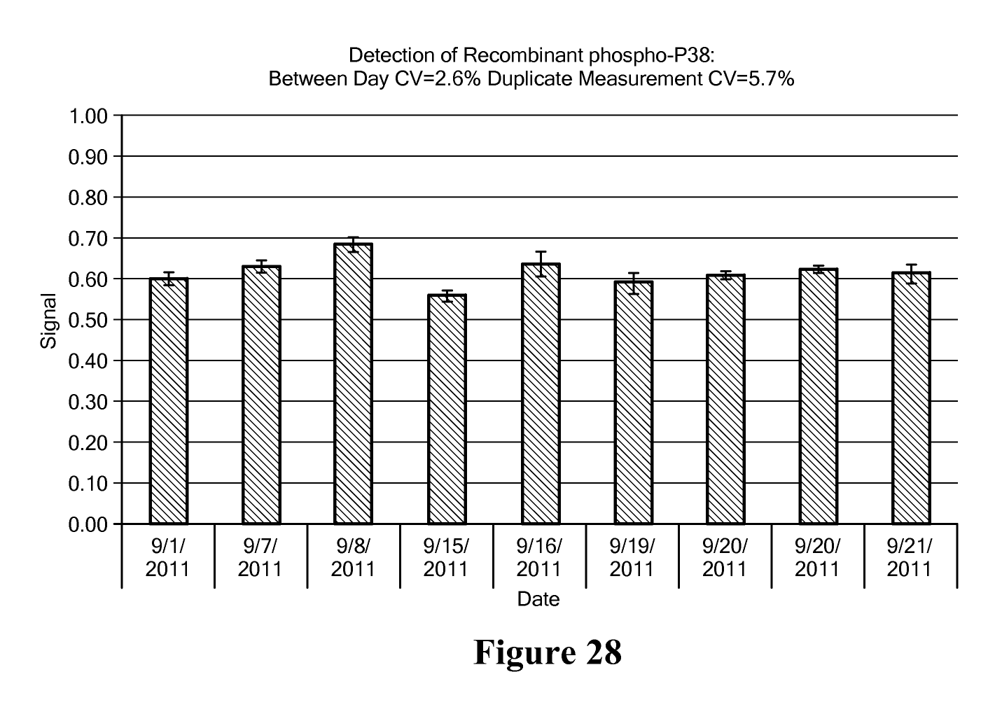

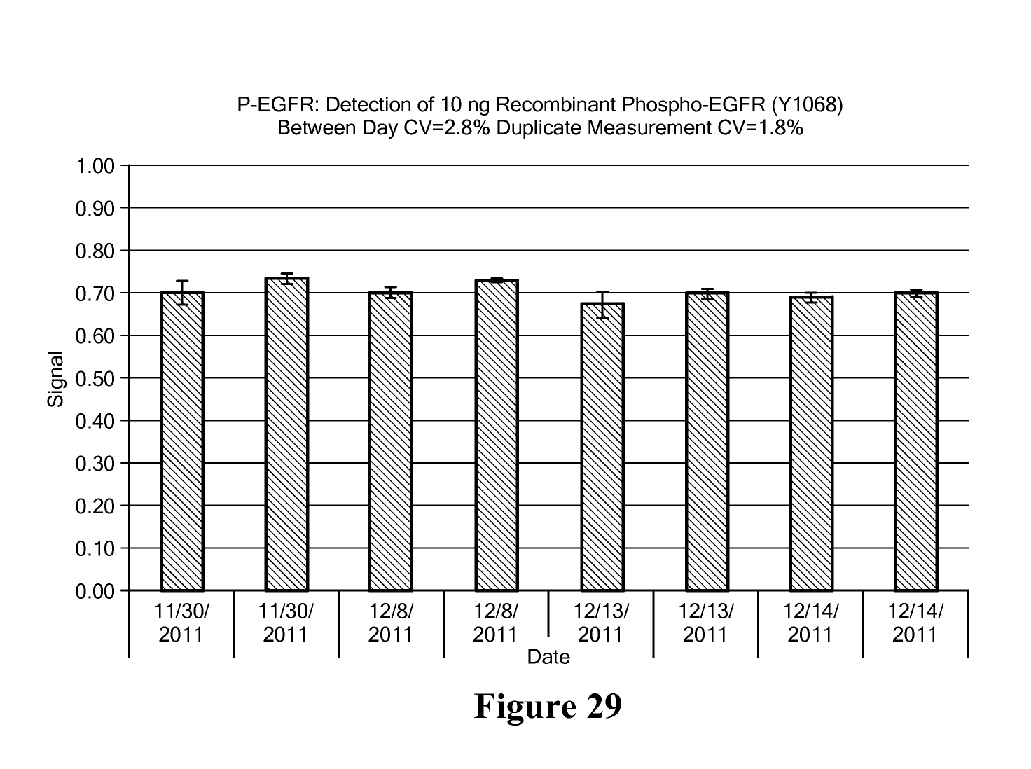

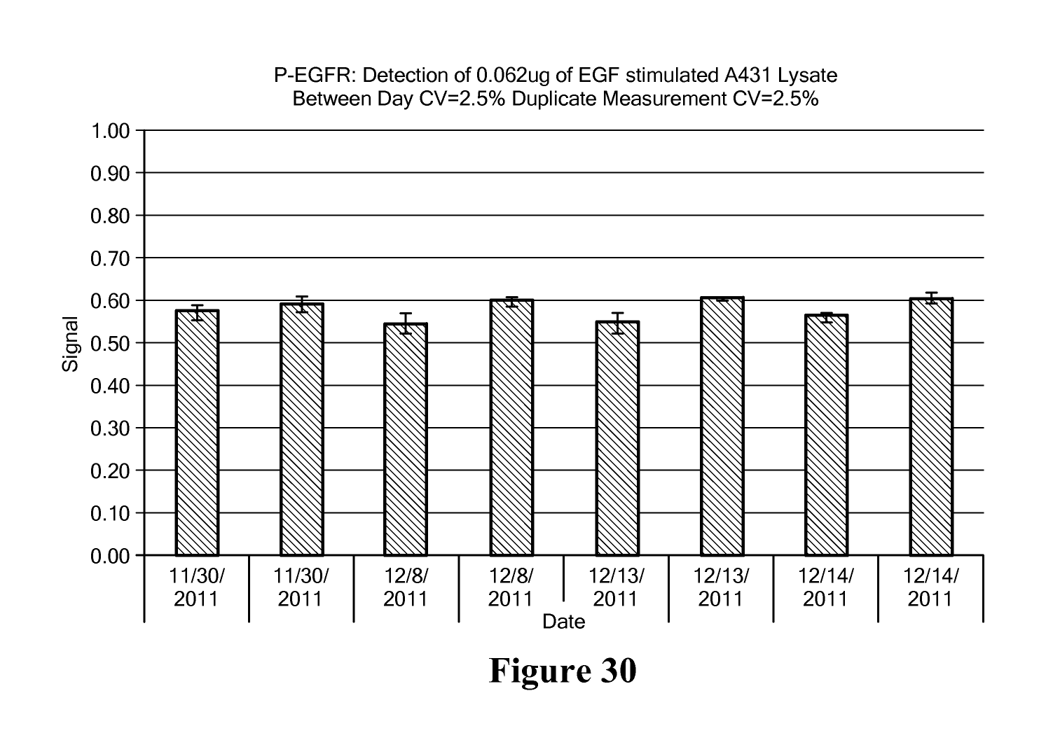

Representative mid-points on the curves were chosen for illustration in the graphs shown in FIGS. 28-30. FIG. 28 shows detection of recombinant phospho-p38 as measured using the AMMP assay over twenty-one days. FIG. 29 shows detection of recombinant phospho-EGFR using the AMMP assay over fifteen days. The phosphorylated EGFR was also detected in cell lysates prepared from EGF-stimulated A431 cells, and FIG. 30 shows the measurements obtained of phospho-EGFR from EGF-stimulated A431 cell lysates over fifteen days. As shown in FIGS. 28-30, percent coefficients of variations between days of measurement were found to be generally less than 3%, and between replicates on the same day (i.e., duplicates) were found to be less than 6%.

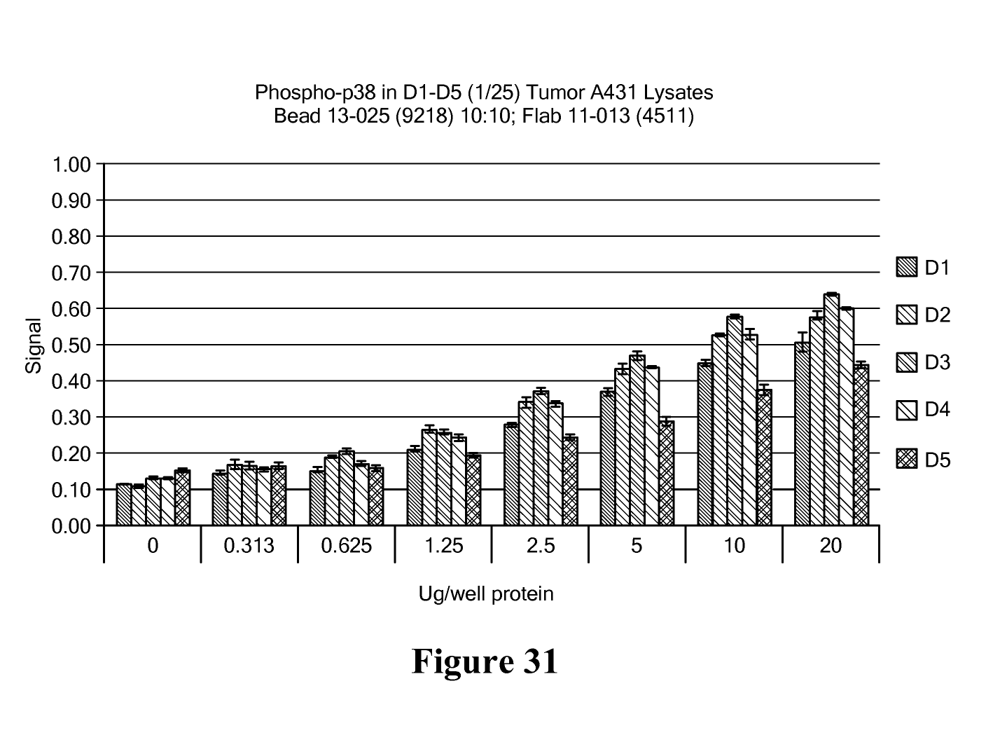

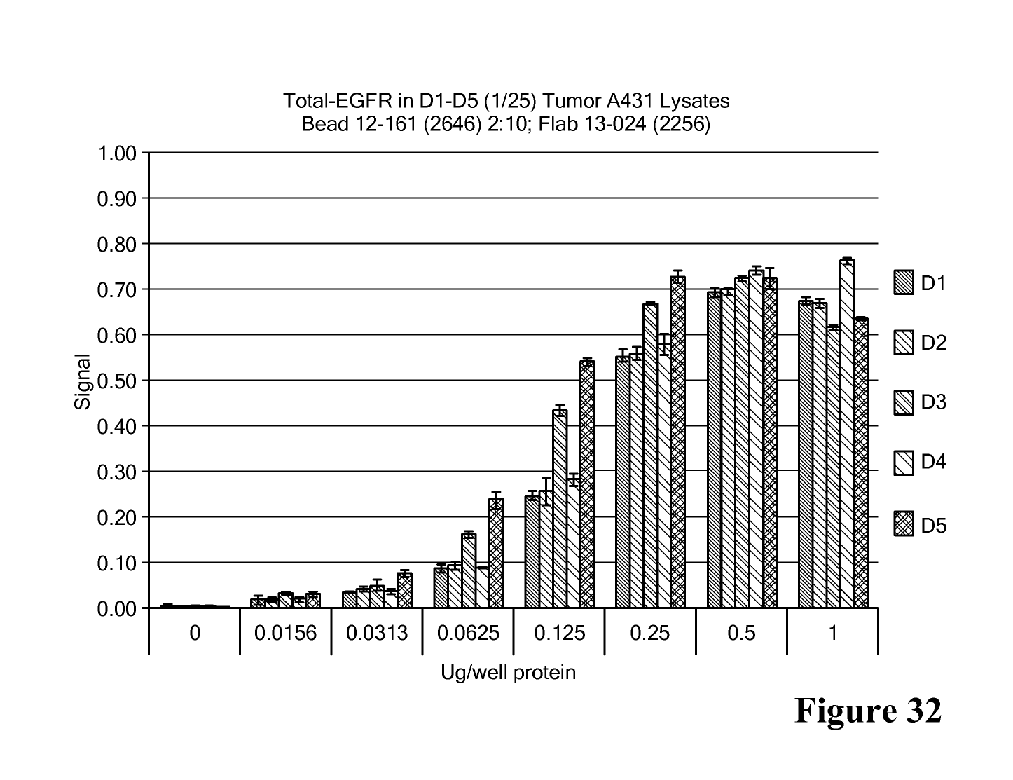

Next, the reproducibility of in vivo sample measurements of phospho-P38 (i.e., phosphorylated p38) and Total EGFR was assessed. For these studies, tumors from 5 of the 10 mice injected with A431 cells were tested using the AMMP assay with replicates for each dilution measured. FIG. 31 shows the amount of phosphor-p38 measured in tumors from D1-D5 mice using the AMMP assay. FIG. 32 shows the amount of total EGFR measured in tumors from D1-D5 mice using the AMMP assay. The reproducibility of the triplicate readings for each tumor and the consistency of the results obtained for each tumor in the tested group lend confidence to the data.

Next, experiments were done to determine the range of specificity of the AMMP assayusing the ViBE apparatus. These studies were done to demonstrate that the AMMP assays on the ViBE can recover and measure various amounts of spiked analyte in a dose dependent manner, when performing the measurement in increasing amounts of cell or tumor lysate. In other words, these studies tested for matrix interference of the analyte measurements, and whether or not the “spiked” analyte could still be accurately recovered and measured using the AMMP assay.

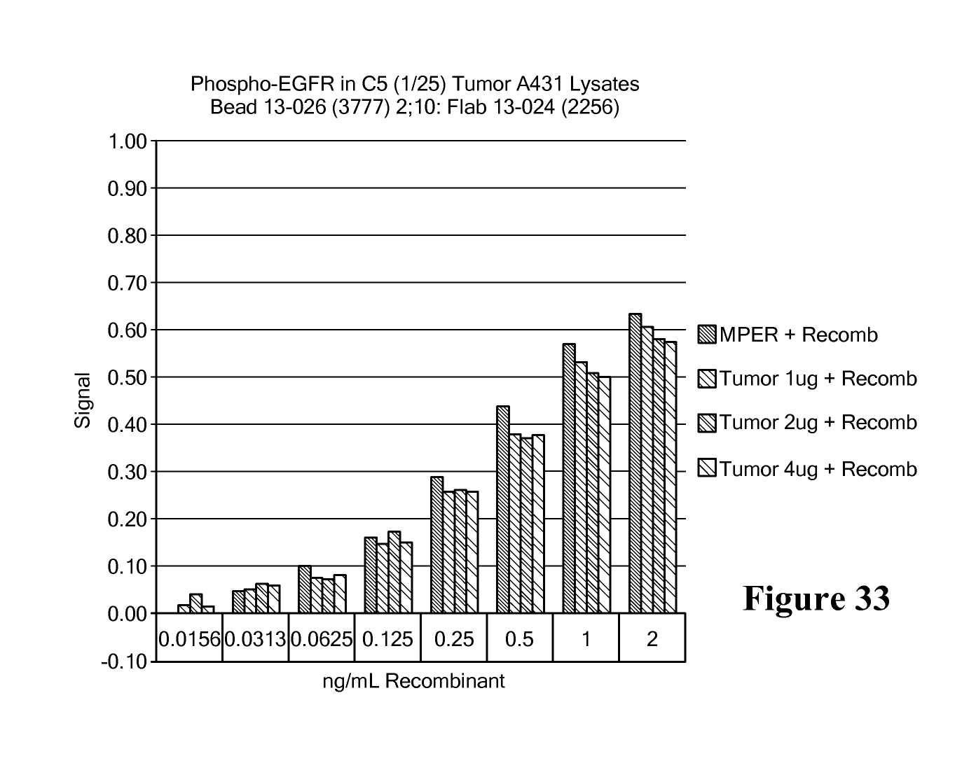

For these studies, different amounts of recombinant phosphorylated EGFR or recombinant phosphorylated p38 was added to different masses of tumor lysates prepared from randomly chosen mice or to MPER buffer alone. In FIG. 33, different amount of recombinant phospho-EGFR were added to MPER buffer, or 1 ug, 2 ug, or 4 ug of tumor lysate mass taken from the C5 mouse. Table 7 shows the recovery of the p-EGFR taken from these studies as compared to from MPER buffer alone.

TABLE 7

| Phospho-EGFR ng/ml | 1 ug lysate | 2 ug lysate | 4 ug lysate |
|---|---|---|---|
| 2 | 92.7 | 91.1 | 83.9 |
| 1 | 92.8 | 87.5 | 99.1 |
| 0.5 | 84.0 | 92.2 | 103.1 |
| 0.25 | 84.8 | 101.5 | 104.0 |

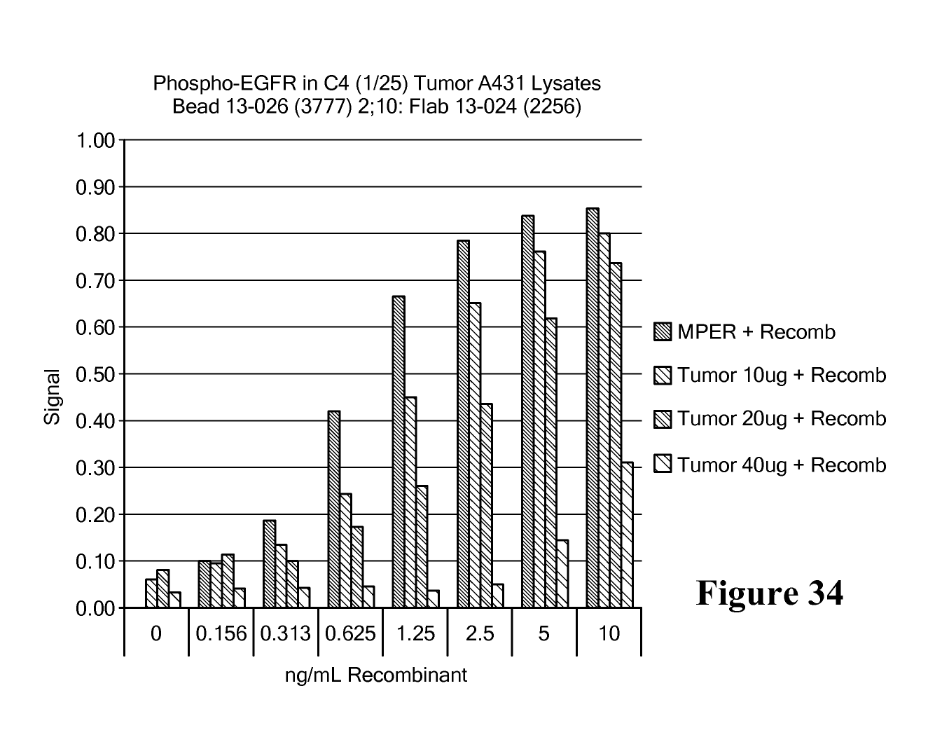

In FIG. 34, different amounts of phospho-EGFR were added to MPER buffer, or 10 ug, 20 ug, or 40 ug of tumor lysate mass taken from the C4 mouse. Table 8 shows the recovery of the p-EGFR taken from these studies as compared to from MPER buffer alone.

TABLE 8

| Phospho-EGFR ng/ml | 10 ug lysate | 20 ug lysate | 40 ug lysate |
|---|---|---|---|
| 10 | 93.5 | 87.2 | 36.6 |
| 5 | 91.1 | 74.3 | 17.7 |
| 2.5 | 82.8 | 56.1 | 6.6 |
| 1.25 | 67.2 | 38.8 | 5.5 |
| 0.625 | 58.2 | 41.9 | 10.8 |
| 0.313 | 72.1 | 51.7 | 22.6 |
| 0.156 | 95.9 | 118.6 | 39.8 |

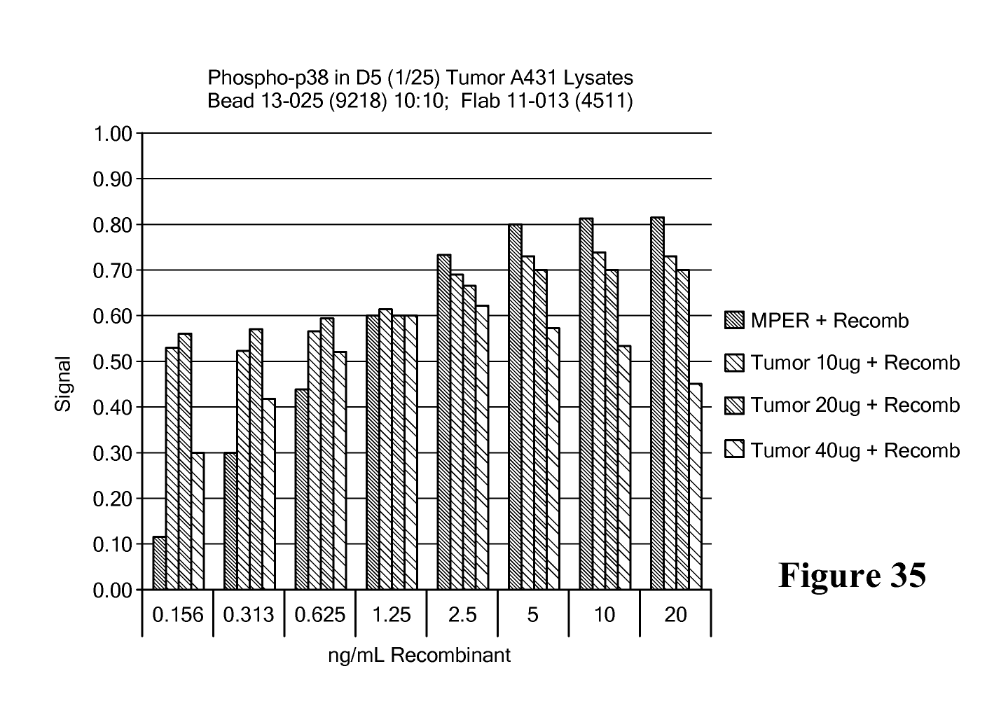

In FIG. 35, different amounts of phospho-p38 were added to MPER buffer, or 10 ug, 20 ug, or 40 ug of tumor lysate mass taken from the D5 mouse. Table 9 shows the recovery of the p-EGFR taken from these studies as compared to from MPER buffer alone.

TABLE 9

| Phospho-p38 ng/ml in | | | |
|---|---|---|---|
| MPER | 10 ug lysate | 20 ug lysate | 40 ug lysate |
| 20 | 89.2 | 84.8 | 55.2 |
| 10 | 90.5 | 86.0 | 65.8 |
| 5 | 91.2 | 87.3 | 71.6 |
| 2.5 | 94.2 | 90.8 | 84.9 |
| 1.25 | 102.4 | 99.8 | 99.2 |
| 0.625 | 129.7 | 136.1 | 119.0 |

Thus, against the background of interference of up to 20 ug of tumor protein, 80-100% of the spiked analyte was recovered and measured using the AMMP assay.

Overall, the results in this Example show the use of AMMP assays, performed on the BioScale ViBE apparatus, for the detection of phosphorylated MAPK protein p38, and phospho and total EGFR in the A431 cell line and A431 cell xenograft tumors in mice. Comparative signal levels were observed for the three markers in the cell and tumor lysates, indicating that the AAMP technology is capable of delivering highly sensitive and reproducible measurements in both matrices.

The phospho-p38, phospho-EGFR, and total EGFR proteins were detectable in human xenografts (in vivo) and whole cell (in vitro) lysates, by western blot, results of which corroborate the results of the AMMP assays. However, compared to western blots run on the same cell lysates, the VIBE assay takes less time and requires less starting material, as the ViBE assays have lower limits of detection than western blots. The cell numbers detectable (lower limit) by the AMMP assays are in the range of 100-1000 cells, which is well within the range of cell numbers required for tumor biopsies. Additionally, recombinant forms of the phosphorylated p38/phosphorylated EGFR proteins were reproducibly detectable when spiked into the cell and tumor lysates with the AAMP assays. At total protein levels of up to 20 μg/test, the spiked proteins are recovered between 80-100%.

As demonstrated by the excellent reproducibility over multiple ViBEs and days of testing, the AMMP assays can be run on different cartridges/days of assays. Assay coefficients of variance (CVs) are less than 3% between days and less than 3% between duplicates run on a single day. Further, the open system nature of the AAMP technology allows multiple analytes to be assayed within a single run of a cartridge, which corresponds to one or more 96 well plates. Thus, multiple AMMP assays can be run on a single ViBE cartridge.

The results showed good reproducibility between five xenograft tumor lysates when tested in both the phosphorylated p38/phosphorylated EGFR assays. The coefficient of variance (CVs) were less than 5%. Further, there is good reproducibility between analyte replicates from a single tumor—for these, the CVs are less than 5%.

Additional studies were next performed to look at members of the MAPK signaling pathway and the PI3K-AKT signaling pathways. The mitogen-activated protein kinase (MAPK) and phosphoinositide 3-kinase (PI3K-AKT) cascades are signal transduction pathways which heavily influence the regulation of cell proliferation, differentiation, and survival. Schematic diagram of these pathways are shown in FIGS. 9, 10A, and 10B. The ability to measure the activation states and molecular interactions of the kinases comprising these pathways are crucial in the efficient and effective development of anti-ontological pharmaceuticals and therapeutics.

AKT (also known as Protein Kinase B) is a serine/threonine-specific protein kinase that plays a key role in multiple cellular processes such as glucose metabolism, apoptosis, cell proliferation, transcription and cell migration. There are three AKT family members. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Akt2 is an important signaling molecule in the Insulin signaling pathway and is required to induce glucose transport. The role of AKT3 is less clear, but it appears to be predominantly expressed in the brain.

For these studies, the non-optical acoustic membrane microparticle (AMMP) technology was used to assess and quantify the activation state of several implicated kinases, namely EGFR, p38, and AKT. Cell lines used were HEK 293 cells, NIH-3T3 cells, WM 266.2 cells, A431 cells, and MCF-7 cells.

Xenograft models were created as described above by injecting HEK293 tumor cells (embryonic kidney cells) into nu/nu mice from Charles River Mice. These groups were called groups G and H, and the members of the groups were identically treated.

Lysates were prepared from several human tumor cell lines, with or without stimulation of surface ligand receptors or mitogen stimulation and compared with lysates prepared from xenografted tumors derived from the same cell lines.

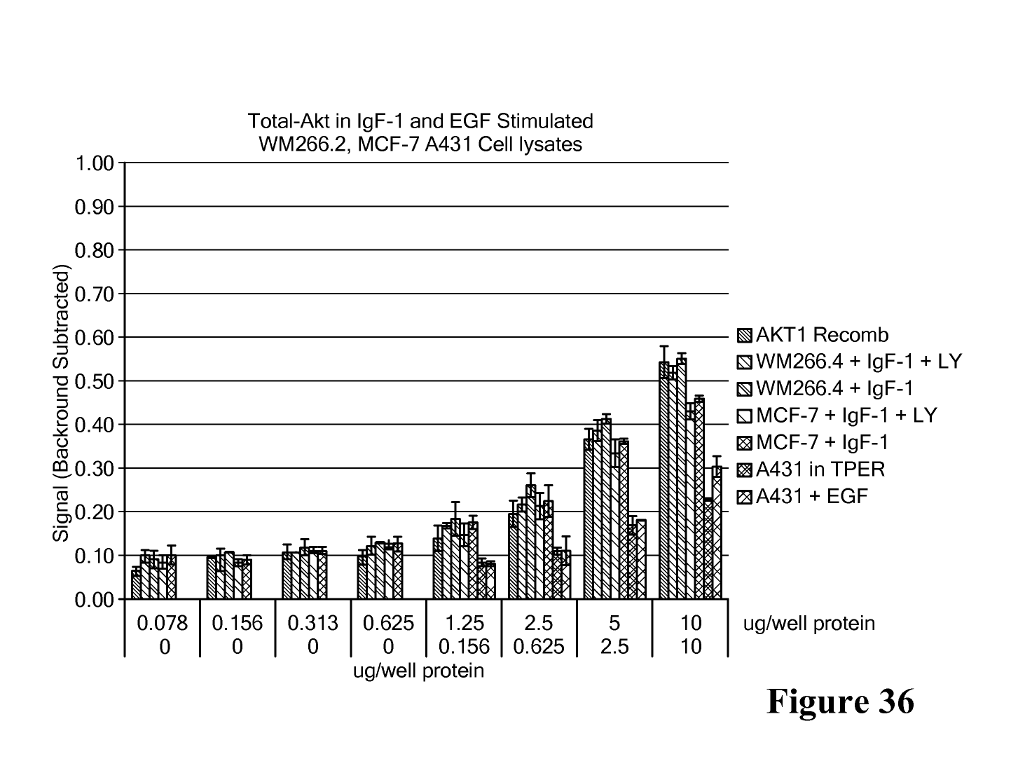

As shown in FIG. 36, in an early study, the AMMP method was able to detect total Akt1 in IgF-1 and EGF stimulated WM266.2, MCF-7, and A431 cell. The control “AKT1 Recomb” is active recombinant AKT, sold as Catalog No. 1775-KS from R&D Systems (Minneapolis, Minn.).

This result was achieved from very low protein levels and cell numbers. Indeed, the lowest cell numbers detected in each cell or tumor lysate approach the 100-1000 cell range, based on the correlation between protein concentration and cell number.

This was surprising because, as shown below in Table 10, other common molecular/biochemical techniques typically require higher cell numbers

TABLE 10

| Technique | Minimum Cell Requirement |
|---|---|
| PCR, RT-PCR, immunohistochemistry, CGH | 1-1000 |
| with whole genome amplification | |
| Western blot | 2000-5000 |
| 2-D protein gel analysis | 50,000-100,000 |
| Southern blot, Northern blot, CGH without | 100,000-200,000 |
| whole genome amplification | |

An assessment was made of the amount of protein used in the methods described in this example based on different lysate preparations and the amount of protein as determined using the bicinchoninic acid (BCA) protein assay reagent (Thermo Scientific) for measuring protein quantity. To do this assessment, data from eight different cell lysate preparations were analyzed. The numbers were used to calculate cell numbers detected. The cells/5 ug protein represents the highest cell number tested, followed by serial two-fold dilutions. The results of these studies are shown in Table 11.

TABLE 11

| | | Cells/ml | Cells/ul | Protein (ug/ml) | Cells/5 ug |
|---|---|---|---|---|---|
| Culture | Date | MPER | lysate | by BCA | protein |
| Jurkat | 28-Sep. | 5.00E+06 | 5000 | 180.7 | 135000 |
| Jurkat | 5-Oct. | 3.75E+06 | 3750 | 194.5 | 96649 |
| HEK | 12-Oct. | 3.20E+06 | 3200 | 652 | 24539 |
| HEK | 26-Oct. | 2.08E+06 | 2080 | 639 | 16275 |
| 3T3 | 12-Oct. | 5.50E+05 | 550 | 258.2 | 10658 |
| 3T3 | 19-Oct. | 5.00E+05 | 500 | 309.5 | 8090 |
| A431 | 28-Oct. | 1.19E+06 | 1190 | 498.6 | 11948 |
| A431 | 4-Nov. | 1.25E+06 | 1250 | 496 | 12600 |

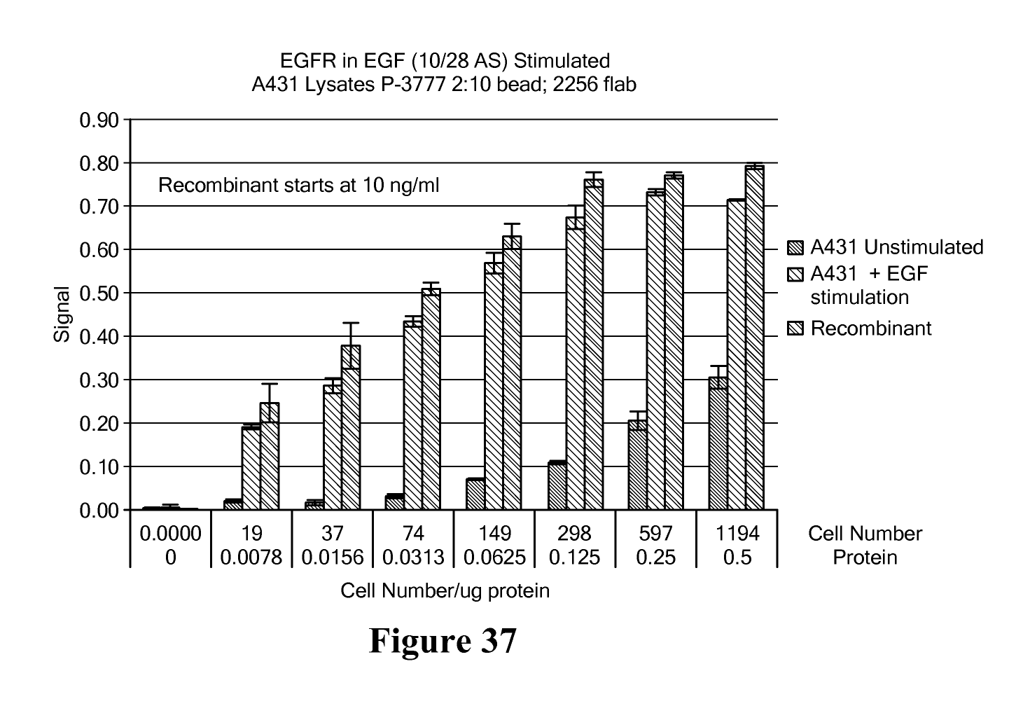

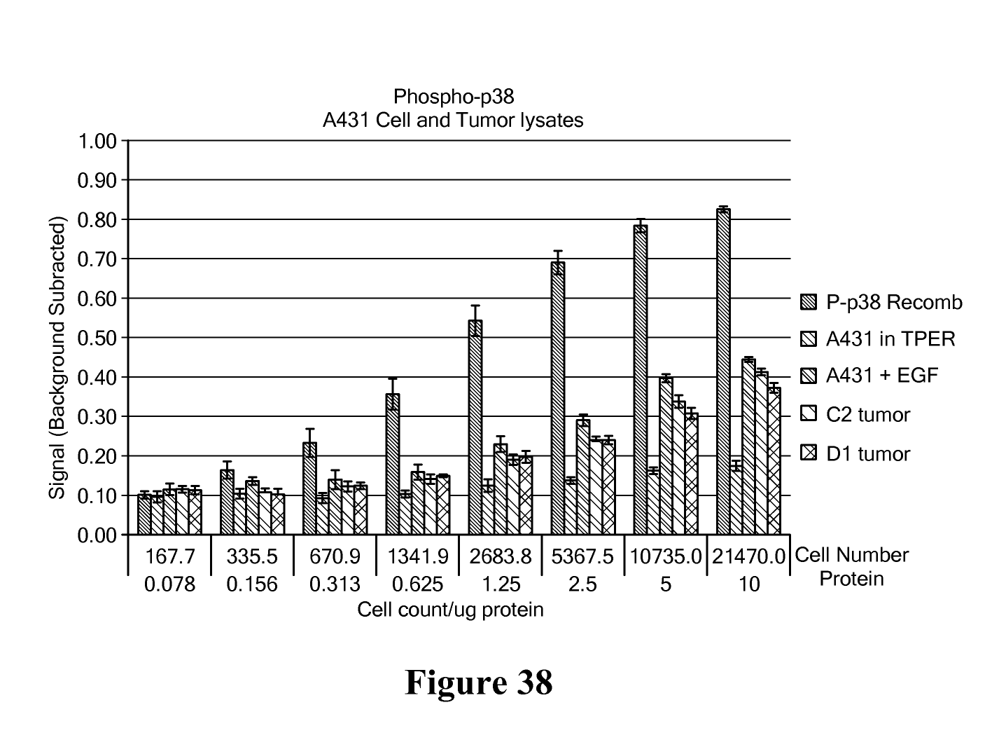

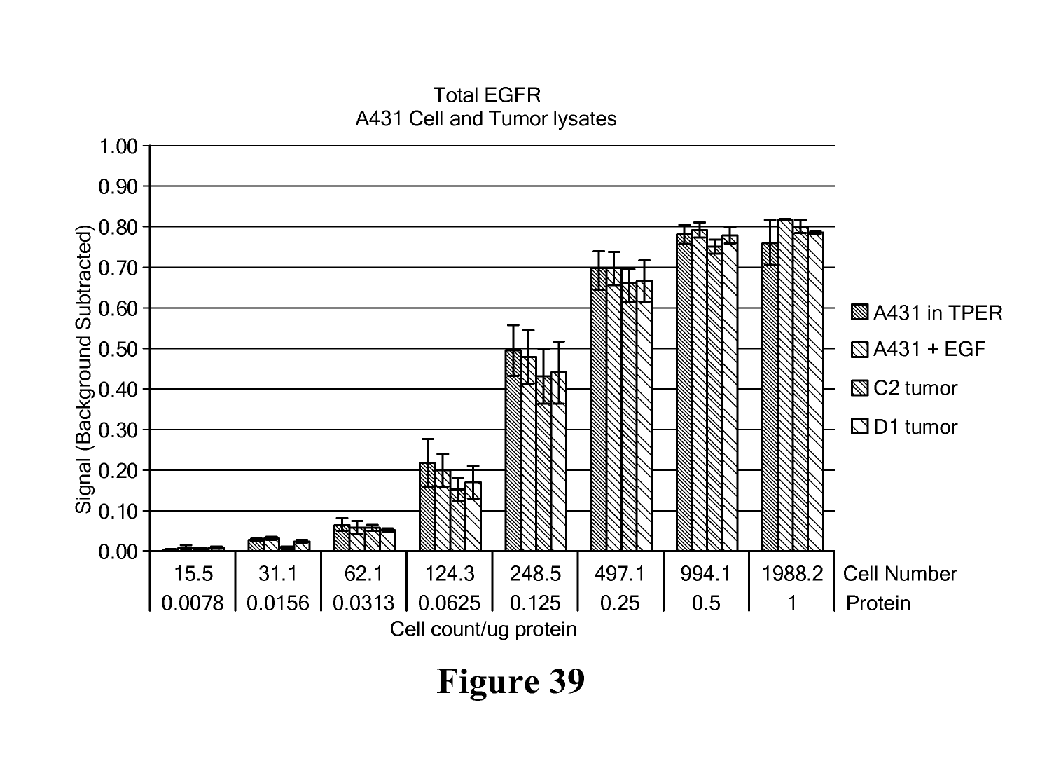

In FIG. 37, the EGF receptor (EGFR) was detected and semi-quantified in lysates made from EGF stimulated A431 cells at very low cell numbers. Similarly, phosphorylated p38 was detected in lysates made from low numbers of EGF-stimulated A431 cells (see FIG. 38). Note in FIG. 38 the dramatic increase in the amount of phosphor-p38 observed in EGF-stimulated cells (red bars) as compared to unstimulated A431 cells (blue bars); this was not the case for the amount of total EGFR observed in these cells (see FIG. 39, compare unstimulated A431 (blue bars) to EGF-stimulated A431 (red bars).

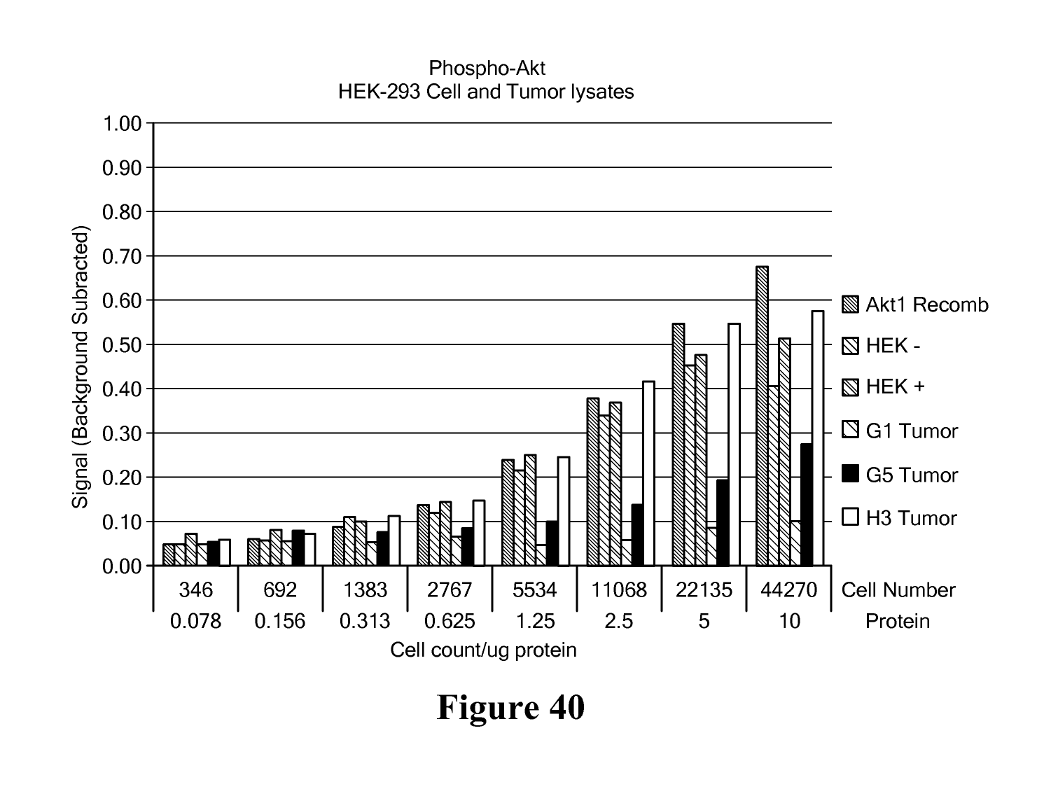

Next, detection of phosphorylated Akt1 and total Akt1 in lysates made from tumor xenografts was attempted. As shown in FIG. 40, phosphorylated Akt1 was detected in both unstimulated HEK cells as well as HEK cells sitmulated with anisomycin (from Sigma-Adrich, St. Louis, Mo.). Interestingly, the amount of phosphorylated Akt1 in the different tumors varied. The Akt1 Recomb control in FIG. 40 was R&D Systems' Active Recombinant AKT, Catalog number 1775-KS, the solid phase (bead)-bound antibody to phosphorylated AKT is Catalog No. 4058 (pAKT s473) (a rabbit monoclonal antibody) from Cell Signaling Technology, Inc. (Danvers, Mass.), and the fluorescein-labeled anti-total AKT antibody is Catalog No. 2697 (a murine monoclonal antibody) from Cell Signaling Technology, Inc.;

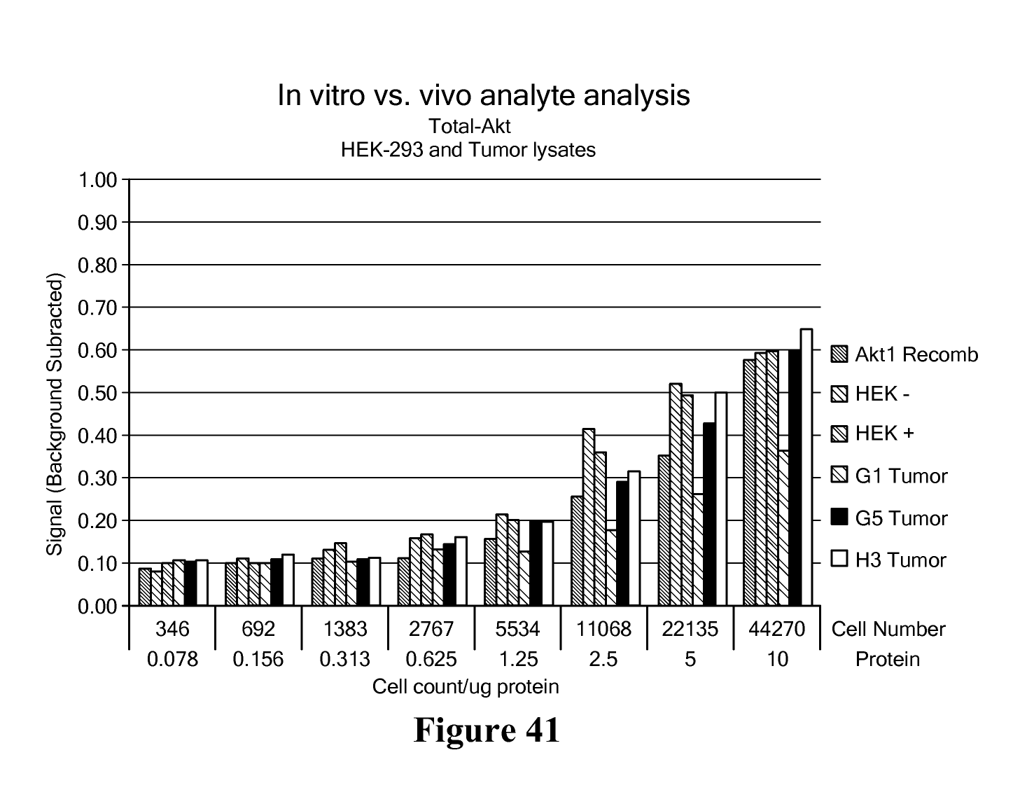

Similar results were obtained for total Akt1 in unstimulated HEK cells and anisomycin-stimulated HEK cells in FIG. 41. However, the amount of total Akt1 expressed in the three tumor lines was higher than phosphorylated Akt1 (compare FIG. 41 to FIG. 40). For these studies, the solid phase (Bead) antibody was Cell Signaling Technology, Inc. catalog no. 2697 (a mouse MAb to total AKT) and the detector (FL labeled ab) was Cell Signaling Technology, Inc. catalog no. 4691 (a mouse MAb to total AKT).

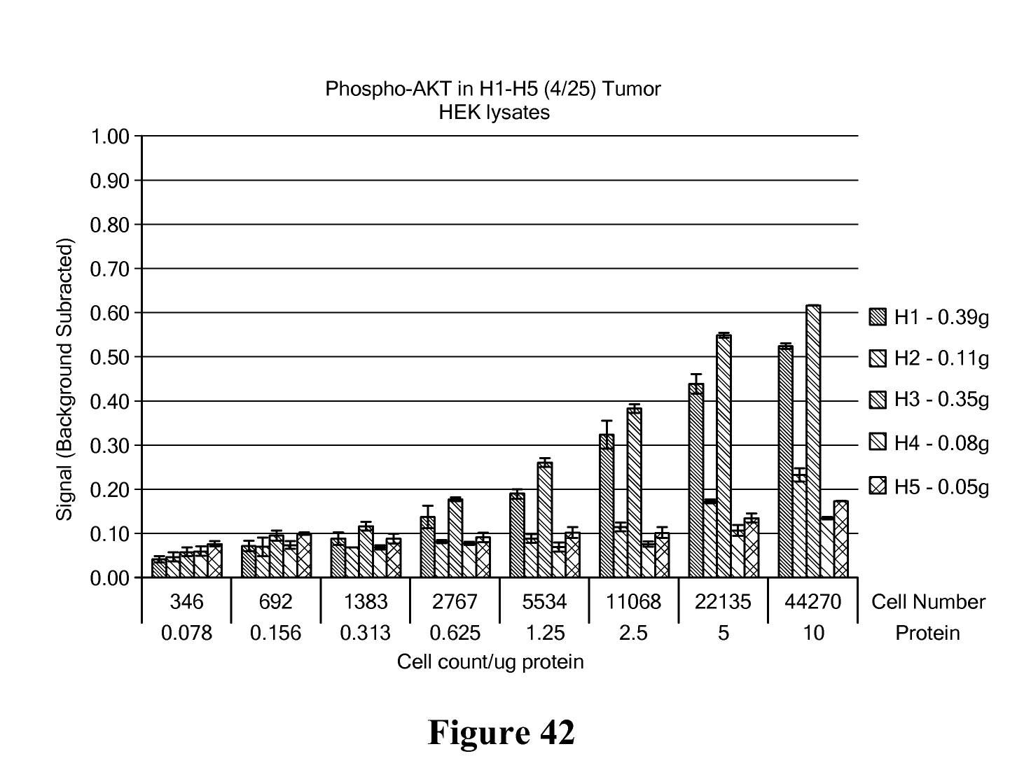

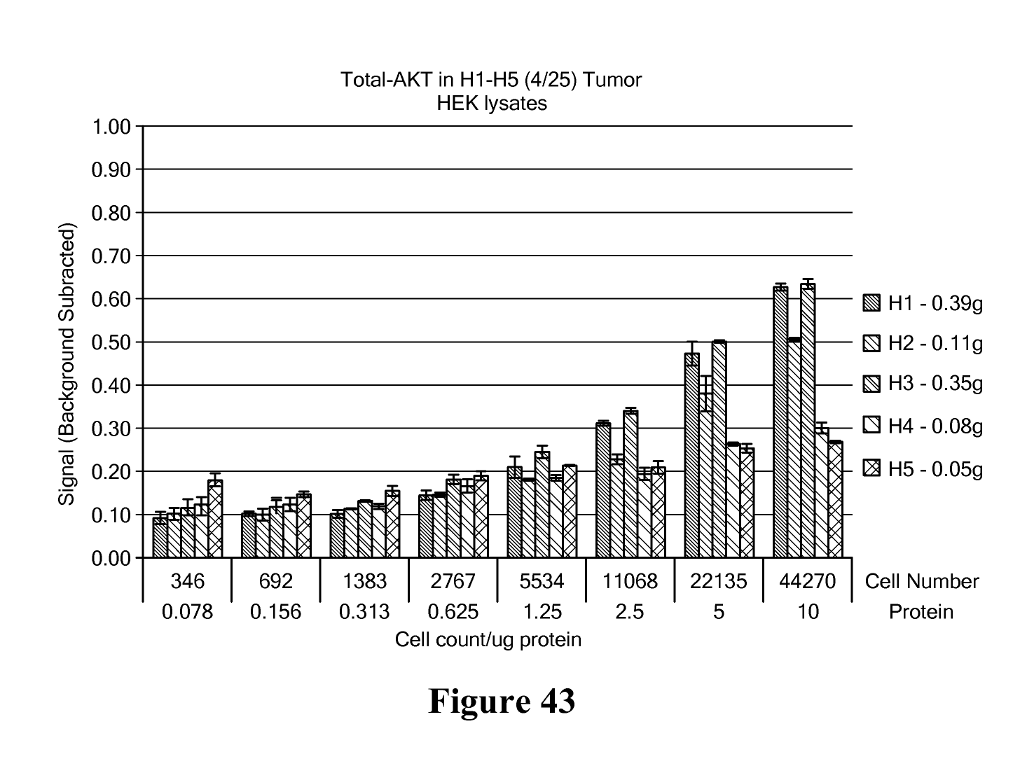

The levels of phosphorylated Akt1 and total Akt1 in the H1, H2, H3, H4, and H5 tumors was next assessed. As shown in FIG. 42, tumors H1 and H3 had the highest levels of phosphorylated Akt1, as well as the highest levels of total Akt1. The weights of the tumors are shown on the right in FIGS. 41 and 42. These data appears to indicate that the larger the tumor mass, the higher the amount of phosphorylated Akt1 expressed by the tumor and, to a slightly lesser extent, the higher the amount of total Akt1 expressed by the tumor.

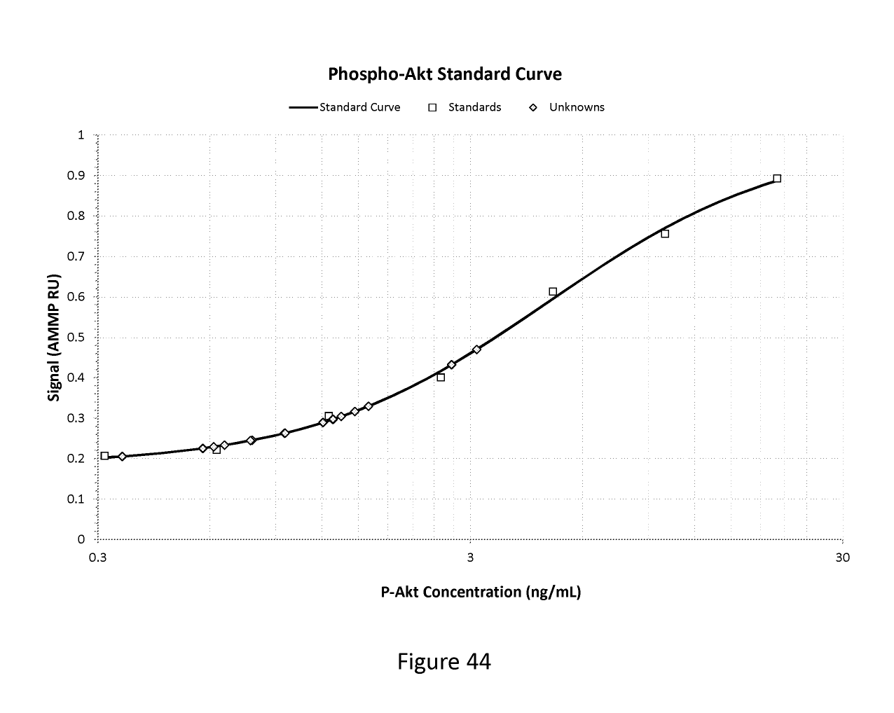

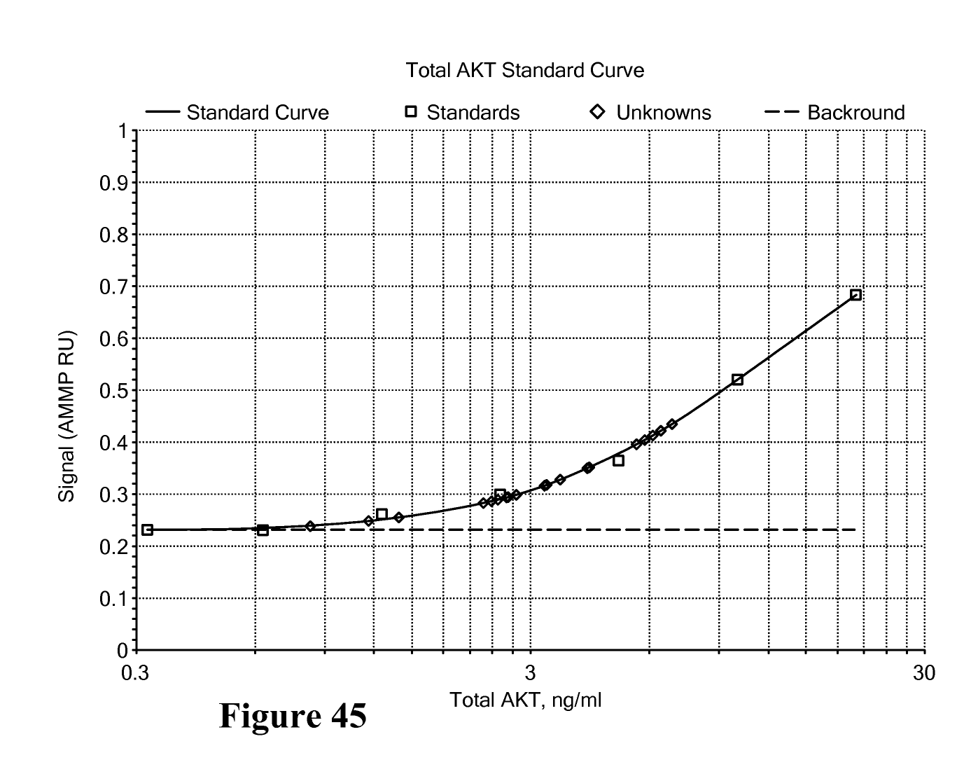

From these data from the AMMP assays using the ViBE instrument, standard curves of phosphorylated Akt1 (FIG. 44) and total Akt1 (FIG. 45) were generated.

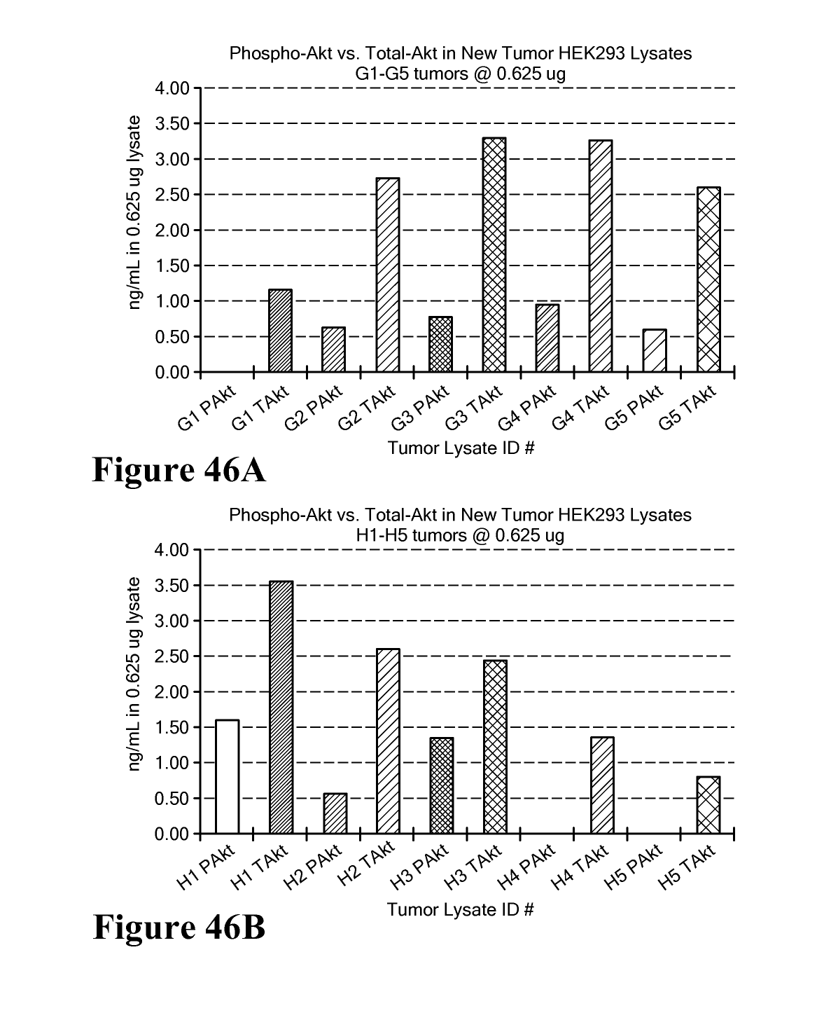

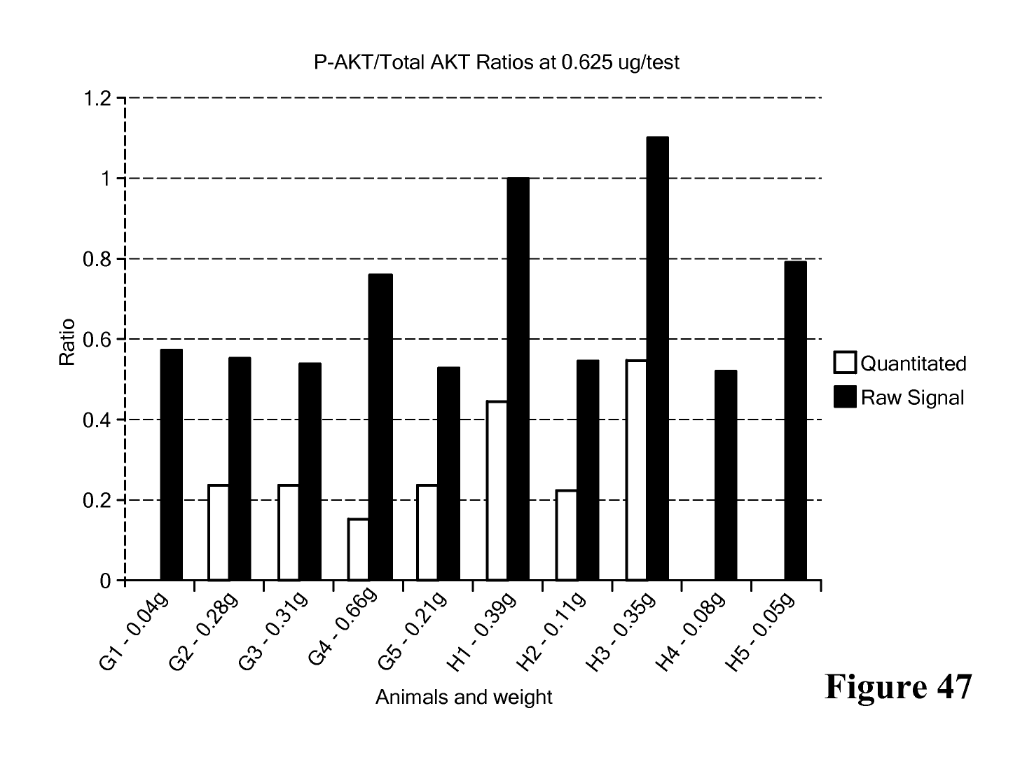

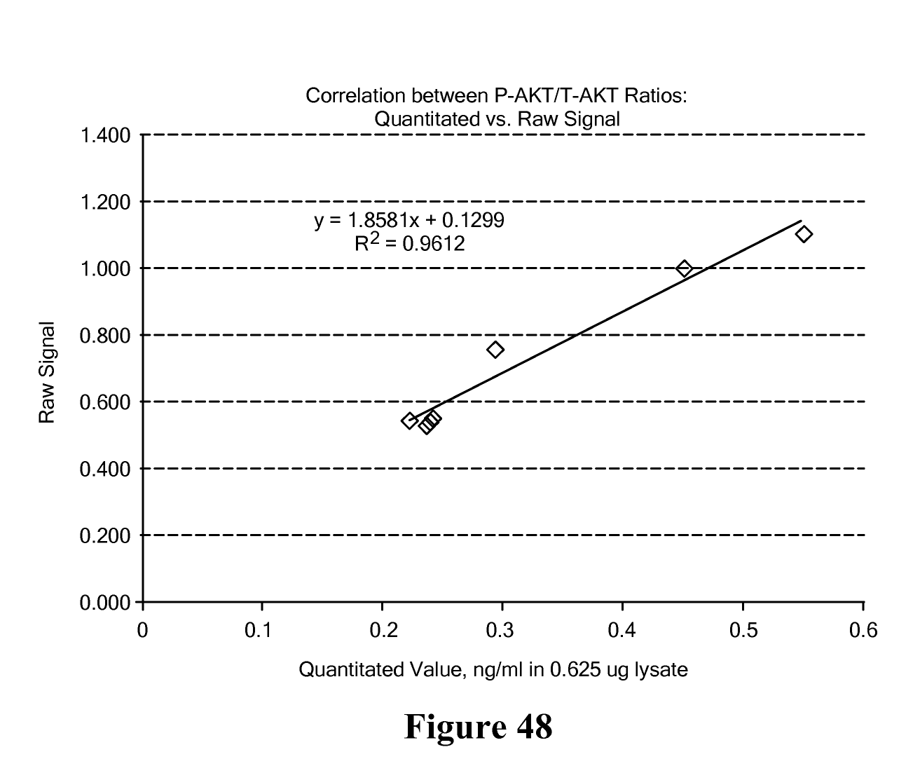

FIGS. 46A and 46B show side-by-side comparisons of phosphorylated Akt1 and total Akt1 in G1, G2, G3, G4, and G5 tumor lysates (FIG. 46A) and in H1, H2, H3, H4, and H5 tumor lysates (FIG. 46B). FIG. 47 shows the ratio of pAKT1/total AKT1 as calculated using either the raw signal (AMMP units; light purple bars) or as calculated from the concentrations of proteins calculated by the ViBE instrument. FIG. 48 show regression analysis of the data shown in FIG. 47 in order to generate the R squared value and the regression equation to demonstrate the relationship between the two methods numerically.

As the results in this Example 5 show, the correlation of measurement of kinases and receptors in tumor cell lines with the same cell line xenografts was excellent. The number of cells contained in specific amounts of cell lysate has been determined, allowing the elucidation of the amounts of these analytes in samples representing very low cell counts. Consequently, the use of AMMP technology has permitted measuring the activation states of multiple kinases, from fewer than 2000 cultured or xenografted tumor cells per assessment. Moreover, the methods described herein provide a quantification method by which phospho and total AKT can be measured in ng/ml quantities and provide correlation data by which quantification was readily and accurately assessed.

Thus, the combination of the ability to detect the phospho and total analytes in small cell number samples, the absence of matrix effects from tumor and cell lysates, and the ease of use make the AMMP assays on the VIBE excellent tools for oncology research, for both in vitro and in vivo models (e.g., xenograft mouse models).

### Example 6

### Detection and Analysis of Biomarkers to Determine Efficacy of a Therapeutic Treatment

There are several candidate small molecule agents currently in development that target the PI3K pathway. Table 12 provides a non-exclusive list of these agents.

TABLE 12

| Agent | Company | Molecular targets |
|---|---|---|
| BYL719 | Novartis | PI3Kα |
| GDC-0032 | Genentech | PI3Kα |
| INK-1117 | Intellikine | PI3Kα |
| XL-147 | Exelixis/Sanofi | Pan-PI3K |
| NVP-BKM120 | Novartis | Pan-PI3K |
| GDC-0941 | Genentech | Pan-PI3K |
| PKI-587 | Pfizer | Pan-PI3K |
| XL-765 | Exelixis/Sanofi | PI3K/mTOR |
| NVP-BEZ-235 | Novartis | PI3K/mTOR |
| NVP-BGT-226 | Novartis | PI3K/mTOR |
| PF-4691502 | Pfizer | PI3K/mTOR |
| AZD8055 | Astrazeneca | mTOR (catalytic) |
| INK 128 | Intellikine | mTOR (catalytic) |
| MK-2206 | Merck | AKT1,2,3 |
| GDC-0068 | Genentech | AKT |

In some embodiments, using the methods described herein, subjects having tumors, such as HEK-293, that expressed activated (i.e., phosphorylated Akt1) can be treated with varying dosages of one or more of these candidate therapeutics. Fine needle aspirates are taken from the tumor tissue before the start of treatment and at various times during treatment. In some embodiments, the methods described herein provide rapid and accurate measurements of the amounts of biomarkers from very small numbers of cells. If there is a reduction in the amount of phosphorylated Akt1 (or another signaling molecule in the PI3 kinase pathway such as PDK1 or S6K), the reduction may indicate that the drug is being used at an effective dosage and/or that it is effective in shrinking the tumor.

In addition, it is well known that certain tumors may initially respond to a therapeutic regimen, but the tumor may by-pass the signaling molecule targeted by the chemotherapeutic and continue to propagate using a parallel signaling pathway. Using the methods described herein, rapid assessments can be made of the levels of biomarkers in salvage pathways by predicting what pathways might be used based on the hallmark pathways identified in Jones et al. (Science 321: 1801-1806, 2008). By being able to quickly and accurately detect changes in levels of active and total protein, in some embodiments, the methods described herein provide a method to tailor a patient's therapeutic regimen and change that patient's drug based on the response made by the patient's cells.

### Example 7

### Analyzing Tumor Cells Treated with Specific Anti-Cancer Agents

The Ras pathway is perhaps one of the most commonly deregulated pathways in human cancer (see, e.g., Courtois-Cox et al., Oncogene 27: 2801-9 (1 May 2008).

Some non-limiting well known signal transduction pathways include the ras/PI3 kinase pathway where activated ras can activate PI3 kinase which in turn activates PDK which in turn activates Akt (by phosphorylating it) which in turn activates a number of targets including mTOR (which activates protein translation), XIAP (which inhibits apoptosis), and IKK (which activates NF-kB). Another well known pathway is the ras/raf pathway, where active ras activates raf which activates MEK1/2 which activates ERK1/2, which then activates transcription. FIG. 49 shows parts of these signal transduction pathways.

Anti-cancer agents can selectively target biomarkers that play roles in a desired signal transduction pathway. For example, active MEK is selectively targeted by PD0325901, AZD6244, and XL418. Similarly, PI3 kinase is selectively targeted by XL 147 and BGT226.

To determine whether or not the targeted treatment of a particular biomarker in the signal transduction pathway of a tumor cell (e.g., a neoplastic, metastatic cancer cell or a benign, hyperplastic cell) with an anti-cancer agent is able to specifically inhibit the target of the anti-cancer agent, tumor cell samples can be treated with various anti-cancer agents to determine whether or not the targeted biomarker shows a reduced expression in cells treated with the agent.

Treating the cell with an agent will, of course, depend upon the agent. Typically, a damaged cell (or normal control cell) can be treated with an agent by simply contacting the cell with the agent. For example, the agent may be an siRNA targeting a biomarker, and treatment with the siRNA may be, for example, simply introducing a dsRNA to a cell culture which is uptaken by the cell and cleaved into siRNA. Working from the signal transduction pathways depicted in FIG. 49, the biomarkers may be, for example, MEK, PI3K, and KRAS. The sequences of nucleic acid molecules encoding KRAS, MEK, and PI3 kinase are known. For example, the sequence of KRAS is provided at GenBank Nos. AF493917.1 and L00049.1 (Shimizu et al., Nature 304: 497-500, 1983; McGrath et al., Nature 304: 501-506, 1983). SiRNA molecules targeting such biomarkers, and cells expressing the same can easily be made by standard methods (see, e.g., Brummelkamp et al., Science 296: 50-553, 2002; Elbashir et al., EMBO J. 20: 6877-6888, 2001; Yu et al., Proc. Natl. Acad. Sci. 99(9): 6047-6052, 2002; Paddison et al., Genes Devel. 16: 948-958, 2002; and U.S. Pat. Nos. 7,078,196 and 8,198,252).

Other agents can be used, such as the agents described here. For example, the MEK-targeting PD03325901 compound (see, e.g., CAS No. 391210-10-9; Henderson et al., Mol. Cancer Ther. 9(7): 1968-1976, 2010) can be purchased commercially as Catalog No. 13034 from Cayman Chemical, Ann Arbor, Mich. Example 6 above likewise lists several agents and their suppliers that selectively target PI3 kinase. These small molecules can be, for example, added to cultured cells which will internalize them. For in vivo delivery, subjects (e.g., human patients) can be treated by oral or injection, and the damaged cells within the subject are thus treated when the subject is treated.

The agent may also be a binding agent (such as an antibody) that specifically binds to the biomarker. For example, the binding agent may be a humanized neutralizing antibody. Treatment of cells with such an agent may entail lipid bilayer fusion, for example, with an encapsulated agent such that the binding agent will be internalized by the cell such that it can specifically bind to the biomarker inside the cell.

It will be understood that the various agents can be used in combination. For example, an agent targeting KRAS may be used in combination with an agent targeting MEK to treat a tumor cell and/or an agent targeting PI3 Kinase. Likewise, an agent targeting PI3 Kinase can used be together with an agent targeting MEK to treat a cell. It should also be noted that if two or more agents are used together, they need not both target nucleic acid or both target a polypeptide. For example, an siRNA targeting a nucleic acid encoding MEK can be used in combination with a small molecule (e.g., the PF-05212384 (also known as PKI-587; CAS NO. 1197160-78-3) compound that targets PI3 kinase.

Finally, tumor cells can be from any source. Mammalian tumor cells (e.g., colon cancer cells, breast cancer cells, leukemia cells, lymphoma cells, prostate cancer cells, lung cancer cells, etc.) are commercially available from the American Type Culture Collection (“ATCC”; Manassas, Va.). Tumor cells taken from tumor biopsies or from other bodily tissues or fluids (e.g., from a fine needle aspirate or blood) may also be used.

Thus, treating the damaged cell with the agent will depend upon the agent and upon the damaged cell. For example, a cell may be treated in vivo, the damaged cells excised (e.g., via a biopsy), and the biomarker presence and level measured using the AMMP methods described herein.

It is expected that a damaged cell such as a tumor cell that is treated with an agent that targets a specific biomarker will show a reduced level of expression of that biomarker. It is also expected that a tumor cell treated with an agent that targets as specific biomarker will show a reduced level of expression of an activated form of a biomarker downstream in the signal transduction pathway that the downstream biomarker shares with the targeted biomarker.

Note that by “downstream” is meant that the downstream biomarker participates in the signal transduction pathway after participation of an earlier biomarker. In some embodiments, the upstream biomarker is activated and its activation leads, directly or indirectly, to the involvement of the downstream biomarker.

By “upstream” is meant that the upstream biomarker participates in the signal transduction pathway before participation of a later downstream biomarker. In some embodiments, the upstream biomarker is activated and its activation leads, either directly or indirectly, to the involvement of the downstream biomarker (e.g., the upstream biomarker once activated may activate the downstream biomarker by phosphorylating it.

For example, the phosphorylation of AKT kinase is downstream of (and activated) by active PI3 kinase. Thus, if PI3K were inhibited (e.g., by treatment with an anti-cancer agent targeting PI3K such as an siRNA that hybridizes (e.g., under stringent conditions or highly stringent conditions) to a nucleic acid sequence encoding PI3K or treatment with the NVP-BKM120 compound from Novartis), it is expected that the phosphorylated form of AKT may be decreased in the treated cell. However, the amount of total AKT (i.e., both phosphorylated and non-phosphorylated) may or may not be decreased in the treated cell.

Likewise, since both phosphorylation of JNK and phosphorylation of p38 occur downstream of RAS activation, it is expected that the amounts of both phosphorylated p38 and phosphorylated JNK will be reduced in tumor cells treated with an agent targeting RAS, but the amount of total protein (i.e., total JNK and total p38) may or may not be similarly reduced in the treated cell as compared to an untreated cell.

Once a signal transduction pathway is inhibited by targeting a biomarker involved in the pathway, the entire pathway may be shut down. Thus, it is also expected that a tumor cell treated with an agent that targets as specific biomarker will show a reduced level of expression of an activated form of a biomarker upstream in the signal transduction pathway that the downstream biomarker shares with the targeted biomarker.

Since phosphorylation of the Epidermal Growth Factor Receptor (EGFR) is upstream of RAS activation, it is expected that the amount of phosphorylated (i.e., active) form of EGFR may be reduced in tumor cells treated with an agent targeting RAS, but the amount of total EGFR (i.e., both phosphorylated and non-phosphorylated forms) may stay the same in the treated cell ((as compared to an untreated cell).

Note that as used herein, by “stringent conditions” is meant that the two complementary single-stranded nucleic acid molecules will hybridize to form a dimer and remain dimerized at 60° C. in 1.0×SSC (standard sodium citrate) buffer, where a 20×SSC solution comprises 3M NaCl and 300 mM Na₃C₆H₅O₇ at a pH of 7.0 (adjusted with HCl). By “high stringent conditions” means that the two complementary single-stranded nucleic acid molecules will hybridize to form a dimer and remain dimerized at 65° C. in 0.1×SSC (standard sodium citrate) buffer, where a 20×SSC solution comprises 3M NaCl and 300 mM Na₃C₆H₅O₇ at a pH of 7.0 (adjusted with HCl).

Additionally, as discussed in Example 6, it is known that tumor cells treated with chemotherapeutic can develop resistance by signaling through alternate signal transduction pathways. For example, in estrogen receptor positive (ER+) advanced breast cancer, endocrine therapy (therapy inhibiting estrogen and progesterone) is largely successful, with approximately 60% of ER+ breast cancers responding (Hynes et al., J Mammary Gland Biol Neoplasia. 2006; 11:53-61; Leary et al., Br. J Cancer. 2006; 95:661-666.). However, hyperactivation of the PI3k/ATK/mTOR signal transduction pathway has been shown to allow tumors to evade endocrine therapy, and thus develop resistance to the therapy (see Miller et al., J Clin Invest. 2010; 120(7):2406-2413)

The methods described herein can be used to detect whether or not a tumor cell may be attempting to resist the treatment with the anti-cancer agent by using an alternate signaling pathway.

For example, in a tumor cell treated with an agent targeting MEK kinase, it is expected that the cells will try to escape the effects of the agent by signaling through an alternative signal transduction pathway. Thus, the amount of phosphorylated JNK phosphorylated p38, and/or phosphorylated AKT is expected to increase in the treated cell (i.e., the cell treated with the agent targeting MEK), while the amount of total JNK, total p38, and/or total AKT may or may not increase in the treated cell (as compared to an untreated cell).

In another example, the MCF7 breast cancer line expresses Estrogen receptor. Endocrine therapy may be used on these cells (using, e.g., the methods described in Beeram et al., Ann. Oncol. 18 (8): 1323-1328, 2007), and the PI3K/ATK/mTOR signal transduction pathway may be analyzed to determine if the biomarkers in the pathway are activated and, if so, if they are present in amounts higher than a treated MCF7 cell line that has just begun treatment with endocrine therapy and is still responding (e.g., still showing slowed growth).

It is expected that using the methods described herein, some number of MCF-7 cells that have been treated for a month, or two month, or three months, or four months with endocrine therapy may begin to express higher levels of phosphorylated AKT kinase than MCF-7 cells that have been treated with endocrine therapy for less than one month or for less than one week.

### Example 8

### AMMP Studies for the Prediction of the Effectiveness of Kinase Inhibitors in the TRK Pathways

Kinase signaling pathways play an integral role in regulating cell growth and survival. Not surprisingly, dysfunction of kinase signaling pathways has been linked to the development and progression of numerous cancers. This general principle is true of cancers linked to a number of tyrosine receptor kinases (TRKs), including fibroblast growth factor receptor (FGFR), insulin-like growth factor-I receptor (IGF-IR) and epidermal growth factor receptor (EGFR). Dysfunction of the TRK signaling pathway in particular is linked to a number of cancers, including non-small cell lung cancer, basal cell carcinoma and esophageal cancer. As a consequence, TRK-linked cancers are frequently targeted therapeutically by monoclonal antibodies and small molecule tyrosine kinase inhibitors (TKIs), such as Gefitinib (IRESSA®), Erlotinib (TARCEVA®) and Lapatinib (TYKERB®), that inhibit different components of the TRK signaling pathway (see Yoshikawa et al., Oncogene 32(1):27-38, 2012). More than twenty small-molecule and monoclonal antibody TKIs are already approved for clinical treatment of a number of cancers, and dozens more are in various stages of clinical development.

Developing cancer therapeutics that target the TRK signaling pathway is complicated on several fronts. First, mutations in the TRK and downstream components of its signaling pathway confer resistance to TKIs, leading to increased pressure to develop drugs that target drug-resistant mutants. Second, the multiplicity of downstream signaling pathways for TRKs complicates the identification of appropriate targets for new therapeutics. Finally, traditional studies of TRK signaling focus on individual signaling pathway components and their individual interactions. However, that approach limits the real-world applications of TRK signaling when devising new therapeutics for kinase signaling-linked cancers.

The EGF family of receptors typifies the complications associated with developing therapeutics for TKR-related cancers. There are four EGFR family members, erbB1-B4, as well as several downstream signal transduction pathways, including the major pathways RAS/RAF/MAPK, RAS/PI3-K/AKT and SRC/STAT1 and STAT3, that also play a role in downstream signaling for other receptor tyrosine kinase growth factors (Chakravarti et al., Int. J. Rad. Oncol., Biol., Phys. 58: 927-931, 2004). Crosstalk between EGFR and other growth factor signaling pathways can lead to acquired resistance against drugs that target only EGFR, as evidenced by crosstalk between EGFR and IGF-1R leading to acquired resistance to drugs targeting only EGFR in the treatment of cancer. Such complications have serious implications for effectively treating cancer. For example, a number of breast cancer anti-estrogen resistance genes associated with different signal transduction pathways have been identified that correlate with tamoxifen resistance in breast cancer, while malignant gliomas exhibit variable responses to treatment depending on the EGFR pathway component that is targeted.

Given the complicated nature of identifying appropriate targets for the development of novel chemotherapeutic agents, this Example 8 describes the development of a series of assays that reveal molecular interactions of components of classic kinase signaling pathways. The assay herein described using the AMMP technology permitted rapid exploration of the interrelated kinase signaling pathways across multiple cell lines, while providing new information regarding the interactions between proteins in kinase pathways. Drugs that target TRK pathways require particularly detailed knowledge interactions between the signaling pathway components. The development of drug-resistant EGFR mutants, combined with crosstalk between kinase signaling pathways, creates a complicated scenario for drug development. Though many drugs have been developed to prevent EGF-led carcinogenesis, many have failed to achieve complete remission of patient disease. This Example 8 provides a panel of assays that revealed molecular interactions of components of classic kinase signaling pathways and was used for predicting both the on-target and off-target effects of the EGFR class of kinase inhibitors.

This Example 8 focuses on exploration of the EGFR signaling pathway as a model for understanding kinase activity and crosstalk between signaling pathways. This assay shed new light on interactions between known components of the intracellular signaling of EGFR through MAPK, MEK, JNK and AKT, while also furthering current knowledge of other receptors that influence the EGFR signal transduction pathway. With a panel of kinase assays such as the panel described in this Example 8, the biology of the EGF class of receptors can be better understood and screening for the efficacy of EGFR inhibitors for wanted and unwanted specificity has been made possible. This study provided a rich foundation for discovering and developing new cancer therapeutics.

Cell Used. For this Example, the following cells were used. Jurkat, HEK-293, 3T3, WM266.4, A431 and MCF-7 cells (purchased from the American Type Culture Collection, Manassas, Va. (ATCC)) were grown at 37° C. in a CO₂ supplemented incubator in basal media containing fetal bovine serum. Cells were cultured as specified by the ATCC.

Methodology Used: For drug treatment, cells were stimulated with or inhibited by one or more combinations of following drugs: epidermal growth factor (EGF) 100 ng/ml (R&D Systems) for 10 minutes, phorbol myristate acetate (PMA) 250 ng/ml (Sigma Aldrich) for 30 minutes, Rapamycin 1 ng/ml (Sigma Aldrich) for 180 minutes, Calyculin 10 ng/ml (Sigma Aldrich) for 30 minutes, insulin-like growth factor 1 (IGF-1) 100 ng/ml (R&D Systems) for 20 minutes, Anisomysin at 200 or 2000 ng/ml for 30 minutes or LY294002 50 uM (Cell Signaling Technology, Inc.) for 20 minutes in basal cell culture media prior to lysis of the treated cells with tissue protein extraction reagent (TPER; ThermoFisher/Pierce). For the EGFR inhibitor studies, cells were treated with EGFR inhibitors Afatinib (13 nM), Gefitinib (37 nM) or Neratinib (92 nM) (Selleckchem), for 2 hours in basal media prior to EGF stimulation (stimulation at 100 ng/ml for 10 minutes). TPER was supplemented with phosphatase and protease inhibitors (ThermoFisher/Pierce) as described by the manufacturer. The cells were lysed by pipetting the TPER on the adhesion cell layer, or onto the cell pellet in the case of Jurkat cells. Cell lysates were collected into conical tubes, placed on ice, and vortexed every 5 minutes for a total of 20 minutes before aliquoting and freezing on dry ice. A standard BCA protein assay was used to determine total protein concentration of the lysates.

AMMP Assay Design: These studies were designed using AMMP® (Acoustic Membrane Microparticle Assay) as run on the ViBE® workstation (BioScale). Assays were constructed so that strepavidin coated magnetic microparticles were loaded with analyte-specific biotinylated antibody and the solution phase sandwich partner antibody was conjugated with fluorescein. The complex of bead-analyte and fluorescien-labeled antibody was detected by the anti-fluorescein antibody-coated acoustic membrane of the VIBE. All of the antibodies used in these assays were procured from Cell Signaling Technology, Inc. (Danvers, Mass.), with the exception of the anti-HER3 MAB3481 which was purchased from R&D Systems. Specifically, for the total EGFR assay, anti-EGFR antibody 2646 (i.e., Cell Signaling Technology, Inc., catalog No. 2646) was coated on beads, and anti-EGFR antibody 2256 labeled with fluorescein. For the pEGFR assay, anti-phospho (tyr1068)-EGFR antibody 3777 was coated on beads, and anti-EGFR antibody 2256 was fluorescein labeled. For the pERK1/2 assay, anti-p44/42 MAPK antibody 4695 was coated on beads, and anti-phospho-p44/42 MAPK (thr202/tyr204) antibody 4376 was fluorescein labeled. For the pMEK1 assay, anti-phospho MEK1/2 (ser217/221) antibody 9154 was coated on beads and anti-MEK1 antibody 2352 was fluorescein labeled. For the pp38 assay, anti-p38-alpha MAPK antibody 9218 was coated on beads, and anti-phospho-p38 MAPK (thr180/tyr182) antibody 4511 was fluorescein labeled. For the pJNK assay, anti-SAPK/JNK antibody 9258 was coated on beads, and anti-phospho-SAPK/JNK (thr183/tyr185) antibody 4668 was fluorescein labeled. For the pAKT1 assay, anti-phospho-AKT1 (ser473) antibody 4058 was coated on beads, and anti-AKT1 (pan) antibody 2967 was fluorescein labeled. For the pHER3 assay, anti phospho-HER3/ErbB3 (tyr1197) 4561 was coated on beads, and anti-HER3 MAB3481 (purchased from R&D Systems) was fluorescein labeled.

Assay Set-Up: Cell lysates were thawed on ice then centrifuged at 14,000 RPM. Forty microliters of diluted cell lysates were pipetted in to the appropriate well of each plate with forty microliters of sample diluent. Also to each well, a total of forty microliters of a mixture of 1.5×10⁵ magnetic beads and 200 ng/ml of fluorescein labeled detector antibody were added. Separate wells were loaded with beads and lysates alone to serve as negative controls. The plates were loaded on the VIBE workstation (BioScale Inc., Lexington, Mass.) and incubated for 4 hours prior to analysis. Results were obtained for each sample by subtracting the AMMP signal of the bead only control from the raw signal assay values derived from the sample and reported in AMMP response units.

Western Blotting Analysis Methods: For these analyses, cell lysates were diluted 1:1 in SDS-PAGE sample buffer (Invitrogen, Carlsbad, Calif.) and were electrophoretically resolved on 8-16% Tris-glycine gels (Invitrogen) under constant current for 2 hours. The resolved proteins were subsequently transferred to PVDF membranes (Invitrogen). The PVDF membranes were blocked for 1 hour in Tris buffered saline containing 5% non-fat dried milk or 5% bovine serum albumin (BSA). PVDF membranes were then incubated with primary antibodies diluted in either of the two blocking buffers overnight at 4° C. The next day each membrane was washed vigorously with Tris buffered saline containing 0.1% Tween, and then incubated with peroxidase labeled anti-species specific secondary antibodies in normal donkey serum for 1 hour at room temperature. The membranes were then incubated with a chemiluminescent substrate reagent (Invitrogen) for 40 seconds and images were captured using a Protein Simple Fluorechem E.

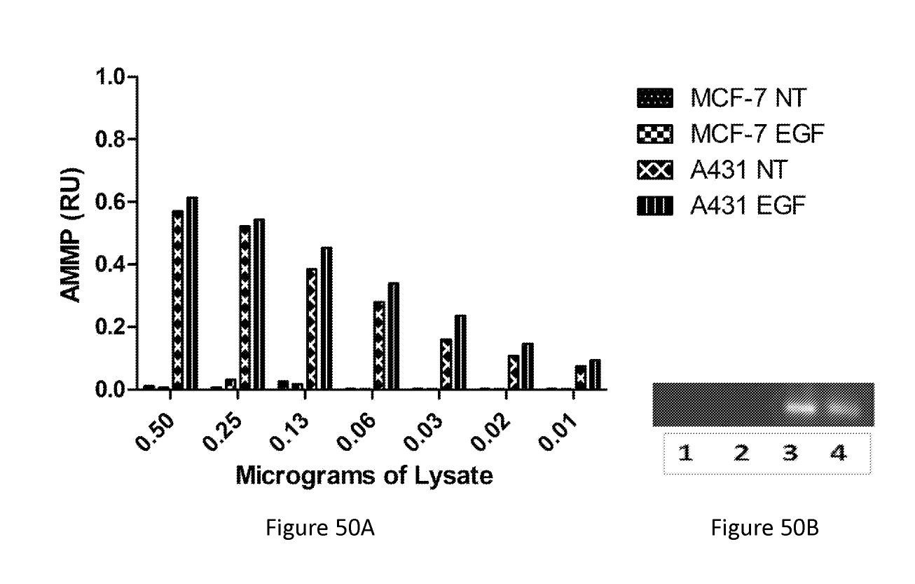

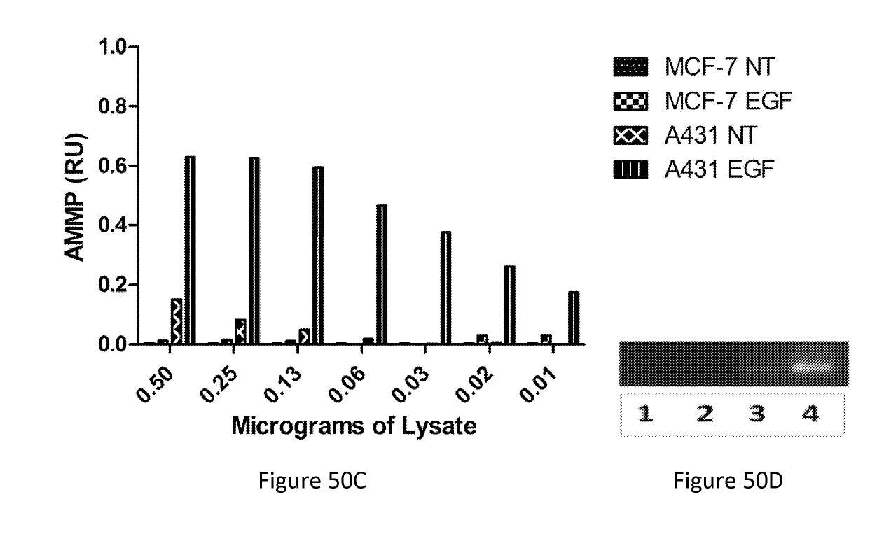

From this Example 8, a number of results were obtained. First, these studies confirmed EGFR expression levels and activity in A431 and MCF-7 cell lines. Using the methods described above, the first step was to identify and confirm both high- and low-expressing EGFR cell lines. The high total EGFR expression reported for the A431 cell line, for both stimulated and non-stimulated cells was confirmed using AMMP assay technology (FIG. 50A)) (“A431 NT” for no treatment and “A431 EGF” for A431 cells treated with EGF). The low total EGFR expression levels of MCF-7 cells (both stimulated and non-stimulated) was also confirmed by AMMP assay technology (FIG. 50A) (“MCF-7 NT” for no treatment and “MCF-7 EGF” for MCF-7 cells treated with EGF). Total EGFR expression was seen in as little as eight nanograms of lysate of the A431 cells with no changes in expression seen following a 10 minute pulse of EGF. Western blotting analyses confirmed these findings as shown in FIG. 50C showing Western blotting analyses for total EGFR expression in non-treated MCF-7 cells in lane 1, EGFR-treated MCF-7 cells in lane 2, non-treated A431 cells in lane 3, and EGF-treated A431 cells in lane 4).

AMMP assay technology was also used to confirm EGFR activity as measured by the level of phosphorylated EGFR. As expected, EGF stimulation of A431 cells caused phosphorylation of the EGFR which was detectable in as little as eight nanograms of lysate in the assay, while minimal EGFR phosphorylation was detected in the MCF-7 cell lines with or without EGF stimulation (FIG. 50C). AMMP assay technology was also used to confirm EGFR activity. As expected, EGF stimulation of A431 cells caused phosphorylation of the EGFR which was detectable in as little as eight nanograms of lysate in the assay, while minimal EGFR phosphorylation was detected in the MCF-7 cell lines with or without EGF stimulation (FIG. 50B). EGFR phosphorylation data was also confirmed by using six micrograms of lysates on the Western blotting analysis (see FIG. 50D, showing phosphorylated EGFR expression in non-treated MCF-7 cells in lane 1, EGFR-treated MCF-7 cells in lane 2, non-treated A431 cells in lane 3, and EGF-treated A431 cells in lane 4).

The results also showed that this assay development could be used to identify activated signaling pathways. Although the essential outlines of kinase signaling pathways are well understood, the specific details of their molecular interactions are frequently debated in the literature. In this study, several different cell lines and stimulation conditions previously reported in the literature as being specific to each analyte were used to confirm performance of the AMMP assay.

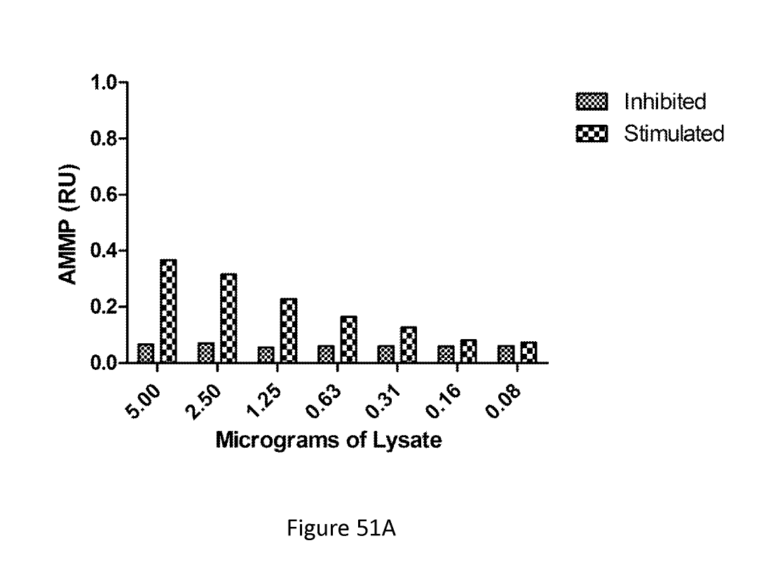

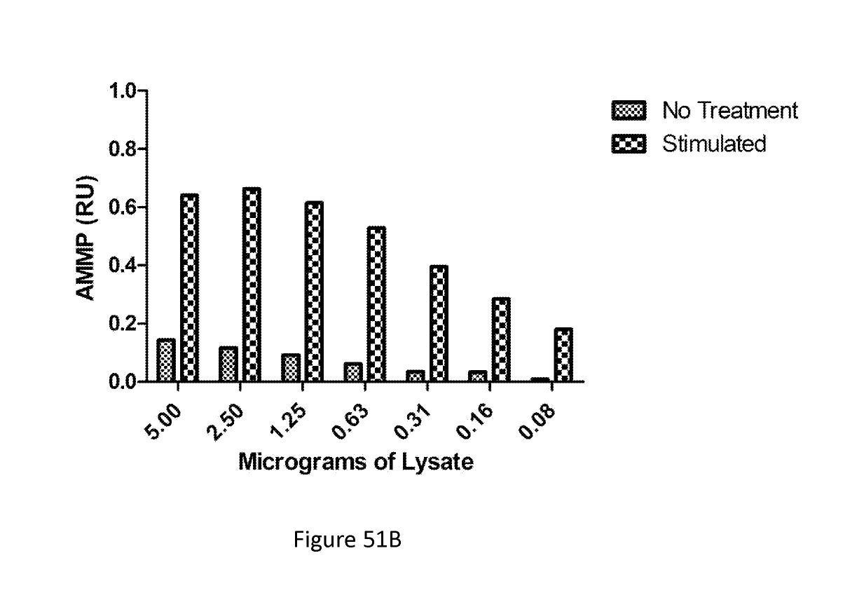

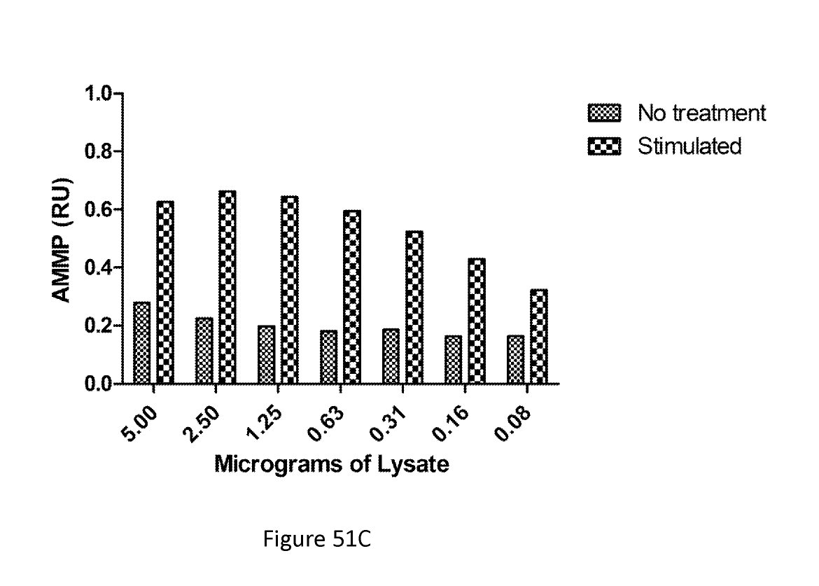

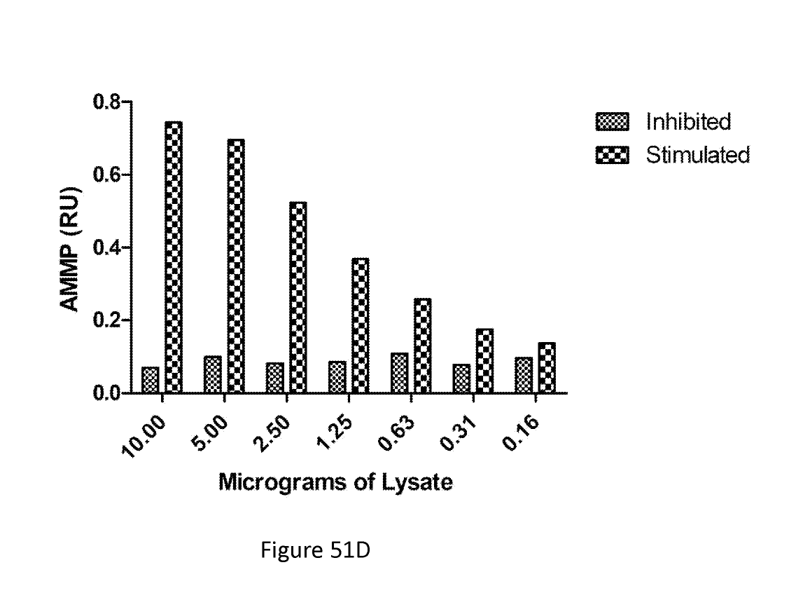

Four AMMP assays were developed to monitor phosphorylation activity in each of the four (ERK1/2, p38, JNK and AKT1) distinct but interrelated pathways (see, e.g., FIGS. 9, 10A, and 10B) that were selected for further exploration. Detection of kinase activity by the AMMP assay was confirmed using agonists known to stimulate each test cell line. For the assay designed to detect of phosphorylation of ERK1/2, Jurkat cells were stimulated with PMA and Calyculin or inhibited with Rapamycin as the negative control to prevent the phosphorylation of ERK1/2 (see FIG. 51A). The assays developed to detect phosphorylation of JNK or p38 utilized Anisomyosin stimulation of HEK293 (see FIG. 51B) or mouse 3T3 (see FIG. 51C), respectively, as positive control cell line lysates. Finally the AMMP assay used to detect the phosphorylation of AKT1 utilized the cell line WM266.4 stimulated with IGF-1 (see FIG. 51D). All results were again confirmed by Western blot (data not shown). As few as one hundred nanograms of lysate were needed to measure each target kinase.

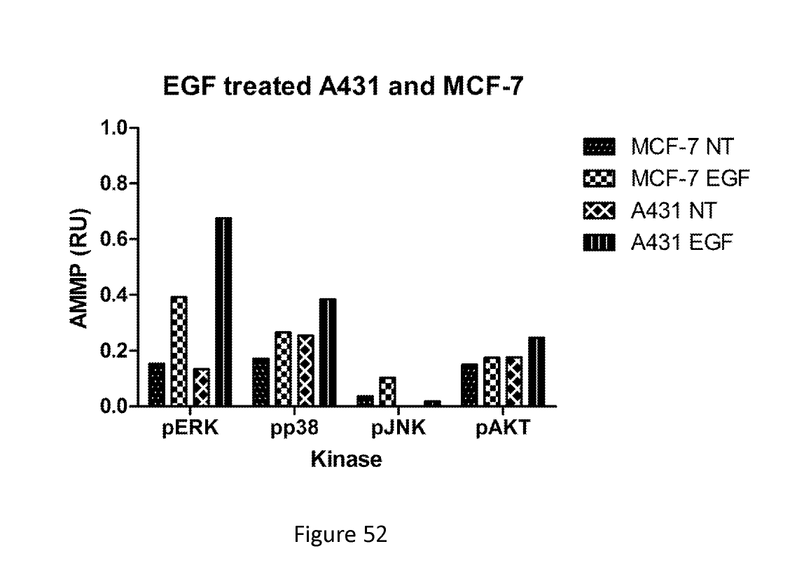

ERK1/2 is suggested by scientific literature to be the primary downstream kinase activated by EGF stimulation (see Seger et al., FASEB J. 9: 725-736, 1995). To confirm this, the AMMP assays were applied in to A431 and MCF-7 cells stimulated by EGF. Data confirmed phosphorylation of ERK1/2, p38 and AKT1, and JNK (see FIG. 52). As shown in FIG. 52, phosphorylation of ERK 1/2 predominated the signaling pathway in EGF-stimulated A431 cells (FIG. 52). Interestingly, the assay detected phosphorylation of ERK1/2 and moderate phosphorylation of p38 in EGF-stimulated MCF-7 cells, despite their low EGFR expression levels. There were only detectable levels of phosphorylation of ERK1/2 in the EGF stimulated MCF-7 cell line with no change in JNK in either cell line. Detection of pp38 under these experimental conditions suggested crosstalk, whereby EGF may activate a receptor other than EGFR to stimulate kinase signaling. Alternatively, another EGF family receptor could be involved.

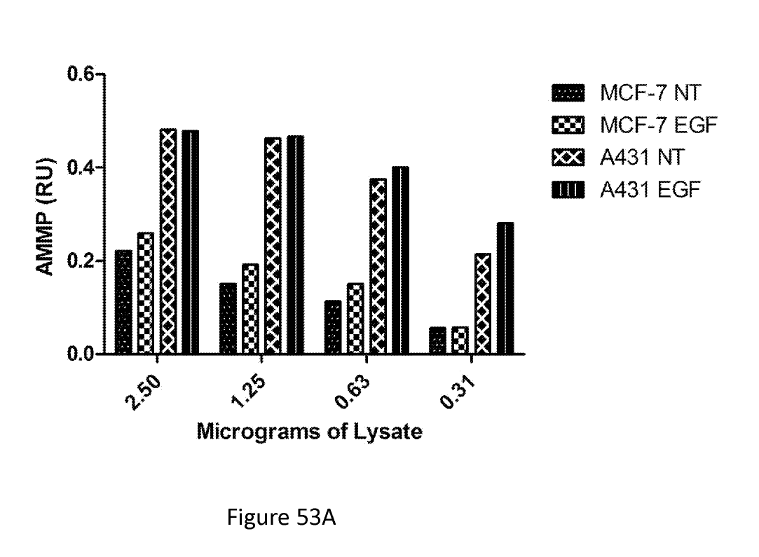

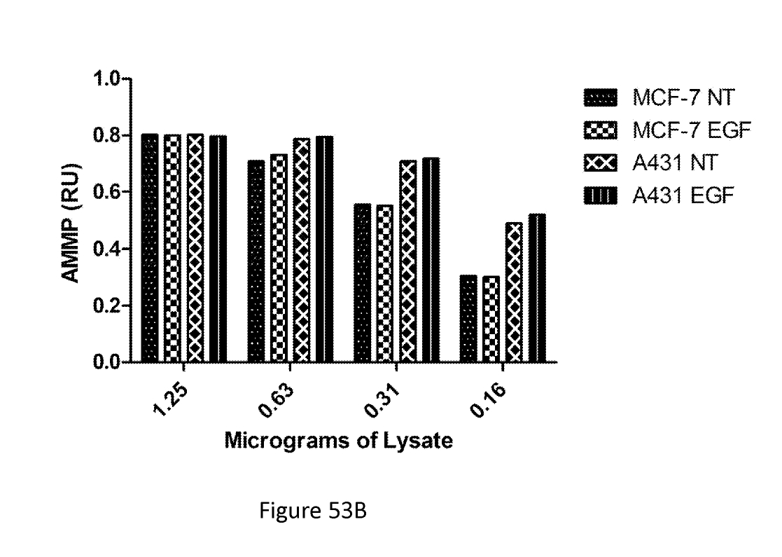

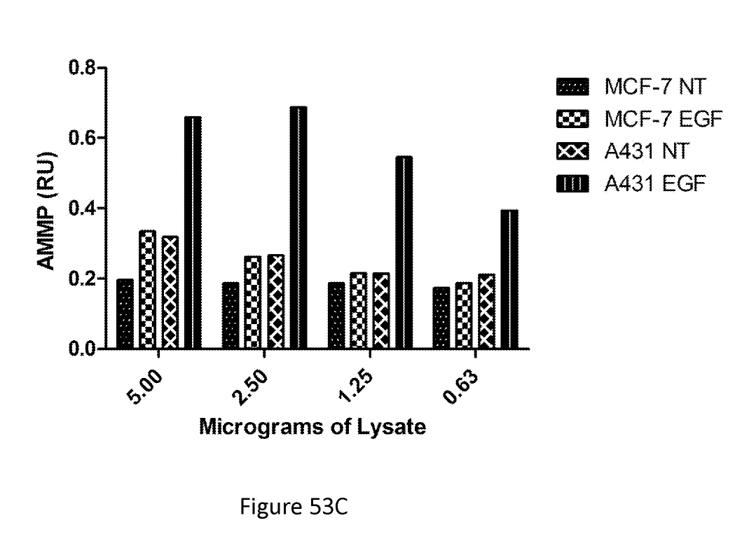

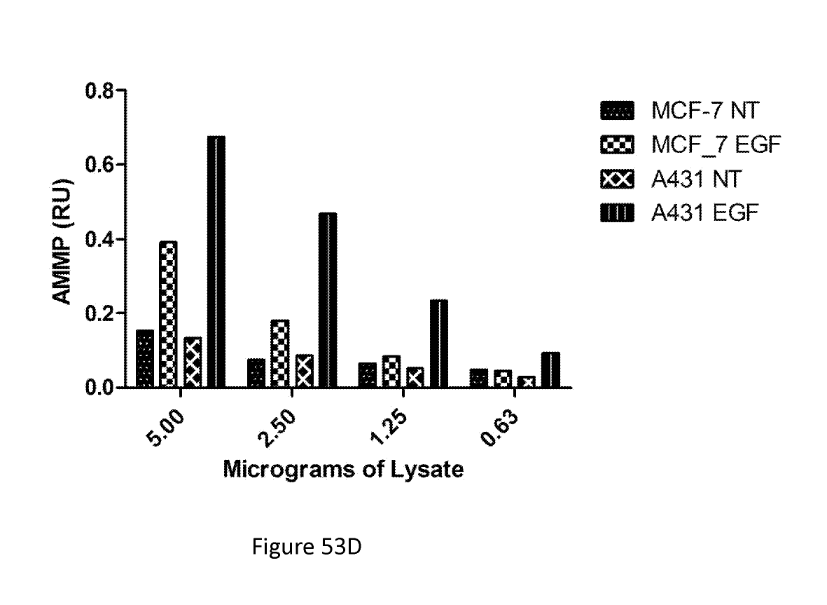

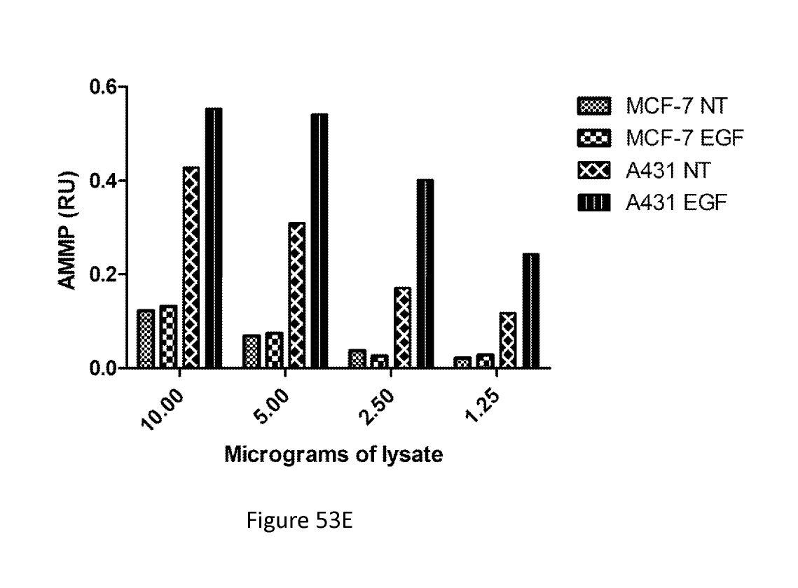

Given that pERK1/2 was identified as the major signaling pathway following EGFR stimulation (i.e., with EGF), another series of AMMP assays were developed to detect MEK1 and ERK1/2. In the absence or presence of EGF, total ERK1/2 levels were virtually undetected (data not shown) whereas total MEK1 was largely similar in expression in both cell lines (FIG. 53B). However, pERK1/2 was easily detected in A431 cells stimulated with EGF (see FIG. 53D). pERK1/2 was also detected to a lesser degree in EGF-stimulated MCF-7 cells (see FIG. 53D). Strong phosphorylation of MEK1 was observed with EGF stimulation of A431 with modest phosphorylation of MEK1 in the MCF-7 (see FIG. 53C). Thus, the data shows that EGF stimulation also caused phosphorylation of MEK1 in A431 and MCF-7 cells. The lack of detectable total ERK1/2 in this experiment was contradicted by the detection of pERK1/2. Further experiments using SDS to denature ERK1/2 revealed the presence of total ERK1/2 in A431 cells (see FIG. 53A). The expression of total ERK1/2 was determined to be higher in the A431 cell line as compared to MCF-7 cell line regardless of EGF stimulation. The requirement to denature ERK1/2 suggests that binding of the antibody required having the antibody be able to access to a normally inaccessible or cryptic epitope, possibly due to ERK1/2 tightly associating with one or more additional proteins as part of its signaling pathway. No change was seen in the MEK1 or pMEK1 levels with SDS treatment (data not shown).

Additionally, to better understand the activity seen by EGF stimulation, an assay was developed to detect the phosphorylation of HER3 as indicative of HER2 and HER3 activity. As shown in FIG. 53E, the data showed that there are high levels of phosphorylation of the HER3 receptors in the A431 cells which increased with EGF stimulation, while little phosphorylation was observed in the MCF-7 cell line.

The results also showed that the AMMP assays are useful for identifying downstream effects of EGFR inhibitors. The utility of the AMMP assay to simultaneously detect multiple kinases in a single panel of assays was further explored using A431 and MCF-7 cells. This panel was constructed to better understand on and off-target effects of EGFR inhibitor drugs in-vitro. The panel of assays consisted of pEGFR, pHER3, pERK1/2, pMEK1, pAKT1, pp38 and pJNK. For this assay, the A431 cell line was utilized to measure on-target effect and efficacy whereas MCF-7 was used to measure off-target effects. EGF mediated EGFR-stimulation was tested for each cell line in the presence or absence of specific EGFR inhibitors, afatinib, gefitinib or neratinib.

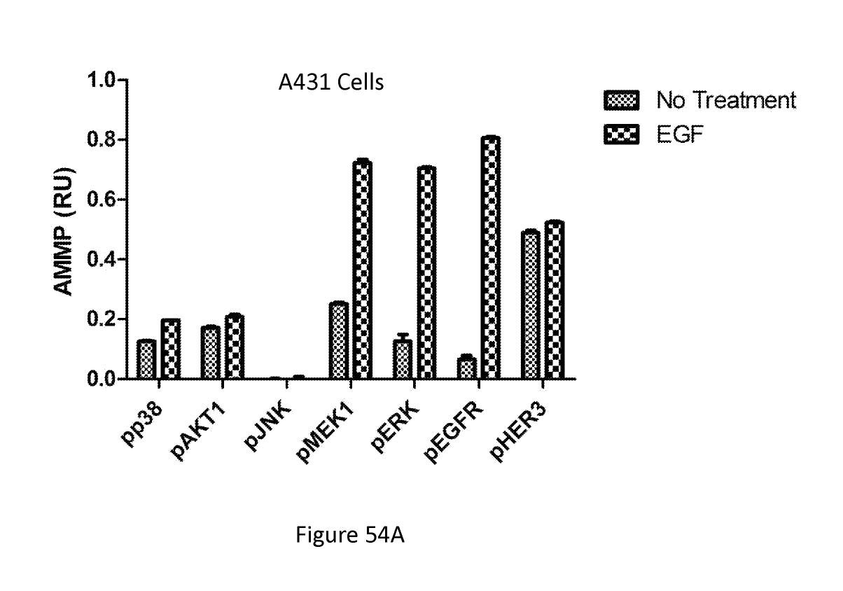

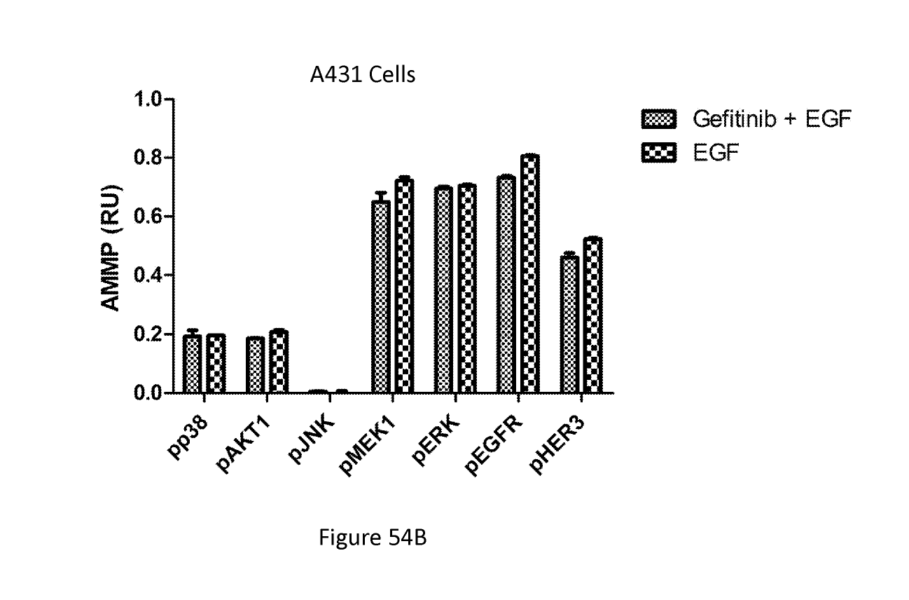

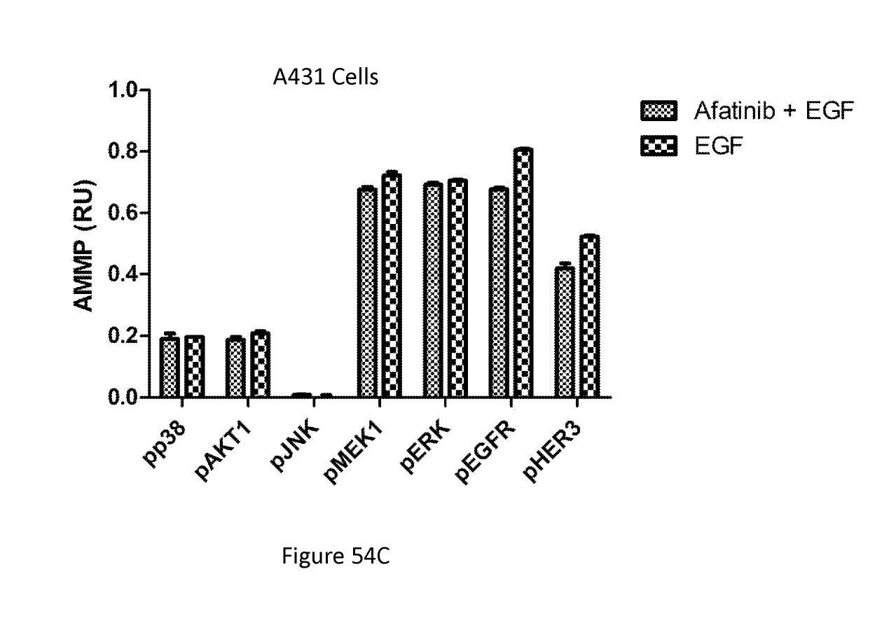

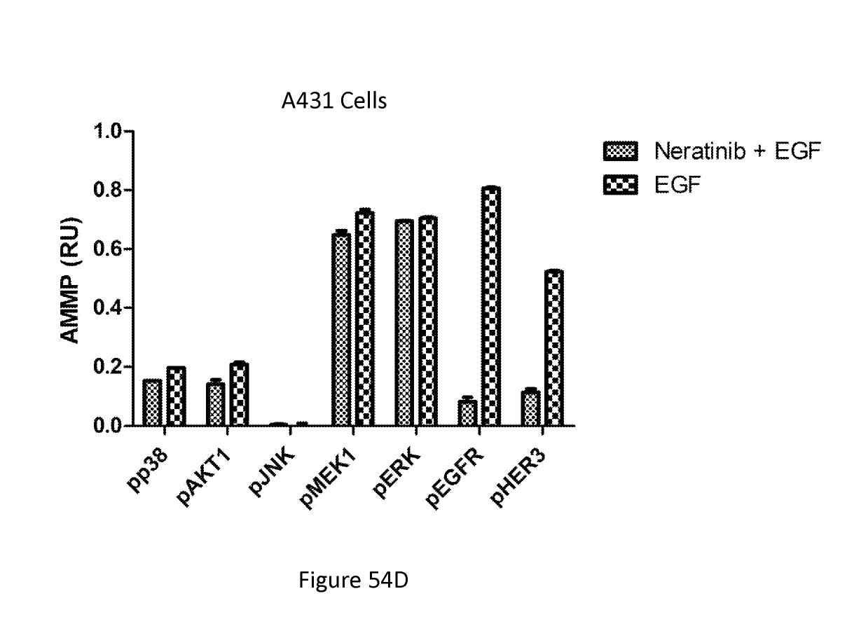

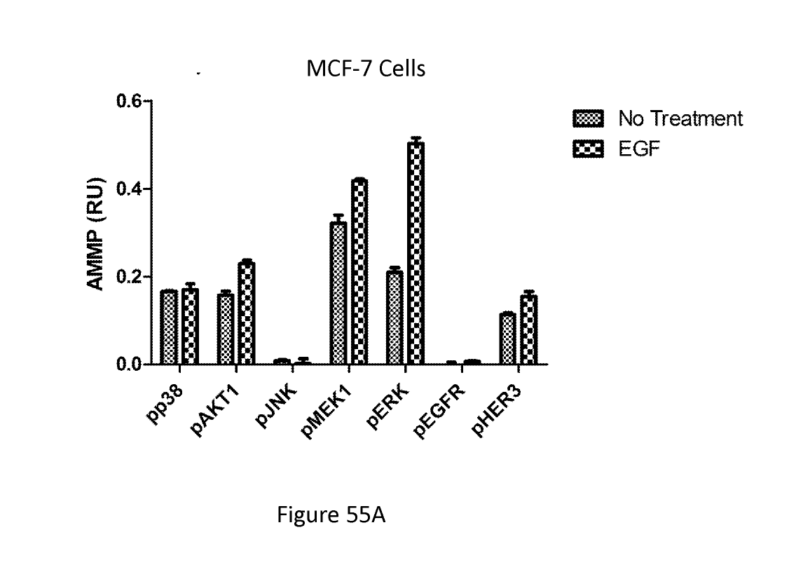

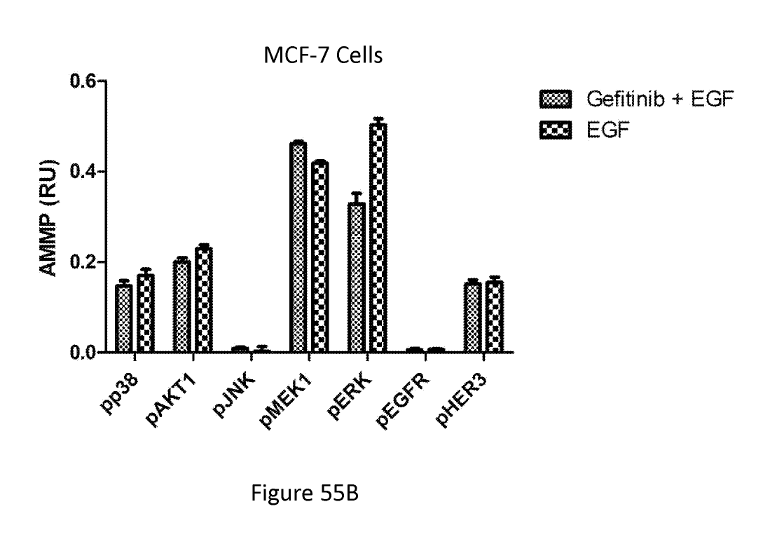

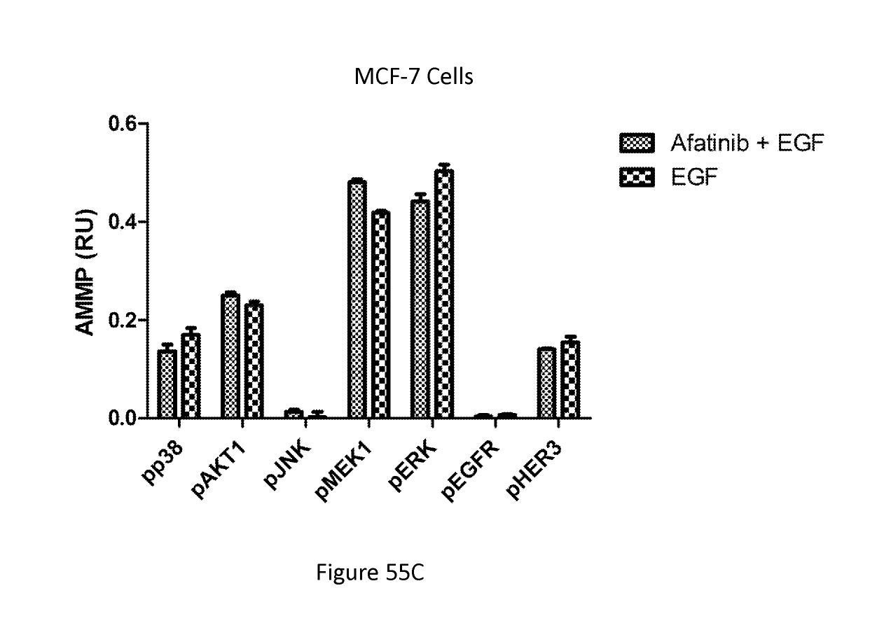

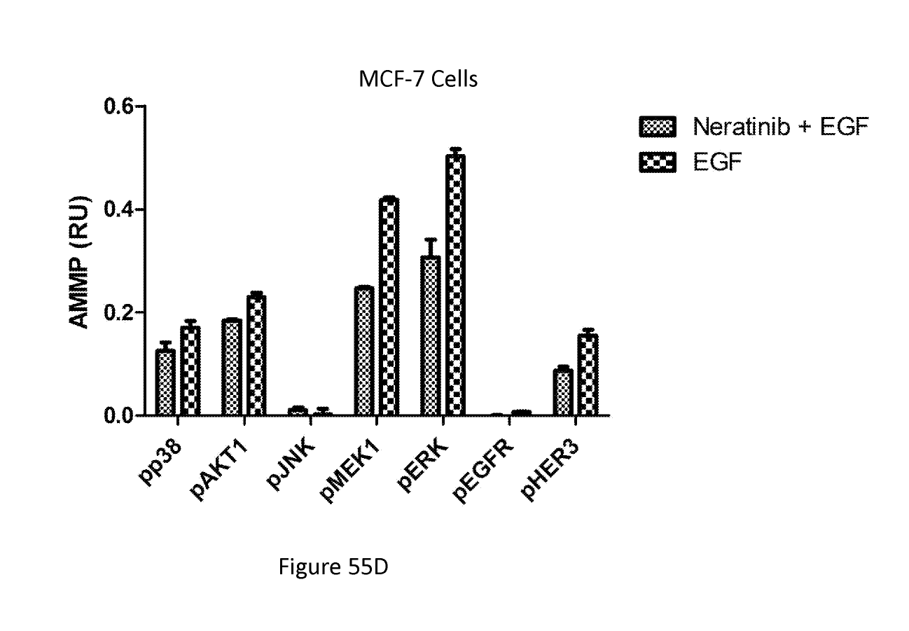

EGF stimulation of MCF-7 and A431 cells was confirmed EGF treatment led to phosphorylation of EGFR, MEK1, ERK1/2 and p38 in A431 cells, while phosphorylation of HER3 receptor was high even in non-treated A431 cells (see FIG. 54A). EGF stimulation of MCF-7 cells resulted in phosphorylation of HER3, MEK1 and ERK1/2 and a small amount of AKT1 phosphorylation which varied between cell preparations (see FIG. 55A). No phosphorylation of JNK was seen in either A431 or MCF-7 cell lines (see FIGS. 54A and 55A, respectively).

The pre-treatment with EGFR inhibitors had varying effects on A431 and MCF-7 cells. Pre-treatment with gefitinib had no significant effect on the EGF stimulated phosphorylation of EGFR, HER3, MEK1, ERK1/2, p38 in A431 cells (see FIG. 54B), however phosphorylation of ERK1/2 in MCF-7 cells was inhibited (see FIG. 55B). Afatinib had little effect on the phosphorylation of EGFR receptors tested or on downstream kinase activity caused by EGF stimulation on either A431 or MCF-7 cell lines (FIGS. 54C and 55C, respectively). Neratinib pre-treatment was able to largely inhibit the phosphorylation of EGFR and the HER3 receptors in EGF-stimulated A431 cells (see FIG. 54D) while HER3 phosphorylation was only partially inhibited in EGF-stimulated MCF-7 cells (see FIG. 55D). Additionally, neratinib had inhibitory effects on MEK1 and ERK1/2 phosphorylation in EGF-stimulated MCF-7 (see FIG. 55D) but no such effect on EGF-stimulated A431 cells (see FIG. 54D). All other kinases tested (i.e., p38, pAKT1, pJNK, pMEK1, pMEK1, and pERK) were largely unaffected in neratinib treated EGF-stimulated A431 cells (see FIG. 54D). In neratinib treated EGF-stimulated MCF-7 cells, a slight loss in phosphorylation of p38 and AKT1 was seen (in addition to the inhibition of pMEK1 and pERK1/2, and the partial inhibition of pHER3 (see FIG. 55D).

Thus, the panel of assays described herein using AMMP detection methods has shown that a drug's efficacy can fail on multiple levels. First, the drug neratinib was highly effective at inhibiting the kinase activity of EGFR and HER3 receptors, whereas gefinitib and afatinib had little efficacy. Downstream from the inhibitor target the effect of the drug neratinib was much less than expected on MEK1 and ERK1/2 in A431 cells. Interestingly, phosphorylation of ERK1/2 was partially inhibited by all three drugs in MCF-7 cells and MEK1 with neratinib pretreatment only. Varying degrees of responsiveness of MCF-7 and A431 cell lines to drug treatment as indicated by MEK1 and ERK1/2 activation may be the result of activation of different EGF responsive receptors in each cell line. The sustained activity of MEK1 and ERK1/2 may be telling to the biology of EGF receptors and the potential lack of efficacy of some EGFR inhibitors. Fully mutated forms of the EGF receptor lacking the tyrosine phosphorylation sites have been reported to fully stimulate MAP kinase phosphorylation upon EGF stimulation (Decker et al., J. Biol. Chem. 268: 9176-9179, 1993). Additionally, genetic alterations have been observed and cited for continued phosphorylation of ERK1/2 following certain EGFR inhibitors treatment regimen (Ecran et al., Cancer Discovery 2: 934-947, 2012).

The assays (e.g., the assays using AMMP assay technology) described herein also shed light on the various activation states of different kinase pathways following stimulation with the same agonist in different cell lines. Most notable was the evidence for crosstalk between multiple kinase signaling pathways. Kinase signaling pathways are frequently studied in isolation from each other, limiting the conclusions that can be drawn therefrom. In contrast, this multiplexing approach provides insight into multiple pathways simultaneously, and permits comparison of the relative contribution of each pathway component in response to a single stimulus. Such an approach provides a new tool for rapid exploration of cell signaling pathways that is critical for future development of new therapeutics for a number of diseases.

Understanding the interactions between intracellular signaling pathway components is required in order to develop effective therapeutics against a number of diseases. Rapid identification and exploration of intracellular signaling pathways has been demonstrated and can be similarly implemented for a number of diseases by analyzing their signaling pathways with multiplex assaying using AMMP detection methods. In some embodiments, the analysis of two or more signaling pathways may be performed simultaneously using the methods described in this Example 6, and in the whole of the present disclosure.

### Example 9

### Detection and Analysis of Biomarkers in Two Different Pathways to Detect Escape from a Therapeutic Treatment by a Tumor Cell Using an Alternate Signaling Pathway

Melanoma patients having the V600E B-raf being treated vemurafenib (Zelboraf) are screened for indication that their melanoma cells are escaping death from the vandetanib treatment by signaling through an alternate pathway.

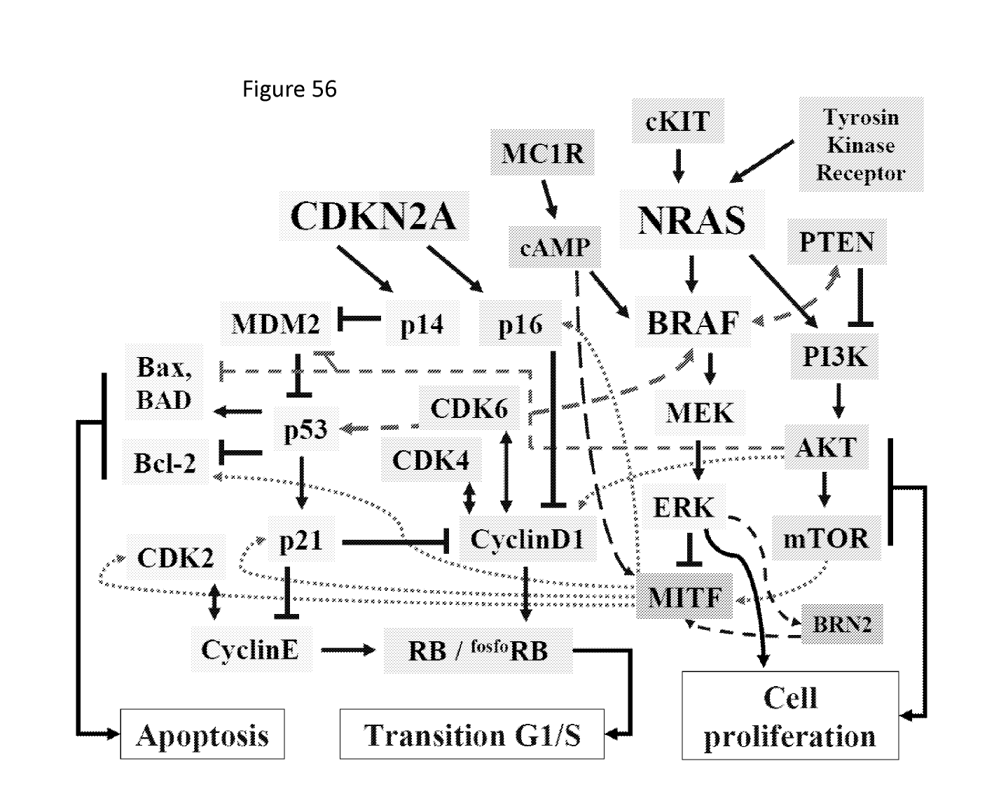

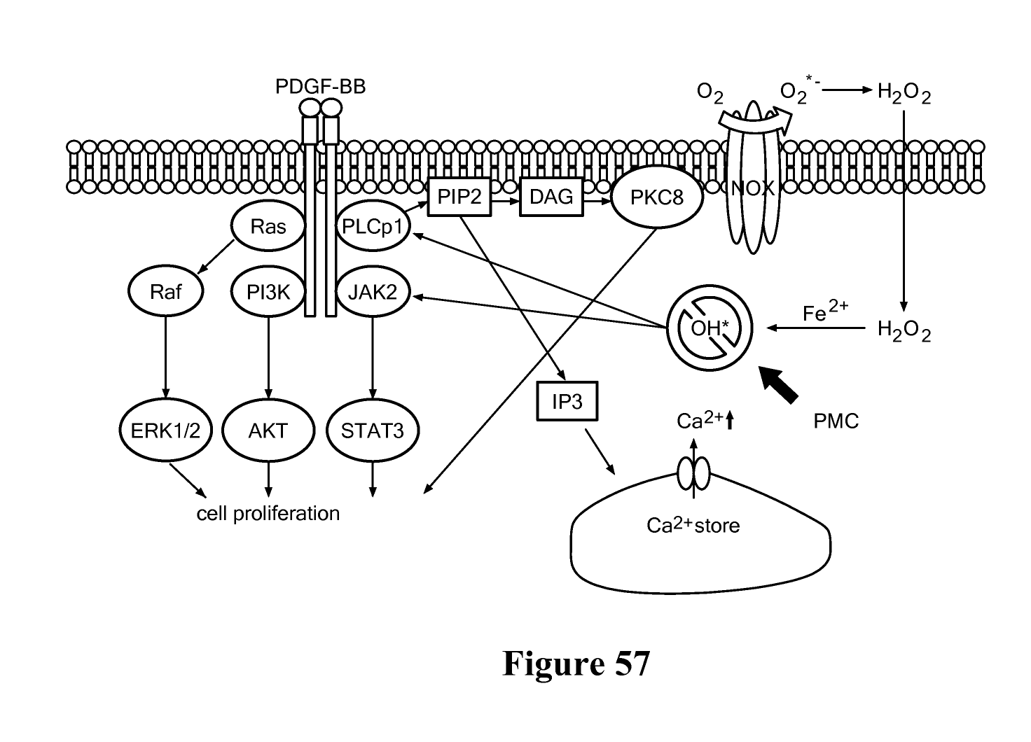

Vemurafenib, which is marketed as Zelboraf, is a small molecule inhibitor of a B-rafkinase having a V600E mutation (i.e., the valine that normally occurs at position 600 is replaced by glutamic acid). As about 60% of melanomas have this mutation. In melanoma cells lines, vemurafenib causes programmed cell death (i.e., apoptosis) by interrupting the B-Raf/MEK/Erik pathway (see FIG. 56), and it is thought to do the same in vivo. In a recent studies, vemurafenib treatment was able to reduce the number of cancer cells in over half of the patients with advanced melanoma who were treated, and increased the survival time (on average) by about six months (Garber, K., Science 326: 1619, 2009; Flaherty et al., N. Engl. J. Med 363: 809-819, 2010). However, some vemurafenib-treated cells area able to escape death by overexpressing the PDGFR-beta receptor tyrosine kinase (see FIG. 57) or mutating a second gene called N-Ras, which reactivates the normal B-raf survival pathway (Nazarian et al., Nature 468: 973-977, 2010).

Given how aggressive and lethal metastatic melanoma is, vemurafenib-treated patients are given weekly screens using the methods described herein. Briefly, samples of melanoma cells are taken from the patients (e.g., from blood samples) and screened for the intact signaling of two pathways.

The first pathway screened is the MEK-ERK pathway by determining whether MEK kinase is phosphorylated using the methods described above (e.g., in Example 8). This phosphorylation event should be missing in cells that are still responsive to vemurafenib, but will resume again if the patient's melanoma cells are able to reactive the normal B-raf pathway, for example, by mutating N-ras. The data from the patient's cells will be compared to data from a healthy person's cells and, if the patient is still responding to vemurafenib, the acoustic output signal from the patient's cells will be lower than the acoutstic output signal from a healthy person's cells.

The second pathway screened the phsophorylation of AKT, again, using the methods described above (e.g., in Example 8). Although some baseline level of AKT phosphorylation may be seen in melanoma cells responding to vemurafenib, higher than normal phosphorylation of AKT (with the normal phosphorylation level of AKT measured in cells taken from healthy people) indicates the patient's cells may have started overexpressing the PDGFR-beta receptor which, when activates, will phosphorylate AKT.

Using the methods described herein, which harness the rapid, highly sensitive, extremely reproducible methods that are based on an acoustic output signals, phosphorylation events (e.g., phosphorylated AKT or phosphorylated MEK) can be readily detected in patients' cells and compared to the phosphorylation events from normal cells. As shown in this Example 9, the change in the acoustic output signal from a normal cell and the acoustic output signal from a diseased or damaged cell (in this case, melanoma) can be a positive or negative number. However, it is the change that is important, whether that change is represented as a ratio, a percentage, etc.

Moreover, in some embodiments, using the methods described herein, more than one pathway can be analyzed simultaneously. Indeed, in some embodiments, using the methods described herein, more than one pathway can be analyzed from the same sample. This in convenient for the patient, and since the sample sizes are small, the patients can be tested frequently (e.g., weekly, monthly, or even daily). The detection of an escape pathway in a diseased or damaged cell (e.g., a melanoma cell) being treated with one targeted therapeutic (e.g., vemurafenib which targets the B-raf V600E mutation) allows a rapid change in the patient's therapy.

For example, a patient whose cells are screened as in this Example 9 where the cells are found to show higher than normal AKT phosphorylation may not yet become symptomatic for melanoma regression. However, that may indeed be happening, but the number of melanoma cells using this escape hasn't increased to a high enough number for the patient to become symptomatic. It is at this point that a second targeted therapy is most useful, because those mutated cells can be “nipped in the bud”, and killed before they spread their mutation to their progeny and ultimately kill the patient. In such a scenario, the patient's therapy may be changed to a therapeutic regimen with a drug that targets the PDGFR-beta receptor (e.g., sunitinib, which is sold under the name Sutent by Pfizer, Inc.) either instead of or in combination with vemurafenib. Because of the early detection of the escape pathway using the methods described herein, the therapeutic regimen for the patient can be changed before the patient show symptoms from melanoma regression.

### Example 10

### Analysis of Impaired Signal Transduction Pathways in siRNA Knockdown Colorectal Cells

To determine whether or colorectal cells treated with siRNA to knock down particular biomarkers in signal transduction pathways actually show a reduction in the expression of the biomarker targeted by the siRNA, human SV40 colorectal cells comprising siRNA targeting KRAS, siRNA targeting MEK, siRNA targeting PI3kinase, or siRNA targeting both PI3Kinase and targeting MEK (i.e., two siRNA in the cell), and a SV48 cell line comprising an irrelevant siRNA molecule were obtained. Such cells can be made by standard methods. For example, the SW620 human colorectal adenocarcinoma cell is commercially available from the American Type Culture Collection (“ATCC”; Manassas, Va.; catalog No. CCL-227). Similarly, the sequences of nucleic acid molecules encoding KRAS, MEK, and PI3 kinase are known. For example, the sequence of KRAS is provided at GenBank Nos. AF493917.1 and L00049.1 (Shimizu et al., Nature 304: 497-500, 1983; McGrath et al., Nature 304: 501-506, 1983).

From the known sequences of MEK, PI3K, and KRAS, siRNA molecules were made according to standard methods (see, e.g., Brummelkamp et al., Science 296: 50-553, 2002; Elbashir et al., EMBO J. 20: 6877-6888, 2001; Yu et al., Proc. Natl. Acad. Sci. 99(9): 6047-6052, 2002; Paddison et al., Genes Devel. 16: 948-958, 2002; and U.S. Pat. Nos. 7,078,196 and 8,198,252).

MEK, PI3K, and KRAS siRNA knock-downs were used because these three biomarkers all play roles in signal transduction pathways. FIG. 49 is a schematic diagram showing two signal transduction pathways in which MEK, PI3K, and KRAS play roles. Because there are other biomarkers upstream and downstream of these three biomarkers in the signaling pathways shown in FIG. 49, expression levels of those biomarkers were also analyzed.

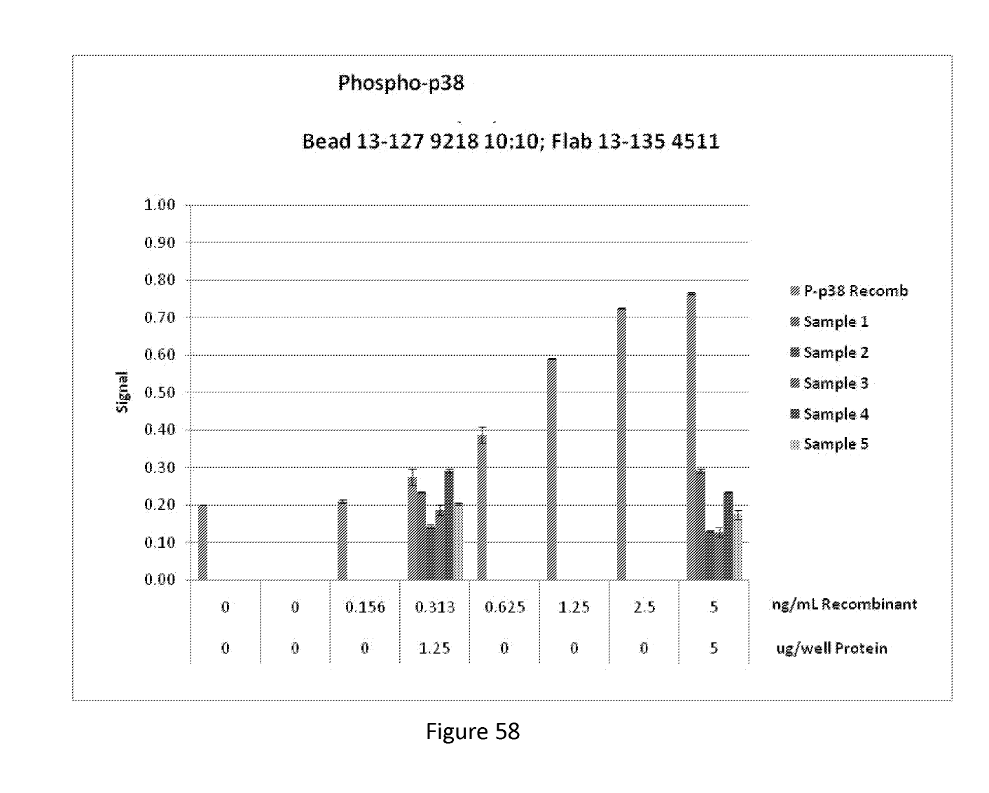

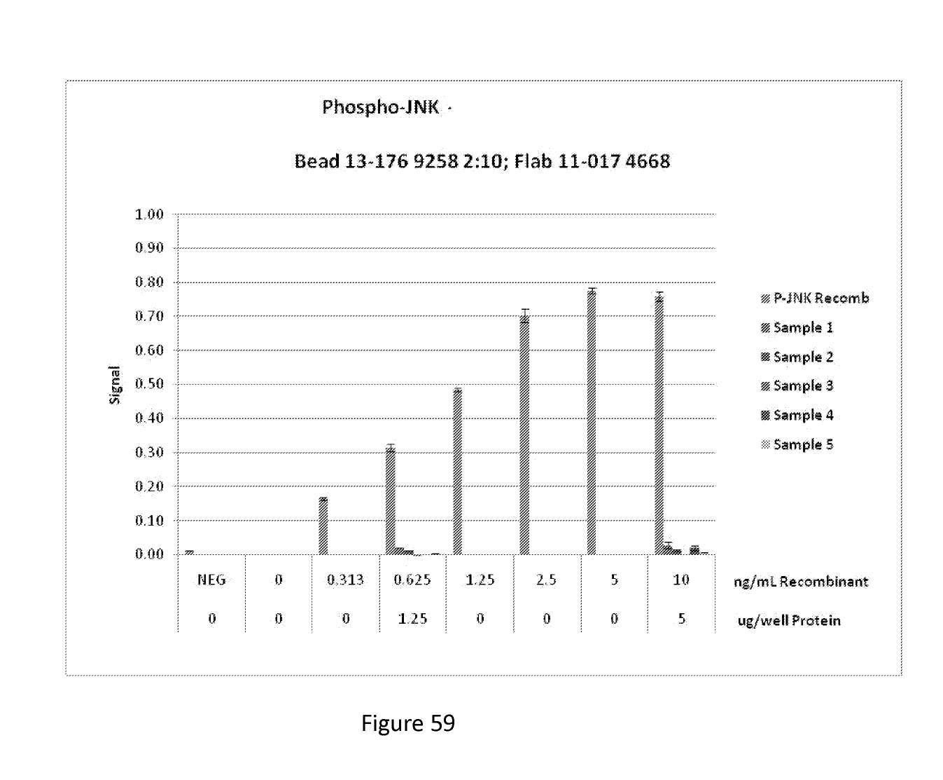

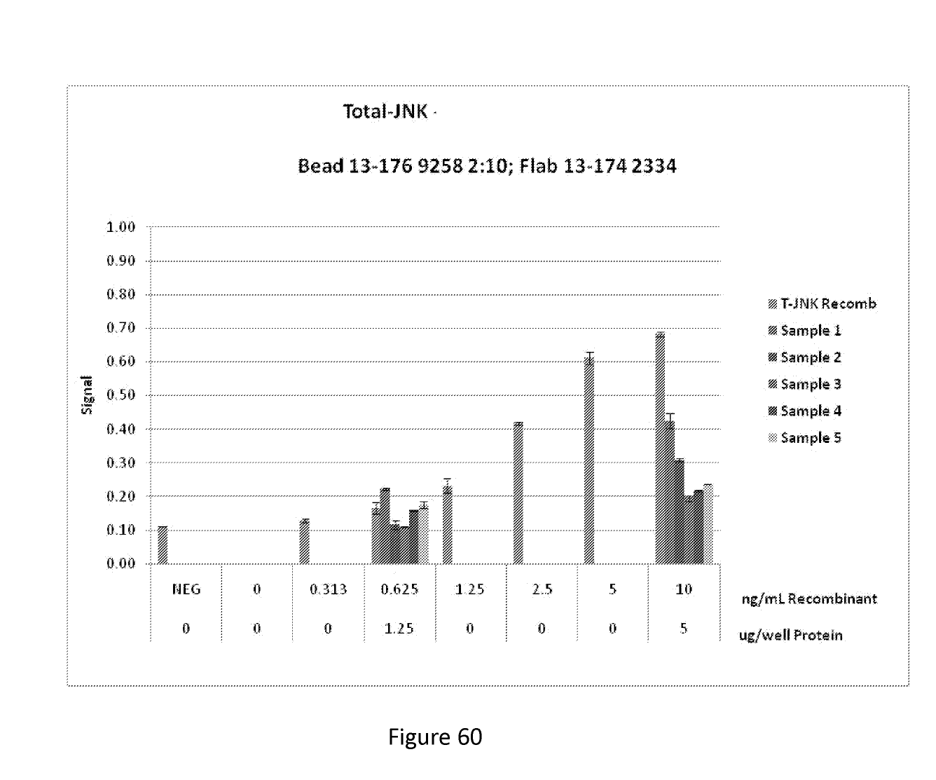

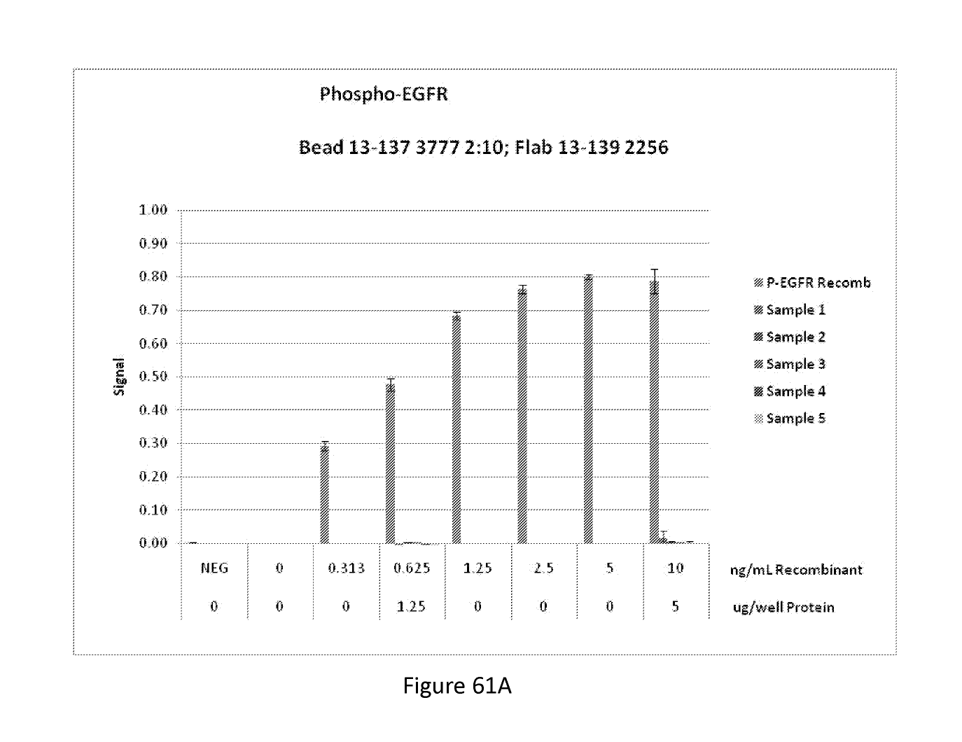

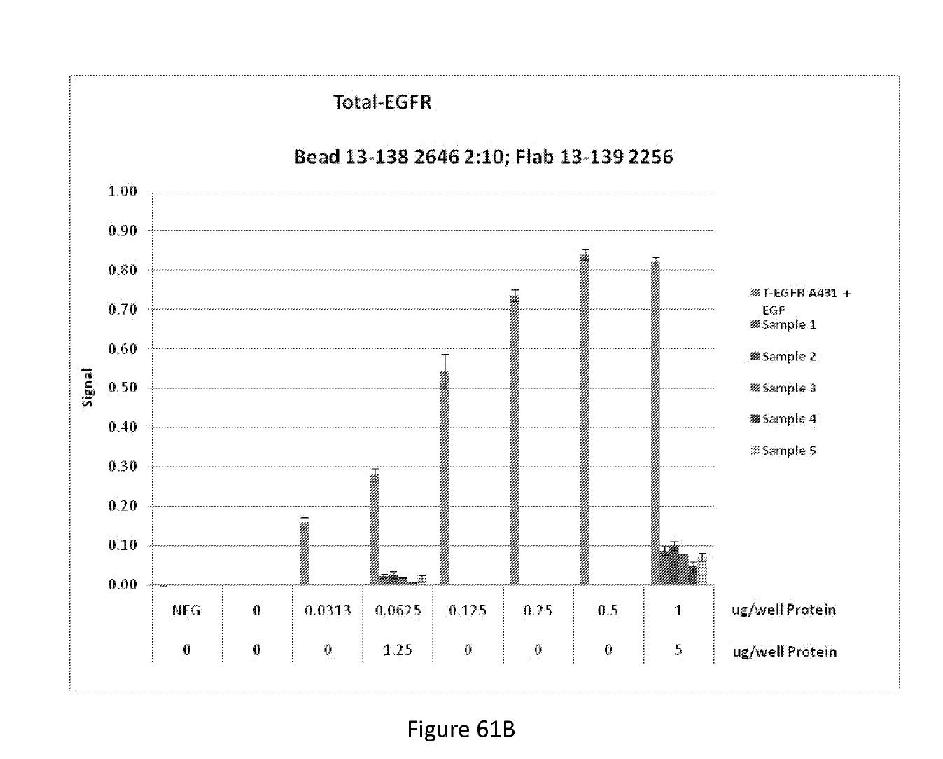

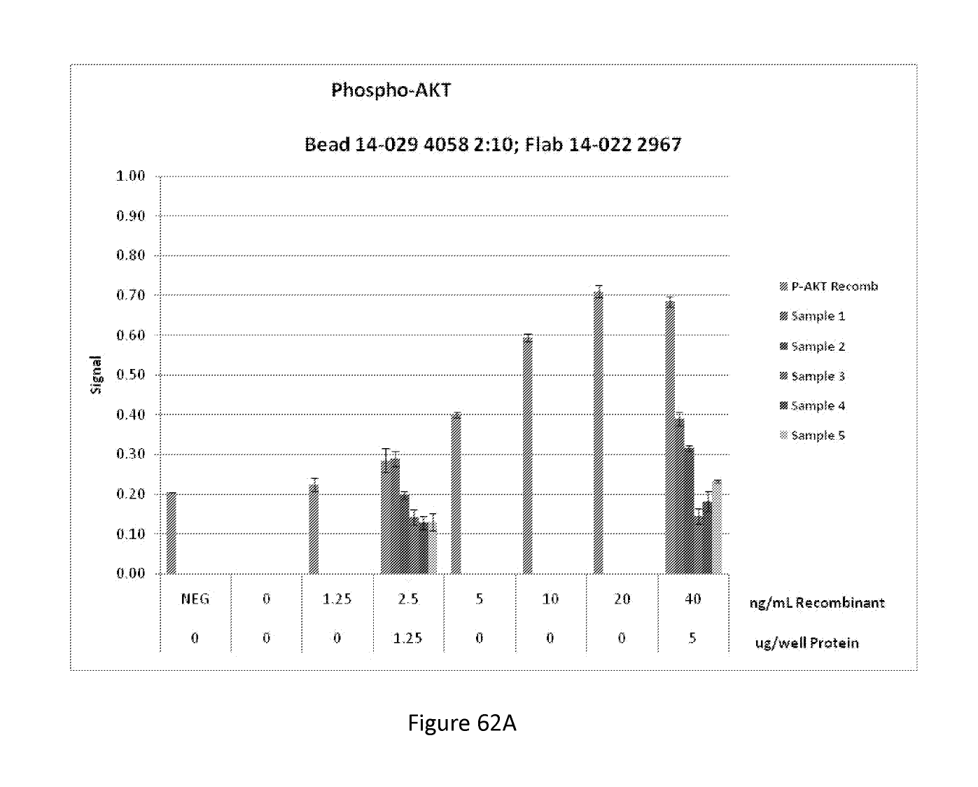

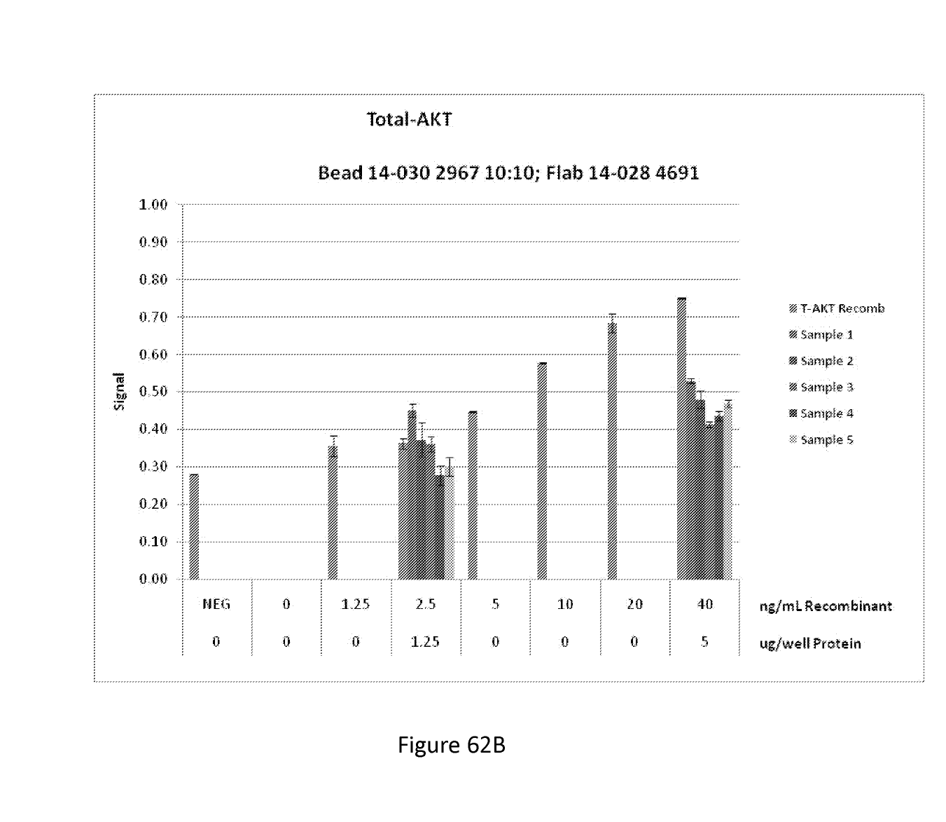

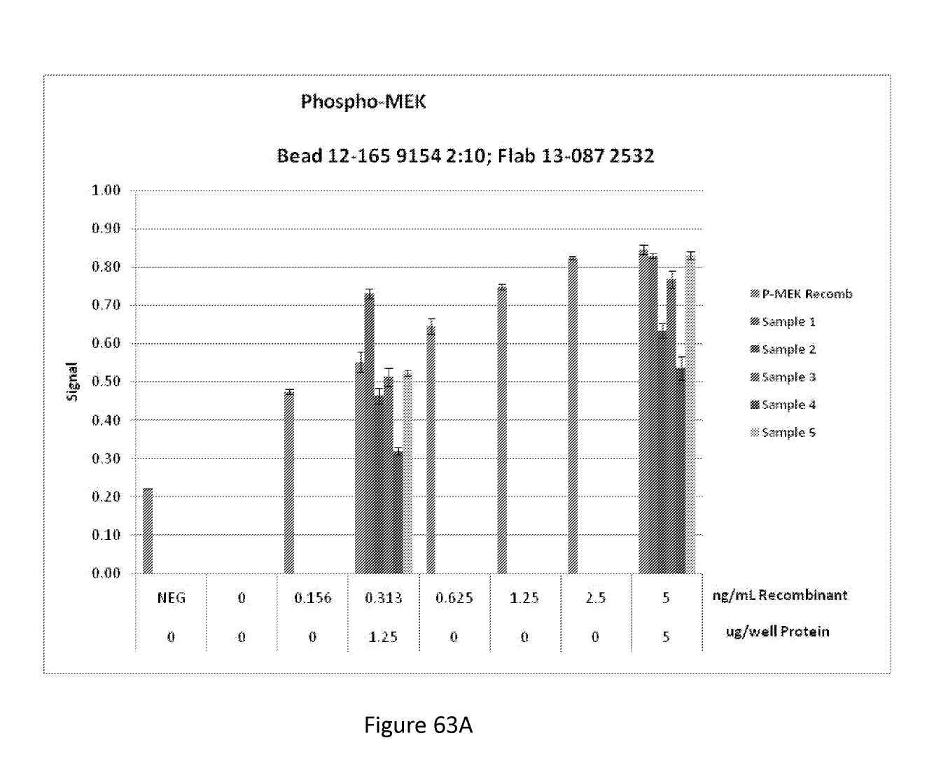

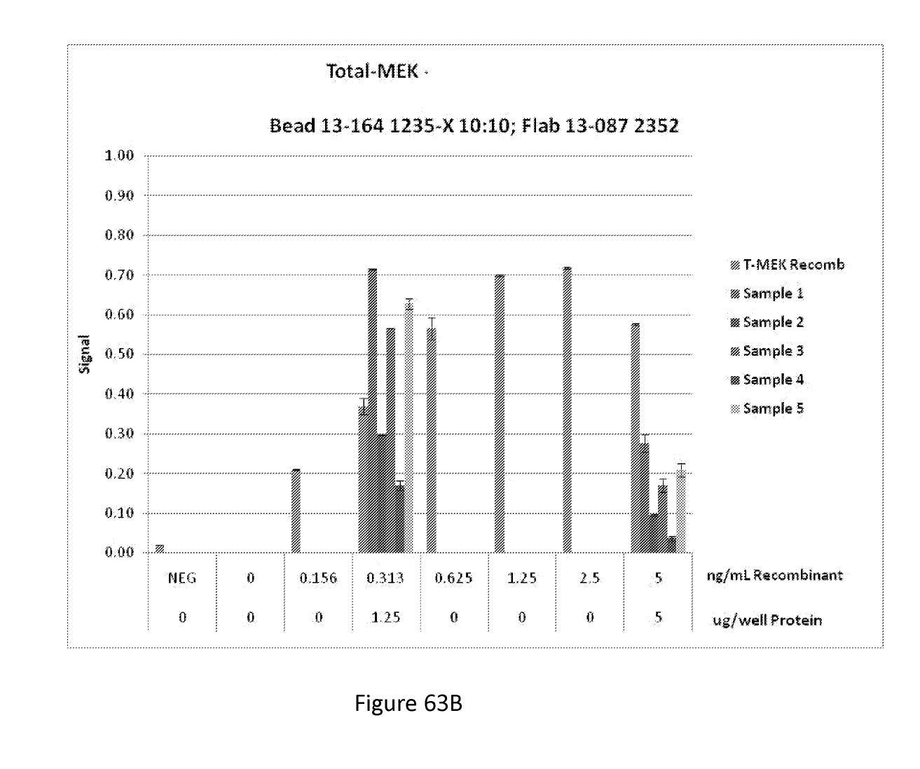

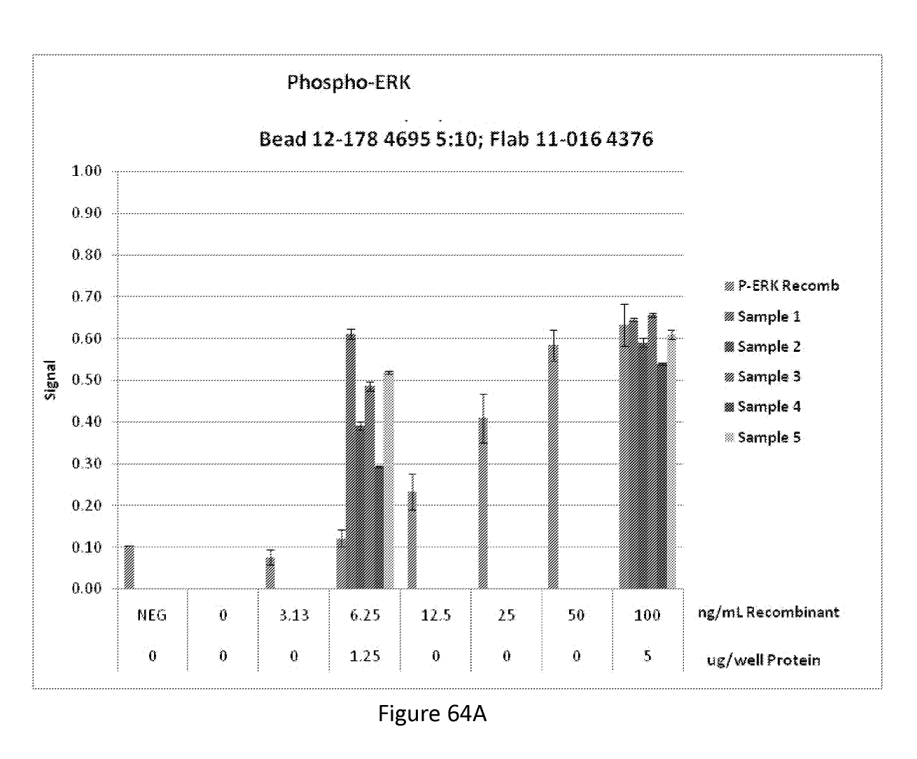

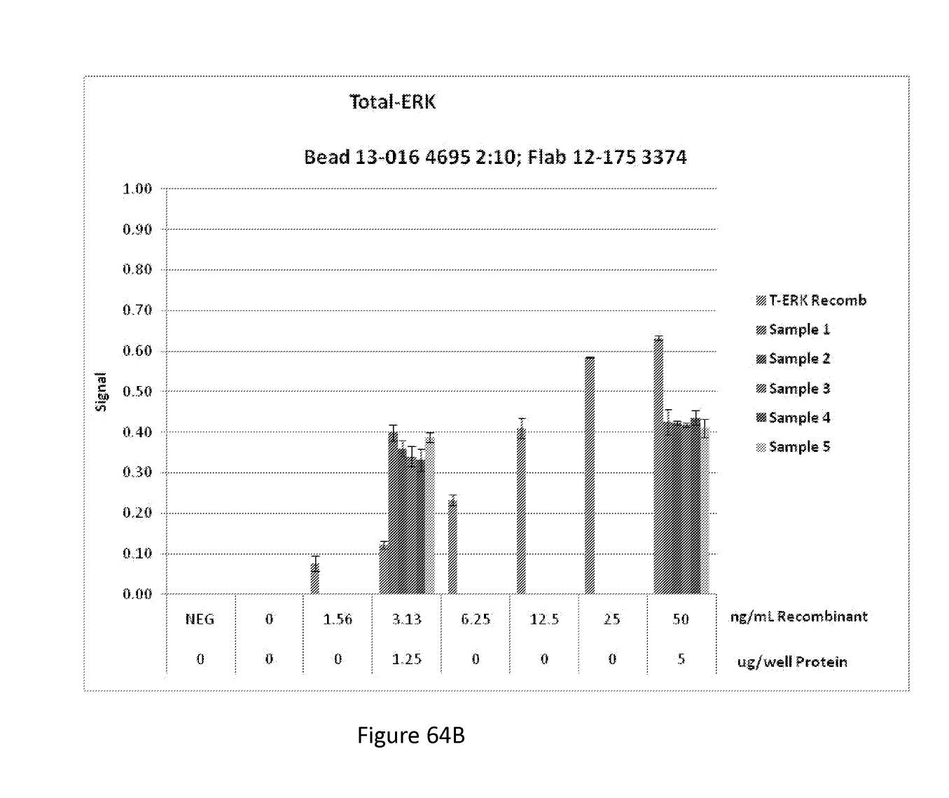

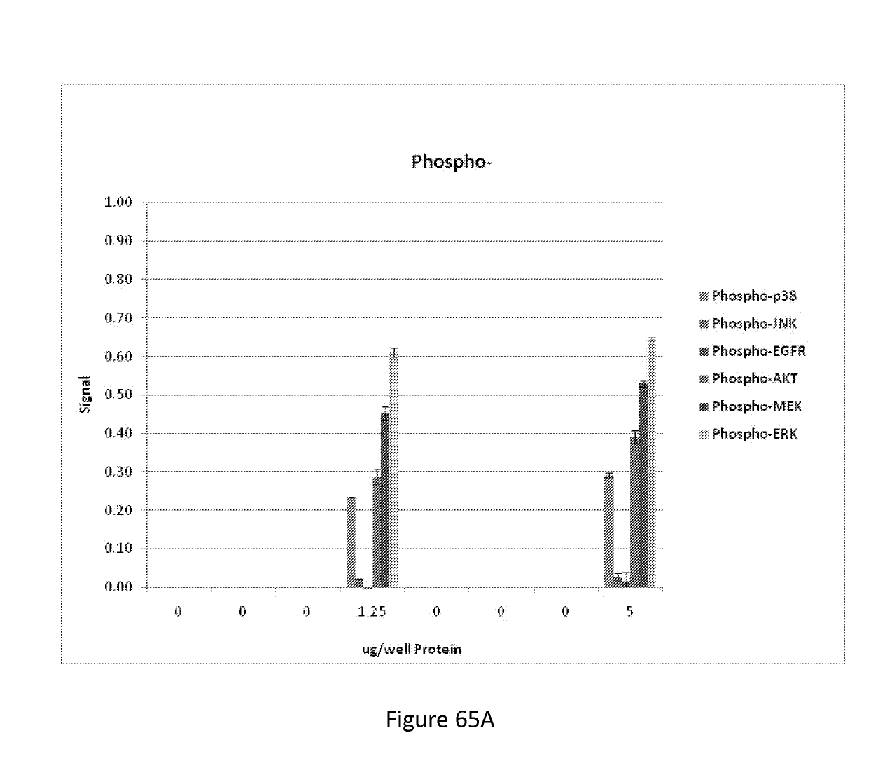

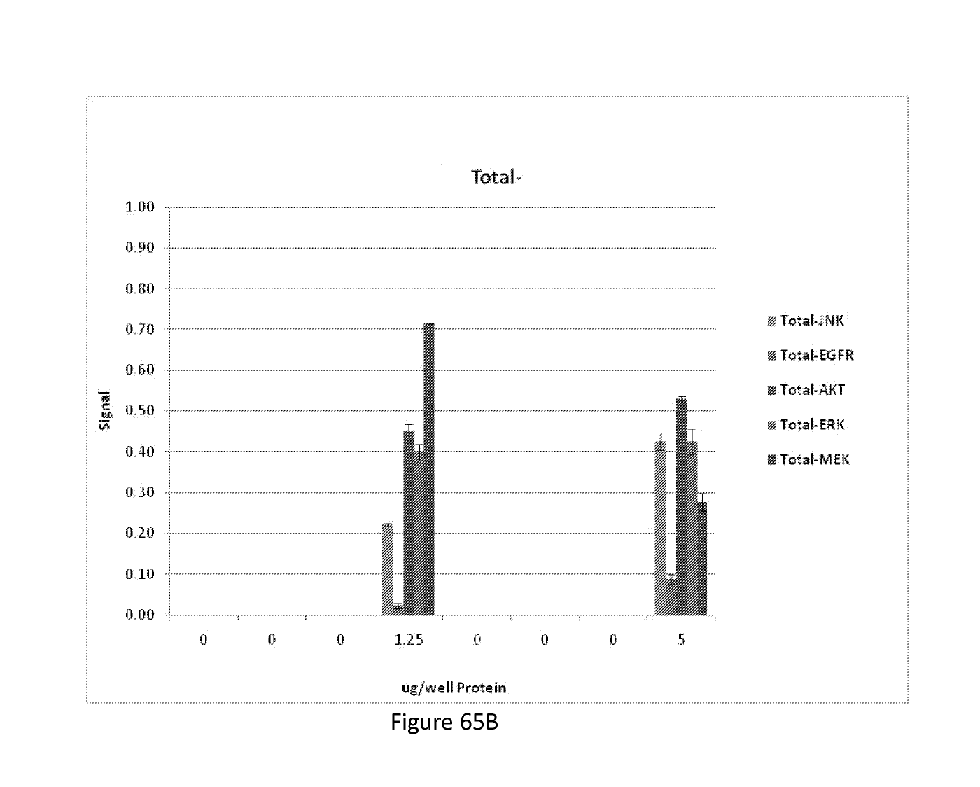

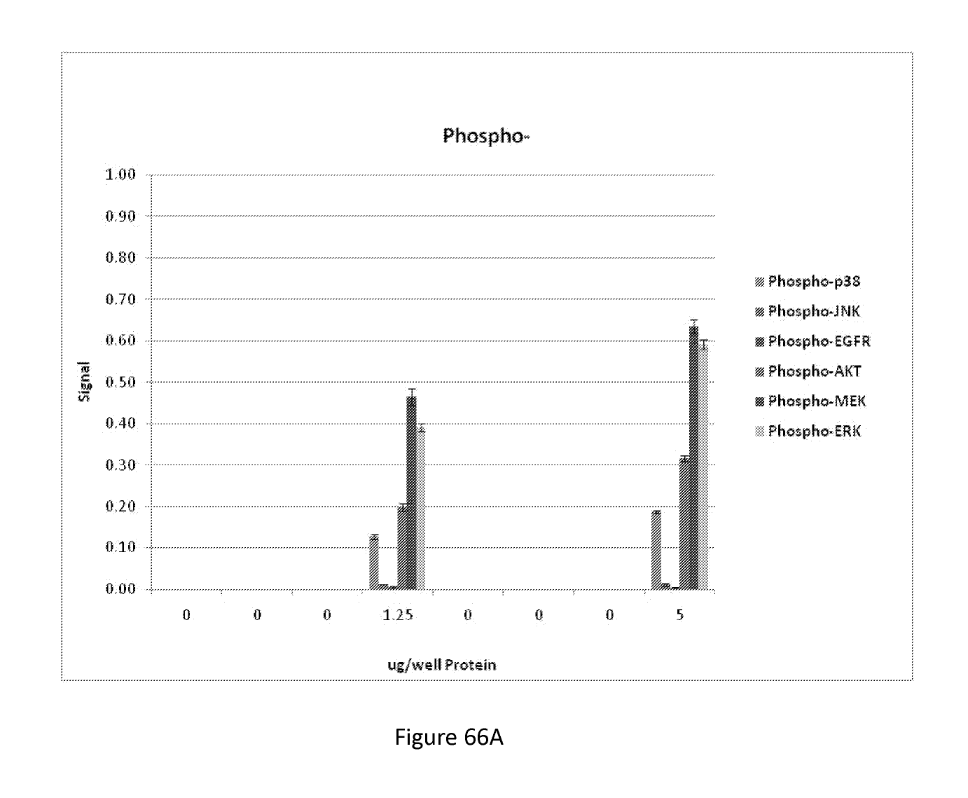

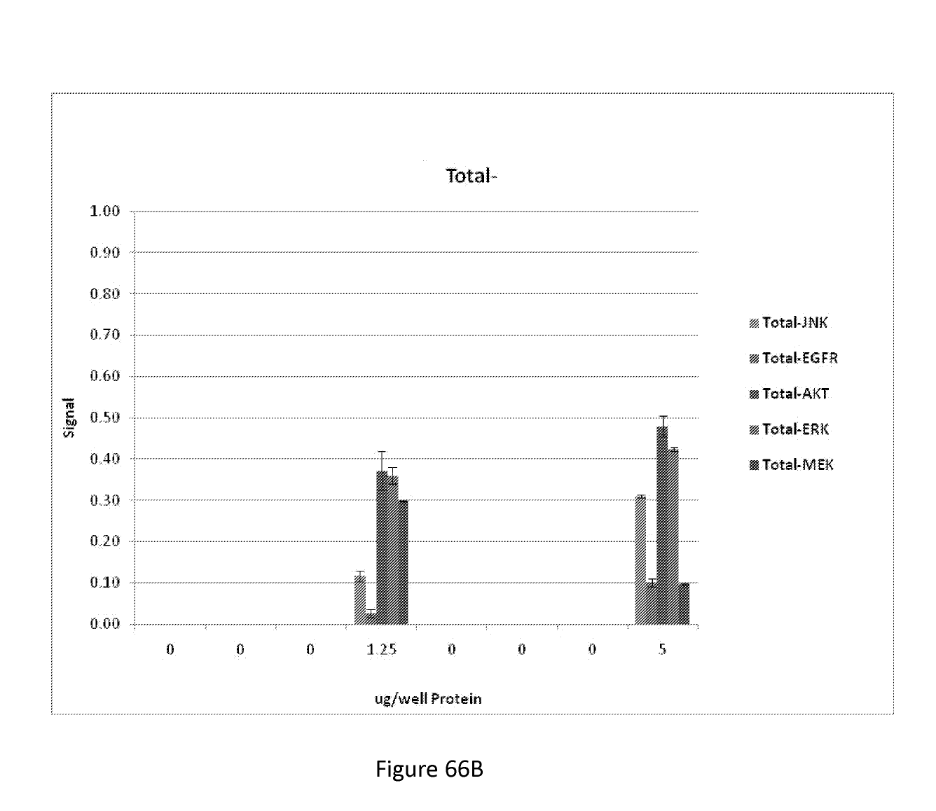

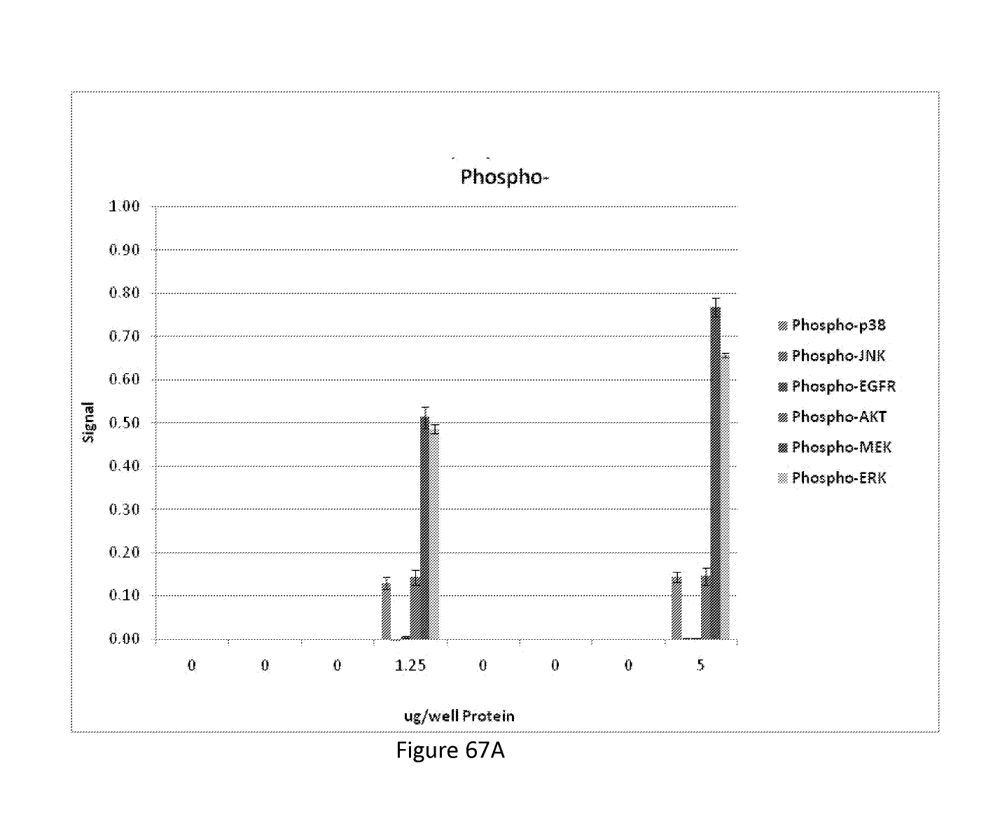

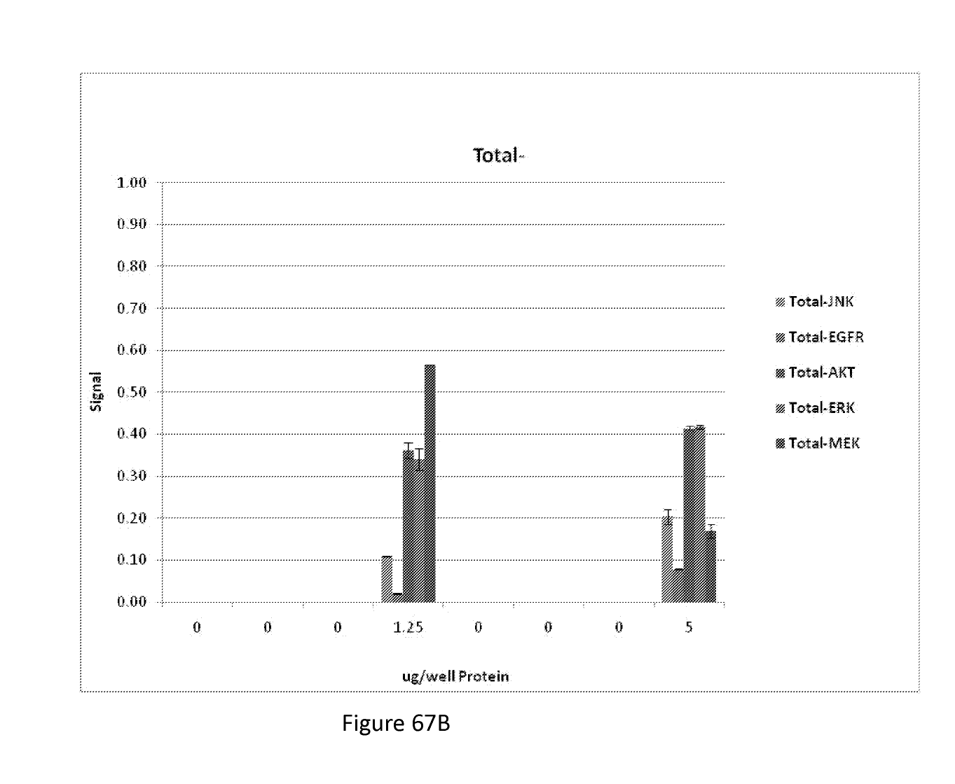

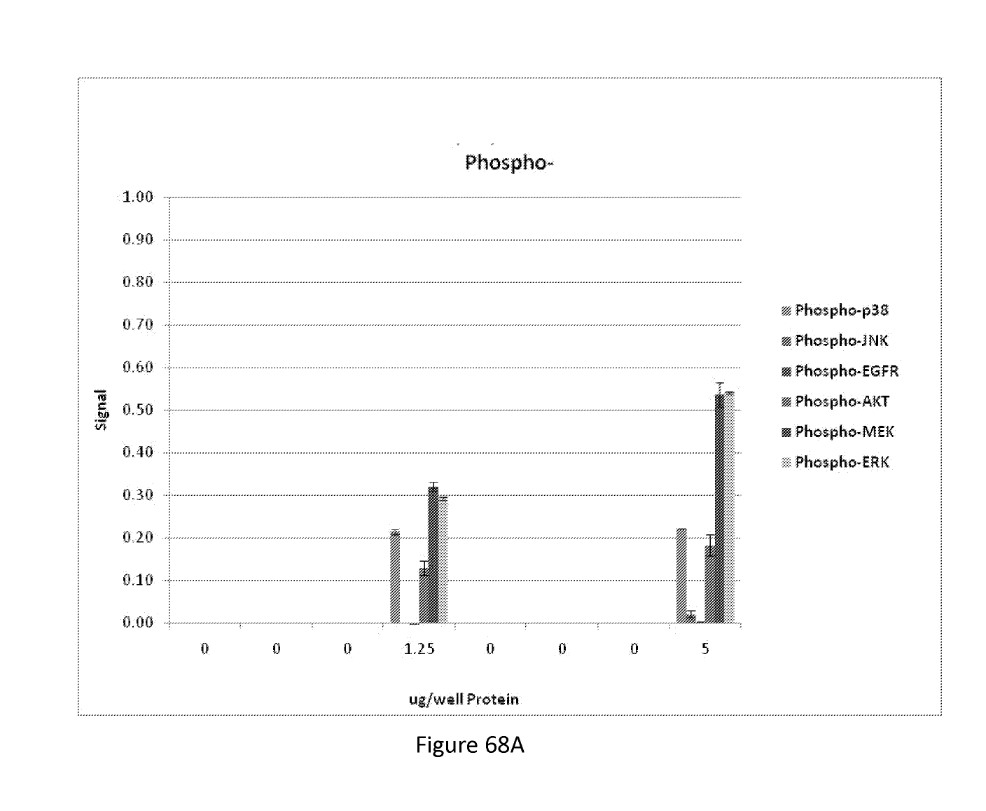

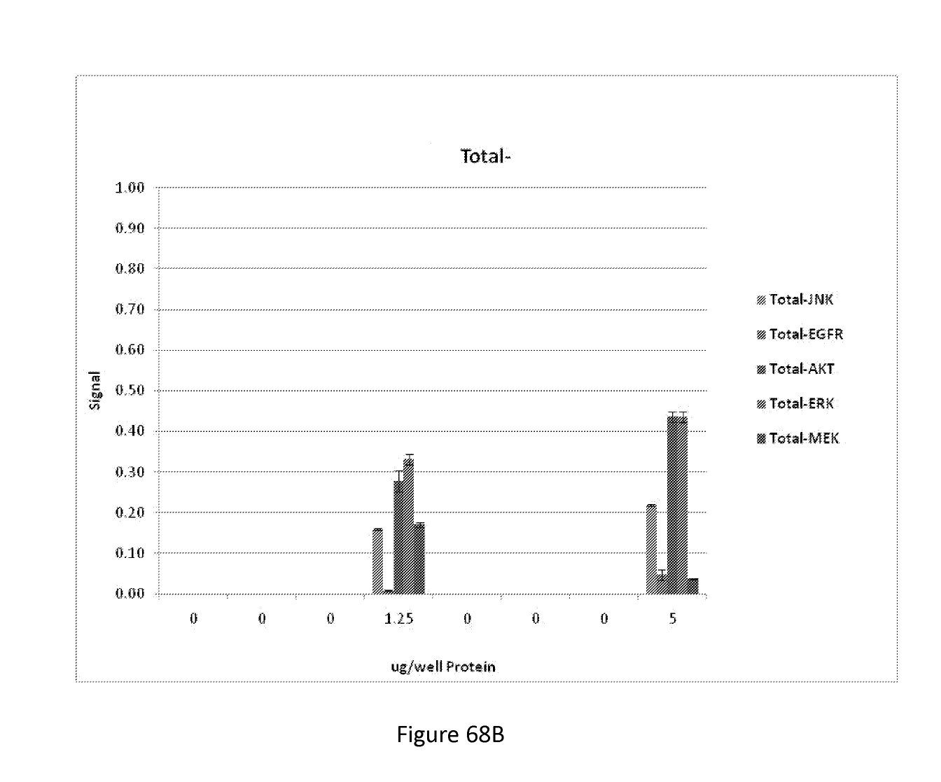

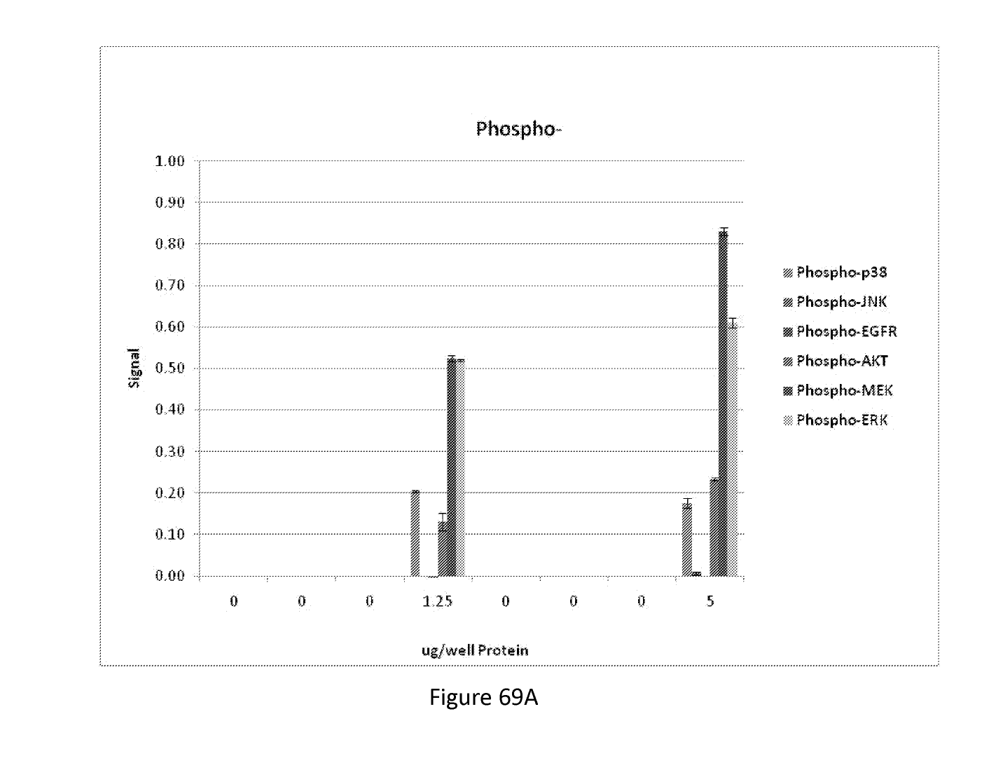

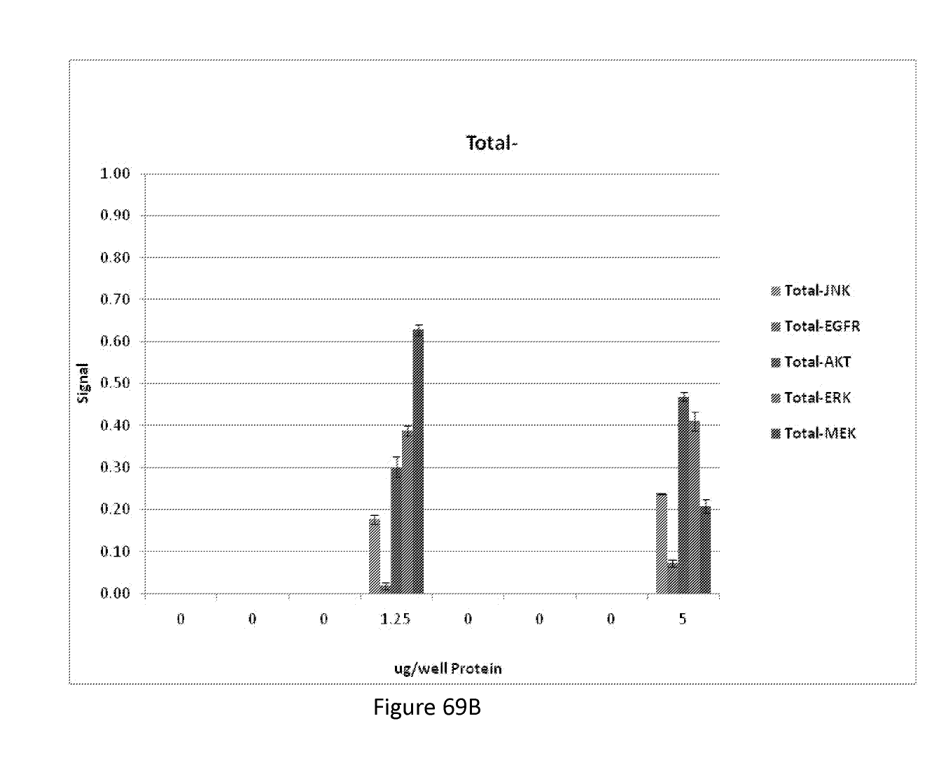

The phosphorylated (i.e., active) form of p38, JNK, EGFR, AKT, MEK, and ERK was measured in each of the different knock-down cells using the AMMP methods described herein. Additionally, to determine if the proteins were present but not phosphorylated, total (i.e., including both phosphorylated and non-phosphorylated forms) protein of JNK, EGFR, AKT, MEK, and ERK were measured. As shown in FIGS. 58-64B, the amounts of phosphorylated protein and total protein different in the treated cells as compared to untreated Sample 5 cells. For example, in comparing FIG. 59 to FIG. 60, expression of phosphorylated JNK (FIG. 59) is decreased in Samples 1 (cells treated with siRNA targeting KRAS), but total JNK (FIG. 60) is largely unaffected. Likewise, by comparing FIG. 62A to FIG. 62B, it is clear that knocking down either PI3K alone (Sample 3, gray bars) or knocking down both PI3K and MEK simultaneously (Sample 4, purple bars) serves to reduce the amount of phosphorylated AKT in the cells (see FIG. 62A) without having a noticeable effect on total AKT (see FIG. 62B). The effect of the different gene knock down on protein expression level is set forth in FIGS. 65A-69B. As shown in FIGS. 65A and 65B, the level of phosphorylated JNK but not total JNK is reduced in the Sample 1 KRAS knock-down cells. Interestingly, in the Sample 4 MEK and PI3K knock-down cells, both total MEK and phospho MEK are reduced compared to control Sample 5.

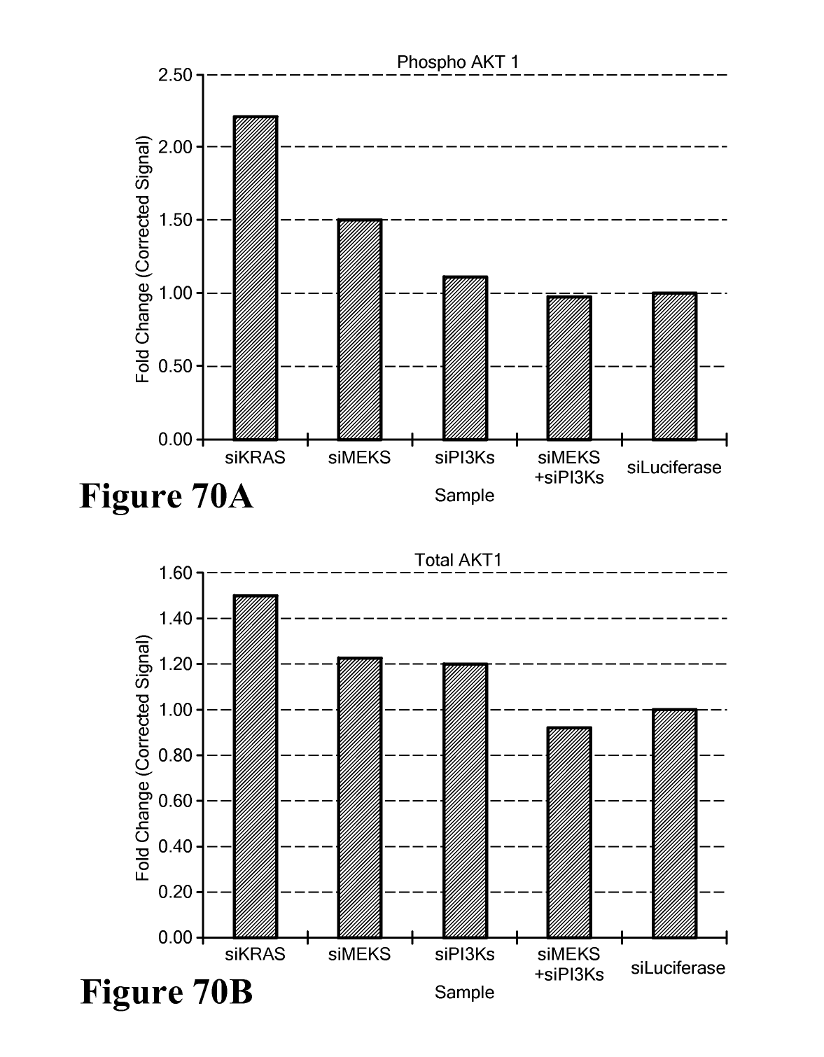

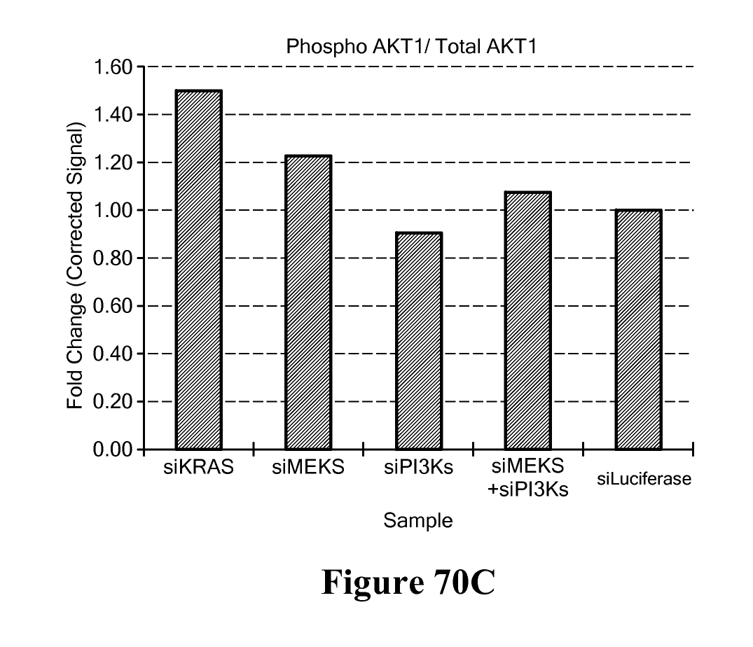

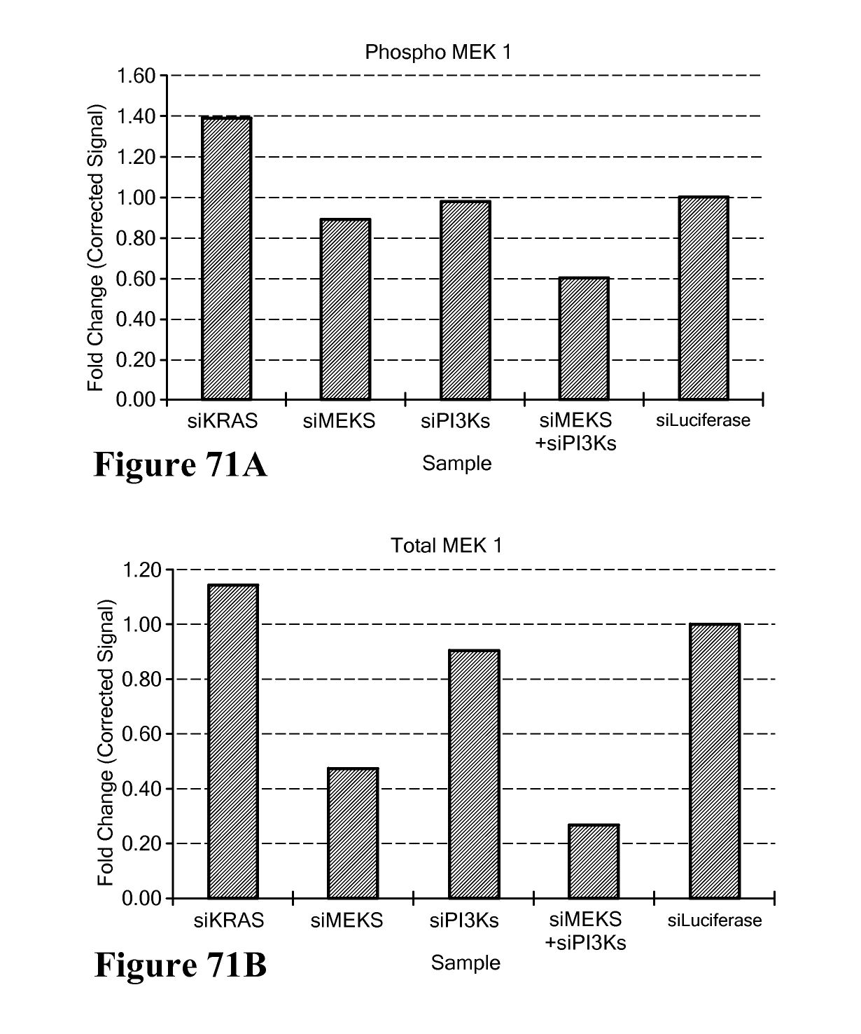

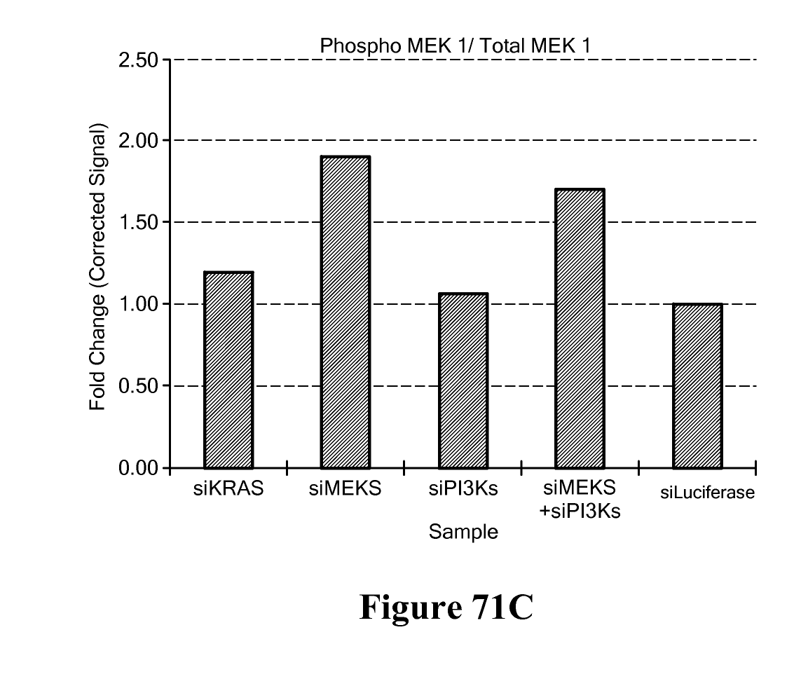

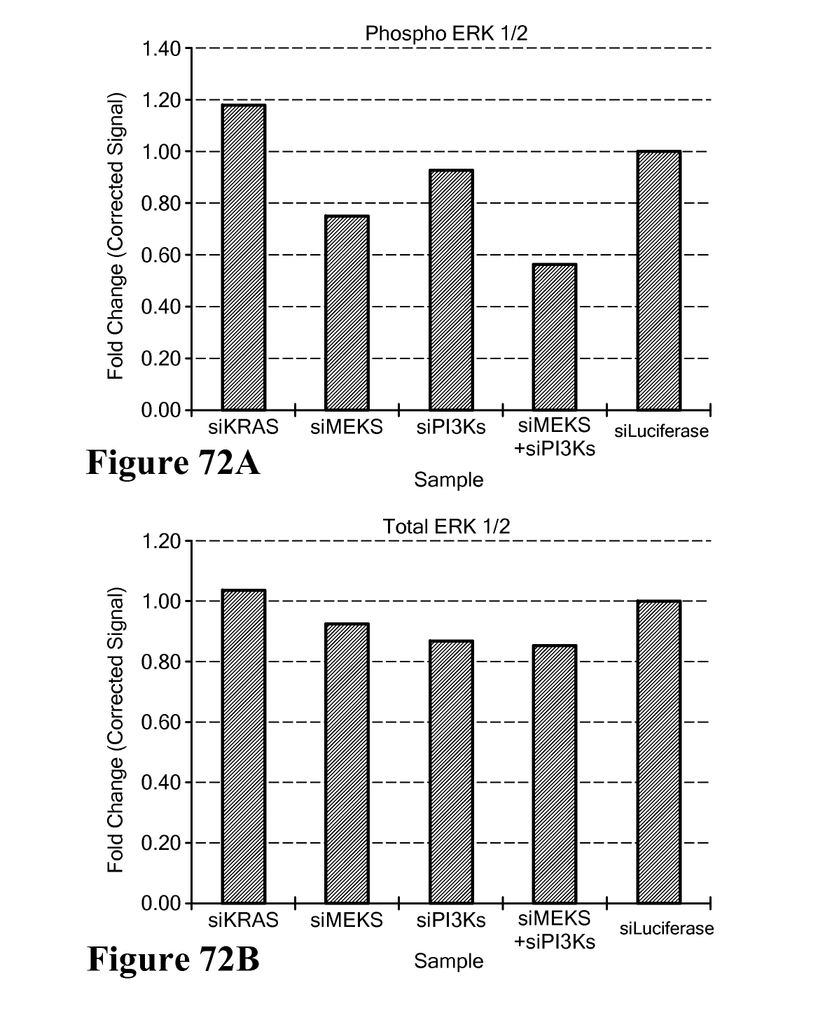

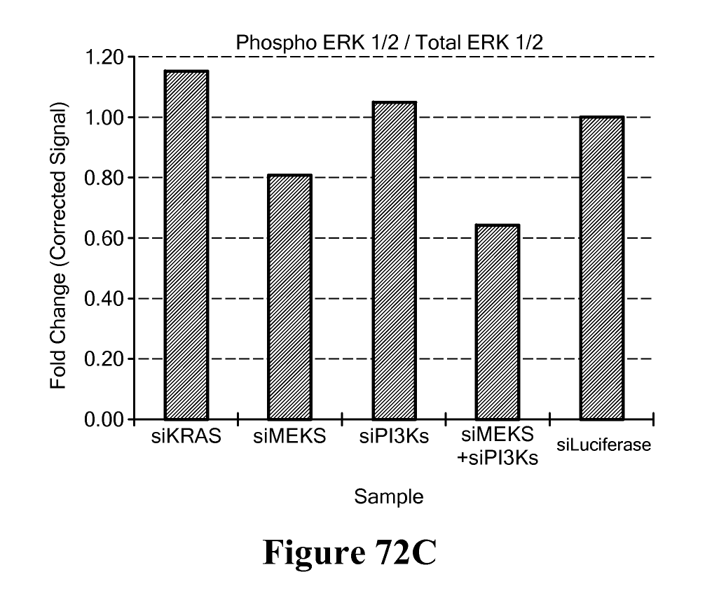

Data from these studies was also represented as ratios of phosphorylated biomarker versus total marker. FIGS. 70A, 70B, and 70C show the fold change of phosphorylated AKT1 (FIG. 70A), total AKT1 (FIG. 70B), and the ratio of phosphorylated AKT1/total AKT1 (FIG. 70C) for cells treated with siRNA targeting KRAS, siRNA targeting MEK, siRNA targeting PI3K, siRNAs targeting both MEK and PI3K, and a luciferase control. FIGS. 71A, 71B, and 71C show the fold change of phosphorylated MEK1 (FIG. 71A), total MEK1 (FIG. 71B), and the ratio of phosphorylated MEK 1/total MEK 1 (FIG. 71C) for cells treated with siRNA targeting KRAS, siRNA targeting MEK, siRNA targeting PI3K, siRNAs targeting both MEK and PI3K, and a luciferase control. FIGS. 72A, 72B, and 72C show the fold change of phosphorylated ERK 1/2 (FIG. 72A), total ERK 1/2 (FIG. 72B), and the ratio of phospho ERK 1/2/total ERK 1/2 (FIG. 72C) for cells treated with siRNA targeting KRAS, siRNA targeting MEK, siRNA targeting PI3K, siRNAs targeting both MEK and PI3K, and a luciferase control.

Thus, these data show that not only the targeted biomarker, but also biomarkers in the same signal transduction pathway as the targeted biomarker, including reference biomarkers such as total versus activated forms, can be quickly detected and quantitated using the methods described herein. As FIGS. 70A-70C, 71A-71C, and 72A-72C show, the data can also be viewed as ratio's of activated and total, or marker to reference marker at a given lysate protein loading for comparison. Being able to finely resolve effects between samples, treatments and markers of the same pathway as the targeted markers, or pathway to pathway elucidates neighboring, correlated or adjunct effects and allows for tailoring of targeted therapies. With the focus on targeted therapeutics in the treatment of cancer, the methods and kits described herein allow the rapid and reliable detection of whether or not the therapeutic is actually hitting its intended target. Thus, rapid and accurate detection and/or quantification of a biomarker (i.e., a biological molecule, such as a protein) would be useful.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for determining whether a cell treated with an agent targeting a targeted biomarker is developing resistance to the agent or responding to the agent comprising:
(a) providing a sample of a cell treated with the agent;
(b) providing a first binding agent that specifically binds to an entity selected from the group consisting of a first epitope on a first biomarker involved in a first signal transduction pathway and a noninterface epitope on the first biomarker, wherein said first biomarker is the same as or is different from the targeted biomarker, and wherein the first binding agent is attached to a solid support;
(c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is involved in the first signal transduction pathway, and wherein said second binding agent is capable of attaching to a sensor;
(d) mixing the sample, the first binding agent, and the second binding agent together under conditions whereby a sandwich assay formation will be formed if the sample contains a component selected from the group consisting of the first biomarker comprising the first epitope and the first alternate epitope and the first biomarker complexed with the first biomarker partner;
(e) presenting the result of step (d) to a sensor that emits an output signal that relates to the sensor's acoustic response to obtain an agent number; and
(f) comparing the agent number with a control number, the control number obtained by performing steps (b)-(e) with a control sample, the control sample selected from the group consisting of an untreated cell and a cell treated with a control agent;
wherein a change in the agent number of (e) as compared to the control number that is a positive number or that is greater than an predetermined fold change indicates the agent-treated cell is either developing resistance to the agent or is responding to the agent and wherein a change in the agent number of step (e) as compared to the control number that is a negative number or that is less than the predetermined fold change indicates the agent-treated cell is either not responding to the agent or not developing resistance to the agent.

2. The method of claim 1, wherein the first alternate epitope and the first epitope are both present only on an activated form or an inhibited form of the first biomarker so that the result of step (e) is either an activated agent number or an inhibited agent number; the method further comprising:
(g) repeating steps (b)-(e), with a first reference binding agent that specifically binds to a first reference epitope on a first reference biomarker within the sample, or a portion of the sample, and a second reference binding agent that specifically binds to a second reference epitope on the first reference biomarker, wherein the first reference epitope and the second reference epitope are not the same, to obtain an agent reference number;
(h) comparing the repeating steps (b)-(f) with a control sample to obtain an activated control number or an inhibited control number and a control reference number, the control sample selected from the group consisting of an untreated cell and a cell treated with a control agent; and
(h) comparing the activated agent number or the inhibited agent number to the agent reference number to obtain a first ratio and comparing an activated control number or an inhibited control number to the control reference number to obtain a control ratio, the activated control number or the inhibited control number and a control reference number obtained by repeating steps (b)-(e) with a control sample selected from the group consisting of an untreated cell and a cell treated with a control agent;
wherein a change in the first ratio to the control ratio that is a positive number or that is greater than an predetermined fold change indicates the agent-treated cell is developing resistance to the agent and wherein a change in the first ratio to the control ratio that is a negative number or that is less than the predetermined fold change indicates the agent-treated cell is not developing resistance to the agent.

3. The method of claim 1, wherein the biomarker or biomarker partner is an intracellular biomarker, a secreted biomarker, a cell surface biomarker, raf, KRAS, MEK, PI3 kinase, braf, MEK1, MEK2, ERK1, ERK2, ERK3, ERK4, ERK5, ERK7, ERK8, JNK1, JNK2, XNK3, MKK3, MKK6, ρ38, ρ38β, ρ38γ, ρ38δ, MKK4, MKK7, MTK1, DLK, TAO1, TA02, RSK1, RSK2, RSK3, MNK1, MNK2, MSK1, MEK5, Fos, Jun, CDK2, p27, SMAD, AKT, PI3K, Bcl-x1, STAT3, STAT5, caspase 8, caspase 9, Bad, Bim, Bax, cdc42, Akkα, FADD, JAK, CDK4, Rb, epidermal growth factor receptor (EGFR), NF-κB, a cytokine, a growth factor, an enzyme, and, a receptor.

4. A method for analyzing two or more signal transduction pathways in a cell treated with an agent comprising:
(a) providing a sample comprising a cell treated with an agent;
(b) providing a first binding agent that specifically binds to an entity selected from the group consisting of a first epitope on a first biomarker involved in a first signal transduction pathway and a noninterface epitope on the first biomarker, wherein the first binding agent is attached to a solid support;
(c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is involved in the first signal transduction pathway, and wherein said second binding agent is capable of attaching to a sensor;
(d) mixing the sample, the first binding agent, and the second binding agent together under conditions whereby a first sandwich assay formation will be formed if the sample contains a component selected from the group consisting of the first biomarker comprising the first epitope and the first alternate epitope and the first biomarker complexed with the first biomarker partner;
(e) presenting the result of step (d) to a sensor that emits an output signal that relates to the sensor's acoustic response to obtain a first number;
(f) providing a third binding agent that specifically binds to an entity selected from the group consisting of a second epitope on a second biomarker in a second signal transduction pathway, wherein the second biomarker is different than the first biomarker, and a noninterface epitope on the second biomarker, and wherein the third binding agent is attached to a solid support;
(g) providing a fourth binding agent that specifically binds to an entity selected from the group consisting of a second alternate epitope on the second biomarker involved in the second signal transduction pathway, wherein the second alternate epitope is different from the second epitope, and a noninterface epitope on a second biomarker partner, wherein said second biomarker partner is involved in the second signal transduction pathway, and wherein said fourth binding agent is capable of attaching to a sensor;
(h) mixing the sample, the third binding agent, and the fourth binding agent together under conditions whereby a second sandwich assay formation will be formed if the sample contains a component selected from the group consisting of the second biomarker comprising the second epitope and the second alternate epitope, and the second biomarker complexed with the second biomarker partner;
(i) presenting the result of step (h) to a sensor that emits an output signal that relates to the sensor's acoustic response to obtain a second number; and
(j) comparing the first number to a first control number to obtain a first difference and comparing the second number to the second control number to obtain a second difference, the first control number and the second control obtained by performing steps (b)-(i) with a control sample, the control sample selected from the group consisting of an untreated cell and a cell treated with a control agent;
wherein said first difference and said second difference are represented as positive numbers; and wherein a first difference larger than a second difference identifies the first signal transduction pathway as the pathway being preferentially targeted by the agent and wherein a second difference larger than a first difference identifies the second signal transduction pathway as the pathway being preferentially targeted by the agent.

5. The method of claim 4, wherein the first alternate epitope and the first epitope are both present only on an activated form or an inhibited form of the first biomarker, and wherein the second alternate epitope and the second epitope are both present only on an activated form or an inhibited form of the second biomarker.

6. The method of claim 4, further comprising:
(k) repeating steps (b)-(e), with a first reference binding agent that specifically binds to a first reference epitope on a first reference biomarker within the sample, or a portion of the sample, and a second reference binding agent that specifically binds to a second reference epitope on the first reference biomarker, wherein the first reference epitope and the second reference epitope are not the same, to obtain a first reference number;
(l) repeating steps (g)-(i), with a third reference binding agent that specifically binds to a second reference epitope on a second reference biomarker within the sample, or a portion of the sample, and a fourth reference binding agent that specifically binds to a second alternate reference epitope on the second reference biomarker, wherein the second reference epitope and the second alternate reference epitope are not the same, to obtain an second reference number; and
(m) comparing the first number to the first reference number to obtain a first ratio, comparing the second number to the second reference number to obtain a second ratio, comparing the control first number to a control first reference number to obtain a control first ratio, and comparing the control second number to a control second reference number to obtain a control second ratio;
wherein a change in the first ratio to the control ratio that is a positive number or that is greater than an predetermined fold change indicates the agent-treated cell is developing resistance to the agent and wherein a change in the first ratio to the control ratio that is a negative number or that is less than the predetermined fold change indicates the agent-treated cell is not developing resistance to the agent.

7. A method for identifying a signal transduction pathway to target with an agent in a diseased or damaged cell, comprising
(a) providing a sample of a diseased or damaged cell;
(b) providing a first binding agent that specifically binds to an entity selected from the group consisting of a first epitope on a first biomarker and a noninterface epitope on the first biomarker, wherein said first biomarker is in a first signal transduction pathway, wherein the first binding agent is attached to a solid support;
(c) providing a second binding agent that specifically binds to an entity selected from the group consisting of a first alternate epitope on the first biomarker, wherein the first alternate epitope is different than the first epitope, and a noninterface epitope on a first biomarker partner, wherein said first biomarker partner is involved in the first signal transduction pathway, and wherein said second binding agent is capable of attaching to a sensor;
(d) mixing the sample, the first binding agent, and the second binding agent together under conditions whereby a first sandwich assay formation will be formed if the sample contains a component selected from the group consisting of the first biomarker comprising the first epitope and the first alternate epitope and the first biomarker complexed with the first biomarker partner;
(e) presenting the result of step (d) to a sensor that emits an output signal that relates to the sensor's acoustic response to obtain a first number;
(f) providing a third binding agent that specifically binds to an entity selected from the group consisting of a second epitope on a second biomarker and a noninterface epitope on the second biomarker, wherein the second biomarker is involved in a second signal transduction pathway, wherein the second biomarker is different than the first biomarker, and wherein the third binding agent is attached to a solid support;
(g) providing a fourth binding agent that specifically binds to an entity selected from the group consisting of a second alternate epitope on the second biomarker involved in the second signal transduction pathway, wherein the second alternate epitope is different from the second epitope, and a noninterface epitope on a second biomarker partner, and wherein said fourth binding agent is capable of attaching to a sensor;
(h) mixing the sample, the third binding agent, and the fourth binding agent together under conditions whereby a second sandwich assay formation will be formed if the sample contains a component selected from the group consisting of the second biomarker comprising the second epitope and the second alternate epitope, and the second biomarker complexed with the second biomarker partner; and
(i) presenting the result of step (h) to a sensor that emits an output signal that relates to the sensor's acoustic response to obtain a second number; and
(j) comparing the first number to the first control number to obtain a first difference and the second number to the second control number to obtain a second difference, the first control number and the second control number obtained by performing steps (b)-(i) with a control sample selected from the group consisting of an untreated cell and a cell treated with a control agent, wherein said first difference and said second difference are represented as positive numbers;
wherein a first difference larger than a second difference identifies the first signal transduction pathway as the pathway to target with the agent in the diseased or damaged cell and wherein a second difference larger than a first difference identifies the second signal transduction pathway as the pathway to target with the agent in the diseased or damaged cell.

8. The method of claim 7, wherein the first alternate epitope and the first epitope are both present only on an activated form or an inhibited form of the first biomarker, and wherein the second alternate epitope and the second epitope are both present only on an activated form or an inhibited form of the second biomarker

9. The method of claim 8, further comprising
(k) repeating steps (b)-(e), with a first reference binding agent that specifically binds to a first reference epitope on a first reference biomarker within the sample, or a portion of the sample, and a second reference binding agent that specifically binds to a first alternate reference epitope on the first reference biomarker, wherein the first reference epitope and the first alternate reference epitope are not the same, to obtain a first reference number;
(l) repeating steps (g)-(i), with a third reference binding agent that specifically binds to a second reference epitope on a second reference biomarker within the sample, or a portion of the sample, and a fourth reference binding agent that specifically binds to a second alternate reference epitope on the second reference biomarker, wherein the second reference epitope and the second alternate reference epitope are not the same, to obtain a second reference number; and
(m) comparing the first number to the first reference number to obtain a first ratio, comparing the second number to the second reference number to obtain a second ratio, comparing the control first number to a control first reference number to obtain a control first ratio, and comparing the control second number to a control second reference number to obtain a control second ratio;
wherein a change in the first ratio to the control first ratio is greater than the change in the second ratio to the control second ratio identifies the first signal transduction pathway as the pathway as the pathway to target with the agent in the diseased or damaged cell and wherein a change in the first ratio to the control first ratio is less than the change in the second ratio to the control second ratio identifies the second signal transduction pathway as the pathway to target with the agent in the diseased or damaged cell.

10. The method of claim 6, wherein the first reference biomarker and the second reference biomarker are the same.

11. The method of claim 9, wherein the first reference biomarker and the second reference biomarker are the same.

12. The method of claim 2, wherein the first reference biomarker is selected from the group consisting of a house-keeping protein and a total first biomarker, wherein the total first biomarker comprises all forms of the first biomarker.

13. The method of claim 6, wherein the first reference biomarker is selected from the group consisting of a house-keeping protein and a total first biomarker, wherein the total first biomarker comprises all forms of the first biomarker.

14. The method of claim 9, wherein the first reference biomarker is selected from the group consisting of a house-keeping protein and a total first biomarker, wherein the total first biomarker comprises all forms of the first biomarker.

15. The method of claim 6, wherein the second reference biomarker is selected from the group consisting of a house-keeping protein and a total second biomarker, wherein the total second biomarker comprises all forms of second biomarker.

16. The method of claim 9, wherein the second reference biomarker is selected from the group consisting of a house-keeping protein and a total second biomarker, wherein the total second biomarker comprises all forms of second biomarker.

17. The method of claim 4, wherein the agent targeting a signal transduction pathway is an agent that targets a targeted biomarker in the pathway.

18. The method of claim 1, wherein at least one of the first epitope and the first alternate epitope or at least one of the second epitope and the second alternate epitope is a phosphorylated amino acid.

19. The method of claim 4, wherein at least one of the first epitope and the first alternate epitope or at least one of the second epitope and the second alternate epitope is a phosphorylated amino acid.

20. The method of claim 7, wherein at least one of the first epitope and the first alternate epitope or at least one of the second epitope and the second alternate epitope is a phosphorylated amino acid.

21. The method of claim 1, wherein the sample is selected form the group consisting of a tissue biopsy sample, a bodily fluid sample a xenograft cell sample, a living cell sample, a fixed cell sample, a fine needle aspirate sample, a circulating tumor cell sample, a blood sample, an exosome sample, a cultured cell sample, a cell lysate sample, a diseased cell sample, a saliva sample, a mucous sample, a tears sample, a blood sample, a tumor cell sample, a synovial fluid sample, a serum sample, a tissue sample, a marrow sample, a lymph fluid sample, an interstitial fluid sample, a buccal cell sample, a pleural effusion sample, a mucosal cell sample, a cerebrospinal fluid sample, a breast milk sample, a semen sample, a feces sample, a plasma sample, and a urine sample.

22. The method of claim 4, wherein the sample is selected form the group consisting of a tissue biopsy sample, a bodily fluid sample a xenograft cell sample, a living cell sample, a fixed cell sample, a fine needle aspirate sample, a circulating tumor cell sample, a blood sample, an exosome sample, a cultured cell sample, a cell lysate sample, a diseased cell sample, a saliva sample, a mucous sample, a tears sample, a blood sample, a tumor cell sample, a synovial fluid sample, a serum sample, a tissue sample, a marrow sample, a lymph fluid sample, an interstitial fluid sample, a buccal cell sample, a pleural effusion sample, a mucosal cell sample, a cerebrospinal fluid sample, a breast milk sample, a semen sample, a feces sample, a plasma sample, and a urine sample.

23. The method of claim 7, wherein the sample is selected form the group consisting of a tissue biopsy sample, a bodily fluid sample a xenograft cell sample, a living cell sample, a fixed cell sample, a fine needle aspirate sample, a circulating tumor cell sample, a blood sample, an exosome sample, a cultured cell sample, a cell lysate sample, a diseased cell sample, a saliva sample, a mucous sample, a tears sample, a blood sample, a tumor cell sample, a synovial fluid sample, a serum sample, a tissue sample, a marrow sample, a lymph fluid sample, an interstitial fluid sample, a buccal cell sample, a pleural effusion sample, a mucosal cell sample, a cerebrospinal fluid sample, a breast milk sample, a semen sample, a feces sample, a plasma sample, and a urine sample.

24. The method of claim 4, wherein the agent is selected from the group consisting of an siRNA molecule that targets the targeted biomarker by hybridizing to a nucleic acid molecule encoding the targeted biomarker, a small molecule, a biologic, and a cocktail of agents targeting the signaling pathway.

25. The method of claim 7, wherein the agent is selected from the group consisting of an siRNA molecule that targets the targeted biomarker by hybridizing to a nucleic acid molecule encoding the targeted biomarker, a small molecule, a biologic, and a cocktail of agents targeting the signaling pathway.

26. The method of claim 4, wherein the biomarker or biomarker partner is an intracellular biomarker, a secreted biomarker, a cell surface biomarker, raf, KRAS, MEK, PI3 kinase, braf, MEK1, MEK2, ERK1, ERK2, ERK3, ERK4, ERK5, ERK7, ERK8, JNK1, JNK2, XNK3, MKK3, MKK6, ρ38, ρ38β, ρ38γ, ρ38δ, MKK4, MKK7, MTK1, DLK, TAO1, TA02, RSK1, RSK2, RSK3, MNK1, MNK2, MSK1, MEK5, Fos, Jun, CDK2, p27, SMAD, AKT, PI3K, Bcl-x1, STAT3, STAT5, caspase 8, caspase 9, Bad, Bim, Bax, cdc42, Akkα, FADD, JAK, CDK4, Rb, epidermal growth factor receptor (EGFR), NF-κB, a cytokine, a growth factor, an enzyme, and, a receptor.

27. The method of claim 7, wherein the biomarker or biomarker partner is an intracellular biomarker, a secreted biomarker, a cell surface biomarker, raf, KRAS, MEK, PI3 kinase, braf, MEK1, MEK2, ERK1, ERK2, ERK3, ERK4, ERK5, ERK7, ERK8, JNK1, JNK2, XNK3, MKK3, MKK6, ρ38, ρ38β, ρ38γ, ρ38δ, MKK4, MKK7, MTK1, DLK, TAO1, TA02, RSK1, RSK2, RSK3, MNK1, MNK2, MSK1, MEK5, Fos, Jun, CDK2, p27, SMAD, AKT, PI3K, Bcl-x1, STAT3, STAT5, caspase 8, caspase 9, Bad, Bim, Bax, cdc42, Akkα, FADD, JAK, CDK4, Rb, epidermal growth factor receptor (EGFR), NF-κB, a cytokine, a growth factor, an enzyme, and, a receptor.
